SEQUENCE LISTING (155 sequences)

SEQ ID NO: 1 - DNA, 195 bp, synthetic construct
  source 1..195: mol_type = other DNA
actccaccaggtagtggccgccgtccttcagcttcagggcctggtggatctcgcccttcagcacgccgtcgcgggggtacaggcgctcggtggaggcctcccagcccatagtcttcttctgcattacggggccgtcgggggggaagttggtgccgcgcatcttcaccttgtagatcagcgtgccgtcctgcaggg

SEQ ID NO: 2 - DNA, 195 bp, synthetic construct
  source 1..195: mol_type = other DNA
aggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacata

SEQ ID NO: 3 - DNA, 228 bp, synthetic construct
  source 1..228: mol_type = other DNA
agccctcggggaaggacagcttcttgtaatcggggatgtcggcggggtgcttcacgtacgccttggagccgtacaggaactggggggacaggatgtcccaggcgaagggcagggggccgcccttggtcaccttcagcttagcggtctgggtgccctcgtaggggcggccctcgccctcgccctcgatctcgaactcgtggccgttcatggagccctccatgcgcacct

SEQ ID NO: 4 - DNA, 25 bp, synthetic construct
  source 1..25: mol_type = other DNA
cctcccagcccatagtcttcttcta

SEQ ID NO: 5 - DNA, 50 bp, synthetic construct
  source 1..50: mol_type = other DNA
gctcggtggaggcctcccagcccatagtcttcttctgcattacggggccg

SEQ ID NO: 6 - DNA, 100 bp, synthetic construct
  source 1..100: mol_type = other DNA
tcagcacgccgtcgcgggggtacaggcgctcggtggaggcctcccagcccatagtcttcttctgcattacggggccgtcgggggggaagttggtgccgca

SEQ ID NO: 7 - DNA, 150 bp, synthetic construct
  source 1..150: mol_type = other DNA
gggcctggtggatctcgcccttcagcacgccgtcgcgggggtacaggcgctcggtggaggcctcccagcccatagtcttcttctgcattacggggccgtcgggggggaagttggtgccgcgcatcttcaccttgtagatcagcgtgccgt

SEQ ID NO: 8 - DNA, 300 bp, synthetic construct
  source 1..300: mol_type = other DNA
gcagttgcacgggcttcttggccatgtagatggtctggaactccaccaggtagtggccgccgtccttcagcttcagggcctggtggatctcgcccttcagcacgccgtcgcgggggtacaggcgctcggtggaggcctcccagcccatagtcttcttctgcattacggggccgtcgggggggaagttggtgccgcgcatcttcaccttgtagatcagcgtgccgtcctgcagggaggagtcctgggtcacggtcaccagaccgccgtcctcgaagttcatcacgcgctcccactggaagc

SEQ ID NO: 9 - DNA, 400 bp, synthetic construct
  source 1..400: mol_type = other DNA
tgtccagcttggtgtccacgtagtagtagccgggcagttgcacgggcttcttggccatgtagatggtctggaactccaccaggtagtggccgccgtccttcagcttcagggcctggtggatctcgcccttcagcacgccgtcgcgggggtacaggcgctcggtggaggcctcccagcccatagtcttcttctgcattacggggccgtcgggggggaagttggtgccgcgcatcttcaccttgtagatcagcgtgccgtcctgcagggaggagtcctgggtcacggtcaccagaccgccgtcctcgaagttcatcacgcgctcccactggaagccctcggggaaggacagcttcttgtaatcggggatgtcggcggggtgcttcacgtacgccttggagcg

SEQ ID NO: 10 - DNA, 195 bp, synthetic construct
  source 1..195: mol_type = other DNA
actccaccaggtagtggccgccgtccgtcagcttcagggcctggtggatctcgcccttcagcacgccgtcgcgggggtacaggcgctcggtggaggcctcccagcccatagtcttcttcggcattacggggccgtcgggggggaagttggtgccgcgcatcttcaccttgtagatcagcgtgccgtcctgcaggg

SEQ ID NO: 11 - DNA, 195 bp, synthetic construct
  source 1..195: mol_type = other DNA
actccaccaggtagtggccgccgtccttctgcttcagggcctggtggatctcgcccttcagcacgccgtcgcgggggtacaggcgctcggtggaggcctcccagcccatagtcttcttctgctttacggggccgtcgggggggaagttggtgccgcgcatcttcaccttgtagatcagcgtgccgtcctgcaggg

SEQ ID NO: 12 - DNA, 195 bp, synthetic construct; identical to SEQ ID NO: 11
  source 1..195: mol_type = other DNA

SEQ ID NO: 13 - DNA, 195 bp, synthetic construct
  source 1..195: mol_type = other DNA
actccaccaggtagtggccgccgtccttcagcttcagggcctggtggatctcgcccttcagcacgccgtcgcgggggtacaggcgctcggtggaggcctcccatcccatagtcttcttctgcgttacggggccgtcgggggggaagttggtgccgcgcatcttcaccttgtagatcagcgtgccgtcctgcaggg

SEQ ID NO: 14 - DNA, 195 bp, synthetic construct; identical to SEQ ID NO: 11
  source 1..195: mol_type = other DNA

SEQ ID NO: 15 - DNA, 195 bp, synthetic construct
  source 1..195: mol_type = other DNA
actccaccaggtagcggccgccgtccttcagcttcagggcctggtggatctcgcccttcagcacgccgtcgtgggggtacaggcgctcggtggtggcctcccagcccatagtcttcttctgcattacggggccgtcgggggggaagttggtgccgcgcatcttcaccttctagatcagcgtgccgtcctgcaggg

SEQ ID NO: 16 - DNA, 195 bp, synthetic construct
  source 1..195: mol_type = other DNA
actccaccaggtagtggccgccgtccttcggcttcagggcctggtggatctcgcccttcagaacgccgtcgcgggggtacaggcgctcggtggaggcctcccagcccatagtcttcttctgcattacggggccgtagggggggaagttggtgccgcgcatcttcaccttgtagagcagcgtgccgtcctgcaggg

SEQ ID NO: 17 - DNA, 195 bp, synthetic construct
  source 1..195: mol_type = other DNA
actccaccaggtggtggccgccgtccttcatcttcagggactggtggatctcgcccttcagcacgccgtcgagggggtacaggcgcgcggtggaggcctcccagcccatagtcttcttcggcattacggggacgtcgggggggaagttggtgctgcgcatcttcacattgtagatcatcgtgccgtcctgcaggg

SEQ ID NO: 18 - DNA, 195 bp, synthetic construct
  source 1..195: mol_type = other DNA
actccaccaggtagtggccgacgtccttcagcttcaggtcctggtggatctcgcccttccgcacgccgtcgcggtggtacaggcgcccggtggaggtctcccagcccatagtcttcttcttcattacggggccgtcggggggtaagttggggccgcgcatcttcaccttgtagatcagtgtgccgtcctgcaggg

SEQ ID NO: 19 - DNA, 195 bp, synthetic construct
  source 1..195: mol_type = other DNA
actccaccaggcagtggccgacgtccttcggcttcaggtcctggcggatctcgcccttccgcacgccgccgcggtggtacaggcgctcagtggaggtctcccagcccatagtcttcatcttcattacggggccgtcgcggggtaagttggggccgcccatcttcacgttgtagatcagtgtgccgacctgcaggg

SEQ ID NO: 20 - DNA, 196 bp, synthetic construct
  source 1..196: mol_type = other DNA
actccaccaggtagtggccgccgtccttcagcttcagggcctggtggatctcgcccttcagcacgccgtcgcgggggtacaggcgctcggtgcgaggcctcccagcccatagtcttcttctgcattacggggccgtcgggggggaagttggtgccgcgcatcttcaccttgtagatcagcgtgccgtcctgcaggg

SEQ ID NO: 21 - DNA, 196 bp, synthetic construct
  source 1..196: mol_type = other DNA
actccaccaggtagtggccgccgtccttcagcttcagggcctggtggatctcgcccttcagcacgccgtcgcgggggtacaggcgctcggtggaggcctcccagcccatagtcttcttctgcagtacggggccgtcgggggggaagttggtgccgcgcatcttcaccttgtagaatcagcgtgccgtcctgcaggg

SEQ ID NO: 22 - DNA, 194 bp, synthetic construct
  source 1..194: mol_type = other DNA
actccaccaggtagtggccgccgtccttcagcttcagggcctggtggatctcgcccttcagcacgccgtcgcgggggtacaggcgctcggtggaggcctcccagcccatagtcttcttctgcattacgggccgtcgggggggaagttggtgccgcgcatcttcaccttgtagatcagcgtgccgtcctgcaggg

SEQ ID NO: 23 - DNA, 196 bp, synthetic construct
  source 1..196: mol_type = other DNA
actccaccaggtagtggccgccgtccttcagcttcagggcctggtggatctcgcccttcagcacgccgtcgcgggggtacaggcgctcggtggaggcctcccagcccatagtcttcttctgcattacggggccgtcgggggggaagttggtgccgcgcatcttcaccttgtagatcagcgtgccgtcctgcagggc

SEQ ID NO: 24 - DNA, 255 bp, synthetic construct
  source 1..255: mol_type = other DNA
actccaccaggtagtggccgccgtccttcagcttcagggcctggtggatctcgcccttcagcacgccgtcgcgggggtacaggcgctcggtggaggcctcccagcccatggtcttcttctgcattacggggccgtcgggggggaagttggtagcgcgcatcttcaccttgtagatcagcgtgccgtcctgcagggaggagtcctgggtcacggtcaccagaccgccgtcctcgaagttcatcacgcgctcccact

SEQ ID NO: 25 - DNA, 238 bp, synthetic construct
  source 1..238: mol_type = other DNA
agttttaaacagagaggaatctttgcagctaatggaccttctaggtctggaaaggagtgggaattggctccggtgcccgtcagtgggcagagcgcacatcgcccacagtccccgagaagttagggggaggggtcggcaattgaaccggtgcctagagaaggtggcgcggggaaaactgggaaagtgaagtcgtgtactggctccgcctttttcccgagggtgggggagaaccgtatat

SEQ ID NO: 26 - DNA, 252 bp, synthetic construct
  source 1..252: mol_type = other DNA
ggtggttttgcggatatctccgtgaatcaatatgtgctcgtggatcaggacgcgcaggtagtgtcccaacaacccccaacaatttttactcatcagatcaataatcgtgtgacctacggtggagccatagacgctcaggacgccaaaaccttcgggccccaaaatgaagagaattaggaacataccccagctcacgaaaaacagccatgccatgccggtcacgacctggcggcaccgaccctttggcacagt

SEQ ID NO: 27 - DNA, 291 bp, synthetic construct
  source 1..291: mol_type = other DNA
ggttctcctgctgtagtcgttgctcaggccggtgaggttgctcaggcggataaggatgacaggacaagtgatgcagccaggttgcatagcgcagccactgcacccggtgtcctgtagcaaggtagagcaagagggattcttaaactcaaaaaagaactcgagaatcaccttaaccatttcaatggcgcacacatagatctcctcccagccgcatgtagatttccaggtttggtaggcatagaacatcagcagcaaaatgctgaatcctgctgcaagccactgcaggacatt

SEQ ID NO: 28 - DNA, 195 bp, synthetic construct; identical to SEQ ID NO: 1
  source 1..195: mol_type = other DNA

SEQ ID NO: 29 - DNA, 228 bp, synthetic construct; identical to SEQ ID NO: 3
  source 1..228: mol_type = other DNA

SEQ ID NO: 30 - DNA, 255 bp, synthetic construct
  source 1..255: mol_type = other DNA
actccaccaggtagtggccgccgtccttcagcttcagggcctggtggatctcgcccttcagcacgccgtcgcgggggtacaggcgctcggtggaggcctcccagcccatagtcttcttctgcattacggggccgtcgggggggaagttggtagcgcgcatcttcaccttgtagatcagcgtgccgtcctgcagggaggagtcctgggtcacggtcaccagaccgccgtcctcgaagttcatcacgcgctcccact

SEQ ID NO: 31 - DNA, 255 bp, synthetic construct
  source 1..255: mol_type = other DNA
actccaccaggtagtggccgccgtccttcagcttcagggcctggtggatctcgcccttcagcacgccgtcgcgggggtacaggcgctcggtagaggcctcccagcccatagtcttcttctgcattacggggccgtcgggggggaagttggtagcgcgcatcttcaccttgtagatcagcgtgccgtcctgcagggaggagtcctgggtcacggtcaccagaccgccgtcctcgaagttcatcacgcgctcccact

SEQ ID NO: 32 - DNA, 447 bp, synthetic construct
  source 1..447: mol_type = other DNA
ggtggttttgcggatatctccgtgaatcaatatgtgctcgtggatcaggacgcgcaggtagtgtcccaacaacccccaacaatttttactcatcagatcaataatcgtgtgacctacggtggagccatagacgctcaggacgccaaaaccttcgggccccaaaatgaagagaattaggaacataccccagctcacgaaaaacagccatgccatgccggtcacgacctggcggcaccgaccctttggcacagtactccaccaggtagtggccgccgtccttcagcttcagggcctggtggatctcgcccttcagcacgccgtcgcgggggtacaggcgctcggtggaggcctcccagcccatagtcttcttctgcattacggggccgtcgggggggaagttggtgccgcgcatcttcaccttgtagatcagcgtgccgtcctgcaggg

SEQ ID NO: 33 - DNA, 447 bp, synthetic construct
  source 1..447: mol_type = other DNA
actccaccaggtagtggccgccgtccttcagcttcagggcctggtggatctcgcccttcagcacgccgtcgcgggggtacaggcgctcggtggaggcctcccagcccatagtcttcttctgcattacggggccgtcgggggggaagttggtgccgcgcatcttcaccttgtagatcagcgtgccgtcctgcagggggtggttttgcggatatctccgtgaatcaatatgtgctcgtggatcaggacgcgcaggtagtgtcccaacaacccccaacaatttttactcatcagatcaataatcgtgtgacctacggtggagccatagacgctcaggacgccaaaaccttcgggccccaaaatgaagagaattaggaacataccccagctcacgaaaaacagccatgccatgccggtcacgacctggcggcaccgaccctttggcacagt

SEQ ID NO: 34 - DNA, 543 bp, synthetic construct
  source 1..543: mol_type = other DNA
ggtggttttgcggatatctccgtgaatcaatatgtgctcgtggatcaggacgcgcaggtagtgtcccaacaacccccaacaatttttactcatcagatcaataatcgtgtgacctacggtggagccatagacgctcaggacgccaaaaccttcgggccccaaaatgaagagaattaggaacataccccagctcacgaaaaacagccatgccatgccggtcacgacctggcggcaccgaccctttggcacagtggttctcctgctgtagtcgttgctcaggccggtgaggttgctcaggcggataaggatgacaggacaagtgatgcagccaggttgcatagcgcagccactgcacccggtgtcctgtagcaaggtagagcaagagggattcttaaactcaaaaaagaactcgagaatcaccttaaccatttcaatggcgcacacatagatctcctcccagccgcatgtagatttccaggtttggtaggcatagaacatcagcagcaaaatgctgaatcctgctgcaagccactgcaggacatt

SEQ ID NO: 35 - DNA, 738 bp, synthetic construct
  source 1..738: mol_type = other DNA
actccaccaggtagtggccgccgtccttcagcttcagggcctggtggatctcgcccttcagcacgccgtcgcgggggtacaggcgctcggtggaggcctcccagcccatagtcttcttctgcattacggggccgtcgggggggaagttggtgccgcgcatcttcaccttgtagatcagcgtgccgtcctgcagggggtggttttgcggatatctccgtgaatcaatatgtgctcgtggatcaggacgcgcaggtagtgtcccaacaacccccaacaatttttactcatcagatcaataatcgtgtgacctacggtggagccatagacgctcaggacgccaaaaccttcgggccccaaaatgaagagaattaggaacataccccagctcacgaaaaacagccatgccatgccggtcacgacctggcggcaccgaccctttggcacagtggttctcctgctgtagtcgttgctcaggccggtgaggttgctcaggcggataaggatgacaggacaagtgatgcagccaggttgcatagcgcagccactgcacccggtgtcctgtagcaaggtagagcaagagggattcttaaactcaaaaaagaactcgagaatcaccttaaccatttcaatggcgcacacatagatctcctcccagccgcatgtagatttccaggtttggtaggcatagaacatcagcagcaaaatgctgaatcctgctgcaagccactgcaggacatt

SEQ ID NO: 36 - DNA, 225 bp, synthetic construct
  source 1..225: mol_type = other DNA
cttcagtctccacccactcagtgtggggaaactccatcgcataaaacccctccccccaacctaaagacgacgtactccaaaagctcgagaactaatcgaggtgcctggacggcgcccggtactccgtagagtcacatgaagcgacggctgaggacggaaaggcccttttcctttgtgtgggtgactcacccgcccgctctcccgagcgccgcgtcctccattttg

SEQ ID NO: 37 - DNA, 249 bp, synthetic construct
  source 1..249: mol_type = other DNA
ctcatagggtgtacactagccactacacttatttcttatgtcatggcaaatagtcaactttcactgcccagtcattttaacccacgtttcaacatgcacatcccagtaatttagaaacattttgtttccaaagattcacttaacattggtttagcaacaggaagctttctatgcaacacaaggactcagtttttggcctgttttagtgacaggcaatcagcaacatgctgcatttctctccagtgttgt

SEQ ID NO: 38 - DNA, 228 bp, synthetic construct
  source 1..228: mol_type = other DNA
gtaaggattaagagtcgttacttggttactaaaacacaaactccagcttcaatttccttgtccccagcccttaattagcagtttccactttacaactccaagtccaaagtgattttagtcactttgggttacagaagcaaccaaaaatcaaacttttataagtcggatcttaactatgaacatccaaatctactcactagcaatacgattacagaagtcaccaaaagc

SEQ ID NO: 39 - DNA, 249 bp, synthetic construct
  source 1..249: mol_type = other DNA
gttagcacttggctccagcatgttgtcaccattccaaccagaaattggcacaaatgctactgtgtcggggttgtagccaattttcttaatgtaagtgctgacttccttaacaatttcctcatatctcttctggctgtagggtagctcagtggaatccattttgttaacaccgacaatgagttgtttcacacccagtgtgtaagccagaagggcatgctctcgggtctgcccattcttggagataccagc

SEQ ID NO: 40 - DNA, 249 bp, synthetic construct
  source 1..249: mol_type = other DNA
agtgaagccagctgcttccattggtgggtcatttttgctgtcaccagcaacgttgccacgacgaacatccttgacagacacattcttgacattgaagcccacattgtccccaggaagagcttcactcaaagcttcatggtgcatttcgacagattttacttccgttgtaacgttgactagagcaaaggtgaccaccataccgggtttgagaacaccagtctccactcggccaacaggaacagtaccaat

SEQ ID NO: 41 - DNA, 249 bp, synthetic construct
  source 1..249: mol_type = other DNA
gtctgtctcatatcacgaacagcaaagcgacctattaaaaaaaaagttaattattacccaaagtactgttcagttgtatttttcatctttaacacaacttttttacatttaagtagtcatccttacccaaaggtggatagtctgagaagctctcaacacacataggcttgccaggaaccatatcaacaatggcagcatcaccagacttcaagaattgagggccatcttccagctttttaccagaacggc

SEQ ID NO: 42 - DNA, 249 bp, synthetic construct
  source 1..249: mol_type = other DNA
gccatcttccagctttttaccagaacggcgatcaatcttttccttcagctcagcaaacttgcatgcaatgtgagccgtgtggcaatccaatacaggggcatagccggcgcttatttggcctagatggttcaggataatcacctgagcagtgaagccagctgcttccattggtgggtcatttttgctgtcaccagcaacgttgccacgacgaacatccttgacagacacattcttgacattgaagcccac

SEQ ID NO: 43 - DNA, 234 bp, synthetic construct
  source 1..234: mol_type = other DNA
aaaatatggaggacgtgtcaagaagacactaggagaaagtatagaattgaaaaaaatattttatggaatttaggtgatttttttaaagaaatacgccataaagggtgttaggggactagaaaatgttctagaaagaaccccaagtgcagagagatcttcagtcaggaagcttccagccccgagagagtaagaaatatggctgcagcagcgaattgcagcgctttaagaactgaa

SEQ ID NO: 44 - DNA, 210 bp, synthetic construct
  source 1..210: mol_type = other DNA
caattgggactgagcattttaactgtccaacaaaagacgggttgtctgcgaccccctgctgaatgcaaatggggttcagagtgctgtctaatccaataggtaggattattggcaattagaattttactcaatgcaaagagttttaggtggccaacacagtacacaagaaaagatcactactcaaaattgggactgggactacagaagcaa

SEQ ID NO: 45 - DNA, 219 bp, synthetic construct
  source 1..219: mol_type = other DNA
ctggtgaggttcacgcccatggccaggttgcggtcgcagcgggaggtgtcgaagccctcagaccgcagggtgagctgcaccaaagagacgtaggacgagtccatgctctgcaggtttacaccgctggagctaatatcccagcaggcctcgttgatgaggtccttgagtgcctccaacaccttcttgaggatggagccctggaccaggcgcgcctcgaac

SEQ ID NO: 46 - DNA, 249 bp, synthetic construct
  source 1..249: mol_type = other DNA
ctgggtgccagggcagtgatctccttctgcatcctgtcggcaatgccagggtacatggtggtgccgccagacagcactgtgttggcgtacaggtctttgcggatgtccacgtcacacttcatgatggagttgaaggtagtttcgtagatgccacaggactccatgcccaggaaggaaggctggaagagtgcctcagggcagcggaaccgctcattgccaatggtgatgacctggccgtcaggcagctcg

SEQ ID NO: 47 - DNA, 237 bp, synthetic construct
  source 1..237: mol_type = other DNA
gggtcatcttctcgcggttggccttggggttcaggggggcctcggtcagcagcacggggtgctcctcgggagccacacgcagctcattgtagaaggtgtggtgccagattttctccatgtcgtcccagttggggacgatgccgtgctcgatagggtacttcagggggaggatgcctctcttgctctgggcctcgtcgcccacataggaatccttctgacccatgcccaccatcacgc

SEQ ID NO: 48 - DNA, 279 bp, synthetic construct
  source 1..279: mol_type = other DNA
cgtgcaagtcacagacttggctgtcccagaatgcaagaagcccagacggaaaccgtagctgccctggtaggttttctgggaagggacagaagatggcaggggccaggagggggctggtgcaggggccgccggtgtaggagctgctagtgcaggggccacggggggagcagcctctggcattctgggagcttcatctggacctgggtcttcagtgaaccattgttcaatatcgtccggggacagcatcaaatcatccattgcttgggacggcaaggggga

SEQ ID NO: 49 - DNA, 240 bp, synthetic construct
  source 1..240: mol_type = other DNA
gagatggagtctctctctgtcacctgggctggagcacagtggcatgatctcagctcactgcaacctctaccttccgggttcaagccattctcctgcctcagtctcccgagtagctaggattacaggcgagtaccaccacacccagctaatttttgtatttttagtagagacagggctttgcatgttggccaggctggtctcgaactccttacttcaggtgatcggcccgcctcagcctcc

SEQ ID NO: 50 - DNA, 354 bp, synthetic construct
  source 1..354: mol_type = other DNA
tcacactggcacgtccagacccaggtactctgggttctctgccgtaggtgtccctttgaaggtgctgggtggagccccccgctctggtgggtcctggtcccagtaatagaggttgtcgaaggctaggctgaaggcaggaggagggtggggctgaggggcagctcctccctggggtgtcaagtactcggggttctccacggcacccccaaaggcaaaaacgtctttgacgaccccattcttccctggggagagagtcttgggcctttccagagtggcaccagcaggtcgggcagcaggcagagggccctctcggggcgaagggggctggggccgaacatctggctggttcacata

SEQ ID NO: 51 - DNA, 354 bp, synthetic construct
  source 1..354: mol_type = other DNA
tcacactggcacgtccagacccaggtactctgggttctctgccgtaggtgtccctttgaaggtgctgggtggagccccccgctctggtgggtcctggtcccagtaatagaggttgtcgaaggctaggctgaaggcaggaggagggtggggctgaggggcagctcctccctggggtgtcaagtactcggggttctccacggcacccccaaaggcaaaaacgtctttgacgaccccattcttccctagggagagagtcttgggcctttccagagtggcaccagcaggtcgggcagcaggcagagggccctctcggggcgaagggggctggggccgaacatctggctggttcacata

SEQ ID NO: 52 - DNA, 273 bp, synthetic construct
  source 1..273: mol_type = other DNA
cagtggccctggtcagctctgaataaccaagagaaggtttcaatgacggtgaaggccacctgtgaggcttcgaagctgcagctcccgcaggcctcctggggaggcccctgtgacaggggtggtattgttcagcgggtctccattgtctggcacggccagggcatagttgtcctcaaagagctgggtgcctcgcacaatccgcagcctctgcagtaggacctgcctcacttggttgtgagcgatgagcacgtagccctgcacctcctggatatc

SEQ ID NO: 53 - DNA, 219 bp, synthetic construct
  source 1..219: mol_type = other DNA
agaacacctcgcgccacacgtgcatctcgcgcttggcccagcgctccaggttggcgatggtctcctggcagcggcacaggcaggccttggtggacttcttccagcgctgcgagtcgctcgtaggcacgtagccgtccaggttgctctccagcttcccgaccgaccggtgcagccccttcagctcgcgctccacctgcttggacacctcggccagcaggt

SEQ ID NO: 54 - DNA, 265 bp, synthetic construct
  source 1..265: mol_type = other DNA
cgcaggaaacgcttgagcttgggctgcagggtgcccaccacgtactggatgatctcctcctcgtccgcgtccacgtggagcgtctggtacaggtcccgcttgcgccacaggaactagtccagcggctcgccctgcttctgcggcaggtccagctcgcgctgggtggcctctcgggacagcgtgccctcgctgtactgcaggaactccttcttgaactccacccagttcttcacggagccctgcttgaactcccaccacttcttgg

SEQ ID NO: 55 - DNA, 276 bp, synthetic construct
  source 1..276: mol_type = other DNA
cttctgctctgggtgtcccatgagcttggtggccgctggcccctgccatccggacacttggttttctggatgtccagagggcctgggtgttgtagcagaatgaggaagctggggtgttctgtgatggcctgggagtgtggagggtggggtccaggccggaaagacagacggagggagggtgatgggtagagccctgcaggggggagggctctttgccgctgggtgcggtgctcacatctggatgacacgagtgtgtgcgagtccgcccggagctcg

SEQ ID NO: 56 - DNA, 384 bp, synthetic construct
  source 1..384: mol_type = other DNA
gaagcgcaggctgccgttctcagaggaatgttccgacgatgtggcgaaaggcgactggcgcgcatccgtgaagcccaggaatgggtccttcatgaagtggcgcgatgctgcgtagcccacgagccactgcgagtacacgttctcagatgggatgagggcggcaggtagcagctccaggtctttctccaccttgatgcgcttggccgcgtgcagcgcgggaccaccgagcccagggctgggcagcggtgctggcttgcgtgggaagagagctggaaagggctctgcgcctggcgcgaaggccccgccgcgcccgttcactgcacccggtgcacccgcgtccccacagccaacagctccggcatcacccgtgtcgcctgcacgctt

SEQ ID NO: 57 - DNA, 252 bp, synthetic construct
  source 1..252: mol_type = other DNA
caccgcagcggctctgttaccggacaactcacactggcatccaaagggagcctccatctgaccctcaccctgagtcccgtcgcccgagatggggcgtgcaccacagcaaggcagcgggaaacagggtaggagaacgcccagggcacgcagaggggaagtaatcacggaggtgggagggtgggaagaggaggcagctatgggggccatcgatggcagctgggcaggcctgcacggccctggagaaaaaacaat

SEQ ID NO: 58 - DNA, 288 bp, synthetic construct
  source 1..288: mol_type = other DNA
acattaagaatcggtctgttcccaggagttctgtctccaaggtcacaccatgtctgggaaggaaaactgttccttcacgcaaatgctcgcagcaacttcccagtaccctttatccttgtgtagccacctgcctcgctctctacactgcagagactgatgaagcactgtgagccagggccatggagcaggagaagggctgctttcacggaaccacagtcagcaagacatcggggattacgtgatttccagcgagggttcaagtatcgagtccataagaaaggtgagtcc

SEQ ID NO: 59 - DNA, 207 bp, synthetic construct
  source 1..207: mol_type = other DNA
ttccgacgatgtggcgaaaggcgactggcgcgcatccgtgaagcccaggaatgggtccttcatgaagtggcgcgatgctgcgtagcccacgagccactgcgagtacacgttctcagataggatgagggcggcaggtagcagctccaggtctttctccaccttgatgcgcttggccgcgtgcagcgcgggaccaccgagcccagggct

SEQ ID NO: 60 - DNA, 345 bp, synthetic construct
  source 1..345: mol_type = other DNA
tgcaatgttctcctgcttgggacagatgcctttcgtgggggacaggaggtggtcatcttcatcaggggtgacctggatcccgatctccactagctcagatactctcctgagctcagagcgagaggagggaggtagactgctcttgtccaggaccttgtcgtagcaggggcccaggcctccacactgtttcttcttgtgctctataaaaaccaggatgtcccccagcgggaagttcatctgacactggccacaggtgaggagatcagggtcggggcctcccaccatcagccccaggctgctaggctcctctatctccagaccctcgtcttcctcgaggatggtagc

SEQ ID NO: 61 - DNA, 411 bp, synthetic construct
  source 1..411: mol_type = other DNA
ttccgacgatgtggcgaaaggcgactggcgcgcatccgtgaagcccaggaatgggtccttcatgaagtggcgcgatgctgcgtagcccacgagccactgcgagtacacgttctcagataggatgagggcggcaggtagcagctccaggtctttctccaccttgatgcgcttggccgcgtgcagcgcgggaccaccgagcccagggcttttgtggtcatcttcatcaggggtgacctggatcccgatctccactggctcagatactctcctgagctcagagcgagaggagggaggtagactgctcttgtccaggaccttgtcgtagcaggggcccaggcctccacactgtttcttcttgtgctctataaaaaccaggatgtcccccagcgggaagttcatctgacactggcc

SEQ ID NO: 62 - DNA, 300 bp, synthetic construct
  source 1..300: mol_type = other DNA
agtcctccagaaatatacagatgcattgtgcccgcccggcacacaggaaaggaagaacaccgactgagacataggagaagggtgtcaaaatattgtttggtcagaaaagaaacaaaaagggacaaaggaaataaactgagaagcaaaacaaaatagggataggcggggggcaaactgaaattgaaattgagaattcaaactaagcagggaggggtgctatccctgggagggatccactgcccgcctcgagcaggcctggcaaggcctctgctccttacagcctttgataaaaataataaa

SEQ ID NO: 63 - DNA, 765 bp, synthetic construct
  source 1..765: mol_type = other DNA
gagcttgctcgcctgcgagcacgcatggtcgcacagctggcacttgtagggcttctcgcccgtgtggctgcgccggtgcacgatgagattgctctggaacttgaaggtcttgccgcagaactcacaggacttgctcttggcaggcggctgcggtggcggtgtgcccgcaggcatgggtggcagcggtggcgtgctgaggaacggggacttgggactgggctggaaagggttcagcagccggtgcatagggttgccacggcctggggacacgggcggcggcgtggagctgttgccggccagttctcgcagccgccgggagaagtccatggcaggagagtctatggccatggggttcaggcgcatgactcggtcgaaggcactggggtgctgggccacgagccccatctcctctgcactgaggcggtgtgggtccaagtgatggcgtagcggtgggctgaagagcggtggcgtacctagcaagcggccctcaccgaagccagggtggtcccgcaggatggggcccgtcatgcgcagcaggttgaaaggattgctgtcccccaggaaattcatgagtggggactgcgccacggtctccggcccgagcggtggcgggatggtgagcctgggcgtgagcgaggtgctggccggcccaggctccaggtagattcggaagccatgtgtgttctgtgcgtgctgcagcaggaaccaggcgctgttgaagggctgcttgcatgttgtgcaaatgtagctggaaggctcatcttt

SEQ ID NO: 64 - DNA, 189 bp, synthetic construct
  source 1..189: mol_type = other DNA
cgcagccaggctggccagtttggcgttctccagcagaaaaagcttggggtgagcagccagggaagtgggggcctgtgcgttgaggaggccggataggaaaaggtggcctccgaggagctccgaaggtgggtaagcggtggagtccaggtagttgaagtagtagagagagccgctggcaggcagccccac

SEQ ID NO: 65 - DNA, 204 bp, synthetic construct
  source 1..204: mol_type = other DNA
gaacttgtccgcagccaggctggccagtttggcgttctccagcagaaaaagcttggggtgagcagccagggaagtgggggcctgtgcgttgaggaggccggataggaaaaggtggcctccgaggagctccgaaggtaggtaagcggtggagtccaggtagttgaagtagtagagagagccgctggcaggcagccccacagcctg

SEQ ID NO: 66 - DNA, 372 bp, synthetic construct
  source 1..372: mol_type = other DNA
gcacctagtcggcccggtcacccatttgcattcaaaggaacagaagcagaacaaagtgcacccaagggtaccaaattaccgcccattaacccggcgcgcaaaggaacccaccagctattgccctaggactatgaaagggggaagaagggggaggctgtacaaaggaaaggagaaggggtagaagaaaggagtctcaaattaacactcccagcccttccctagacccaggccttcgccacgcgtgctgggaactgggcactgaaagagcaaagtgaaagagggccggaagaaccacctcgcttttacctctttcccccaccgccaaggagatgcgttccgagccatgcagcgtgtctcttctgtcacattttg

SEQ ID NO: 67 - DNA, 753 bp, synthetic construct
  source 1..753: mol_type = other DNA
cttgccgcacacttcgcaggtgaagtttttgggtttgctgtcagtagagccccccgggagtttgctgtggctcttgactccccctcgttcagctgtcaaggccgagttctccttcagcacctgctccagtggcgcatgcaagcgctccttatggggataggaagctgggtgggggaacttgtccgcagccaggctggccagtttggcgttctccagcagaaaaagcttggggtgagcagccagggaagtgggggcctgtgcgttgaggaggccggataggaaaaggtggcctccgaggagctccgaaggtaggtaagcggtggagtccaggtagttgaagtagtagagagagccgctggcaggcagccccacagcctggttgatgacctgcggtttgatgaccctgccggcgggcagcgcagaaggcgcgaggcccagttcggccttgcagcacacgccacagttggttttgcacaagccgctggcgccgcacactggggccccaccgccgctgcctcctcctccaccgccgcccgcccggaggctgctcttccagaactccgagtaactgagcagcgtcttggacggcacctcgtagccgagaggctggagagggatcatacagggcagcggcgagcagaggttgagcagtttcttgctctggctgctgtctgcctcgaacgcagcaggccggggctcgaaaggcgctcggggctcggacgtcttggccatgatgcgctcgatagagaaagc

SEQ ID NO: 68 - DNA, 708 bp, synthetic construct
  source 1..708: mol_type = other DNA
ttatctctggtcggtttcggccggtgcagccttgctcttgtcggcattctcttcctcggcctccaccttgtacatctcctccgagccttcctcactgtcattctcctcggactcggtcagcagctcctcgtccttatactctgccacgtccacggcggagcccaggtgctccttcagcttgccatggtgcttcacgtggtacctctggttctggaagaacttgatgatggtgtgtttggggagatccagctgggcggagagtgtgtggatggcttcctggtcgggatagaggcctacatcatggatgaagctttgaaggatgcccagggcttccaaagagatctttgtgcgagaccgaggctttttggcacagctgtcttctgttagaggaggcggtaggggtgcttcttctctgggaggggagctctccttagttggctgggactgctgtcgatgaaggacctgcacgggctcaggtgggagctggaccacaggctgcatgcgttcactgtggtgaggtcgagattcttcctcatagatcacatcccgctcaggttggggaagattcaggaaacggcggatggtgcagagattctcccaaagggtgcggttttctgggctggggttctccttccaacgaagcagttcgcaaagccagccctgacttttgtttgcagccactttggcaaacagggcttgagacacctt

SEQ ID NO: 69 - DNA, 294 bp, synthetic construct
  source 1..294: mol_type = other DNA
gcggggcaggcagatgcgcgccagcaggctgcgcgcctggctgtccttgtcgcccgcacgcgcctcgctgttgagcgccaggtacgcgtagggctgcgcgctgtgcggtagcaccgccgggtgcaagaaggcacgcacgaagcccagcttgtgctgctccttggcgccctgcttgatcttcaggatgttatcgtccgacgggttaaggtcgaaggtgaagagcttggccaagtctccacgcgcgtccagggagcggatagcgatctcgggcgtgttctcgaagcggtggtcttc

SEQ ID NO: 70 - DNA, 393 bp, synthetic construct
  source 1..393: mol_type = other DNA
tacgatctcagacattgttatcccgggagcgaactgcccgttgttgaaagagctataggtaaacaagttgggtacagatgtgaggtcatagataggttcttgctttatctgtttgatcccagtcatgtccagctcctgactgatcgggagacggctgaccggaatctatgctgtaataaccttcggacttaggcaggtcaatgcgtgcccgttggcgtatgtgccgccggtctcggtgttcaggttgctgaggccattgctaatgctgctgctgtacaccctggcgagggcctccgcctccccactctgctgctgccgctgctcctgcagcctttgctgaggcttctgcacctcagcatacaggctgtcccgctgcttcttggacatcctccc

SEQ ID NO: 71 - DNA, 300 bp, synthetic construct
  source 1..300: mol_type = other DNA
cctggtccttggagccagagggtggcagaggagcgccgccgcgctgataatgaatgtctccctcgacgggagcttctgcgccctcgtctccgcagggctcgccttccgatttcaggatgtccatctgcaggccctggcgatgctcaaagtcgagatcgtcgcagtgcgcgaagcccaccgcttcctcatcggtagccgcctgaaaccccatcctagcaaacatgccgctcaccttggcctgggacttgttggacacggaggtggccacattggtcagcttgctgcggagcagggtggcca

SEQ ID NO: 72 - DNA, 192 bp, synthetic construct
  source 1..192: mol_type = other DNA
atgagaagcagaccttggagtcggggctgttggccagagcatagcaggcaggggctgaggacgtcagctctgccttgttgcgtggggttggagctgccaggtcccagatagacagtgtactagcttcccctccaacaattaaggtgcggccatcagggagcaatctgcaggagcggatgtagttatccctgt

SEQ ID NO: 73 - DNA, 255 bp, synthetic construct
  source 1..255: mol_type = other DNA
ggccacatcgaagaagacctggtgccacagcagcttgcgccagagaagacgcaagtggaagaggctgggcagcaggaagcagaggccggctcccgtgaggctgcccgtgaggcccatgagcagcgcgaagtgtagcacgtagatggccatgagcagcgtgaagaccaccagcgcgcagcgcagcgtcagcccccaggacttaaggcgaccgtcgccaccgtagcaggcggggaagaaggcacgactgccttcctg

SEQ ID NO: 74 - DNA, 264 bp, synthetic construct
  source 1..264: mol_type = other DNA
ggtaagctctgtggggatgtctccaacatattcttcgacatggtgacactagacaccagatttagaggtttgggagatggtttgggctctgagggtcgcatgtgcaagtaggtcatcattgctggaagacgttcgcgttctttagaaagctgtatttctaactgttgcaccacctgcatttgcactcgacacggagcagtgcttcggtcatccaatgcgtgttcattgttaaggtgctttaaaaactgtccaaaatcttcacaa

SEQ ID NO: 75 - DNA, 279 bp, synthetic construct
  source 1..279: mol_type = other DNA
ctggccatttgcaaactccatggccatagagagtgtgagaggccccagtctcctcatgtgacgagctgtctcgtcttgcacttagaactgaagactgtccattcactatagaatgatgagttattggtggtggtgctttagaagtgttggaggaggaagtcgaggaggaattgtgagtggtgcggtctagccctccatgttgaatgccattgtcttccatactgtgaactccagtcacttctttccataactgctgaatctcagcaggacttaagccag

SEQ ID NO: 76 - DNA, 202 bp, synthetic construct
  source 1..202: mol_type = other DNA
aggacttcaggcgcccgtcgccgctgtagcaggccgggaaaaaggcgcggctgccttcctggaagagcgacttctccagcacctcgacagcggcaaagaatagcagaggataggacaacagcgccttggccaccagaaagatgttgaccacggcgcggatggagccgggcaggttatccgtgatgacctccttggtctcgtc

SEQ ID NO: 77 - DNA, 225 bp, synthetic construct
  source 1..225: mol_type = other DNA
agggatgtctgctccacctgcagctccgacagtttgccctgcaaatggttgcgctgctcctgtaacccggacgtttccaagtccgaggctgaaccggggctgcccagcgggtaggaacgacctcccaggaaagccagatgcaagaagagcaggagcaggagcgcccgggaaggtgctgtctggggatccatctctctggagggactgcggaggctgctgctgctg

SEQ ID NO: 78 - DNA, 225 bp, synthetic construct
  source 1..225: mol_type = other DNA
tccggtccatcttcctcccaaagcagccagacccttgcaccatcttggggcttcgtggtgcccgcagggtgtagaggaccattttgcggtgcccacggatgccctcggtagctacctcccgggacttccagacacctgtgggacgggggctctcctggaggggctccagggatctctgctccacctgcagctccgacagtttgccctgcaaatggttgcgctgct

SEQ ID NO: 79 - DNA, 300 bp, synthetic construct
  source 1..300: mol_type = other DNA
ggcaatcacggtggccgcacagacctacaggacagccccgccatccccgcccccgagggaccatccctttgtctcgctccctccaccgccttcccctccttccttctcccgcccggtgattggcagcctgccagaaggggcaggaaactttctgaaagtttggaggactagctctcattgtgcccaagggccttcaactgcacacaaagtggtagttttaagaaatctgtaactggaaacggagcgtccttgggcaatgtgtggggacagaggaggaaatgcgaactgcaaggtctgggt

SEQ ID NO: 80 - DNA, 300 bp, synthetic construct
  source 1..300: mol_type = other DNA
tttcggggaccagatgtgatcagcgacctccgtgggactgggaaactcagctttggactcagggcaaagagctatgaggggagaagcccatttccagctggagaactctccttcctaccacacacatggatggcctagaaaccgatcttgcccagatcccaggggactgagtgccacagtttcccacttgttaagtcaaagccgtgtttccttcatagcccactgaggaccccaccctgcatttctgctggaaggagcctcgtggaggctgtcctcgtgccccaggcccaggcagccaag

SEQ ID NO: 81 - DNA, 300 bp, synthetic construct
  source 1..300: mol_type = other DNA
tggccaccccatccaaactcactccaagtcatgccacctcagacagggagaagcctgactgaaggacccctccgcccatgtcccttgcacactgcaggtttaacagcaggagaaagcttgcgtgcttaggaagtagagggatttcaggggaaaggcttcttctgactaggcccaccctgtgcattcagaggctgccccagcgtgccagccctggggagctcgcccgccagtacagggtgttggctggcaaagaaaggcttcctttgtcccaaaggcacaactcagggcaaaggggtaaaa

SEQ ID NO: 82 - DNA, 300 bp, synthetic construct
  source 1..300: mol_type = other DNA
agggaagggagtgaagagcacaaagaaatatcctggcaagagggcaggccagctgtgagggtgggactactgatgatgcttcaaaggaagaggaagcagcaatattttaggactgggtaccagcagagaaggcagggaggagggagcgggggatcaggctagagggcgcgagttcaccatgaggctgaggaacgattctctctcgtggggtccaagactagcccaggaaacagggagtcgcagggggactggagaggagacctgggccagcgggaggcagtgttctccagagggtcagat

SEQ ID NO: 83 - DNA, 234 bp, synthetic construct
  source 1..234: mol_type = other DNA
gaagtttcactccttccgcatttattggagaaaacaggcagaaggttgggagaggggctctaccggtggccagtggagcagctggggtgggaagactgggcctagcacatctcacttagccgcaggagccggtttctcctcaaggttcattcaactgctgaagaagttcaccagactagttcctgctgtcctgacaaggggtgtcagctgctcgtgtgtcttctcaaagtatgc

SEQ ID NO: 84 - DNA, 243 bp, synthetic construct
  source 1..243: mol_type = other DNA
cactgggtgtgcatcaccctgaactggccgtcttgtttcaggatgtggaagtcacagtctccctccaccgcgtgctcagtcagctgcctcacagaacagtttgccagcggggtggggtccaaagcatagcaagtggtctccagtgtgtcaacttccatctcatacaccactccgaagggccgccgagaccacaccttgactttgtcgatctcattcaagacctgtttgaatccctcaagaaga

SEQ ID NO: 85 - DNA, 253 bp, synthetic construct
  source 1..253: mol_type = other DNA
gaactgtggtggctgggcagctcccacccacccacccacccaccaaccaaccacaaggcagcactcacgtgctcagtcagctgcctcacagaacagtttgccagcggggtggggtccaaagcatagcaagtggtctccagtgtgtcaacttccatctcatacaccactccgaagggccgctgtggagccggacagaagggagaggtgagcaaacgccctgctccgctgatccaatgcatcgcacctagtgtgt

SEQ ID NO: 86 - DNA, 270 bp, synthetic construct
  source 1..270: mol_type = other DNA
gatgtcttctggcaacttcatgcaaatagtgtcccatacaggtggttgggttttccttacaggaggtgcacatggcctcagcctctggcctttcaaatggtggcaggctagggttgtcatctttgtgttgcaggaaacattcgtttctttcgggctcttgttttgtacagcagtcagccagttcaccatagttttcacggaggtttggaatggcacacaacttatctccaaaaagagtgtgaagggatttgtcacagttggcggcagact

SEQ ID NO: 87 - DNA, 270 bp, synthetic construct
  source 1..270: mol_type = other DNA
ggtgtcttcttgaatattcatacaagaacgtgcccaggaagacatccttggcctcagcatagttcttgcacacttcctggtcctcaacaaaatcagcagcaatggcaggcagatcagcaggcatagtgtcatgctccacctcactacgacagtgggctttcttcaacagtggtttatcgcagcaagtctgcagtttgctggagatactcgcctggttttcacacatgtacttggcaagttccgccctgtcatctgcgcattccagcaggt

SEQ ID NO: 88 - DNA, 252 bp, synthetic construct
  source 1..252: mol_type = other DNA
ttctctgcaattgaagccgaatctgccctaatccccacccctgggttcagacttctgtgggacctctcatcttcccttctttctctccacagttccacagcccctgaaccccttgccctgagacttacttagcctcggcctgaagctctgggctctggaccttctccatcaggtccttgccatagtcagtcacggtctggaagtactgagaaaccaggctctccacacgtggctcctttgcctgtctccgaa

SEQ ID NO: 89 - DNA, 300 bp, synthetic construct
  source 1..300: mol_type = other DNA
gagctgccagtaacacatgggagtctgggggtgagccagctggagggagtccaggtgcgccaagtggaggggacggaggtgcatctgggtccaccacgggtgtggggactgcttcattagcaccttctggttggggctgtgagctcacgggctgtgtttggaacaccatgcaggtcactgcaacctctgccccaccaagcttctcacagagtgttgccttacaaaagccatattgcttttctgccagcaggttacacttggctgcctctgtggcctcttgagcaacacagtcagtgccag

SEQ ID NO: 90 - DNA, 300 bp, synthetic construct
  source 1..300: mol_type = other DNA
tttactagattaaaaaacattgaaataggccgggcgcggtggctcactcctgtaatcccagcactttgggaggccgaggtgggcagatcacgatgtcaggagattgagaccatcctggcacacggtgaaaccccatctctactaaaaatacaaaagattagccgggcgggatggcgggcgcctgtagtcccagctactcgggaggctgaggcaggagaatagcgtgaacccgggaggcggagtttgcagggagccgagattgcgccactgcactccagcctgggcgacagagcgagaccc

SEQ ID NO: 91 - DNA, 216 bp, synthetic construct
  source 1..216: mol_type = other DNA
ttatatcctcatgcatctgttctaccatgtcatttttccacatgtgaaaattttctgtcacatttaccaatactacttcttgtgggttggggtctgtaggtacacaggcatgtgtggcccaaacattatgtacctctgtatcatatgctttggcatctggtgcacaaaatagagtggtggttgcttccttccacacaggtaccccataatagactg

SEQ ID NO: 92 - DNA, 387 bp, synthetic construct
  source 1..387: mol_type = other DNA
ggagaaccctggacctgaattcgtagtttactgcgcaactactcataatgacactgtttttatctgtaatgggttcagggtagtaataggaactgcctgtgaacttccattctccatcatcttgaacaaaatatccagctttaggtgcgaatcctctatcaccagaaatgcaaagtccaggactcacatttgcggttgtaaaggatattagcacatagctgaagtgtataaaatagaagccataaggcgcattctggacaagagagaatatatgattaccattgccacagaaattaatacgcgtggtttggctcttaacgcactcattgaccttttctatggcctgagcagcactaactttaatggcgcgccagagggcagaggaag

SEQ ID NO: 93 - DNA, 348 bp, synthetic construct
  source 1..348: mol_type = other DNA
ggagaaccctggacctgaattccaaagtccaggactcacatttgcggttgtaaaggatattggcacagagctgaagtgtataaaatataagccagaaggcgcattctggacaagagataatataggattaccattgccacagaaattaatacgcgtggtttagctcttaacgcactcattgaccttttcttatggcctgagcagcactaactttaataagcgtactatcactaagttgcttggatatatacgcattaagtgcagtgaacctgccattaataagacgatctatctgggcttttgcttctacagcctcaagccgagtggcgcgccagagggcagaggaag

SEQ ID NO: 94 - DNA, 303 bp, synthetic construct
  source 1..303: mol_type = other DNA
ggagaaccctggacctgaattcggaggcggtttgcatattatccaagaggttattaacctcattaagaatggcattaacattaacacagaaagagccatactcaaccaactgctgcctgcatgcagtgttatcaccacagacaaatgcagcacaatctatagtcacctttagagatctagtttgaatgaactcctcgtggtgcccaatagtaaaattggttggtatttgcatctcatagaatccatcaacggattggacactatcattaactttccaggaggcgcgccagagggcagaggaag

SEQ ID NO: 95 - DNA, 351 bp, synthetic construct
  source 1..351: mol_type = other DNA
ggagaaccctggacctgaattccacacaaattgggggttttacatccgtgtgccaaaaccctataagcttattaccattagtgttaggcttacaatcagtgtaagggaacagacaaatggtatactggcacacggaggccattataacaccattatatagctctattacaacggtataggaagtatagccaaacaaactacctataactatagtaggaaaatatgcagttgcaccggatggcgtacttgtcttaaggttctgcaccttcgcaaatatgccatcattaaactgactgaaatagggtggttgaaaccacgacaagctaacggcgcgccagagggcagaggaag

SEQ ID NO: 96 - DNA, 306 bp, synthetic construct
  source 1..306: mol_type = other DNA
ggagaaccctggacctgaattcgccagcacccatacggagatcacaattaggaagcgcctcatccgtgcggttatcagcattaacaacacaacccaaataactatcaaagtaattaagtaggttctcctcacgggaaatattattgctaaaaacatagctacaatttatattacgatagagcagagccggttcgggtgcatctttgtgaaatgcagcagaaacacggccactataacagctccttatcgtataagtcttgttagtggtaagatcacgaaaaccggcgcgccagagggcagaggaag

SEQ ID NO: 97 - DNA, 360 bp, synthetic construct
  source 1..360: mol_type = other DNA
ggagaaccctggacctgaattcaatgcatcagcattaacattaagcgtgaaatttcgctttaacacacaaattgggggttttacatccgtgtgccaaaaccctataagcttattaccattagtgttaggcttacaatcagtggaaggtaacagacaaatggtatactagcacactgaggccaattataacaccattatatagctctattacaacggtataggaagtatagccaaacaaactacctataactatagtaggaaaatatgcagttgcaccggatggcgtacttgtcttaaggttctgcaccttcgcaaatatgccatcattaaactgactggcgcgccagagggcagaggaag

SEQ ID NO: 98 - DNA, 390 bp, synthetic construct
  source 1..390: mol_type = other DNA
ggagaaccctggacctgaattcgcagttaacctgccattaataagacgatctatctgggcttttgcttctacagcctcaagccgagttagaatttcttgtaaagaagcactaatagcaccaaacctgttagaaagtggatttagtaagttattgagtgcttcagcatttgcattaacaacggactagatcttacctaaagcagaattggttgcatcaaacccatcctagatagcacccagcgcattgttaaaagcactagcaatcatcttttggttctcactaagcacattcatagtgacacctaaaccattaattctatatggaacacttaaactaaatggcacaccggcagctgctgaccacggtggcgcgccagagggcagaggaag

SEQ ID NO: 99 - DNA, 387 bp, synthetic construct
  source 1..387: mol_type = other DNA
ggagaaccctggacctgaattcgcttttgcttctacagcctcaagccgagttagaatttcttgtaaagaagcactaatagcaccaaacctgttagaaagtggatttagtaagttattgagtgcttcagcatttgcattaacaacggactggatcttacctaaagcagaattggttgcatcaaacccatcctagatagcacccagcgcattgttaaaagcactagcaatcatcttttagttctcactaagcacattcatagtgacacctaaaccattaattctatatggaacacttaaactaaatggcacaccggcagctgctgaccacggtgggaacatagctgccgcagtagcaccggttgtgggcgcgccagagggcagaggaag

SEQ ID NO: 100 - DNA, 366 bp, synthetic construct
  source 1..366: mol_type = other DNA
ggagaaccctggacctgaattcagttgcatattatccaagaggttattaacctcattaagaatggcattaacattaacacagaaagagccatactcaaccaactgctgcctgcatgcagtgttatcaccacagacaaatgcagcacaatctatagtcacctttggagatctagttggaatgaactcctcatagtgcccaatagtaaaattggttggtatttgcatctcatataatccatcaacggattagacactatcattaactaacatcggagtgtatggctcaaatgtagttaaccgagagccagtagaaactgatcggtcagccctgcgtgattttgaaggcgcgccagagggcagaggaag

SEQ ID NO: 101 - DNA, 438 bp, synthetic construct
  source 1..438: mol_type = other DNA
ggagaaccctggacctgaattcgaggaaatattatgtatcacaatactgtcctggtgtcctccatactcatcacaacaatttccaaaatctaactcctccttaaagtcgggtggattaggtattgaagtgttcaagaaaacttcaggtgccaaaagttgcttggatatatacgcattaagtgcagttaacctgccattaataagacgatctatctaggcttttgcttctacagcctcaagccgagttagaatttcttgtaaagaagcactaatagcaccaaacaaacttaaactaaatggcacaccggcagctgctgaccacggtgggaacatagctgccgcagtagcaaaaccgtctggcagtctcgagcttatagtaacaccctgcattaatgcactagcaacttgtagttgcggcgcgccagagggcagaggaag

SEQ ID NO: 102 - DNA, 498 bp, synthetic construct
  source 1..498: mol_type = other DNA
ggagaaccctggacctgaattcgaggaaatattatgtatcacaatactgtcctggtgtcctccatactcatcacaacaatttccaaaatctaactcctccttaaagtcgggtggattaggtattgaagtgttcaagaaaacttcaggtgccaaaagttgcttggatatatacgcattaagtgcagttaacctgccattaataagacgatctatctaggcttttgcttctacagcctcaagccgagttagaatttcttgtaaagaagcactaatagcaccaaacaaaaaggtgtcgagaagaggagaacaatatgctaaatgttgttctcgtctcctcgacaccaaacttaaactaaatggcacaccggcagctgctgaccacggtgggaacatagctgccgcagtagcaaaaccgtctggcagtctcgagcttatagtaacaccctgcattaatgcactagcaacttgtagttgcggcgcgccagagggcagaggaag

SEQ ID NO: 103 - DNA, 501 bp, synthetic construct
  source 1..501: mol_type = other DNA
ggagaaccctggacctgaattcgaggaaatattatgtatcacaatactgtcctggtgtcctccatactcatcacaacaatttccaaaatctaactcctccttaaagtcgggtggattaggtattgaagtgttcaagaaaacttcaggtgccaaatcgacaaagccaacatcagataatttgaccttgtcaaataacaaatcctctatagcagaaaaactcatccgtgcggttatcagcattaacaacacaacccaaataactatcaaagtaattaagtaggttctcctcacgggaaatattattgctaaaaacatagctacaatttatattacgatagagcagagcaaaccatttaacaatatattagcaaaaatttggcagcgatcattaacaaggcaggtatcatgtaaatggtttttgccaaagacgccagcatcattaaagccatacctcctattccaagacgaggggttatggggcgcgccagagggcagaggaag

SEQ ID NO: 104 - DNA, 642 bp, synthetic construct
  source 1..642: mol_type = other DNA
ggagaaccctggacctgaattcgaggaaatattatgtatcacaatactgtcctggtgtcctccatactcatcacaacaatttccaaaacgtataagtcttgttagtggtaagatcacgaaaaccattcaaattaccattaacatcatacagaagggtttgccagctattataatagtcaaaccttatgtggatcagcattgccacaattatcttctaaaacacctaagccttcacaatggtcacaaagcctacttcggggtacagcaaacttatcaactgaaatactaccaaagcacctgccatacactttagaagcatcgatattattacaaaacaaactctcagcctgaacataacgtaacaggctgcttaaataaaaaggtgtcgagaagaggagaacaatatgctaaatgttgttctcgtctcctcgacaccaaagctctgggtcttacattttatttcactggtataactactagcacaatcaacagcactagtaataaaacaaattgggggttttacatccgtgtgccaaaaccctataagcttattaccattagtgttaaaaacttcaacggtctcagtgctaatgcttggagcactaacattagcaccgttggcgcgccagagggcagaggaag

SEQ ID NO: 105 - DNA, 618 bp, synthetic construct
  source 1..618: mol_type = other DNA
ggagaaccctggacctgaattcgtcttgttagtggtaagatcacgaaaaccattcaaattaccattaacatcatacagaagggtttgcaaacccttatgtggatcagcattgccacaattatcttctaaaacacctaagccttcacaatggtcacccaaattagcagtctgcagaaatccagagttaccaagctgtaaatcaacttgcctacttcggggtacagcaaataaagtcttacgctcccagttgagaggggaggggactgacctagcagtaagccactcctctataaaaagcttattaccattagtgttaggcttacaatcagtggaaggtaacagacaaatggtatactagcacactgaggccaattataacaccattatatagctctattacaacggtataggaagtatagccaaacaaactacctataactatagtaggaaatataaccacgacaagctaactgagttagttcccgtaagagcgaggtttctaaacttagaaccatcgaccgggaaaagctggatacatctaaaatcaccaatataccctaaacaagagggcaaaaatagaataaacacgaacagcggcgcgccagagggcagaggaag

SEQ ID NO: 106 - DNA, 816 bp, synthetic construct
  source 1..816: mol_type = other DNA
ggagaaccctggacctgaattcatattatgtatcacaatactgtcctggtgtcctccatactcatcacaacaatttccacacaaattcttaaaccatttatctaactcctccttaaagtcgggtggattaggtattgaagtgttcaagaaaactaaatatccagctttaggtgctaatcctctatcaccagaaatgcaaagtccaggactcacatttgcgaaaaaggtgtcgagaagaggagaacaatatgctaaatgttgttctcgtctcctcgacaccaaagcattctggacaagagataatataggattaccattgccacagaaattaatacgcgtggtttagctcttaacgcactcattgaccttttcttatggcctgagcagcactaactttaataagaaagtggaagaggagaacaataggctaactgttgttctcgtctcccactttaaactgctgcctgcatgcagtgttatcaccacagacaaatgcagcacaatctatagtcacctttagagatctagtttgaatgaactcctcgtggtgcccaatagtaaaattggttggtatttgcatctcataaaattgttagtggtaagatcacgaaaaccattcaaattaccattaacatcatacagaagggtttgccagctaaaaccagtaaccacttcagtattaggcaactgcaaatctgtggaacatgtggtaccactattaaaaacgtcatgttggtttttgccaaagacgccagcatcattaaagccatacctcctattccaagacgagaaaggcgcgccagagggcagaggaag

SEQ ID NO: 107 - DNA, 300 bp, synthetic construct
  source 1..300: mol_type = other DNA
aaagcatgcggtaagaaacttagggaagaaactatgagagtacttcttagggtgagactcagagtctgagttgggatggaatgggcttggagccagggctcccagttacggatgtgcagccagccccatcagtagcttcatcctctgtactgggctcctgcatctccggggctgcttcccacccagcccccacattcccaggaagagtacgctagagttcctcacctgttccaccttgcccctcctgccaatgctctgcgctcggcctcctgtcatggtcttcacaacgccagccctgga

SEQ ID NO: 108 - DNA, 249 bp, synthetic construct
  source 1..249: mol_type = other DNA
gatcctcagcaagagaacaaagaagagcccagtctgaaagcatgcggtaagaaacttagggaagaaactatgagagtacttcttagggtgagactcagagtcggagttgggatggaatgggcttagagccagggctcccagttacggttgtgcagccagccccatcagtggcttcatcctctgtactgggctcctgcatctccggggctgcttcccacccagcccccacattcccaggaagagtacgct

SEQ ID NO: 109 - DNA, 300 bp, synthetic construct
  source 1..300: mol_type = other DNA
actgggaacaatacacactccatgcgttttgctacatctccggaagaggtaaggactcgagtccacacatggatgctttcaagtcctcgaggcccacagcctggtgtgtttcacgcacagataaggtcctccctaggtctacaggaaccctctagggaagatgtgtttctcctctctgtaatgcaccagctcgggcagtagcacttgtgggtgccggctgcctccccttccctgccccctcacagggccagcagcgtgggtgagctgagcgagtcagaggaaggctcattgctgctgctg

SEQ ID NO: 110 - DNA, 252 bp, synthetic construct
  source 1..252: mol_type = other DNA
ttaacctcagtaatactttatctttactattggaggcatgtcacatggagtccatgttgtctacttacatctgctgcatgaacagtcccaaaacagtgcgctgaagagaagaaaggtgcttctgcaaggtttggagaggagacaactttgacctagccggctggcttggcgcaggggcggtgcggagtcagcggagagcccgagtgggaatgcggcaggtcgtctttgcagtgccctggcgggagccgcggc

SEQ ID NO: 111 - DNA, 300 bp, synthetic construct; identical to SEQ ID NO: 71
  source 1..300: mol_type = other DNA

SEQ ID NO: 112 - DNA, 249 bp, synthetic construct
  source 1..249: mol_type = other DNA
tcctctgcgttggttcggtgggcctcgatgagaccctcgagtgaatgcacgaagccggacacgctgcagatgccgccgatgacgaagatggccacatcgaagaagacctagtgccacagcagcttgcgccagagaaggcgcaagtagaagaggctgggcagcaggaagcagaggccggcgcctgtgaggctgcccgtgaggcccatgagcagcgcgaagtgtggcacggagatggccatgagcagcgtg

SEQ ID NO: 113 - DNA, 291 bp, synthetic construct
  source 1..291: mol_type = other DNA
aaacggaaggaagccacaaaggaagaaaatactcagacgctttaggtagtagacacagggtcttggggaacggagacgaggtgtagatataggtttggaaataagaactacatagaagaagactagagttgggaaaccctaagatgggtgttaaatgggatctgcagagaggatcagaatagagacccctccaacccacaagccctacgctgcaggaagcctaaaccgcaggtctctagaaactgaggagaggggagggccaaaagctgcgcaatatggtttctaatttac

SEQ ID NO: 114 - DNA, 396 bp, synthetic construct
  source 1..396: mol_type = other DNA
gtttctgcggtgcagccgggatttccagcaagaggagctagtcttcgcagcttcgtactgcggagtccctctaccatagacgttcctatgtacctgtatctgtatgtgtccaacagagctgcaccagccaagtaggtgctgtctgctttagtgtgtgtgtgcgtgtgcgtgcgtgtgcgtgcatgtgtgtgtgctcacgagttaagattaagctaatgcagaggcacttgctgtgtaaaggtattgctgctattgcagtgtcttgtggtatagtcaagtgcatttagggtaaccctgcgaactataaatagatgcacgtcatatgcctgtttgttagtgaggaaggggaccgtgtctagccctgtccgcgtgcacgacttctgtttgtgtcacgag

SEQ ID NO: 115 - DNA, 282 bp, synthetic construct
  source 1..282: mol_type = other DNA
gcggcggcggggcgggagaaaaggacagaagggtctggggacgcggcgggcgcgcgtcaaggcagcgaggactgctatctccgctgggtctccgcgcggcgccccagggcgctctggctcatgaccctcgcgcctgacgttcgaggctagccccggtcacgacagtctggctcttggtaggaccggcccgagaggcgacgagtgcactgcagagctggtgcgggtgccggctgggctgcggagggcggcggcgacggcagcgtcagaacagctacgcgaggc

SEQ ID NO: 116 - DNA, 363 bp, synthetic construct
  source 1..363: mol_type = other DNA
tatacatttcatcttattcaccacgagcacaccacacgcacagtatacagttccacgaccggatacattgcacaagatgagtttgggttcctgaaaaccggcaggcaaacggggcccctgccgcggcttctccagagtgaagtcgcccgcccgaaatagatttcccggtcaacctttcaggccgcaaggttgaaatgtccagggcatggggagcgctcttgctagaaatggctggacgctgtagattccaagcactggctggctgtgaaatggccccaccccaccctcaaggtcaagtttccaagcctgcgaactttccctcccttcccacccccgtcgttgacaggagccaaaatttggtgg

SEQ ID NO: 117 - DNA, 255 bp, synthetic construct; identical to SEQ ID NO: 73
  source 1..255: mol_type = other DNA

SEQ ID NO: 118 - DNA, 210 bp, synthetic construct
  source 1..210: mol_type = other DNA
atggccatgagcagcgtgaagaccaccagcgcgcagcgcagcgtcagcccccaggacttaaggcgaccgtcgccaccgtaccaggcggggaagaaggcacgactgccttcctagaagagagacttctccagcacttcgacggccgcgaagaagggcaacgggtacgacagcagcgccttggccaccaggaagatcttgaccacggcgcgg

SEQ ID NO: 119 - DNA, 204 bp, synthetic construct
  source 1..204: mol_type = other DNA
gaccaggacttctgcgacacgggcagccccgggaaactgttgtacatgaggttgccgctcaccactacgtacaagatacacgtcatcaccagctcgatgatctaggccacattgaccacgcggccgcccagcgtggggaatcgaggagcgcagcacgcgttagctatcgccacatacgagtccctcacgcgcaccacctcacca

SEQ ID NO: 120 - DNA, 193 bp, synthetic construct
  source 1..193: mol_type = other DNA
cgtccccgcagggctcgccttccgatttcaggatgtccatctgcaggccctggcggtgctcaaagtcgagatcgtcgcagtgcgcgaagcccaccgcctcctcatccgtagccgcctgaaaccccatcctagcgaacatgccgctcaccttggcctgggacttgttggacacagaggtggccacgttggtcav

SEQ ID NO: 121 - DNA, 342 bp, synthetic construct
  source 1..342: mol_type = other DNA
catgagcagcgtgaagacgacaagcgcgcagcgcagcgtcagcccccaggacttcaggcgcccgtcgccgccgtagcaggccgggaaaaaggcgcggctgccttcctggaagagcgacttctccagaacctcgacagcggcgaagaatagcagaggataggacaacagcgccttggccaccagaaagatgttgaccacggcgcggatggagccaggcaggttatccgtgattacctccttggtctcgtcggcccaggtgaggtaggcgacgagtgcgaagaggcccttgagcacgcaggcggcgatgtgcgtccagttcatcatgcagtggaactcgctggg

SEQ ID NO: 122 - DNA, 237 bp, synthetic construct
  source 1..237: mol_type = other DNA
aacttgaccttctcccaggcccagtcgcgtgcccgcgatagacagtgggctatgactaggatattgatgacgaagtaggccagagtgcacagcagactgaacttggacacggccttgaggttcttaaggaaggcgcaaggcaggagcacggccgtggcgataatggaccaggacttctgcgacacgggcagccccgggaagctgttgtacatgaggttgccactcaccaccacgtac

SEQ ID NO: 123 - DNA, 255 bp, synthetic construct
  source 1..255: mol_type = other DNA
ggccacatcgaagaagacctggtgccacagcagcttgcgccagagaaggcgcaagtggaagaggctgggcagcaggaagcagaggccggcgcctgtgaggctgcccgtgaggcccatgagcagcgcgaagtgtagcacgtagatggccatgagcagcgtgaagaccaccagcgcgcagcgcagcgtcagcccccaggacttaaggcgaccgtcgcctccatagcaggcggggaagaaggcgcgactgccttcctg

SEQ ID NO: 124 - DNA, 204 bp, synthetic construct
  source 1..204: mol_type = other DNA
aaaggacttcaggcgcccgtcgccgctgtagcaggccgggaaaaaggcgcggctgccttcctggaagagcgacttctccagcacctcgacagcggcaaagaatagcagaggataggacaacagcgccttggccaccagaaagatgttgaccacggcgcggatggagccgggcaggttatccgtgatgacctccttggtctcgtc

SEQ ID NO: 125 - DNA, 345 bp, synthetic construct
  source 1..345: mol_type = other DNA
caggatgtgaaagtcttccagaagaaattcttgcagccagctttgcgttctcggggtgccatagccgggtttgagttagcagatctctgcagctcaagcctcatttcatcctgctcagcagcctaggacagatcttcaggttccagggcatcattctccgtctggttgggttcagacagcagctctgccaagaagtacttggccagttcctgcttccccgcggcagcagccagggacttctgcagaaacggacggagtctggggtccgagggagcgccggtgacacagcccagggccaggacggtggacagcgcagccagcgcgcactggaggcggcaggacagc

SEQ ID NO: 126 - DNA, 327 bp, synthetic construct
  source 1..327: mol_type = other DNA
ctttcagccaccagagtggagagttcgtcaaccccaattttgccgtccccatctttgtctccagcagccatcagcatcttggtttctttagcagacaggtctctggcatctggggagaagccttttaggatgaatcccagctcatcctcctcgatgaagccacttttgtccttgtccagcatgtggaacaccttcttcacatcatccgcactctttttcttcaggccgaccatttagaagaactttttgtggtcgaaggagtcggtagcgctaaaggctcccaccgccttcttgatgtcctcagcgttcagcaagtctgtcatcgac

SEQ ID NO: 127 - DNA, 228 bp, synthetic construct
  source 1..228: mol_type = other DNA
aagaaggcgcgcggacctgctaccactcctgcaccgccaggccaggggtccgcgggatcccaggggctgcggccagggcacgagggaaggggccacctctaggatttagggggcactggcgtcaccagctgggtctggaaagtccacctgccgtcaaggacacgcaggaggtgcgccgtctcagatctgggaaccttggcggatgtcctgccgcgtgggggaagatcc

SEQ ID NO: 128 - DNA, 345 bp, synthetic construct
  source 1..345: mol_type = other DNA
gtgtgcagaggtgacgtgttcaaggtcacagctggcaaggggcagagaaggagcagagcatgggggtctgggctttacactcaggcagcggatgtttatgaagcacctgctctgtgtggggtattggctagatactggggctggaaggatgaatgcaacccaatccctagttttaagttttacatccacttatatgttggatgtaagtaggtagaacgtgaagattctagacagaaggtacccttgcgaagggacctagttttaaaaaaattagctaacaaatacatatataacatttactctgtgccagacactgttctaggcactttacatattaacacatct

SEQ ID NO: 129 - DNA, 372 bp, synthetic construct
  source 1..372: mol_type = other DNA
ccactcctgtctgtgtcgtacttccgccaagcctccataaactcggcgctggagcccacgtgctgcctgaagcacagaaggaagttctcttcggttggcaggatctgcgccagctctgccatctcgattttcccatctgagtttttatcatacttctgcatgaactccttcatcttttctccaaagttgtcactctttgacatcatgccagagcctttccttgccttctccagctcttagaaaaagttttctagctctttaccttcaatatacccatttccgtctgcgtcaaagtgcttccatatttccaggaactgggacgccgtcagctcggccaggtgcagggaagggggctgctgctgcgggccagcc

SEQ ID NO: 130 - DNA, 381 bp, synthetic construct
  source 1..381: mol_type = other DNA
ccgcttctatccttgtcggaaaatgtgaagatcgcgttaaactcctctgaggtcagcttcatgcccggaaatttaagcaggaagttttcctggacaggcaggagtcgggacatctctgagaggcccaatttgccatccccgttcaagtcaaacatccgtagtatggtttgggtgtattcctagagcttgggctcatcgtacggccggttcgccttcttcagcaggtctgacaggaatcccttgagctcattggcttcgattcagccactcctgtctgtgtcgtacttccgccaagcctccataaactcggcgctggagcccacgtgctgcctgaagcacagaaggaagttctctggcgcgccagctactaacttcagcctg

SEQ ID NO: 131 - DNA, 300 bp, synthetic construct
  source 1..300: mol_type = other DNA
gtaaaaatggacgaatacggccgggcgtggtggctcacgcctgtgttcccagcactttgggaggccaaggtgggcagaacacaacgtcaggagatcgagaccatcctggctaacacggtgaatccccgtctctacgaaaaatacaaaaacaaaattagccaggtgtagtggcgggtacctgtagtcccagctaccctggaggctgaggtgggagaatggcgtgaacccgggaggcggagcttgcagggagccaaaatcgcgccacggcactccagcctgggcaacagagggagactctgt

SEQ ID NO: 132 - DNA, 300 bp, synthetic construct
  source 1..300: mol_type = other DNA
gttagagaaataaaactgtattggccaggcgctgtggctcacgcctgtaatcgcagcactttgggaggccgaggcgggcagattacgaggtcaggagatcgagaccatcctggcgaacgcggtgaaaccccgtctctactaaaaatacaaaaaattagccgggcgccttagcgggcacctgtagttccagctactccggaggctgaggcgggagaatggcgtgaacccgggaggcggagcttgcagggagccgagatcgcgtcactgcactccagtctgggtgacagagcaagacttcgt

SEQ ID NO: 133 - DNA, 300 bp, synthetic construct
  source 1..300: mol_type = other DNA
aaggcaaaccaaaaaggggaaaaaggatataaaagctgcccctcagtcgaggtgactgtccagaaaagcaatgtaacagggacagtggagggcgtgtgtccccattctgtccccaccctgtctatagacaccatcgagaaagtcaccctcctactcccagaccactagcttctctgcctgctcgggagctcgggaggcaggagcctggtggggacggagggaggtggaggcaggggtgtagacagcccctctaagggaaaaggtggcctcacactccccagaagtcagccagcctgtggc

SEQ ID NO: 134 - DNA, 300 bp, synthetic construct
  source 1..300: mol_type = other DNA
aatggttctctttgggggctgggagccttggctcaggcctgtaatcccagcactctgggaggccgaggcagcaggcgggagcatcactttgagctcaagagctgtaggccagcctgggcaacatagcgaaatcccatctctacaaaaatacaaaaaaatgagcccggcgttggtggcttgcacctgtggtcccagctacttaggaggctgaggttggagaatcgcttgagcccaggaagcagaggttgcagtgagccaagagtgcaccccctgcactccagtctgggtgacacagcaaga

SEQ ID NO: 135 - DNA, 255 bp, synthetic construct; identical to SEQ ID NO: 31
  source 1..255: mol_type = other DNA

SEQ ID NO: 136 - DNA, 435 bp, synthetic construct
  source 1..435: mol_type = other DNA
actccaccaggtagtggccgccgtccttcagcttcagggcctggtggatctcgcccttcagcacgccgtcgcgggggtacaggcgctcggtggaggcctcccagcccatagtcttcttctgcattacggggccgtcgggggggaagttggtgccgcgcatcttcaccttgtagatcagcgtgccgtcctgcagggcttcctctacggcagcaagaccttcatcaaccacacccagggcatggatgtcggcggggtgcttcacgtacgccttggagccgtacaggaactggggggacaggatgtcccaggcgaagggcagggggccgcccttggtcaccttcagcttagcggtctgggtgccctcgtaggggcggccctcgccctcgccctcgatctcgaactcgtggccgttcatggagccctccatgcgcacct

SEQ ID NO: 137 - DNA, 435 bp, synthetic construct; identical to SEQ ID NO: 136
  source 1..435: mol_type = other DNA

SEQ ID NO: 138 - DNA, 441 bp, synthetic construct
  source 1..441: mol_type = other DNA
actccaccaggtagtggccgccgtccttcagcttcagggcctggtggatctcgcccttcagcacgccgtcgcgggggtacaggcgctcggtggaggcctcccagcccatagtcttcttctgcattacggggccgtcgggggggaagttggtgccgcgcatcttcaccttgtagatcagcgtgccgtcctgcagggcttcctctacggcagcaagaccttcatcaaccacacccagggcatgcgcaggtagtgtcccaacaacccccaacaatttttactcatcagatcaataatcgtgtgacctacggtggagccatagacgctcaggacgccaaaaccttcgggccccaaaatgaagagaattaggaacataccccagctcacgaaaaacagccatgccatgccggtcacgacctggcggcaccgaccctttggcacagt

SEQ ID NO: 139 - DNA, 675 bp, synthetic construct
  source 1..675: mol_type = other DNA
cggggaggcggcccaaagggagatccgactcgtctgagggcgaaggcgaagacgcggaagaggccgcagagccggcagcaggccgcgggaaggaaggtccgctagattgagggccgaagggacgtagcagaaggacgtcccgcgcagaatccaggtggcaacataggcgagcagccaaggaaaggaaatcaaggtggtcgagggcggccctctccccttcgccttcgacatcctggctacactccaccaggtagtggccgccgtccttcagcttcagggcctggtggatctcgcccttcagcacgccgtcgcgggggtacaggcgctcggtggaggcctcccagcccatagtcttcttctgcattacggggccgtcgggggggaagttggtgccgcgcatcttcaccttgtagatcagcgtgccgtcctgcagggctacggcagcaagaccttcatcaaccacacccagggcatgcgcaggtagtgtcccaacaacccccaacaatttttactcatcagatcaataatcgtgtgacctacggtggagccatagacgctcaggacgccaaaaccttcgggccccaaaatgaagagaattaggaacataccccagctcacgaaaaacagccatgccatgccggtcacgacctggcggcaccgaccctttggcacagt

SEQ ID NO: 140 - DNA, 225 bp, synthetic construct; identical to SEQ ID NO: 36
  source 1..225: mol_type = other DNA

SEQ ID NO: 141 - DNA, 249 bp, synthetic construct; identical to SEQ ID NO: 37
  source 1..249: mol_type = other DNA

SEQ ID NO: 142 - DNA, 228 bp, synthetic construct; identical to SEQ ID NO: 38
  source 1..228: mol_type = other DNA

SEQ ID NO: 143 - DNA, 237 bp, synthetic construct; identical to SEQ ID NO: 47
  source 1..237: mol_type = other DNA

SEQ ID NO: 144 - DNA, 237 bp, synthetic construct; identical to SEQ ID NO: 47
  source 1..237: mol_type = other DNA

SEQ ID NO: 145 - DNA, 234 bp, synthetic construct; identical to SEQ ID NO: 43
  source 1..234: mol_type = other DNA

SEQ ID NO: 146 - DNA, 219 bp, synthetic construct; identical to SEQ ID NO: 45
  source 1..219: mol_type = other DNA

SEQ ID NO: 147 - DNA, 250 bp, synthetic construct
  source 1..250: mol_type = other DNA
ccatgtagatggtctggaactccaccaggtagtggccgccgtccttcagcttcagggcctggtggatctcgcccttcagcacgccgtcgcgggggtacaggcgctcggtggaggcctcccagcccatagtcttcttctgcattacggggccgtcgggggggaagttggtgccgcgcatcttcaccttgtagatcagcgtgccgtcctgcagggaggagtcctgggtcacggtcaccagaccgccgtcctg

SEQ ID NO: 148 - DNA, 1029 bp, synthetic construct
  source 1..1029: mol_type = other DNA
atggcggcttctcaggcggtggaggaaatgcggagccgcgtggttctgggggagtttggggttcgcaatgtccatactactgactttcccggtaactattccggttatgatgatgcctgggaccaggaccgcttcgagaagaatttccgtgtggatgtagtacacatggatgaaaactcactggagtttgacatggtgggaattgacgcagccattgccaatgcttttcgacgaattctgctagctgaggtgccaactatggctgtggagaaggtcctggtgtacaataatacatccattgttcaggatgagattcttgctcaccgtctggggctcattcccattcatgctgatccccgtctttttgagtatcggaaccaaggagatgaagaaggcacagagatagatactctacagtttcgtctccaggtcagatgcactcggaacccccatgctgctaaagattcctctgaccccaacgaactgtacgtgaaccacaaagtgtataccaggcatatgacatggatccccctggggaaccaggctgatctctttccagagggcactatccgaccagtgcatgatgatatcctcatcgctcagctgcggcctggccaagaaattgacctgctcatgcactgtgtcaagggcattggcaaagatcatgccaagttttcaccagtggcaacagccagttacaggctcctgccagacatcaccctgcttgagcccgtggaaggggaggcagctgaggagttgagcaggtgcttctcacctggtgttattgaggtgcaggaagtccaaggtaaaaaggtggccagagttgccaacccccggctggataccttcagcagagaaatcttccggaatgagaagctaaagaaggttgtgaggcttgcccgggttcgagatcattatatctgtaagaaagatttgctggctgcggtggctcacacctgtaatcccagcactttgggaggctgaggcgggtggatcacgaggtcaggagatcgagaccatcctggctaa

SEQ ID NO: 149 - skipped

SEQ ID NO: 150 - DNA, 809 bp, synthetic construct
  source 1..809: mol_type = other DNA
cccgctgctctgctgcctgcccggggtaccaacatggcccagaagcgtcctgcctgcaccctgaagcctgagtgtgtccagcagctgctggtttgctcccaggaggccaagaagtcagcctactgcccctacagtcactttcctgtgggggctgccctgctcacccaggaggggagaatcttcaaagggtgcaacatagaaaatgcctgctacccgctgggcatctgtgctgaacggaccgctatccagaaggccgtctcagaagggtacaaggatttcagggcaattgctatcgccagtgacatgcaagatgattttatctctccatgtggggcctgcaggcaagtcatgagagagtttggcaccaactggcccgtgtacatgaccaagccggatggtacgtatattgtcatgacggtccaggagctgctgccctcctcctttgggcctgaggacctgcagaagacccagtgacagccagagaatgcccactgcctgtaacagccacctggagaacttcataaagatgtctcacagccctggggacacctgcccagtgggccccagccctacagggactgggcaaagatgatgtttccagattacactccagcctgagtcagcacccctcctagcaacctgccttgggacttagaacaccgccgccccctgccccacctttcctttccttcctgtgggccctctttcaaagtccagcctagtctggactgcttccccatcagccttcccaaggttctatcctgttccgagcaacttttctaattataaacatcacagaacatcctgga

SEQ ID NO: 151 - skipped

SEQ ID NO: 152 - DNA, 2353 bp, synthetic construct
  source 1..2353: mol_type = other DNA
agtgcagacgcggctcctagcggatgggtgctattgtgaggcggttgtagaagttaataaaggtatccatggagaacactgaaaactcagtggattcaaaatccattaaaaatttggaaccatcatacatggaagcgaatcaatggactctggaatatccctggacaacagttataaaatggattatcctgagatgggtttatgtataataattaataataagaattttcataaaagcactggaatgacatctcggtctggtacagatgtcgatgcagcaaacctcagggaaacattcagaaacttgaaatatgaagtcaggaataaaaatgatcttacacgtgaagaaattgtggaattgatgcgtgatgtttctaaagaagatcacagcaaaaggagcagttttgtttgtgtgcttctgagccatggtgaagaaggaataatttttggaacaaatggacctgttgacctgaaaaaaataacaaactttttcagaggggatcgttgtagaagtctaactggaaaacccaaacttttcattattcaggttattattcttggcgaaattcaaaggatggctcctggttcatccagtcgctttgtgccatgctgaaacagtatgccgacaagcttgaatttatgcacattcttacccgggttaaccgaaaggtggcaacagaatttgagtccttttcctttgacgctacttttcatgcaaagaaacagattccatgtattgtttccatgctcacaaaagaactctatttttatcactaaagaaatggttggttggtggttttttttagtttgtatgccaagtgagaagatggtatatttggtactgtatttccctctcattttgacctactctcatgctgcagagggtactttaagacatactccttccatcaaatagaaccactatgaagctacctcaaacttccagtcaggtagttgcaattgaattaaattaggaataaataaaaatggatactggtgcagtcattatgagaggcaatgattgttaatttacagctttcatgattagcaagttacagtgatgctgtgctatgaattttcaagtaattgtgaaaaagttaaacattgaagtaatgaatttttatgatattccccccacttaagactgtgtattctagttttgtcaaactgtagaaatgatgatgtggaagaacttaggcatctgtgggcatggtcaaaggctcaaacctttattttagaattgatatacacggatgacttaactgcatttttagaccatttatctgggattatggttttgtgatgtttgtcctgaacacttttgttgtaaaaaaataataataatgtttaatattgagaaagaaactaatattttatgtgagagaaagtgtgagcaaactaacttgacttttaaggctaaaacttaacattcatagaggggtggagttttaactgtaaggtgctacaatgcccctggatctaccagcataaatatcttctgatttgtccctatgcatatcagttgagcttcatataccagcaatatatctgaagagctattatataaaaaccccaaactgttgattattagccaggtaatgtgaataaattctataggaacatatgaaaatacaacttaaataataaacagtggaatataaggaaagcaataaatgaatgggctgagctgcctgtaacttgagagtagatggtttgagcctgagcagagacatgactcagcctgttccatgaaggcagagccatggaccacgcaggaagggcctacagcccatttctccatacgcactggtatgtgtggatgatgctgccagggcgccatcgccaagtaagaaagtgaagcaaatcagaaacttgtgaagtggaaatgttctaaaggtggtgaggcaataaaaatcatagtactctttgtagcaaaattcttaagtatgttattttctgttgaagtttacaatcaaaggaaaatagtaatgttttatactgtttactgaaagaaaaagacctatgagcacataggactctagacggcatccagccggaggccagagctgagccctcagcccgggaggcaggctccaggcctcagcaggtgcggagccgtcactgcaccaagtctcactggctgtcagtatgacatttcacgggagatttcttgttgctcaaaaaatgagctcgcatttgtcaatgacagtttcttttttcttactagacctgtaacttttgtaaatacacatagcatgtaatggtatcttaaagtgtgtttctatgtgacaattttgtacaaatttgttattttccatttttatttcaaaatatacattcaaacttaaaatta

SEQ ID NO: 153 - DNA, 2668 bp, synthetic construct
  source 1..2668: mol_type = other DNA
agttggctactcgccatggacgaagcggatcggcggctcctgcggcggtgccggctgcggctggtggaagagctgcaggtggaccagctctgggacgccctgctgagccgcgagctgttcaggccccatatgatcgaggacatccagcgggcaggctctggatctcggcgggatcaggccaggcagctgatcatagatctggagactcgagggagtcaggctcttcctttgttcatctcctgcttagaggacacaggccaggacatgctggcttcgtttctgcgaactaacaggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcataaggccagggcccctcaccctgccttatcttgcaccccaaagctttcctgccccaggcctgaaagaggctgaggcctggactttcctgcaactcaaggactttgcagccggcacagggtctgctctttctctgccagtgacagacaggctcttagcagcttccagattgacgacaagtgctgaacagtggaggaagagggacagatgaatgccgtggattgcacgtggcctcttgagcagtggctggtccagggctagtgacttgtgtcccatgatccctgtgttgtctctagagcagggattaacctctgcactactgacatgtggggccaggtcaccctttgctgtgaggctgtcctgtacattgtgggatgttcagcactgtcccttgcctcaatgccagtaacgcgtcttcctgagtggtgccaaacaaaaaggttctcaggtgttgccaaatatgtcctggggtataaaactttcctcgcctgacaaccactggtctgtagggatttttggctacacacaaaccagtatcgctcatagatcagcaaaccggggcctactagagtctgaacagctgtaatctatgaattctaagtgaaattttaaaaattgttaatttttcctatattgcattaattttaaaaaataaatctgaggcaaatatggactctcttttgcctatttcttccctcattttgctccaactctttcttcttccttacaaaagagacttttgcttttttcgaaacatttccccatgtttttctggggtctcgctatgttgcccaggctggtctcaaactcctgggctcaagtgaccctcccaagtagctcttactacaggcgtgcaccattgcacccagccccatttattcatgtcttatttcacttgatccttatcccatcccaggaaggcaacaagggtgagaaccctgtgctcagggaggttaggtctcttgtccaagggaaaacgattatccagagaagagacctggccagaacctgggtcccctgagtcctagccatgcttcccatgtgccttacttgctgaagcacccccggactgcagtgtgaacgtgctgtgcaatagtgacacgctgggcttccccacaaggctccaccctgaggtcttttaagctgtccttatgccagcctatttcttgttttttgggcctttttttttggagatagggtctcactctgtcgcccaggctggagtgcaatgacgcaatcttggcttattgcagtctcgacctcctgggctcaagagatccttccacctcagccacctgagtagcttggactacaggtgtgcaccacctctcccagttaatttttgtatttttagtagagacagagttatgccatgttactcaggctggtcttgaactcctggactcaagcgatcagcctgccttagcctcccaaagtgcaggggttacaggcttgagccattgcgcctgacctatttctggttcttagggccctggatgttaggatggatttctgaattaataataataataaaaccctcatcaaga

SEQ ID NO: 154 - DNA, 7423 bp, synthetic construct
  source 1..7423: mol_type = other DNA
gcgctcccgcccgccgctctccgtggccccgccgcgctgccgccgccgccgctgccagcgaaggtgccggggctccgggccctccctgccggcggccgtcagcgctcggagcgggctgcgcggcgggagctccgggaggcggccgtagccagcgccgccgcgcaggaccaggaggaggagaaagggtgcgcagcccggaggcggggtgcgccggtggggtgcagcggaagagggggtccaggggggagaacttcgtagcagtcatcctttttaggaaaagagggaaaaaataaaaccctcccccaccacctccttctccccacccctcgccgcaccacacacagcgcgggcttctagcgctcggcaccggcgggccaggcgcgtcctgccttcatttatccagcagcttttcggaaaatgcatttgctgttcggagtttaatcagaagaggattcctgcctccgtccccggctccttcatcgtcccctctcccctgtctctctcctggggaggcgtgaagcggtcccgtggatagagattcatgcctgtgcccgcgcgtgtgtgcgcgcgtgtaaattgccgagaaggggaaaacatcacaggacttctgcgaataccggactgaaaattgtaattcatctgccgccgccgctgccttttttttttctcgagctcttgagatctccggttgggattcctgcggattgacatttctgtgaagcagaagtctgggaatcgatctggaaatcctcctaatttttactccctctccccgcgactcctgattcattgggaagtttcaaatcagctataactggagagtgctgaagattgatgggatcgttgccttatgcatttgttttggttttacaaaaaggaaacttgacagaggatcatgctgtacttaaaaaatacaagtaagttctctgcacaggaaattggtttaatgtaactttcaatggaaacctttgagattttttacttaaagtgcattcgagtaaatttaatttccaggcagcttaatacattctttttagccgtgttacttgtagtgtgtatgccctgctttcactcagtgtgtacagggaaacgcacctgattttttacttattagtttgttttttctttaacctttcagcatcacagaggaagtagactgatattaacaatacttactaataataacgtgcctcatgaaataaagatccgaaaggaattggaataaaaatttcctgcatctcatgccaagggggaaacaccagaatcaagtgttccgcgtgattgaagacaccccctcgtccaagaatgcaaagcacatccaataaaatagctggattataactcctcttctttctctgggggccgtggggtgggagctggggcgagaggtgccgttggcccccgttgcttttcctctgggaaggatggcgcacgctgggagaacagggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgggccacaagtgaagtcaacatgcctgccccaaacaaatatgcaaaaggttcactaaagcagtagaaataatatgcattgtcagtgatgtaccatgaaacaaagctgcaggctgtttaagaaaaaataacacacatataaacatcacacacacagacagacacacacacacacaacaattaacagtcttcaggcaaaacgtcgaatcagctatttactgccaaagggaaatatcatttattttttacattattaagaaaaaaagatttatttatttaagacagtcccatcaaaactcctgtctttggaaatccgaccactaattgccaagcaccgcttcgtgtggctccacctggatgttctgtgcctgtaaacatagattcgctttccatgttgttggccggatcaccatctgaagagcagacggatggaaaaaggacctgatcattggggaagctggctttctggctgctggaggctggggagaaggtgttcattcacttgcatttctttgccctgggggctgtgatattaacagagggagggttcctgtggggggaagtccatgcctccctggcctgaagaagagactctttgcatatgactcacatgatgcatacctggtgggaggaaaagagttgggaacttcagatggacctagtacccactgagatttccacgccgaaggacagcgatgggaaaaatgcccttaaatcataggaaagtatttttttaagctaccaattgtgccgagaaaagcattttagcaatttatacaatatcatccagtaccttaagccctgattgtgtatattcatatattttggatacgcaccccccaactcccaatactggctctgtctgagtaagaaacagaatcctctggaacttgaggaagtgaacatttcggtgacttccgcatcaggaaggctagagttacccagagcatcaggccgccacaagtgcctgcttttaggagaccgaagtccgcagaacctgcctgtgtcccagcttggaggcctggtcctggaactgagccggggccctcactggcctcctccagggatgatcaacagggcagtgtggtctccgaatgtctggaagctgatggagctcagaattccactgtcaagaaagagcagtagaggggtgtggctgggcctgtcaccctggggccctccaggtaggcccgttttcacgtggagcatgggagccacgacccttcttaagacatgtatcactgtagagggaaggaacagaggccctgggcccttcctatcagaaggacatggtgaaggctgggaacgtgaggagaggcaatggccacggcccattttggctgtagcacatggcacgttggctgtgtggccttggcccacctgtgagtttaaagcaaggctttaaatgactttggagagggtcacaaatcctaaaagaagcattgaagtgaggtgtcatggattaattgacccctgtctatggaattacatgtaaaacattatcttgtcactgtagtttggttttatttgaaaacctgacaaaaaaaaagttccaggtgtggaatatgggggttatctgtacatcctggggcattaaaaaaaaaatcaatggtggggaactataaagaagtaacaaaagaagtgacatcttcagcaaataaactaggaaatttttttttcttccagtttagaatcagccttgaaacattgatggaataactctgtggcattattgcattatataccatttatctgtattaactttggaatgtactctgttcaatgtttaatgctgtggttgatatttcgaaagctgctttaaaaaaatacatgcatctcagcgtttttttgtttttaattgtatttagttatggcctatacactatttgtgagcaaaggtgatcgttttctgtttgagatttttatctcttgattcttcaaaagcattctgagaaggtgagataagccctgagtctcagctacctaagaaaaacctggatgtcactggccactgaggagctttgtttcaaccaagtcatgtgcatttccacgtcaacagaattgtttattgtgacagttatatctgttgtccctttgaccttgtttcttgaaggtttcctcgtccctgggcaattccgcatttaattcatggtattcaggattacatgcatgtttggttaaacccatgagattcattcagttaaaaatccagatggcaaatgaccagcagattcaaatctatggtggtttgacctttagagagttgctttacgtggcctgtttcaacacagacccacccagagccctcctgccctccttccgcgggggctttctcatggctgtccttcagggtcttcctgaaatgcagtggtgcttacgctccaccaagaaagcaggaaacctgtggtatgaagccagacctccccggcgggcctcagggaacagaatgatcagacctttgaatgattctaatttttaagcaaaatattattttatgaaaggtttacattgtcaaagtgatgaatatggaatatccaatcctgtgctgctatcctgccaaaatcattttaatggagtcagtttgcagtatgctccacgtggtaagatcctccaagctgctttagaagtaacaatgaagaacgtggacgtttttaatataaagcctgttttgtcttttgttgttgttcaaacgggattcacagagtatttgaaaaatgtatatatattaagaggtcacgggggctaattgctggctggctgccttttgctgtggggttttgttacctggttttaataacagtaaatgtgcccagcctcttggccccagaactgtacagtattgtggctgcacttgctctaagagtagttgatgttgcattttccttattgttaaaaacatgttagaagcaatgaatgtatataaaagcctcaactagtcatttttttctcctcttcttttttttcattatatctaattattttgcagttgggcaacagagaaccatccctattttgtattgaagagggattcacatctgcatcttaactgctctttatgaatgaaaaaacagtcctctgtatgtactcctctttacactggccagggtcagagttaaatagagtatatgcactttccaaattggggacaagggctctaaaaaaagccccaaaaggagaagaacatctgagaacctcctcggccctcccagtccctcgctgcacaaatactccgcaagagaggccagaatgacagctgacagggtctatggccatcgggtcgtctccgaagatttggcaggggcagaaaactctggcaggcttaagatttggaataaagtcacagaattaaggaagcacctcaatttagttcaaacaagacgccaacattctctccacagctcacttacctctctgtgttcagatgtggccttccatttatatgtgatctttgttttattagtaaatgcttatcatctaaagatgtagctctggcccagtgggaaaaattaggaagtgattataaatcgagaggagttataataatcaagattaaatgtaaataatcagggcaatcccaacacatgtctagctttcacctccaggatctattgagtgaacagaattgcaaatagtctctatttgtaattgaacttatcctaaaacaaatagtttataaatgtgaacttaaactctaattaattccaactgtacttttaaggcagtggctgtttttagactttcttatcacttatagttagtaatgtacacctactctatcagagaaaaacaggaaaggctcgaaatacaagccattctaaggaaattagggagtcagttgaaattctattctgatcttattctgtggtgtcttttgcagcccagacaaatgtggttacacactttttaagaaatacaattctacattgtcaagcttatgaaggttccaatcagatctttattgttattcaatttggatctttcagggattttttttttaaattattatgggacaaaggacatttgttggaggggtgggagggaggaagaatttttaaatgtaaaacattcccaagtttggatcagggagttggaagttttcagaataaccagaactaagggtatgaaggacctgtattggggtcgatgtgatgcctctgcgaagaaccttgtgtgacaaatgagaaacattttgaagtttgtggtacgacctttagattccagagacatcagcatggctcaaagtgcagctccgtttggcagtgcaatggtataaatttcaagctggatatgtctaatgggtatttaaacaataaatgtgcagttttaactaacaggatatttaatgacaaccttctggttggtagggacatctgtttctaaatgtttattatgtacaatacagaaaaaaattttataaaattaagcaatgtgaaactgaattggagagtgataatacaagtcctttagtcttacccagtgaatcattctgttccatgtctttggacaaccatgaccttggacaatcatgaaatatgcatctcactggatgcaaagaaaatcagatggagcatgaatggtactgtaccggttcatctggactgccccagaaaaataacttcaagcaaacatcctatcaacaacaaggttgttctgcataccaagctgagcacagaagatgggaacactggtggaggatggaaaggctcgctcaatcaagaaaattctgagactattaataaataagactgtagtgtagatactgagtaaatccatgcacctaaaccttttggaaaatctgccgtgggccctccagatagctcatttcattaagtttttccctccaaggtagaatttgcaagagtgacagtggattgcatttcttttggggaagctttcttttggtggttttgtttattataccttcttaagttttcaaccaaggtttgcttttgttttgagttactggggttatttttgttttaaataaaaataagtgtacaataagtgtttttgtattgaaagcttttgttatcaagattttcatacttttaccttccatggctctttttaagattgatacttttaagaggtggctgatattctgcaacactgtacacataaaaaatacggtaaggatactttacatggttaaggtaaagtaagtctccagttggccaccattagctataatggcactttgtttgtgttgttggaaaaagtcacattgccattaaactttccttgtctgtctagttaatattgtgaagaaaaataaagtacagtgtgagatactg

SEQ ID NO: 155 - DNA, 2326 bp, synthetic construct
  source 1..2326: mol_type = other DNA
cttccttggagccccgccgcccgccgctcactctgcacacgcagcagaagggacgtggtgttccccaggctctggccccccaggacctgcgcggatctggcccagggcgcctcgccgacttccgtaaactgggcggagggatgaaccccgacccaggggacggaggcgctcgccctctcgctgcagggttctgccctcaacacttctggccccgcctgtgaatgggggccggagcgatggggcggggccggcctccctccctcctcccaggctgacctctgccctccttcgagcacttcccgttcggggtgatgattgaagaactgagtgtggacagagcacctgtcctcgccagcgcccagctggaagctgagttatttgtggctgcagcattccaacgcacacaccaccagaaatccccttcagcatactggcctcagacctaccagggcagagaccttgtcttgtttgctgctagaacccaggatcgctggaaaatcacctagcagcagccaggtgcggtgactcacgcctgtaatcccagcactttggaaggccaaggcgggtggatagcaaggtcaagagatcgagaccagcctggccaacatgctctgggcacctccagctcctgctcgccggacggctcccagggagagcagacgcgccagacgcgccaccctcggggcgccgacggtcacggagcatggggtcggcctttgagcgggtagtccggagagtggtccaggagctggaccatggtggggagttcatccctgtgaccagcctgcagagctccactggcttccagccctactgcctggtggttaggaagccctcaagctcatggttctggaaaccccgttataagtgtgtcaacctgtctatcaaggacatcctggagccggatgccgcggaaccagacgtgcagcgtggcaggagcttccacttctacgatgccatggatgggcagatacagggcagcgtggagctggcagccccaggacaggcaaagatcgcaggcggggccgcggtgtctgacagctccagcacctcaatgaatgtgtactcgctgagtgtggaccctaacacctggcagactctgctccatgagaggcacctgcggcagccagaacacaaagtcctgcagcagctgcgcagccgcggggacaacgtgtacgtggtgactgaggtgctgcagacacagaaggaggtggaagtcacgcgcacccacaagcgggagggctcgggccggttttccctgcccggagccacgtgcttgcagggtgagggccagggccatctgagccagaagaagacggtcaccatcccctcaggcagcaccctcgcattccgggtggcccagctggttattgactctgacttggacgtccttctcttcccggataagaagcagaggaccttccagccacccgcgacaggccacaagcgttccacgagcgaaggcgcctggccacagctgccctctggcctctccatgatgaggtgcctccacaacttcctgacagatggggtccctgcggagggggcgttcactgaagacttccagggcctacgggcagaggtggagaccatctccaaggaactggagcttttggacagagagctgtgccagctgctgctggagggcctggagggggtgctgcgggaccagctggccctgcgagccttggaggaggcgctggagcagggccagagccttgggccggtggagcccctggacggtccagcaggtgctgtcctggagtgcctggtgttgtcctccggaatgctggtgccggaactcgctatccctgttgtctacctgctgggggcactgaccatgctgagtgaaacgcagcacaagctgctggcggaggcgctggagtcgcagaccctgttggggccgctcgagctggtgggcagcctcttggagcagagtgccccgtggcaggagcgcagcaccatgtccctgccccccgggctcctggggaacagctggggcgaaggagcaccggcctgggtcttgctggacgagtgtggcctagagctgggggaggacactccccacgtgtgctgggagccgcaggcccagggccgcatgtgtgcactctacgcctccctggcactgctatcaggactgagccaggagccccactagcctgtgcccgggcatggcctggcagctctccagcagggcagagtgtttgcccaccagctgctagccctaggaaggccaggagcccagtagccatgtggccagtctaccatggggcccaggagttggggaaacacaataaaggtggcatacgaaggaaaaaaaaaaaaa

(19) United States
(12) Patent Application Publication
(10) Pub. No.: US 2025/0011765 A1
(43) Pub. Date: Jan. 9, 2025

(54) COMPOSITIONS AND SYSTEMS FOR RNA-PROGRAMABLE CELL EDITING AND METHODS OF MAKING AND USING SAME

(71) Applicants: Duke University, Durham, NC (US); Cold Spring Harbor Laboratory, Cold Spring Harbor, NY (US)

(72) Inventors: Z. Josh Huang, Durham, NC (US); Yongjun Qian, Durham, NC (US)

(21) Appl. No.: 18/649,873

(22) Filed: Apr. 29, 2024

Related U.S. Application Data
(63) Continuation-in-part of application No. PCT/US22/79008, filed on Oct. 31, 2022.
(60) Provisional application No. 63/273,343, filed on Oct. 29, 2021.
(60) Provisional application No. 63/343,669, filed on May 19, 2022.

(51) Int. Cl. C12N 15/11; A61P 25/08; A61P 25/28

(52) U.S. Cl. CPC C12N 15/11; A61P 25/08; A61P 25/28

(57) ABSTRACT

A readrRNA (RNA sensing by Endogenous ADAR) molecule is a modular RNA molecule that facilitates sensing and detection of a cell type or cell status of a cell, including a cell of a mammalian nervous system, such as a neuronal or non-neuronal cell of the mammalian central and/or peripheral nervous system, and/or facilitates delivery of an effector protein to the selected cell. A composition that includes such a modular RNA molecule and another nucleic acid (linked or unlinked to the modular RNA molecule) is a CellREADR (Cell access through RNA sensing by Endogenous ADAR). CellREADR senses the presence of a selected cell RNA in a cell of a mammalian nervous system via readrRNA and leverages RNA editing mediated by ADAR (adenosine deaminase acting on RNA) for coupling the detection of a cell-defining RNA with translation of one or more effector proteins in a cell of a mammalian nervous system.

### CROSS-REFERENCE TO RELATED APPLICATIONS

This U.S. application is a continuation-in-part application of international application No. PCT/US2022/079008, filed Oct. 31, 2022, which claims the benefit of U.S. provisional application No. 63/343,669 filed May 19, 2022, and U.S. provisional application No. 63/273,343 filed Oct. 29, 2021, the contents of each of which are incorporated herein in their entirety.

### STATEMENT REGARDING FEDERALLY SPONSORED RESEARCH OR DEVELOPMENT

This invention was made with Government support under Federal Grant nos. U19MH114821-03 and DP1MH129954 awarded by the National Institute of Health. The Federal Government has certain rights to this invention.

### SEQUENCE LISTING

The instant application contains a Sequence Listing which has been submitted as a WIPO Standard ST.26 XML file via Patent Center. Said XML file, created on Apr. 29, 2024, is entitled “123658-10930.xml” and is 193,913 bytes in size. The sequence listing is incorporated herein by reference.

### BACKGROUND

The diversity of cell types underlies the diversity of life forms and of physiological systems within individual organisms [1]. Cell types defined by their distinct gene expression profiles, morphological and physiological properties, and tissue function are the intermediates through which genetic information shapes an organism's phenotypes and behaviors. All bodily functions emerge from interactions among cell types, and aberrant cell type physiology and function give rise to myriad diseases [2]. Recent high-throughput single-cell genomic approaches promise to identify all molecularly-defined major cell types in the human body and in many other organisms [3][4]. Beyond molecular profiling, it is necessary to monitor and manipulate each and every cell type in order to identify their specific roles in tissue architecture and system function across levels of biological organization.

In the context of cell type access and manipulation in multicellular organisms, most if not all genetic approaches to date attempt to recapitulate cell type specific mRNA expression by engineering DNA regulatory elements in the genome either through the germline or in somatic cells, or by using transcriptional enhancer-based viral vectors. To date, almost all genetic approaches to cell types rely on DNA-based transcription regulatory elements for expressing tool-genes (e.g. markers, sensors, effectors) that mimic certain cell-specific RNA expression, mostly through germline engineering in only a handful of organisms [5-7]. All germline approaches, including CRISPR [8-10], are inherently cumbersome, slow, difficult to scale and generalize, and raise ethical issues especially in primates and humans [11, 12]. Recently, transcriptional enhancer based viral vectors show promise for targeting cell types in animals [13-19]; but such enhancers are difficult to identify and validate, often requiring large scale effort [13] due to their complex relationship to target genes and cell types. Somatic cell engineering, usually by CRISPR/Cas9 technology, is currently of low efficiency and tend to include unintended off-targets. The enhancer viral vector approach also has major limitations rooted in its complex relationships to gene expression and cell types; it requires large-scale effort, special expertise, extensive validation, and is unlikely to be truly scalable, user friendly, and generalizable across species. Thus, though transcriptional enhancer-based viral vectors show promise for targeting somatic cell types in animals; such enhancers are difficult to identify and validate, often requiring large-scale effort, due to their complex relationship to target genes and cell types. In essence, all DNA- and transcription-based approaches are inherently indirect in attempting to mimic and leverage cell type specific RNA expression patterns.

### SUMMARY OF THE INVENTION

The present disclosure is based, in part, on the discovery of a new class of cell type technology that bypasses DNA-based transcriptional process and directly engages cell type-defining RNAs. In some embodiments, this novel technology (termed CellREADR: Cell access through RNA sensing by Endogenous ADAR [adenosine deaminase acting on RNA]), which harnesses an RNA sensing and editing mechanism ubiquitous to all animal cells for detecting the presence of cellular RNAs and switching on the translation of an effector protein to monitor and manipulate the cell type. The compositions and methods provided herein are deployable as a single RNA molecule operating through Watson-Crick base pairing that targets a specific cell type by virtue of the presence of a target RNA. Therefore, CellREADR is inherently specific, easy to build and use, scalable, programmable, and general across species.

Accordingly, the present disclosure provides a modular RNA molecule, comprising: (i) a 5′ region comprising a sensor domain comprising a stretch of consecutive nucleotides that is complementary to a corresponding stretch of consecutive nucleotides of a cellular RNA of a cell of the mammalian central nervous system, where the sensor domain comprises a stop codon editable by ADAR; and (ii) a 3′ region comprising a domain encoding a protein, where the protein coding domain is downstream of and in-frame with the sensor domain, where, upon introduction of the modular RNA into the cell of the mammalian central nervous system comprising an Adar enzyme, the stretch of consecutive nucleotides of the sensor domain and the corresponding nucleotide stretch of the cellular RNA form an RNA duplex comprising the stop codon, where the stop codon comprised in the RNA duplex is edited by ADAR in the cell, thereby to permit translation of the protein.

Also disclosed herein is a modular RNA molecule comprising: (i) a 5′ region comprising a sensor domain comprising a stretch of consecutive nucleotides that is complementary to a corresponding stretch of consecutive nucleotides of a cellular RNA of a cell of a mammalian peripheral nervous system, where the sensor domain comprises a stop codon editable by ADAR; and (ii) a 3′ region comprising a domain encoding a protein, where the protein coding domain is downstream of and in-frame with the sensor domain, where, upon introduction of the modular RNA into the cell of the mammalian peripheral nervous system comprising an Adar enzyme, the stretch of consecutive nucleotides of the sensor domain and the corresponding nucleotide stretch of the cellular RNA form an RNA duplex comprising the stop codon, where the stop codon comprised in the RNA duplex is edited by ADAR in the cell, thereby to permit translation of the protein. In the modular RNA molecule, the stretch of consecutive nucleotides of the sensor domain is able to form an RNA duplex with at least a portion of an mRNA. In the modular RNA molecule, the protein coding region encodes an effector protein. In one aspect of the modular RNA molecule, it further encodes a self-cleaving 2A peptide positioned between the sensor domain and effector RNA region. In one aspect of the modular RNA molecule, the self-cleaving 2A peptide is selected from the group consisting of one or more of T2A peptide, P2A peptide, E2A peptide, and F2A peptide. In one aspect of the modular RNA molecule, the efRNA-encoded protein comprises a protein selected from the group of a label and a transcriptional activator or repressor.

Also disclosed herein is a composition comprising (i) a first nucleic acid comprising a modular RNA molecule comprising: (a) a sensor domain comprising a stretch of consecutive nucleotides that is complementary to a corresponding stretch of consecutive nucleotides of cellular RNA of a mammalian cell of the central nervous system, where the sensor domain comprises a stop codon editable by ADAR; and (b) a first protein-coding domain encoding an effector protein, where the first protein-coding region is downstream of and in-frame with the sensor domain, and (ii) a second nucleic acid comprising a second protein coding domain. Also disclosed herein is a composition comprising (i) a first nucleic acid comprising a modular RNA molecule comprising: (a) a sensor domain comprising a stretch of consecutive nucleotides that is complementary to a corresponding stretch of consecutive nucleotides of cellular RNA of a mammalian cell of the peripheral nervous system, where the sensor domain comprises a stop codon editable by ADAR; and (b) a first protein-coding domain encoding an effector protein, where the first protein-coding region is downstream of and in-frame with the sensor domain, and (ii) a second nucleic acid comprising a second protein coding domain. In one aspect of the composition, the first and second nucleic acids comprise a single nucleic acid molecule. In one aspect of the composition, where the first and second nucleic acids comprise two nucleic acid molecules. In one aspect of the composition, the first and second nucleic acid are covalently linked. In one aspect of the composition, the second protein coding domain is comprised within a gene comprising a transcription control element, optionally, a promoter. In one aspect of the composition, the effector protein binds to the gene. In one aspect of the composition, expression of the second protein coding domain is modulated by the effector protein. In one aspect of the composition, the first protein-coding domain encodes a transcriptional activator or transcriptional repressor. In one aspect of the composition, the first protein-coding domain encodes a DNA recombinase. In one aspect of the composition, the brain cell comprises a mammalian cortex cell. In one aspect of the composition, the brain cell comprises a mammalian schwann cell or a mammalian satellite cell of the peripheral nervous system. In one aspect of the composition, the first protein-coding domain encodes an effector protein comprises the tetracycline dependent transcription activator tTA2, and where the stretch of consecutive nucleotides of the sensor domain is complementary to a stretch of consecutive nucleotides of a selected cellular RNA of a brain cortex cell encoded by the Fezf2 gene, and where the second nucleic acid comprises a second protein coding region under the transcriptional control of a TRE-3G eukaryotic inducible promoter. In one aspect of the composition, the first protein-coding domain encodes an effector protein comprises the tetracycline dependent transcription activator tTA2, and where the stretch of consecutive nucleotides of the sensor domain is complementary to a stretch of consecutive nucleotides of a selected cellular RNA of a peripheral CNS cell, and where the second nucleic acid comprises a second protein coding region under the transcriptional control of a TRE-3G eukaryotic inducible promoter. In one aspect of the composition, the first protein-coding region encodes a cell killing protein selected from the group consisting of: thymidine kinase (TK) and cytosine deaminase (CD), a programmed cell death protein selected from the group consisting of CASP3, CASP9, BCL, GSDME, GSDMD, GZMA and GZMB, a neural activating protein selected from the group consisting of: ChR2 and DREADD-M3Dq, an immunity enhancer protein selected from the group consisting of IFNB, IFNG, TNFA, IL2, IL12, IL15 and CD40L, a physiological editing protein selected from the group consisting of NaChBac and Kir2.1, a protein synthesis inhibition protein including ricin, a neural inhibitor protein selected from the group consisting of DREADD-hM4D, NpHR and GtACR1, a protein involved in neural cell fate such as NEUROD, a protein involved with cell regeneration including Epidermal growth factors.

Also disclosed herein, is a nucleic acid delivery vehicle comprising the modular RNA molecule described herein or the composition described herein or DNA encoding the modular RNA molecule described herein. In one aspect, the nucleic acid delivery vehicle can be a nanoparticle, a liposome, a vector, an exosome, a micro-vesicle, a gene-gun, and a Selective Endogenous encapsulation for cellular Delivery (SEND) system. In one aspect, the nucleic acid delivery vehicle comprises a viral vector, where the viral vector includes but is not limited to consisting of adeno-associated virus (AAV), adenovirus, retrovirus, lentivirus, herpes virus, vesicular stomatitis virus.

Also disclosed herein, is a modular RNA molecule, where the modular RNA molecule is encoded by a DNA vector. Also disclosed herein, is a composition where the modular RNA molecule is encoded by a DNA vector. Also disclosed herein, is a delivery vehicle, where the delivery vehicle comprises a modular RNA molecule is encoded by a DNA vector. Also disclosed herein, is a pharmaceutical composition comprising a pharmaceutically acceptable carrier, excipient and/or diluent together with the modular RNA molecule described herein, and/or the composition comprising the modular RNA molecule described herein, and/or the delivery vehicle described herein. Also disclosed herein, is a cell comprising the modular RNA molecule described herein, and/or the composition comprising the modular RNA molecule described herein, and/or the delivery vehicle described herein. Also, the cell disclosed herein, is a mammalian cell. Also disclosed herein, is a kit comprising the modular RNA molecule described herein, and/or the composition comprising the modular RNA molecule described herein, and/or the delivery vehicle described herein, and packaging therefore.

Also disclosed herein, is a method of introducing a modular RNA or a nucleic acid composition encoding the modular RNA, or a delivery vehicle comprising the modular RNA or the nucleic acid composition encoding the modular RNA, into a selected cell of a mammal, the method comprising: contacting a cell of the mammal with the modular RNA molecule, the nucleic acid composition encoding the modular RNA, or the delivery vehicle comprising the modular RNA or the nucleic acid composition encoding the modular RNA, under conditions which permit the cell of the mammal to comprise the modular RNA or the delivery vehicle comprising the modular RNA or the nucleic acid composition encoding the modular RNA, described herein.

Also disclosed herein, is a method of eliciting ADAR editing of a stop codon to allow expression of an in-frame, downstream encoded protein in a cell of a mammal comprising a selected target cellular RNA, the method comprising introducing into the cell of the mammal a modular RNA molecule under conditions in which the modular RNA is comprised in the cell, the modular RNA comprising (i) a 5′ region comprising a sensor domain comprising a stretch of consecutive nucleotides that is complementary to a corresponding stretch of consecutive nucleotides of the selected cellular RNA, where the sensor domain comprises a stop codon editable by ADAR; and (ii) a 3′ region comprising a domain encoding an effector protein, where the protein coding region is downstream of and in-frame with the sensor domain, where upon introduction of the modular RNA molecule to the mammal, an RNA duplex is formed in the cell between the stretch of consecutive nucleotides of the sensor domain and the corresponding stretch of consecutive nucleotides of the cellular RNA, where the RNA duplex comprises the ADAR-editable stop codon, and where ADAR edits the stop codon comprised in the RNA duplex, thereby permitting translation of the protein, and where the protein is produced in the mammal.

Also disclosed herein, is a method for treating a disease or disorder in a mammal, the method comprising: providing an agent, the agent comprising a modular RNA molecule, a composition comprising a modular RNA molecule, and/or a nucleic acid composition, and/or a delivery vehicle all as described herein; and administering the agent to the mammal in a therapeutically effective amount to permit translation of the effector protein in selected cells of the mammal, thereby to produce the protein in the cells, where production of the protein in the cells provides for treatment of the disease or disorder in the mammal. In an aspect of the method, the agent comprises the composition comprising the agent comprising a modular RNA molecule, and/or a nucleic acid composition, and/or a delivery vehicle described herein, is administered and the first protein coding region encoding the effector protein comprised in the agent encodes a transactivator protein that activates expression of the second protein coding region, and where expression of the second protein coding region in the selected cells is therapeutically effective in treating the disease or disorder.

Also disclosed herein, is a method for stimulating L5b/L6 corticofugal projection neurons (CFPN) signals in the forelimb somatosensory cortex of a mouse as measured by photometry in response to mechanical stimulation of the forepaw, comprising administering the mice with the composition where the sensor domain comprises a stretch of consecutive nucleotides that is complementary to a corresponding stretch of consecutive nucleotides of cellular RNA encoding Ctip2, where the first protein-coding domain encodes an effector protein comprises the tetracycline dependent transcription activator tTA2, and where the second nucleic acid comprises a gene encoding the calcium indicator GCaMP6s under the transcriptional control of a TRE-3G eukaryotic inducible promoter.

Also disclosed herein, is a method for stimulating forelimb movement in response to light stimulation of the caudal forelimb areas (CFA) of mice comprising administering the mice with the composition where the sensor domain comprises a stretch of consecutive nucleotides that is complementary to a corresponding stretch of consecutive nucleotides of cellular RNA encoding Ctip2, where the first protein-coding domain encodes an effector protein comprises the tetracycline dependent transcription activator tTA2, and where the second nucleic acid comprises a gene encoding the light-activated ion channel channelrhodopsin-2 under the transcriptional control of a TRE-3G eukaryotic inducible promoter. In one aspect of the method, a second gene is operably linked to the TRE-3G eukaryotic inducible promoter, the second gene encoding enhanced yellow fluorescent protein (eYFP).

Also described herein, is a pharmaceutical composition comprising an agent comprising a modular RNA molecule, a composition comprising a modular RNA molecule, and/or a nucleic acid composition, and/or a delivery vehicle all as described herein and a pharmaceutically acceptable carrier, excipient and/or diluent. Also described herein is a cell comprising a modular RNA molecule, a composition comprising a modular RNA molecule, and/or a nucleic acid composition, and/or a delivery vehicle all as described herein. Also described herein is a kit comprising a modular RNA molecule, a composition comprising a modular RNA molecule, and/or a nucleic acid composition, and/or a delivery vehicle all as described herein.

Also described herein, is a method of identifying GABAergic neurons in mammalian cortex in vivo, comprising targeting vesicular GABA transporter (vGAT) mRNA in mammalian cortex in vivo, the method comprising administering into the S1 barrel cortex and hippocampus of the mammal a composition comprising modular RNA molecule, and/or a nucleic acid composition, and/or a delivery vehicle all as described herein, where the stretch of consecutive nucleotides of the sensor domain is complementary to a stretch of consecutive nucleotides of a selected cellular RNA of a cortex cell encoded by the vGAT gene, and where the first protein-coding domain encodes an effector protein comprising the tetracycline dependent transcription activator tTA2 and a second effector protein comprising the gene encoding smV5, and where the second nucleic acid comprises a coding region for green fluorescent protein under the transcriptional control of a TRE-3G eukaryotic inducible promoter, where the first nucleic acid comprising the modular RNA molecule is under the trantriptional control of the hSyn promoter and where the modular RNA molecule comprises the gene encoding mCherry upstream of the sensor domain and under the trantriptional control of the hSyn promoter, where upon the administration, vGAT mRNA in the S1 barrel cortex and hippocampus of the mammal is co-labeled with magenta and GFP, indicating identification of GABAergic neurons in the mammalian cortex. In one aspect of the method, the sesRNA targeting vGAT mRNA is complementary to all or part of exon 1 of vGAT mRNA.

Also described herein, is a method of identifying GABAergic neurons in mammalian cortex in vivo, comprising targeting Transducin-like enhancer protein 4 (Tle4) mRNA, the method comprising administering into the S1 barrel cortex of the mammal a composition comprising modular RNA molecule, and/or a nucleic acid composition, and/or a delivery vehicle all as described herein, where the stretch of consecutive nucleotides of the sensor domain is complementary to a stretch of consecutive nucleotides of a selected cellular RNA of a cortex cell encoded by the Tle4 gene, and where the first protein-coding domain encodes an effector protein comprising the tetracycline dependent transcription activator tTA2 and a second effector protein comprising the gene encoding smFlag, and where the second nucleic acid comprises a coding region for green fluorescent protein under the transcriptional control of a TRE-3G eukaryotic inducible promoter, where the first nucleic acid comprising the modular RNA molecule is under the trantriptional control of the hSyn promoter and where the modular RNA molecule comprises the gene encoding mCherry upstream of the sensor domain and under the trantriptional control of the hSyn promoter, where upon the administration, Tle4 mRNA in the S1 barrel cortex of the mammal is co-labeled with magenta and GFP, indicating identification of TLE4 positive projection neurons in the mammalian cortex. In one aspect of the method, the sesRNA targeting Tle4 mRNA is complementary to all or part of exon 15 of Tle4 mRNA.

Also described herein is a method of determining in an organotypic culture platform cortical layers which express human FOXP2, the method comprising administering to the culture the a composition comprising modular RNA molecule, and/or a nucleic acid composition, and/or a delivery vehicle all as described herein, where the stretch of consecutive nucleotides of the sensor domain is complementary to a stretch of consecutive nucleotides of a selected cellular RNA of a cortex cell encoded by the forkhead box protein P2 (FOXP2) gene, and where the first protein-coding domain encodes an effector protein comprising the tetracycline dependent transcription activator tTA2 and a second effector protein comprising the gene encoding smV5, and where the second nucleic acid comprises a coding region for green/yellow fluorescent protein (mNeon) under the transcriptional control of a TRE-3G eukaryotic inducible promoter, where the first nucleic acid comprising the modular RNA molecule is under the trantriptional control of the hSyn promoter and where the modular RNA molecule comprises the gene encoding ClipF upstream of the sensor domain and under the trantriptional control of the hSyn promoter, where 5 days post administration, mNeon-labeled cell bodies observed in upper and deep layers of the organotypic culture platform cortical layers, indicating expression of human FOXP2 in the upper and deep layers.

Also described herein, is a method to suppress neural activities that promote focal epileptic seizure comprising targeting pathophysiological neural ensembles (PNE) comprising cell type targeted modulation of neural activity of PNE that participate in seizure activity, the method comprising: (i) providing a composition comprising modular RNA molecule, and/or a nucleic acid composition, and/or a delivery vehicle all as described herein, where the stretch of consecutive nucleotides of the sensor domain is complementary to a stretch of consecutive nucleotides of a selected cellular RNA of a cortex cell encoded by the human SST gene, and where the first protein-coding domain encodes an effector protein comprising the tetracycline dependent transcription activator tTA2 and a second effector protein comprising the gene encoding osmFlag, and where the second nucleic acid comprises a coding region for green fluorescent protein (GFB) under the transcriptional control of a TRE-3G eukaryotic inducible promoter, where the first nucleic acid comprising the modular RNA molecule is under the trantriptional control of the hSyn promoter and where the modular RNA molecule comprises the gene encoding mCherry upstream of the sensor domain and under the transcriptional control of the hSyn promoter, further providing a gene encoding a designer receptors exclusively activated by designor drugs (DREADD) or a gene encoding pharmacologically selective actuator module/pharmacologically selective effector molecule (PSAM), and (ii) administering the agent and the designer receptor gene to the mammal in a therapeutically effective amount to permit translation of the effector protein in selected cells of the mammal upon administration of an oral dose of olanzapine or clozapine-n-oxide which activates DREADD or an oral dose of varencline activates PSAM, thereby to produce the protein in the cells, where production of the protein in the cells provides for treatment of epilepsy in the mammal.

Also described herein is a method to suppress neural activities that promote focal epileptic seizure comprising targeting pathophysiological neural ensembles (PNE) comprising cell type targeted modulation of neural activity of PNE that participate in seizure activity, the method comprising (i) providing an agent, the agent comprising a composition comprising modular RNA molecule, and/or a nucleic acid composition, and/or a delivery vehicle all as described herein; and where the stretch of consecutive nucleotides of the sensor domain is complementary to a stretch of consecutive nucleotides of a selected cellular RNA of a cortex cell encoded by the human intermediate early growth cfos gene, and where the first protein-coding domain encodes an effector protein comprising the tetracycline dependent transcription activator tTA2 and a second effector protein comprising the gene encoding osmFlag, and where the second nucleic acid comprises a coding region for green fluorescent protein (GFB) under the transcriptional control of a TRE-3G eukaryotic inducible promoter, where the first nucleic acid comprising the modular RNA molecule is under the trantriptional control of the hSyn promoter and where the modular RNA molecule comprises the gene encoding mCherry upstream of the sensor domain and under the transcriptional control of the hSyn promoter, (ii) further providing a gene encoding a designer receptors exclusively activated by designor drugs (DREADD) or a gene encoding pharmacologically selective actuator module/pharmacologically selective effector molecule (PSAM), and (iii) administering the agent and the designer receptor gene to the mammal in a therapeutically effective amount to permit translation of the effector protein in selected cells of the mammal upon administration of an oral dose of olanzapine or clozapine-n-oxide which activates DREADD or an oral dose of varencline activates PSAM, thereby to produce the protein in the cells, where production of the protein in the cells provides for treatment of the disease or disorder in the mammal.

Also described herein is a method of treating chronic pain in a mammal, comprising (i) providing an agent, the agent comprising a composition comprising modular RNA molecule, and/or a nucleic acid composition, and/or a delivery vehicle all as described herein; where a first module of the modular RNA comprises the stretch of consecutive nucleotides of the sensor domain which is complementary to a stretch of consecutive nucleotides of selected cellular RNA encoded by either the human TRPV1 gene or the human NaV1.8 gene, and a the first protein-coding domain encoding an effector protein comprising the tetracycline dependent transcription activator tTA2, where the first module of the modular RNA molecule is under the transcriptional control of the hSyn promoter and further comprises a spacer upstream of the sensor domain, where the modular RNA further comprises a second module, where the second module is upstream of the first module, where the second module encodes shRNA for NaV1.7 or NaV1.8 and is under the transcriptional control of a TRE-3G eukaryotic inducible promoter, and (ii) administering the agent to the mammal in a therapeutically effective amount to permit translation of the effector protein in selected cells of the mammal, thereby to produce the protein in the cells, where production of the protein in the cells provides for treatment of chronic pain in the mammal.

Also described herein is a method of treating method of treating Alzheimer's Disease in a mammal, the method comprising, comprising (i) providing an agent, the agent comprising a composition comprising modular RNA molecule, and/or a nucleic acid composition, and/or a delivery vehicle all as described herein; where a first module of the modular RNA comprises the stretch of consecutive nucleotides of the sensor domain which is complementary to a stretch of consecutive nucleotides of selected cellular RNA encoded by encoded by one of group consisting of the human astrocyte marker genes of aldehyde dehydrogenase family 1 member L1 (Aldh1L1), glial fibrillary acidic protein (GFAP), excitatory amino acid transporter 1 (EAAT1), and microglial genes: C-X3-C Motif Chemokine Receptor 1 (CX3CR1), transmembrane protein 119 (TMEM119), Ionized calcium binding adaptor molecule 1 (IBA-1), and a the first protein-coding domain encoding an effector protein comprising the tetracycline dependent transcription activator tTA2, where the first module of the modular RNA molecule is under the transcriptional control of the hSyn promoter and further comprises a spacer upstream of the sensor domain, where the modular RNA further comprises a second module, where the second module is upstream of the first module, where the second module encodes shRNA for ApoE4 and is under the transcriptional control of a TRE-3G eukaryotic inducible promoter, and (ii) administering the agent to the mammal in a therapeutically effective amount to permit translation of the effector protein in selected cells of the mammal, thereby to produce the protein in the cells, where production of the protein in the cells provides for treatment of Alzheimer's disease in the mammal.

### BRIEF DESCRIPTION OF THE DRAWINGS

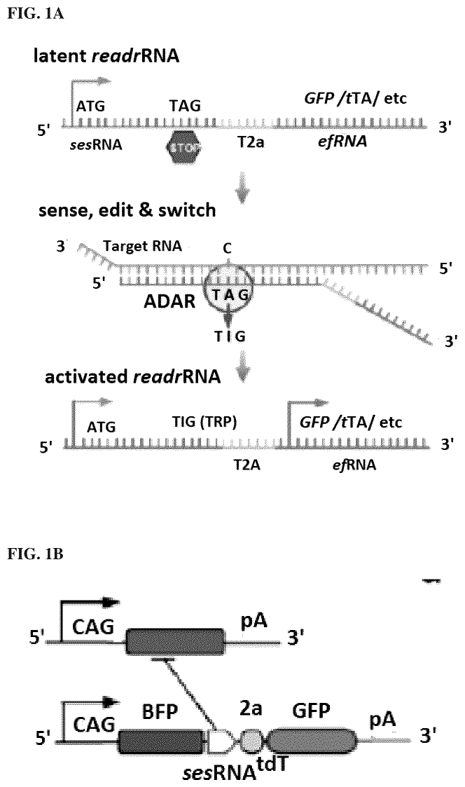

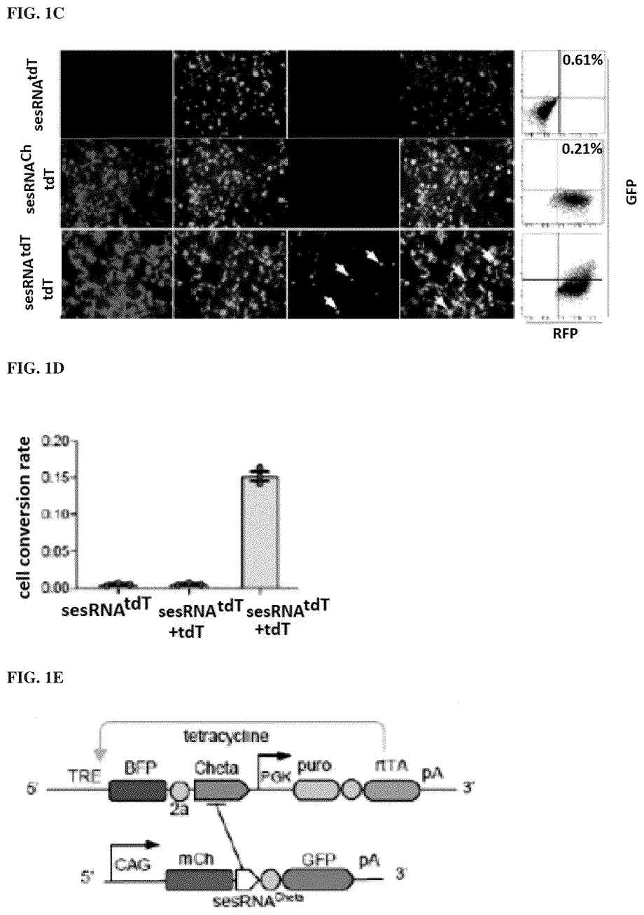

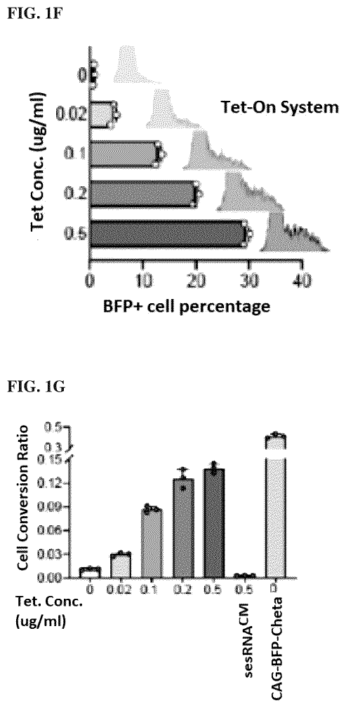

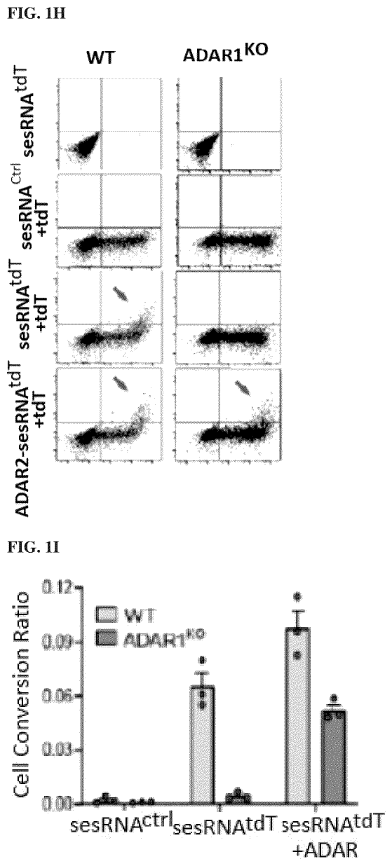

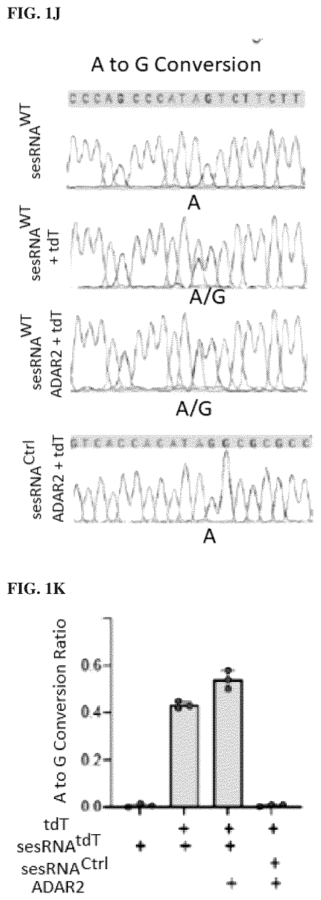

### FIG. 1A-1K: CellREADR Design and Implementation in Mammalian Cells

FIG. 1a, CellREADR is a single modular readrRNA molecule, consisting of a translationally in-frame 5′-sensor domain (sesRNA) and 3′-effector domain (efRNA), separated by a T2a coding region. sesRNA is complementary to a cellular RNA and contains an in-frame STOP codon that prevents efRNA translation. Base pairing between sesRNA and target RNA recruits ADARs, which mediate A->I editing and convert the UAG STOP to a UGG Trp codon, switching on translation of effector protein. FIGS. 1b-d, FIG. 1b, CAG-tdT (top) expresses the tdTomato target RNA from a CAG promoter and READR^{tdT-GFP} (bottom) expresses a readrRNA consisting of a BFP coding region followed by sesRNA^{tdT} and efRNA^{GFP}, driven by a CAG promotor. FIG. 1c, 293T cells transfected with both CAG-tdT and READR^{tdT-GFP} showed robust GFP expression co-localized with BFP and RFP (bottom). Cells transfected with READR^{tdT-GFP} only (top) or CAG-tdT and control READR ^{Ctrl} expressing a sesRNA^{Ctrl} with a scramble coding sequence containing a STOP codon (middle), showed almost no GFP expression. Representative FACS analysis of GFP and RFP expression is shown to the right. FIG. 1d, Quantification of CellREADR efficiency by FACS. FIGS. 1e-g, Effect of target RNA levels on CellREADR. FIG. 1e, Expression vectors for quantifying the effect of target RNA levels on CellREADR. In rtTA-TRE-ChETA (top), a BFP-ChETA fusion target RNA is transcribed from TRE, driven by constitutively expressed rtTA in a tetracycline concentration dependent manner. READR^{ChETA-GFP} (bottom) expresses readrRNA consisting of a mCherry coding region followed by sesRNA^{ChETA} and efRNA^{GFP}. FIG. 1f, in 293T cells co-transfected with rtTA-TRE-ChETA and READR^{ChETA-GFP}, increasing tetracycline concentrations in medium resulted in increased percentage of BFP⁺ in RFP⁺ cells revealed by FACS analysis. FIG. 1g, GFP⁺ to RFP⁺ ratio increased with increasing tetracycline concentration. CAG-ChETA constitutively expressing BFP-ChETA RNA served as a positive control, and READR ^{Ctrl} expressing sesRNA^{Ctrl} served as a negative control. FIGS. 1h-1i, ADAR1 is necessary for CellREDAR function. FACS analysis (FIG. 1h) of wild-type or ADAR1^{KO} 293T cells expressing sesRNA^{tdT} only, sesRNA^{Ctrl} and tdT, sesRNA^{tdT} and tdT, or sesRNA^{tdT} and tdT with ADAR2 over expression. Arrows indicate GFP⁺/RFP⁺ populations. I, Quantification of cell conversion ratio. FIG. 1j, Electropherograms of Sanger sequencing showing A-to-G conversion at the intended editing site in different samples as indicated. FIG. 1k, Bar graph shows the quantification of the A-to-G conversion rate at the targeted editing site. Error bars in FIGS. 1d, 1f, 1g, and 1k are mean values±s.e.m. n=3; n represents the number of independent experiments performed in parallel.

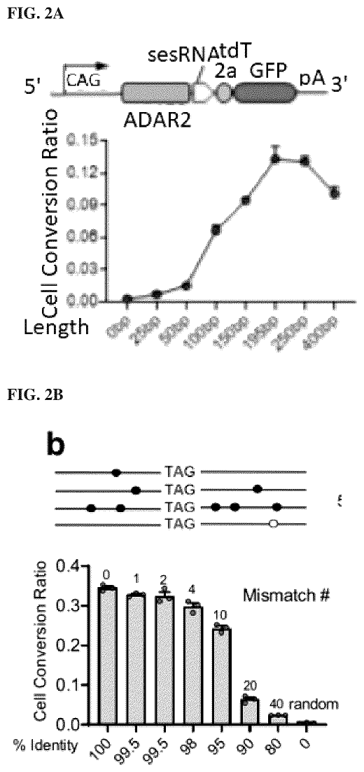

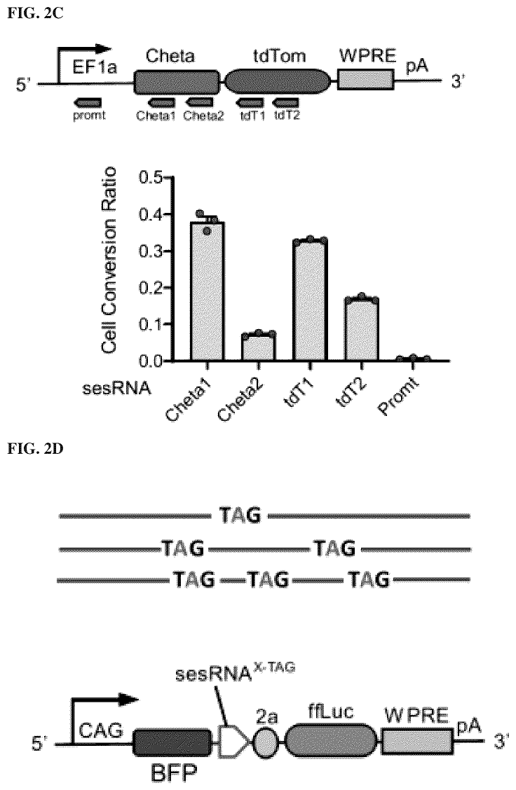

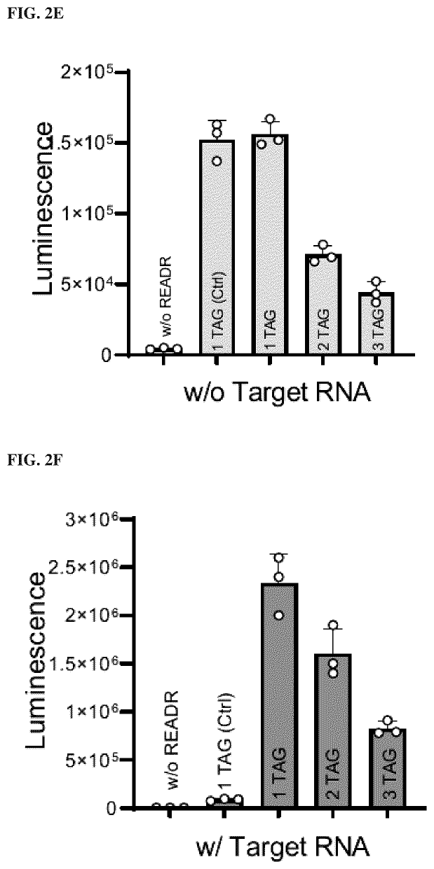

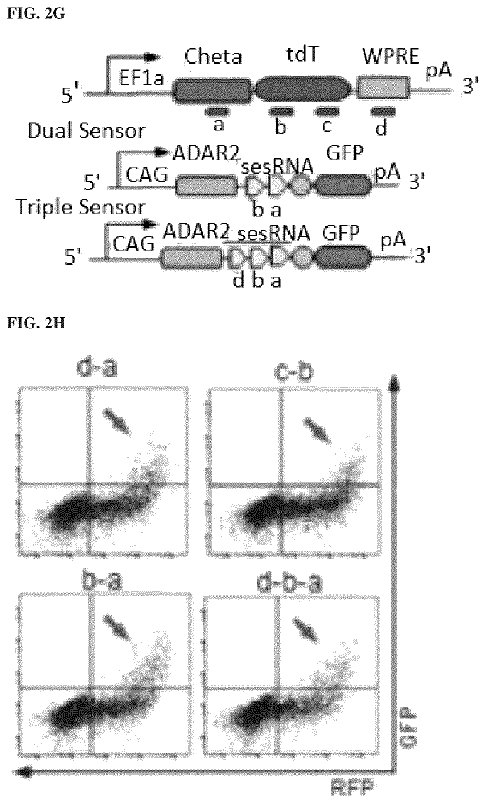

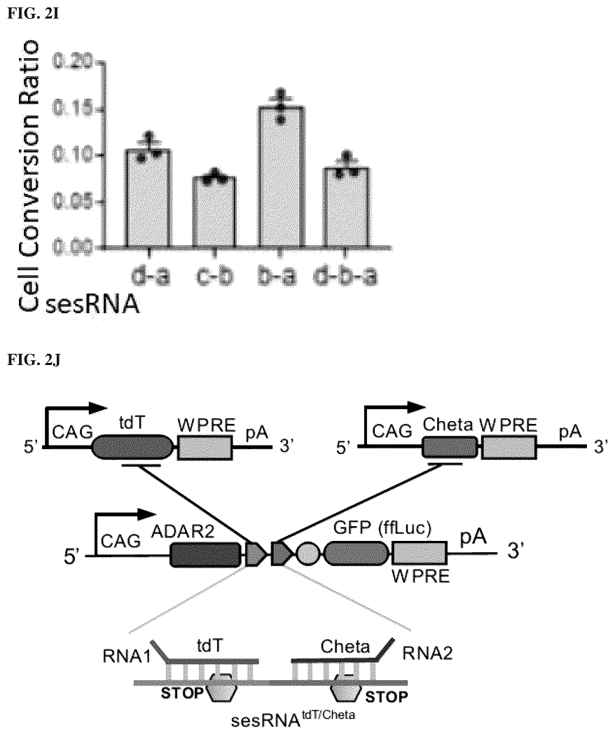

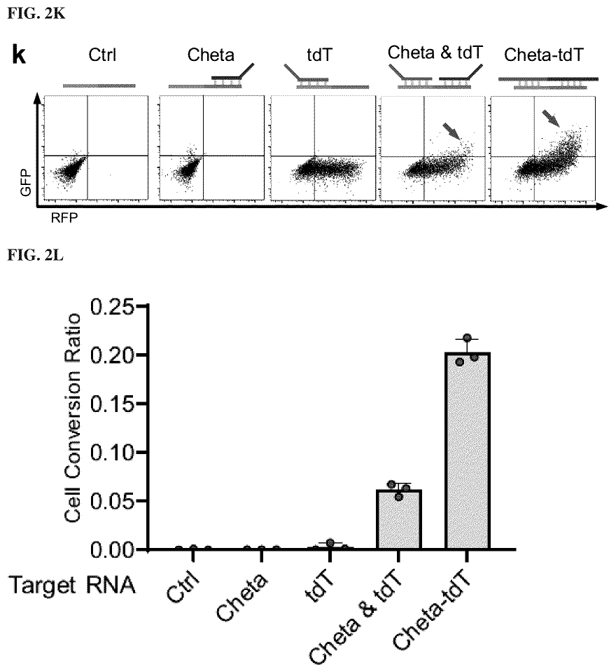

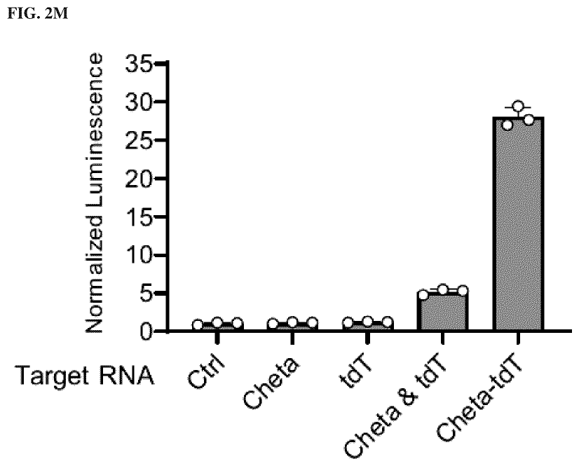

### FIGS. 2A-M: SesRNA Properties.

FIG. 2a, The optimal sesRNA length is ˜200-300 nt. Using READR^{BFP-tdT-GFP} and tdT as target RNA, sesRNA^{tdT} with variable lengths were tested to identify an optimal length, quantified as cell convertion ratio in FACS assay. FIG. 2b, Effect of mismatches between sesRNA and target RNA base pairing. Number of mismatches (top) and percent identity between sesRNA^{tdT} and tdT target RNA (bottom) are shown for each case. FIG. 2c, Effect of sesRNA sensing of different locations and sequences within the target RNA (e.g. different ChETA and tdT coding regions) expressed from a EF1a-ChETA-tdT vector; promoter region was used as negative control. readrRNA efficiencies were shown (bottom). FIG. 2d, To improve stringency, sesRNA^{tdT} was designed to contain 1 to 3 STOP codons (X-TAG) in READR^{tdT-Luc} vector. FIGS. 2e-2f, Luminescence of 293T cells transfected with READR^{tdT-Luc} only (FIG. 2e) or co-transfected with CAG-tdT (FIG. 2f). FIG. 2g, Schematic of dual and triple sensor READR^{ChETA/tdT-GFP} vectors with sesRNA arrays targeting different regions of a ChETA-tdT fusion transcript expressed from EF1a-ChETA-tdT FIG. 2h, FACS analysis of 293T cells co-transfected EF1a-ChETA-tdT and a dual or triple READR^{tdT-GFP} vector. Arrows indicate GFP₊/RFP₊ populations. FIG. 2i, Quantification of the efficiency of various dual or triple READR_{tdT-GFP} vector in (FIG. 2h). FIG. 2j, Scheme for intersectional targeting of cells expressing two target RNAs (ChETA and tdT) using a dual-sensor READR^{ChETA/tdT-GFP} or READR^{ChETA/tdT-Luc} vector; each sesRNA in the dual-sensor array contains an editable STOP. FIG. 2k, FACS analysis showed that co-transfection of 293T cells with READR^{Chet/atdT-GFP} and either CAG-ChETA or CAG-tdT resulted in almost no GFP translation, and only triple transfection resulted in GFP expression. EF1a-ChETA-tdT (expressing a ChETA-tdT fusion transcript) co-transfection was used as a positive control. Quantifications were shown with FACS (FIGS. 2l) and luciferase assay (FIG. 2m). Error bars in FIGS. 2a-2c, 2e-2f, 2i, and FIGS. 2l-2m are mean values±s.e.m. n=3, n represents the number of independent experiments performed in parallel.

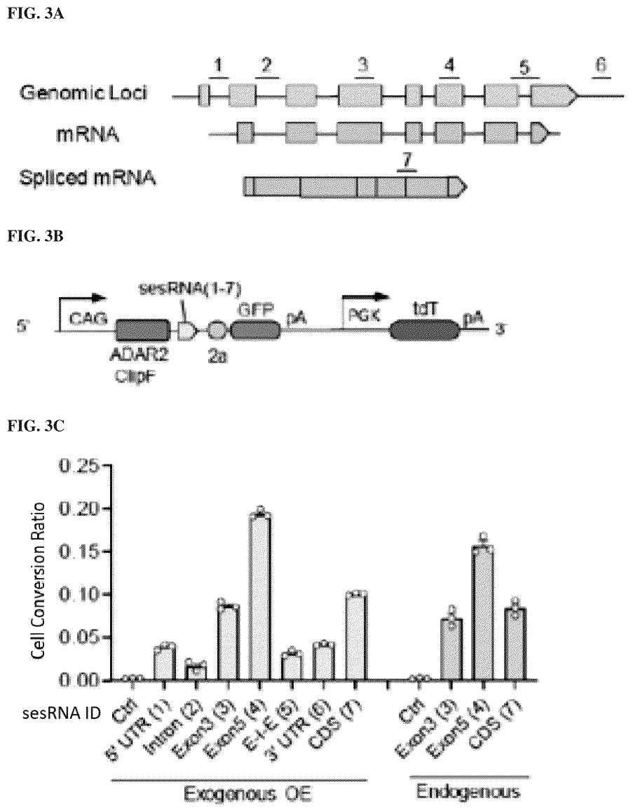

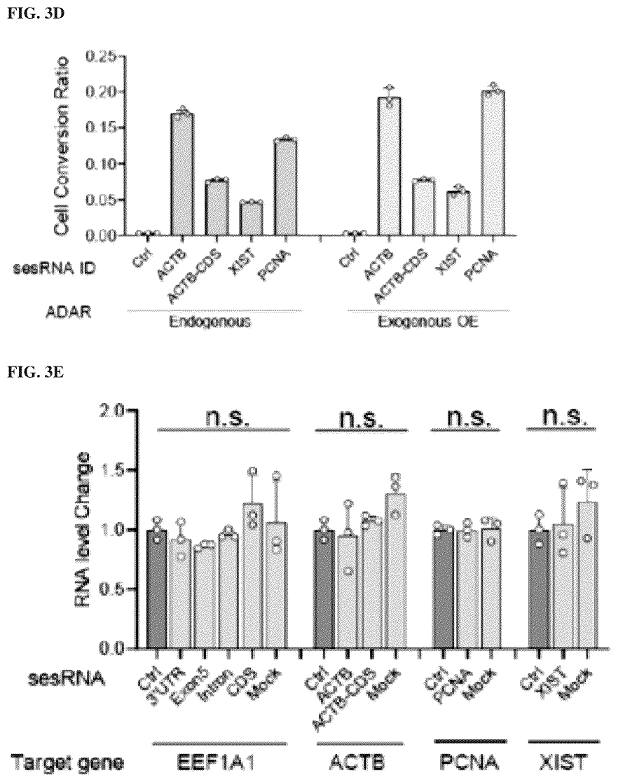

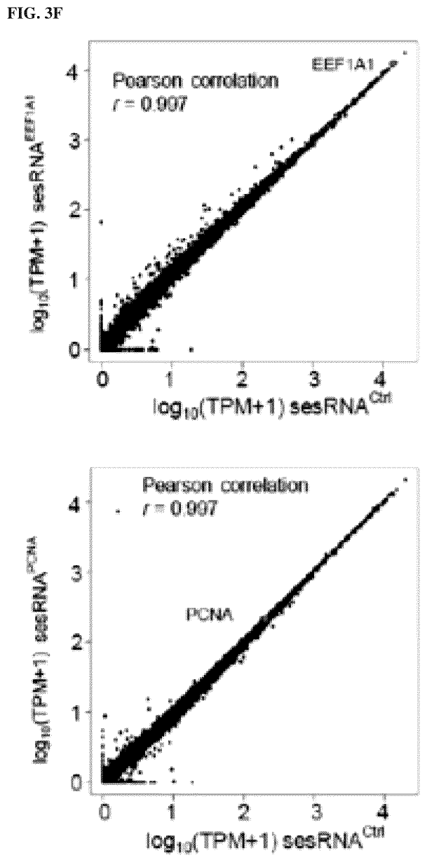

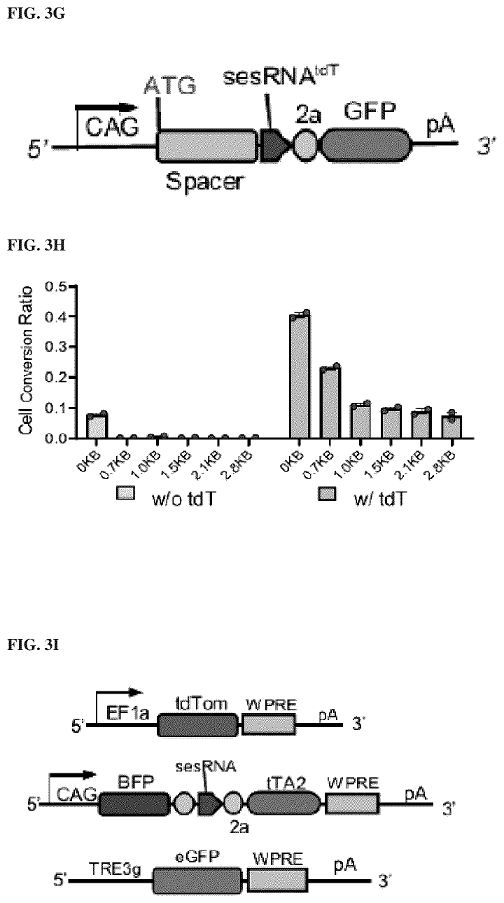

FIGS. 3A-3J. Endogenous RNA Sensing with CellREADR.

FIG. 3a, Schematic of the genomic loci of human EEF1A1 gene, showing exon and intron structures (not in scale), pre-mRNA (middle), and mRNA (bottom). sesRNA^{EEF1A1} (1-7) were designed to sample across the EEF1A1 transcripts including the coding sequence (CDS). FIG. 3b, Schematic of the READR_{EEF1A1-GFP} vector for testing sesRNA^{EEF1A1} (1-7); the BFP expression cassette labels all transfected cells. FIG. 3c, Quantification of the efficiency of sesRNA^{EEF1A1} (1-7) in 293T cells. FIG. 3d, High sensitivity of CellREADR. Quantification of CellREADR efficiency for several endogenous cellular RNAs with different expressing levels; TPM (transcript per million) from RNAseq data was used to indicate cellular RNA expression levels. 1-3 sesRNAs were designed for each target. E, Schematic for intersectional targeting of two endogenous RNAs (EEF1A1 and ACTB) using a dual-sensor READR^{EEF1A1/ACTB-GFP} vector; each sesRNA in the dual-sensor array contains an editable STOP. Dual-sensor READR_{Ctrl/Ctrl-GFP}, READR^{EEF1A1/Ctrl-GFP} or READR^{Ctrl/ACTB-GFP} were used as controls. Quantifications of efficiency showed that only 293T cells with READR^{Chet/atdT-GFP} transfection resulted in significant GFP expression. FIG. 3f, CellREADR did not alter cellular transcriptome assayed by RNA sequencing. Comparisons of transcriptomes between cells transfected with sesRNA^{EEF1A1(7)} or sesRNA^{Ctrl} (top) and with sesRNA_{PCNA} or sesRNA^{Ctrl} (bottom). FIG. 3g. Volcano plot of differential gene expression analysis between sesRNA_{EEF1A1} or sesRNA^{Ctrl} (top), sesRNA^{PCNA} or sesRNA^{Ctrl} (bottom). Genes with adjusted P value <0.01 and log 2(fold change)>2 were defined as significantly differentially expressed genes, labelled in red. One gene OAS2 was detected with significantly increased expression in the sesRNA^{EEF1A} group. FIG. 3h, Transcriptome-wide analysis of the effects of sesRNA^{EEF1A1} (top) or sesRNA^{ACTB} (bottom) on A-to-I editing by RNA-sequencing. Pearson's correlation coefficient analysis was used to evaluate the differential RNA editing rate. Error bars in c-e are mean values±s.e.m. n=3, n represents the number of independent experiments performed in parallel.

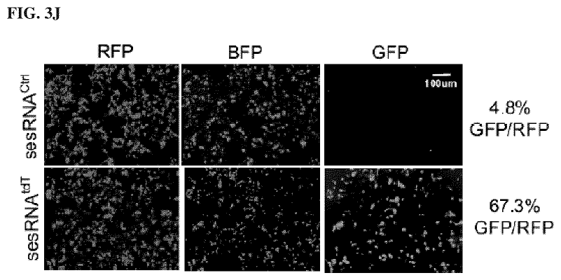

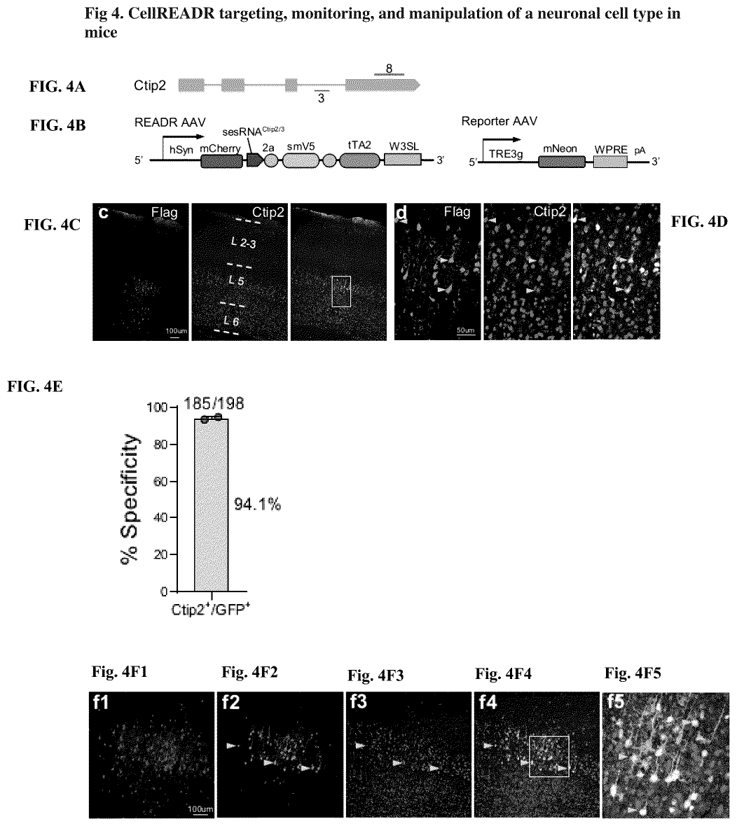

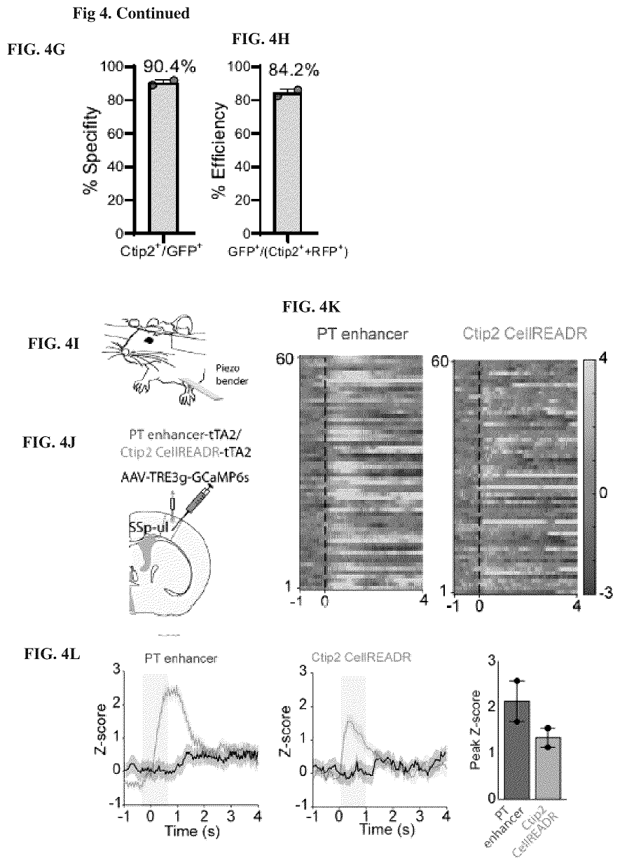

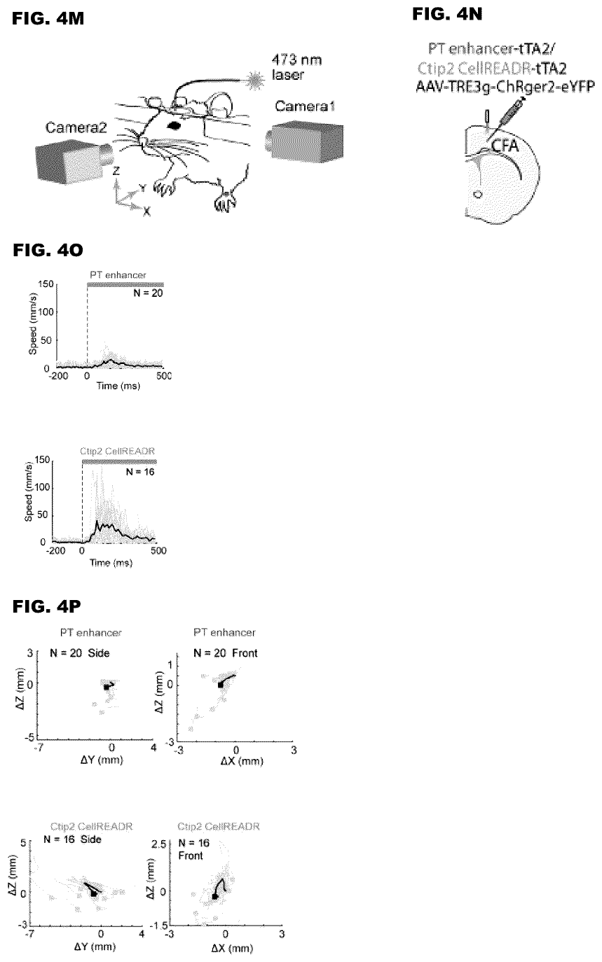

FIGS. 4A-P. CellREADR targeting, monitoring, and manipulation of a neuronal cell type in mice FIG. 4a. Genomic structure of the mouse Ctip2 genes with locations of sesRNA3 and sesRNA8 as indicated. FIG. 4b. Schematic of singular and binary AAV vectors for targeting Ctip2 neurons in brain tissues. In singular READR^{Ctip2-smFlag/tTA2} vector, a hSyn promoter drives expression of mCherry followed by sequences coding for sesRNA^{Cti}ₚ_{²}, smFlag and tTA2 effectors. In binary vectors, a Reporter vector drives mNeonGreen expression from a TRE promoter in response to tTA2 from ^{READRCtip2-smFlag/tTA2}. FIG. 4c, Coronal section of S1 cortex injected with READR^{Ctip2(3)-smFlag/tTA2} AAVs. Immunofluorescence with FLAG (left) and CTIP2 (middle) antibodies indicated that READR-labeled cells (FLAG⁺, green) showed high colocalization with CTIP2⁺ PNs. FIG. 4d, Magnified view of boxed region in ©. Arrowheads show representative co-labeled cells. FIG. 4e, Specificity of READR^{Ctip2(3)} delivered with singular vector. FIG. 4f, In S1 cortex co-injected with binary AAV vectors, neurons infected by READR^{Ctip2} expressed mCherry (FIG. 4f1). In a subset of these neurons, sesRNA^{Ctip2(3)} triggered translation of tTA2 and activation of mNeon expression in L5b PNs (FIG. 4f2). The specificity of mNeon expression in Ctip2 PNs was assessed with CTIP2 immunofluorescence (FIG. 4f3-f5). FIG. 4f5 is a boxed region in FIG. 4f4. Arrowheads indicate the co-labeled cells. FIG. 4g, Specificity of READR^{Ctip2(3)} delivered with binary vectors. FIG. 4h, Efficiency of binary READR^{Cti}ₚ_{²⁽³⁾}, calculated as GFP⁺ cells among mCherry and CTIP2 expressing cells. FIGS. 4i-l, READR^{Ctip2(3)} enabled calcium imaging of a specific cortical neuron type in vivo. FIGS. 4i-j, Schematic of left-paw stimulation in anesthetized mice (i) and fiber photometry recording (FIG. 4j). FIG. 4j, AAV-PTenhancer-tTA2 or AAV-Ctip2-CellREADR-tTA2 was co-injected with AAV-TRE-GCaMP6s into primary somatosensory cortex, upper limb area (SSp-ul). FIG. 4k, Heatmap of neuronal activity aligned to the onset of left-paw stimulation. FIG. 4l, Mean calcium signal aligned to the onset of left-paw stimulation (left two panels). Peak Z-score of the responses was shown (right panel, n=2 mice each). Gray area indicates is of stimulation. Black traces are from data aligned to shuffled onset times. Shades around mean denote±s.e.m. Data are mean±s.e.m. FIGS. 4m-4p, READR^{Ctip2(3)} enabled optogenetic manipulation of a specific cortical neuron type in behaving mice. FIGS. 4m-4n, Schematic of optogenetic stimulation experiment in head-fixed mice (FIG. 4m) and viral injection (FIG. 4n). Two cameras were used to record induced movements. Nose tip is the coordinate origin. X, Y, and Z axes correspond to medial-lateral, anterior-posterior, and dorsal-ventral axes, respectively. A reflective marker was attached to the back of left paw for tracking (red dot). FIG. 4n, AAV-PTenhancer-tTA2 or AAV-Ctip2-CellREADR-tTA2 was co-injected with AAV-TRE-ChRger2-eYFP into caudal forelimb area (CFA). FIG. 4o, Speed of left paw before and after stimulation onset (n=20 trials for PT enhancer mouse (top); n=16 trials for Ctip2 CellREADR mouse (bottom)). Black traces indicate the average. Red line denotes stimulation onset. Blue bar represents 500 ms of stimulation. (also see suppl video 1). FIG. 4p, Movement trajectories of left paw during optogenetic stimulation (n=20 trials for PT enhancer mouse (top row); n=16 trials for Ctip2 CellREADR mouse (bottom row)). Side (left) and front (right) trajectories were normalized to the start position of left paw. Black trajectories indicate the average. Squares indicate left-paw positions when stimulation stopped.

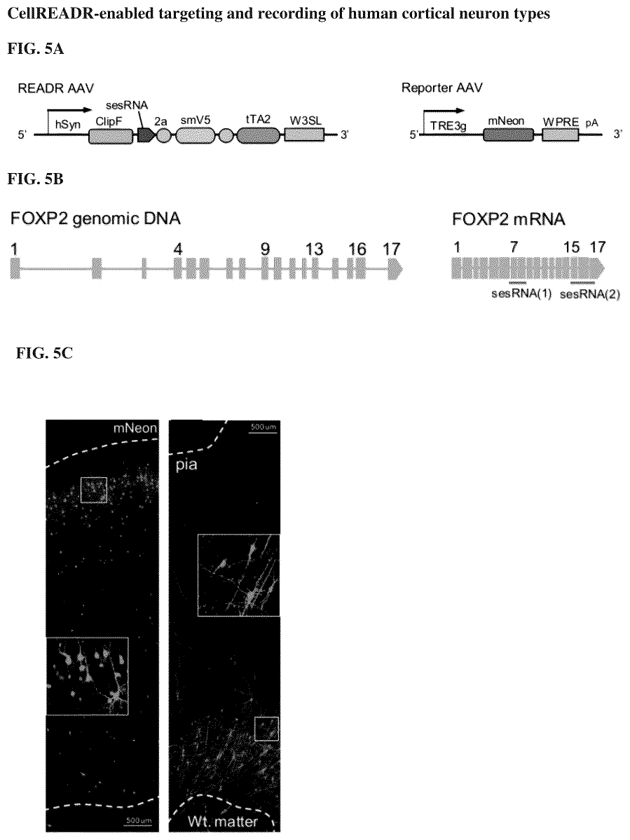

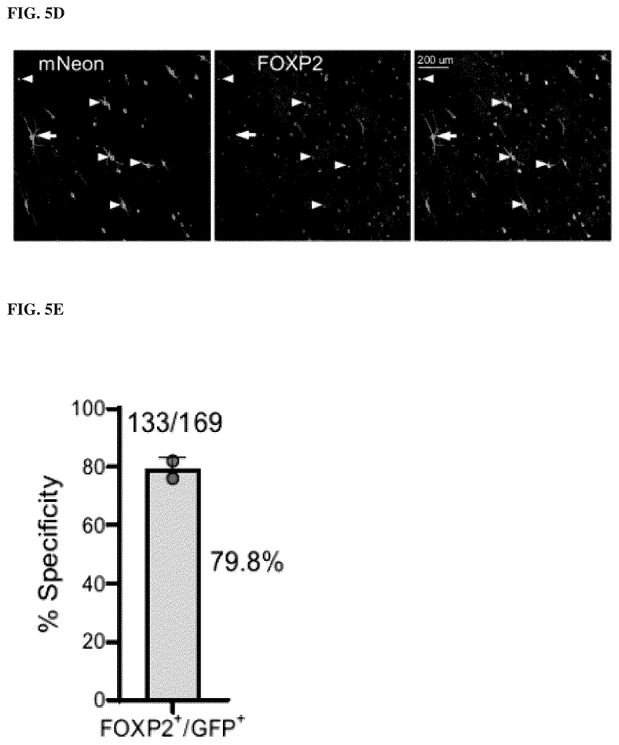

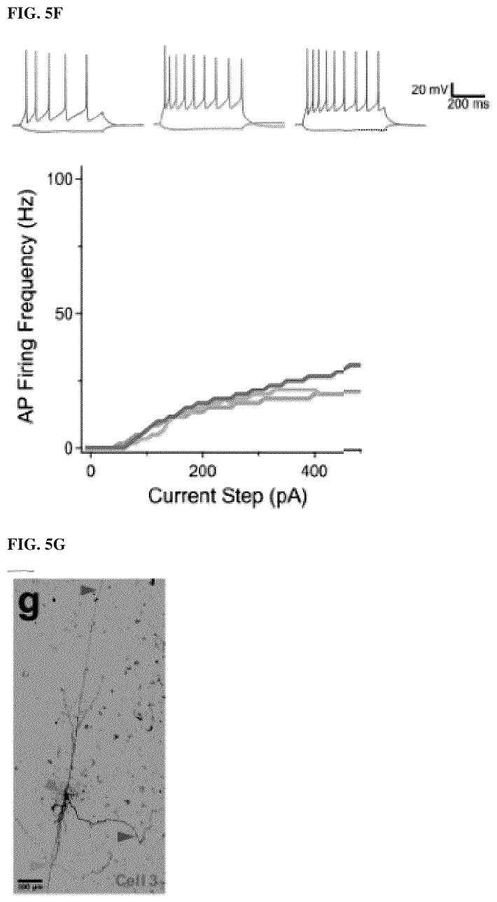

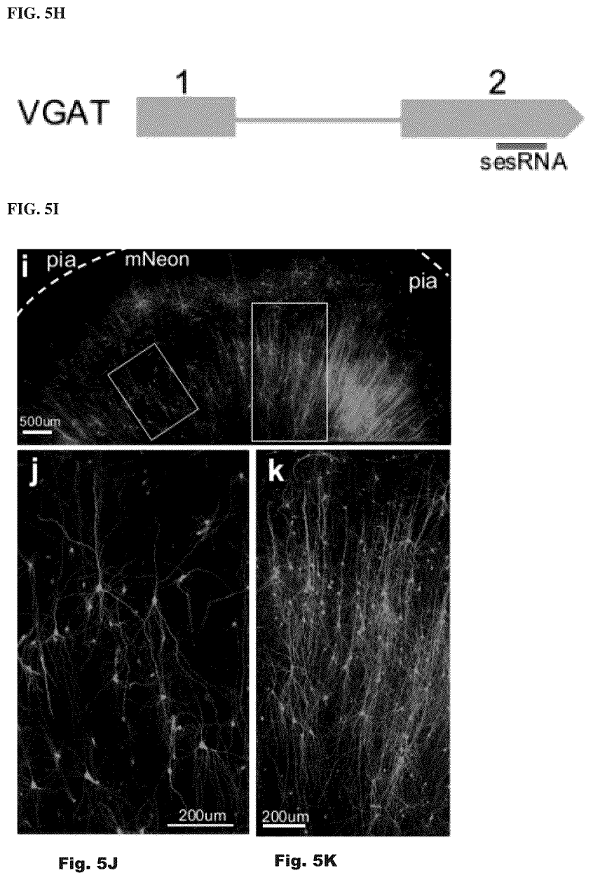

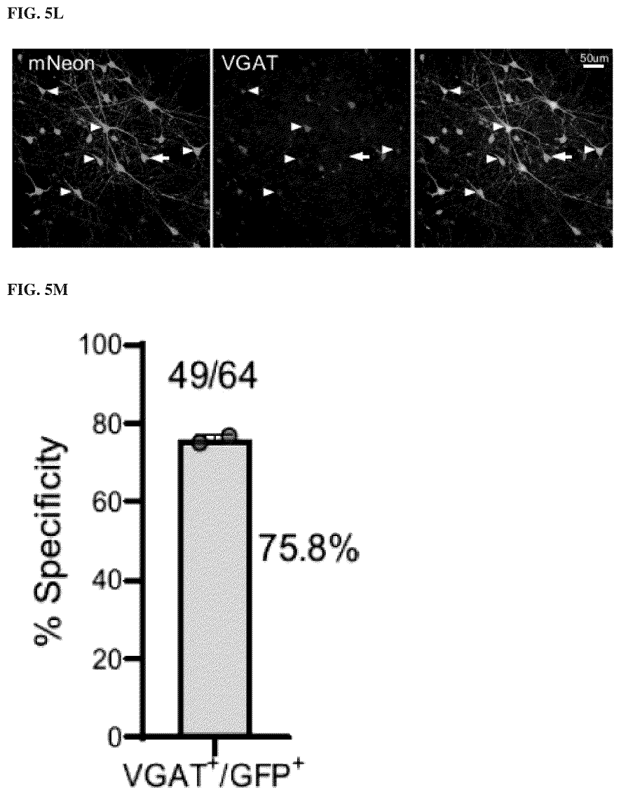

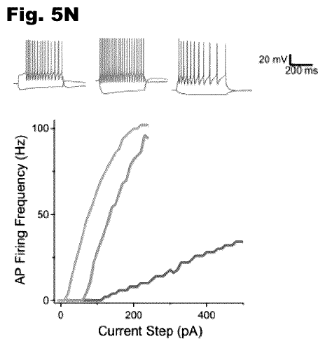

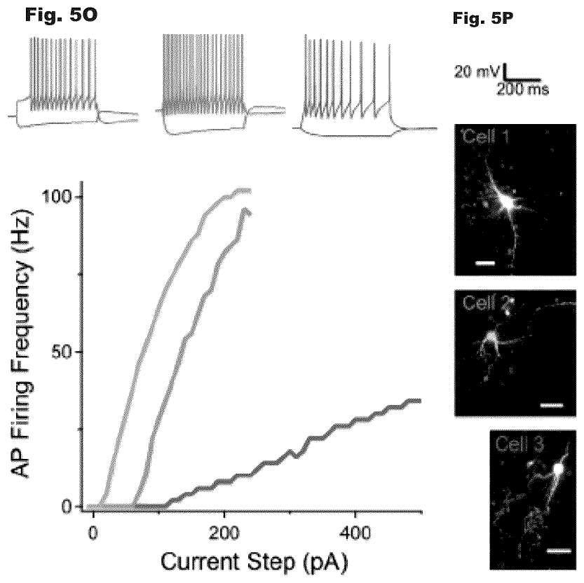

### FIGS. 5A-5O. CellREADR-Enabled Targeting and Recording of Human Cortical Neuron Types

FIG. 5a, Schematic of binary AAV vectors for targeting neuron types in human brain tissues. In the READR vector, a hSyn promoter drives transcription of ClipF-Tag followed by sequences coding for sesRNA^{FOXP2}, smV5, and tTA2. In TRE3g-mNeon, the TRE promoter drives mNeon in response to tTA from the READR virus. FIG. 5b, The genomic structure of human FOXP2 gene. Two sesRNAs were designed to target the FOXP2 mRNA. FIG. 5c, READR targeting of human FOXP2 cells. Upper layer FOXP2 cells (mNeon, native fluorescence) targeted with READR^{FOXP2(1)} AAVs in organotypic slices from temporal neocortex (left). Deeper layer FOXP2 cells targeted with READR^{FOXP2(2)} from parietal neocortex (right). Inserts showed magnified view of boxed region. Dashed lines delineate pia and white matter. FIG. 5d, Immunostaining of FOXP2 in READR^{FOXP2(2)} labeled neurons. Arrowheads indicate neurons co-labeled with mNeon native fluorescence (left) and FOXP2 immunofluorescence (middle); the arrow indicates a cell labeled only by mNeon. FIG. 5e, Specificity of READR^{FOXP2(2)} assayed by FOXP2 immunofluorescence. Error bars are mean values±s.e.m. n=2 biological replicates performed. FIG. 5f, Current-clamp recording traces of three mNeon-labeled neurons (top). Input-output curves for cells depict similar spiking behaviors (top). FIG. 5g, Morphology of labeled neuron recorded in (FIG. 5f) (blue curve). The patched cell was filled with biocytin for post-hoc recovery and was visualized by silver stain in bright field. Brown arrowhead, cyan arrowhead and purple arrowheads indicate the cell soma, axons and dendrites, respectively. FIG. 5h, SesRNA designed to target exon2 of the human VGAT mRNA. FIG. 5i, An organotypic slice from human temporal neocortex co-infected with AAV-READR^{VGAT}; TRE3g-mNeon and visualized by native fluorescence 8 days post-infection. FIGS. 5j and 5k are expansion of the 2 inserts of FIG. 5I, Morphologies of AAV-READR^{VGAT}-labeled interneurons. FIG. 5l, Colocalization of mNeon native fluoescence (left) with VGAT mRNA by in situ hybridization (middle). Arrowheads indicate co-labeled neurons; arrow indicates a cell labeled only by mNeon. FIG. 5m, Specificity of READR^{VGAT} assayed by VGAT mRNA in situ. Error bars are mean values±s.e.m. n=2 biological replicates performed. FIG. 5n, Current-clamp recording traces (top) of three labeled neurons, filled with biocytin for post-hoc recovery. Streptavidin dye was used for visualization. Input-output curves (bottom) depict distinct spiking behaviors (accommodating (blue), fast (green), delayed onset (red)). FIG. 5o, Morphologies of READR^{VGAT} labeled cells recorded in (FIG. 5n).

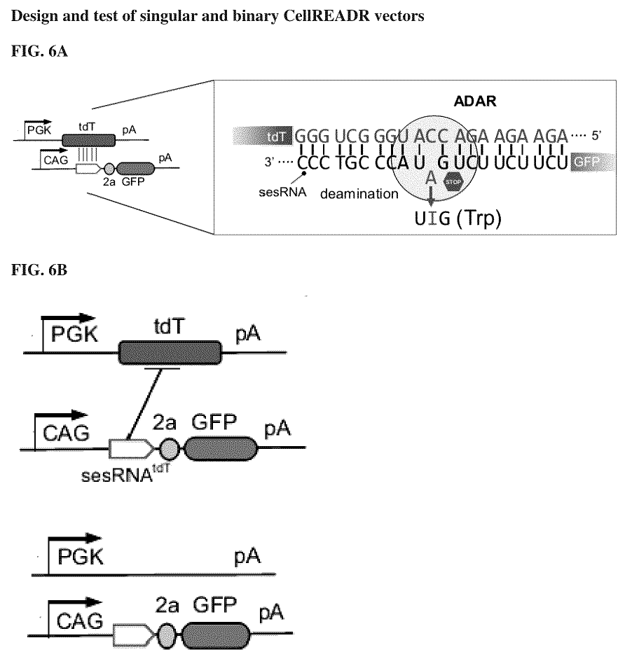

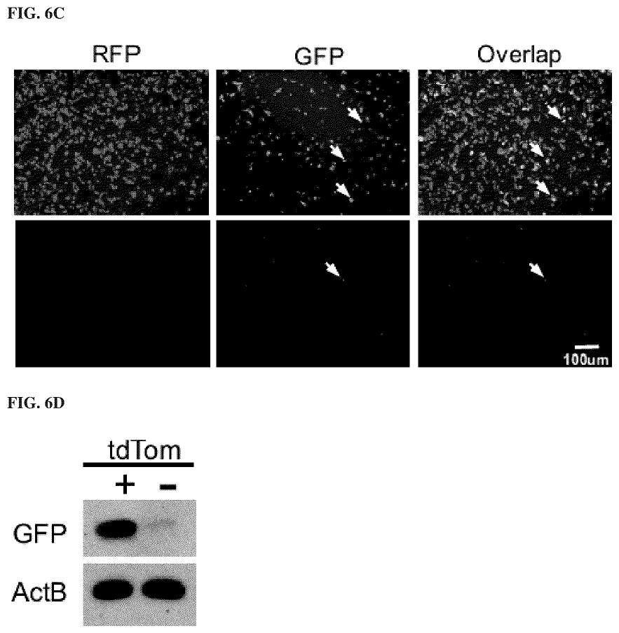

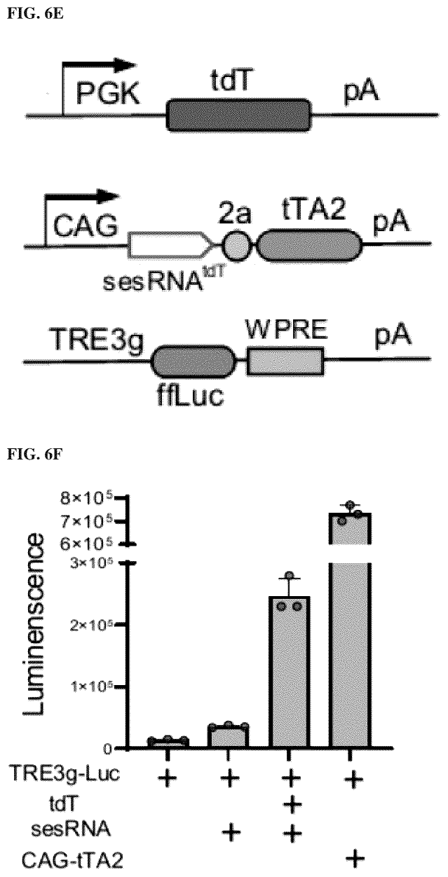

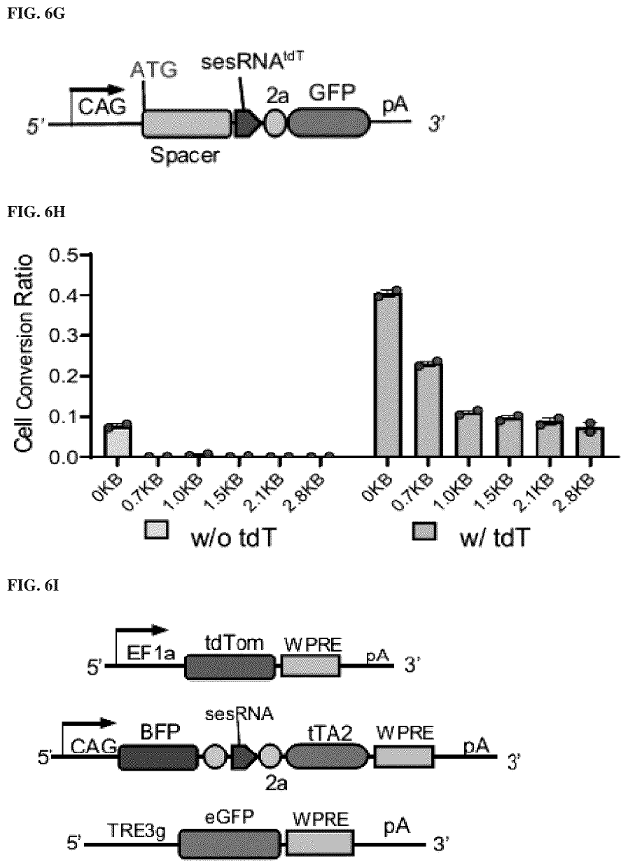

### FIGS. 6A-6J. Design and Test of Singular and Binary CellREADR Vectors

FIG. 6a, Schematic of a singular CellREADR vector. Left, PGK-tdT expresses the tdTomato target RNA from a PGK promoter. READR^{tdT-GFP} expresses a READR RNA consisting of sesRNA^{tdT} and efRNA^{GFP}, driven by a CAG promotor. Vertical dashed lines indicate the complementary base pairing region between tdT mRNA and sesRNA^{tdT}, with sequence surrounding the editable STOP codon shown on the right. At the editing site, the editable adenine in sesRNA^{tdT} (cyan) is mismatched to a cytosine in the tdT mRNA. TdTomato is a tandem repeat of two dTomato genes, thus a tdT RNA contains two copies of target sequence for sesRNA^{tdT} base pairing. FIGS. 6b-6d, Validation of the READR^{tdT-GFP} vector. In 293T cells co-transfected with READR^{tdT-GFP} and PGK-tdT (FIG. 6b), many switched on GFP translation and fluorescence (c, upper arrows). In cells co-transfected with control empty vector, few cells showed GFP expression (c, lower). GFP expression was further assayed by Western blotting (FIG. 6d). FIG. 6e, A binary vector design for CellREADR luciferase assay. READR^{tdT-tTA2} expresses a readrRNA consisting of sesRNA^{tdT} and efRNA^{tTA2}, and TRE-ffLuc expresses the luciferase RNA upon tTA2 activation. FIG. 6f, Luciferase activity dramatically increased only in cells transfected with three vectors in (FIG. 6e). Co-transfection of TRE-ffLuc with CAG-tTA2, which constitutively expresses tTA2, served as a positive control. FIG. 6g, Schematic READR^{tdT-GFP} vector in which a spacer sequences is inserted before sesRNA^{tdT} coding region. FIG. 6h, 293T cells were transfected with READR^{tdT-GFP} vector encoding viable length of spacers without (gray) or with (pink) tdT target RNA expression, respectively. Quantification of conversion ratio calculated as percentage of GFP⁺ cells among RFP⁺ cells. FIG. 6i, A binary vector design for CellREADR assay. FIG. 6j, Representative images of GFP conversion with binary vectors in (i). In cells co-transfected with sesRNA^{Ctrl} vector, few GFP⁺ cells were observed. Conversion percentages are shown on the right. Error bars in FIGS. 6f and 6h are mean values±s.e.m. n=3 for f, n=2 for FIG. 6h, n represents the number of independent experiments performed in parallel.

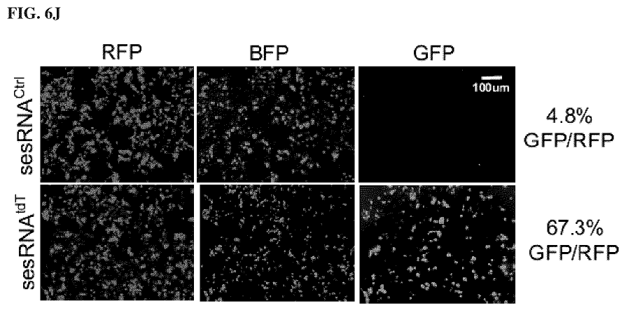

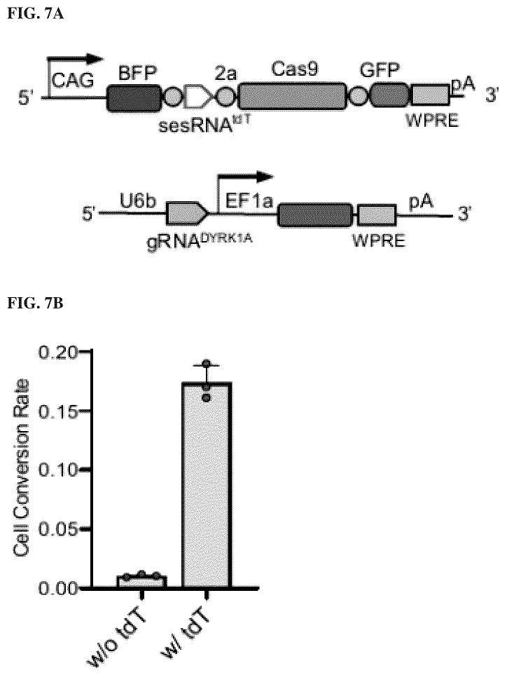

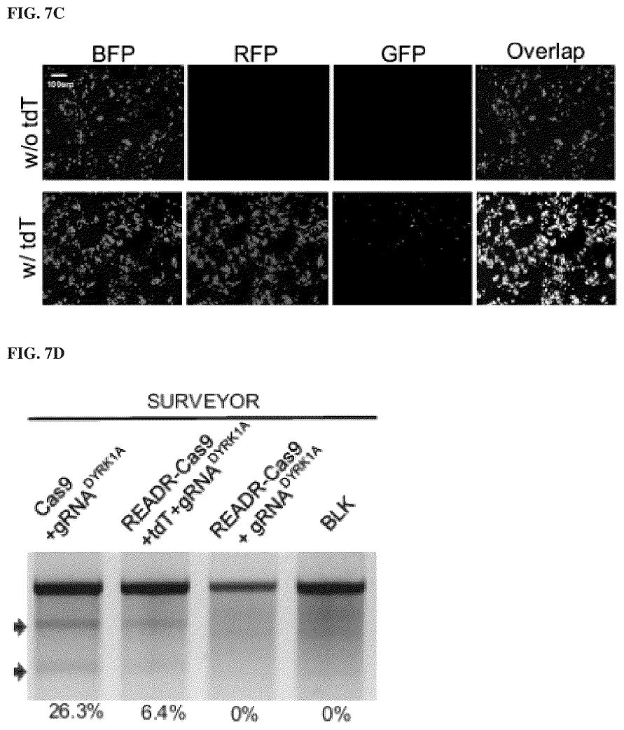

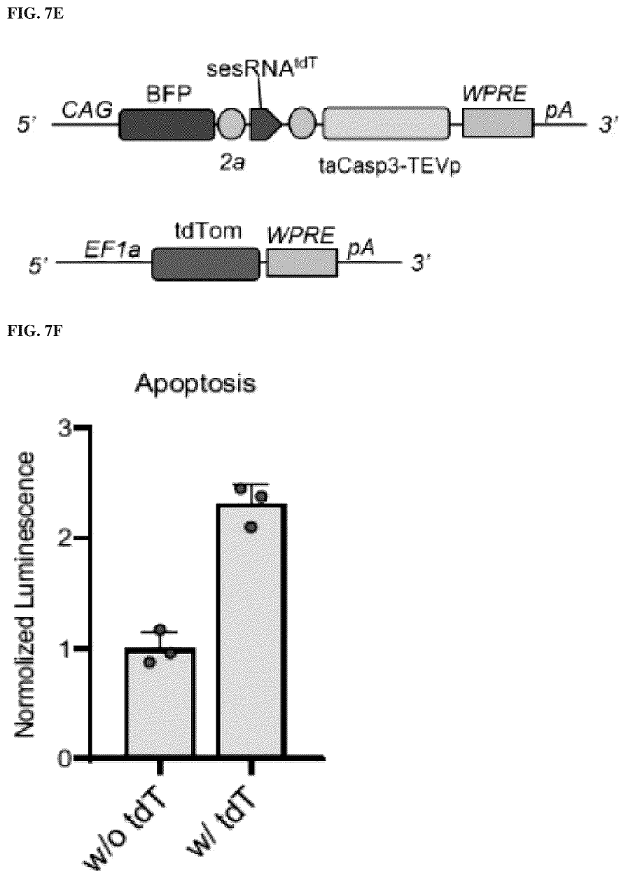

FIGS. 7A-7E. CellREADR Enables RNA Sensing Dependent Gene Editing and Cell Ablation

FIG. 7a, Vector design for CellREADR-mediated and target RNA-dependent gene editing. In READR_{tdT-Cas9/GFP}, a CAG promoter drives expression of BFP followed by sequences coding for sesRNA^{tdT}, Cas9, and eGFP effectors. In another vector, EF1a promoter drives tdT expression and U6b promoter drives the expression of a guide RNA (gRNA) targeting the DYRK1A gene in 293T cells. FIG. 7b, Quantification of READR^{tdT-Cas9/GFP} efficiency as percent of GFP among RFP and BFP expressing cells with or without tdT target RNA. FIG. 7c, Cells transfected with the both U6b-gRNA^{DYRK1A}-CAG-tdT and READR_{tdT-Cas9/GFP} showed robust GFP expression co-localized with BFP and RFP (bottom). Cells transfected with READR _{tdT-Cas9/GFP} only (top) showed almost no GFP expression. FIG. 7d, SURVEYOR assay showed Cas9-mediated cleavage in the human DYRK1A locus. DNA cleavage was observed in cell lysates transfected with U6b-gRNA^{DYRK1A}-CAG-tdT and READR^{tdT-Cas9/GFP}, but not in U6b-gRNA^{DYRK1A} and READR_{tdT-Cas9/GFP} that lacked tdT target RNA. CAG-Cas9 with U6b-gRNA_{DYRK1 A} cell lysate and 293 T cell lysate without plasmid transfection were used as positive control and negative control, respectively. Arrows indicate cleavage products. FIG. 7e, Vector design for CellREADR-mediated and target RNA-dependent cell death induction. In READR^{tdT-taCasp3-TEVp}, a CAG promoter drives expression of BFP followed by sequences coding for sesRNA^{tdT} and taCasp3-TEVp as effector to induce cell death. FIG. 7f. Cell apoptosis level measured by luminescence was increased in the cells transfected READR^{tdT-taCasp3-TEVp} and EF1a-tdT compared with cells with no tdT RNA. Error bars in b and f are mean values s.e.m. n=3, n represents the number of independent experiments performed in parallel.

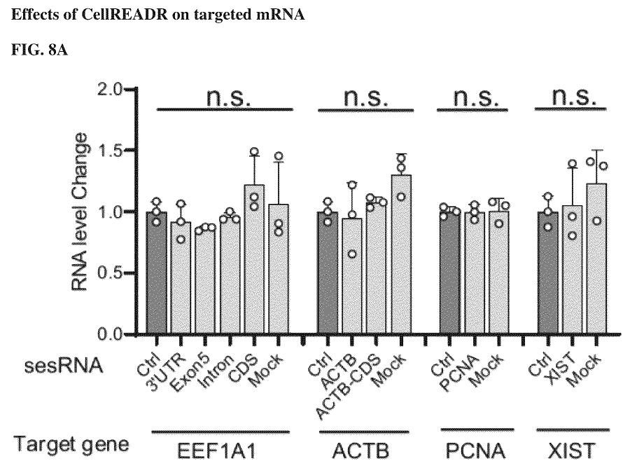

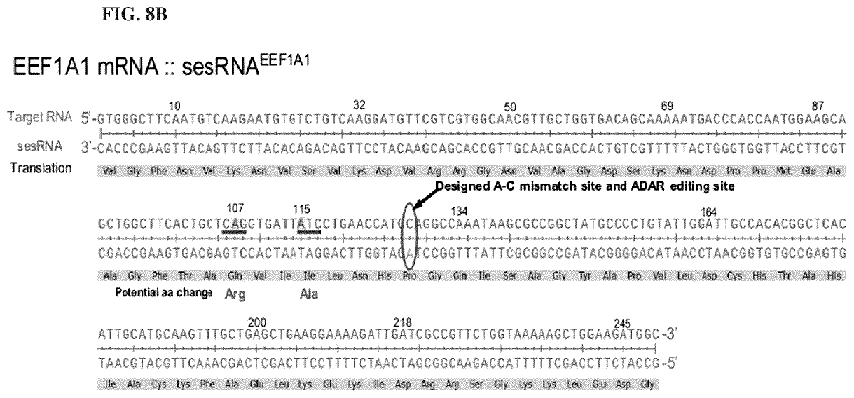

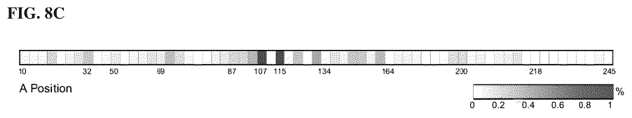

FIGS. 8A-8C. Effects of CellREADR on Targeted mRNA

FIG. 8a, Quantitative PCR showing that CellREADR-mediated sesRNA expression did not impact the expression levels of targeted RNAs. FIG. 8b, Base pairing of EEF1A1 mRNA and sesRNA^{EEF1A1-CDS}. EEF1A1 mRNA or sesRNA was represented in blue and red, respectively. Peptide translated from EEF1A1 mRNA was highlighted in brown. Targeted region of EEF1A1 mRNA was analyzed by RNAseq. FIG. 8c, The ratios of A-to-G changes in EEF1A1 mRNA at each adenosine position was quantified and shown in heatmap. Two adenosines (A107 and A115) showed higher rate of A-to-G editing (FIG. 8c). Off-target editing of two sensitive adenosines can induce potential animo acid change (underlined in FIG. 8b). Error bars are mean values±s.e.m. n=3, n represents the number of biological replicates performed.

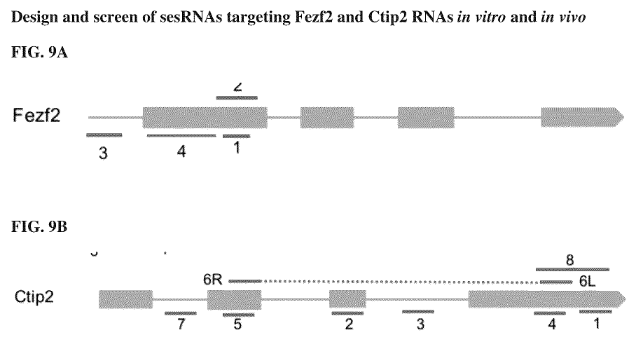

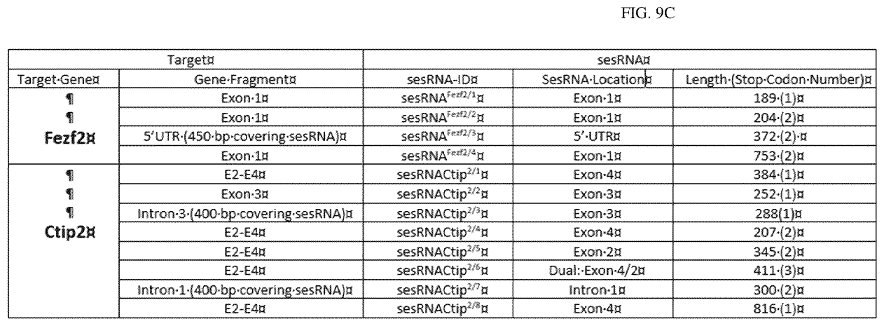

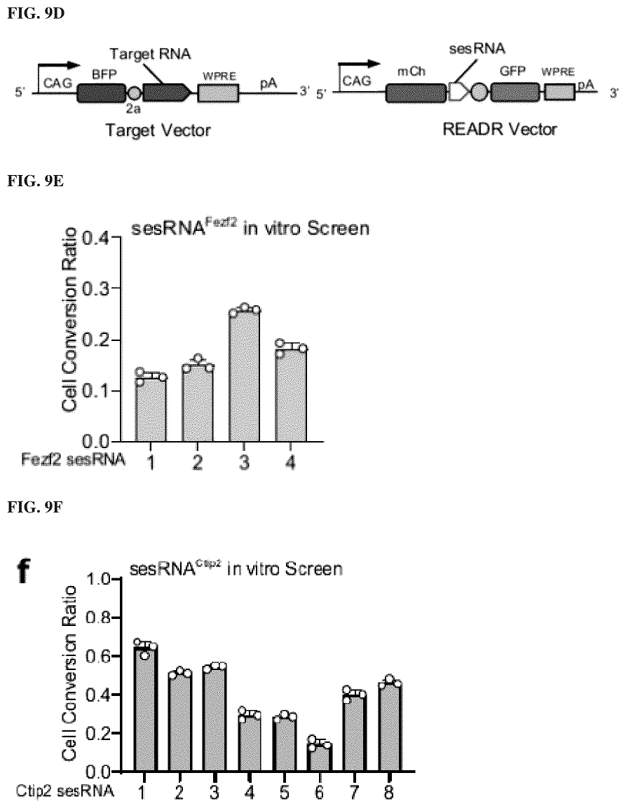

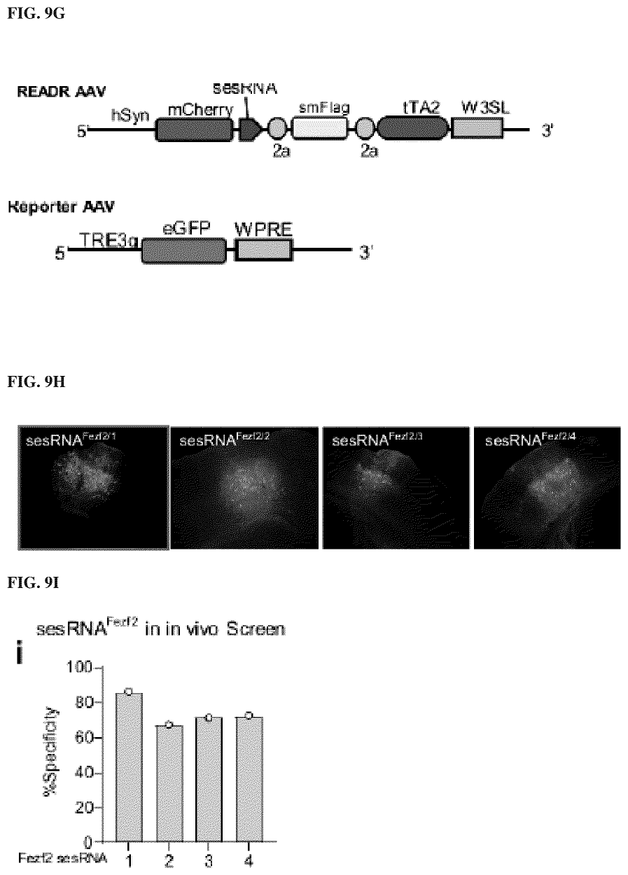

FIGS. 9A-9K Design and Screen of sesRNAs Targeting Fezf2 and Ctip2 RNAs In Vitro and In Vivo.

FIGS. 9a-9b, Genomic structures of mouse Fezf2 (FIG. 9a) and Ctip2 (FIG. 9b) genes with locations of various sesRNAs as indicated. FIG. 9c, List of sesRNAs and Fezf2 and Ctip2 target gene fragments used for sesRNA screen. FIG. 9d, In Target vectors CAG-BFP-Fezf2 or CAG-BFP-Ctip2, a 200-3000 bp genomic region of the Fezf2 or Ctip2 gene containing sequences complementary to a sesRNA in (FIGS. 9a, 9b) were cloned downstream to the BFP and T2a coding region driven by a CAG promoter. In READR vectors, READR^{Fezf2-GFP} or READR^{Ctip2-GFP} expresses corresponding sesRNAs shown in (FIGS. 9a, 9b). FIGS. 9e-9f, Quantification of efficiencies READR^{Fezf2-GFP} (e) or READR^{Ctip2-GFP} (f) as GFP conversion ratio by FACS assay of 293T cells co-transfected with CAG-BFP-Fezf2 or CAG-BFP-Ctip2 target vector, respectively. FIG. 9g, Schematic of binary READR AAV vectors. In READR vector, a hSyn promoter drives expression of mCherry followed by sequences coding for sesRNA^{Cti}ₚ_{²}, smFlag and tTA2 effectors. In Reporter vector, TRE promoter drives mNeonGreen in response to tTA2 from the READR vector. FIG. 9h, Coronal sections of mouse cortex injected with binary READR^{Fezf2} vectors. mNeonG indicated READR^{Fezf2} labeled cells. Four Fezf2 sesRNAs were screened. i, Quantification of specificity of 4 Fezf2 sesRNAs in (FIG. 9h). For Fezf2 sesRNA in-vivo screen, the specificity of each sesRNA was calculated by co-labeling by READR AAVs and CTIP2 antibody (due to lack of FEZF2 antibody); as Ctip2 represents a subset of Fezf2+ cells (not shown), CTIP2 antibody gives an underestimate of the specificity of Fezf2 sesRNA. SesRNA1 showed highest specificity. FIG. 9j, Coronal sections of mouse cortex injected with binary READR^{Ctip2} vectors. mNeonG indicated binary READR labeled cells. Eight Ctip2 sesRNAs were screened. FIG. 9k, Quantification of specificity of 8 sesRNAs in (FIG. 9j). The specificity of each sesRNA was calculated by co-labeling by binary READR^{Ctip2} AAVs and CTIP2 antibody (not shown). SesRNA3 and sesRNA8 showed highest specificity.

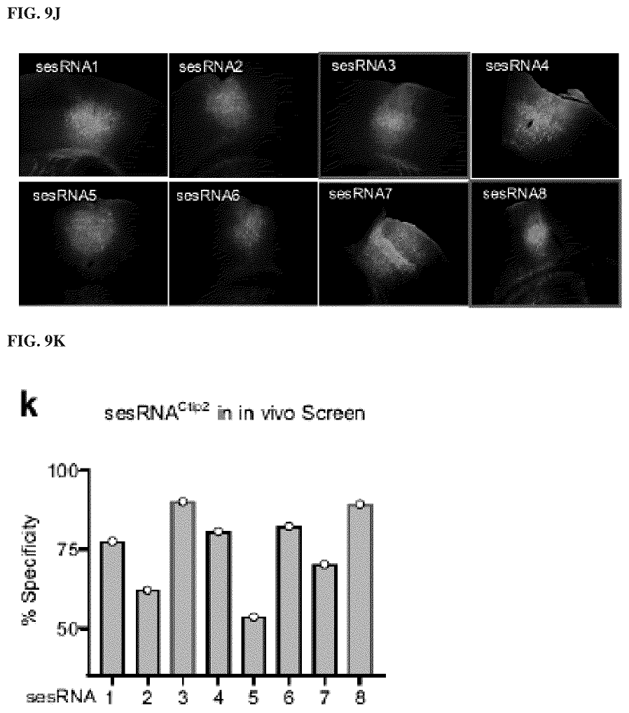

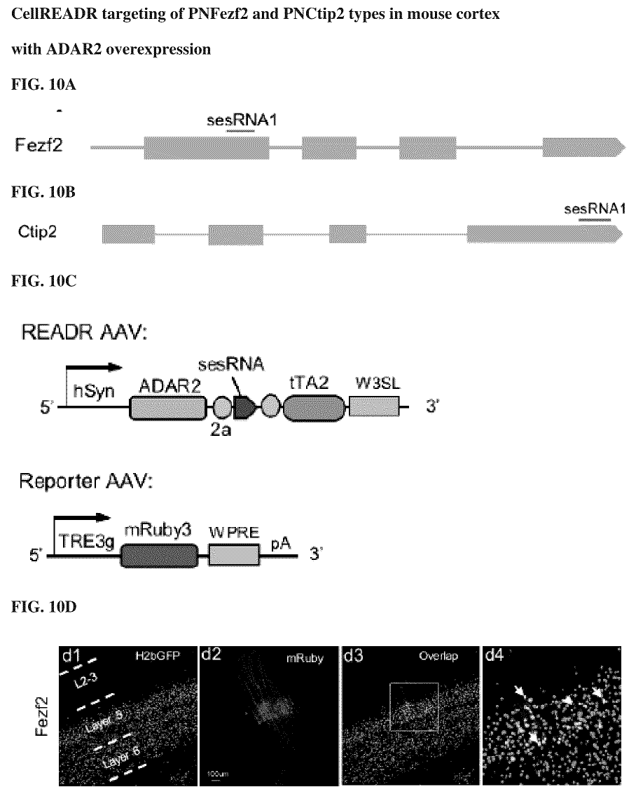

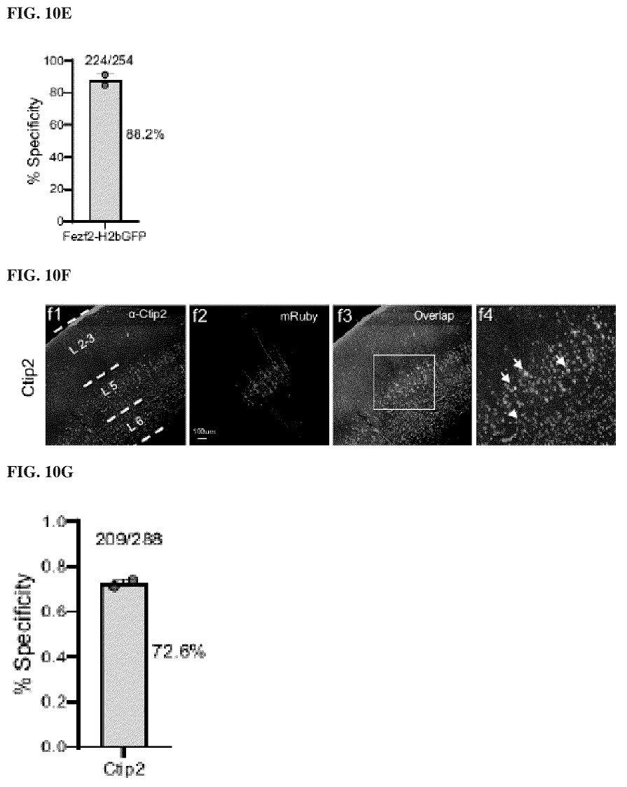

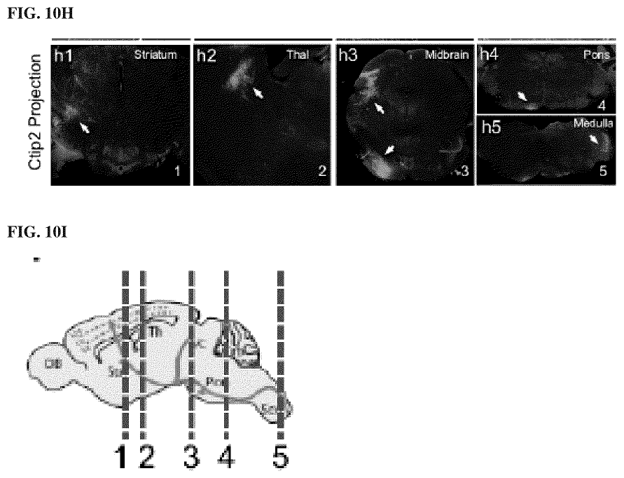

FIGS. 10A-10I CellREADR Targeting of ^{PNFezf2} and ^{PNCtip2} Types in Mouse Cortex with ADAR2 Overexpression

FIGS. 10a-b, Genomic structures of mouse Fezf2 (FIG. 10a) and Ctip2 (FIG. 10b) genes with locations of sesRNAs as indicated, respectively. FIG. 10c, Schematic of binary AAV vectors for targeting neuron types. In READR^{Fez}f^{2-tTA2}, a hSyn promoter drives expression of ADAR2 followed by sequences coding for sesRNA^{Fez2(1)}, T2a, and tTA2 effector. In TRE-mRuby, the TRE promoter drives mRuby3 in response to tTA2 from the READR virus. FIG. 10d, Image of coronal section from a Fezf2-CreER; LoxpSTOPLoxp-H2bGFP mouse brain, showing the distribution pattern of Fezf2⁺ PNs in S1 somatosensory cortex (FIG. 10d). Rodrigo Munoz-Castaneda et al. Cellular anatomy of the mouse primary motor cortex Nature volume 598, pages 159-166 (2021) Cre-reporter transgenic mice were created by crossing ‘knock-in’ Cre drivers with reporter mice (CAG-LoxP-STOP-LoxP-H2B-GFP) as described previously in Kim, Y. et al. Brain-wide maps reveal stereotyped cell-type-based cortical architecture and subcortical sexual dimorphism. Cell 171, 456-469.e422 (2017). Co-injection of AAVs READR_{Fezf2(1)-tTA2} and TRE-mRuby specifically labeled PNs in L5b and L6 (FIG. 10d2). Co-labeling by CellREADR AAVs and H2bGFP (FIG. 10d3) with magnified view in (FIG. 10d4). Arrows indicate co-labeled cells; arrowhead shows a neuron labeled by CellREADR AAVs but not by Fezf2-H2bGFP (FIG. 10d4). FIG. 10e, Specificity of READR_{Fezf2(1)-tTA2}. FIG. 10f, Coronal section of WT brain immuno-stained with a CTIP2 antibody, showing the distribution pattern of Ctip2+ PNs in S1 cortex (FIG. 10f1). Co-injection of AAVs READR^{Ctip2(1)} and TRE-mRuby specifically labeled PNs in L5b (FIG. 10f2). Co-labeling by CellREADR AAVs and CTIP2 antibody (FIG. 10f3) with magnified view in (FIG. 10f4). Arrows show the co-labeled cells; arrowhead showed a mis-labeled cells by READR^{Ctip2(1)} (FIG. 10f4). g, Specificity of READR^{Cti}ₚ_{²⁽¹⁾}. FIG. 10h, Axonal projection pattern of AAV READR_{Ctip2(1)-tTA2} and TRE-eYFP infected PNs in S1 cortex. Representative images showing projections to striatum, thalamus, midbrain, pons and medulla (arrows). FIG. 10i, Schematic locations of coronal sections are shown on the right panel.

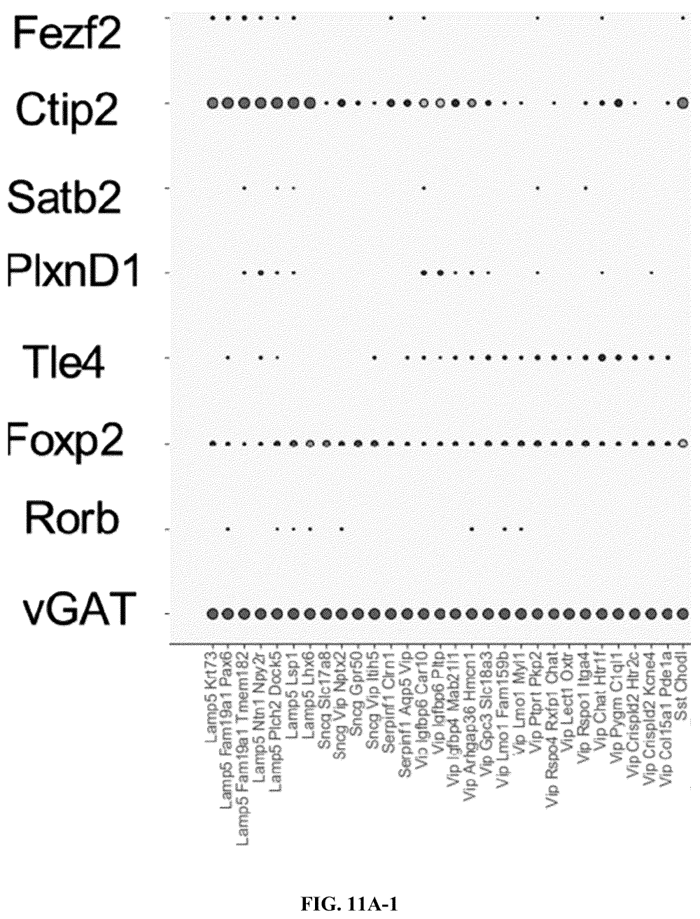

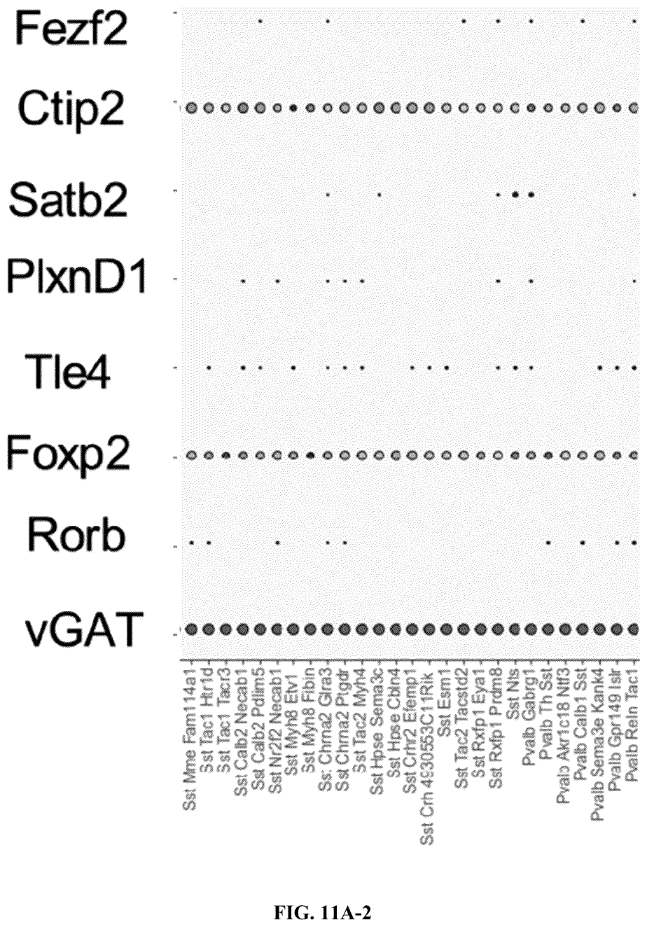

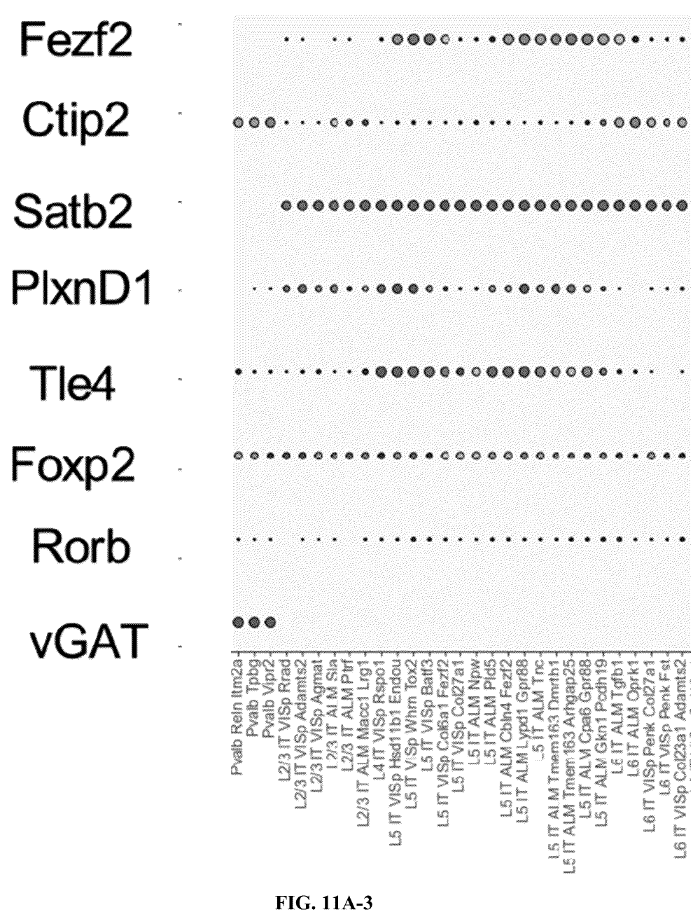

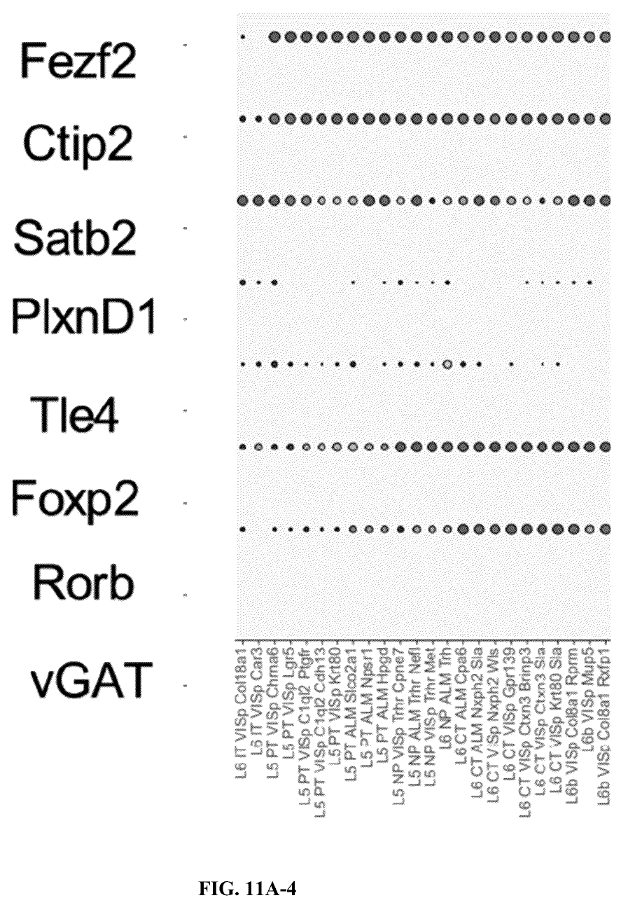

### FIGS. 11A-11B Expression Level and Laminar Distribution of Cortical Cell Type Markers in Mice.

FIG. 11a. Group plot of selected genes in transcriptomic cell type clusters, based on dataset from the Allen Institute for Brain Science. Gene expression level and cortical distribution were shown. FIG. 11b, gene expression level of major cell type marker genes. The plots were generated with scRNAseq data⁴⁰.

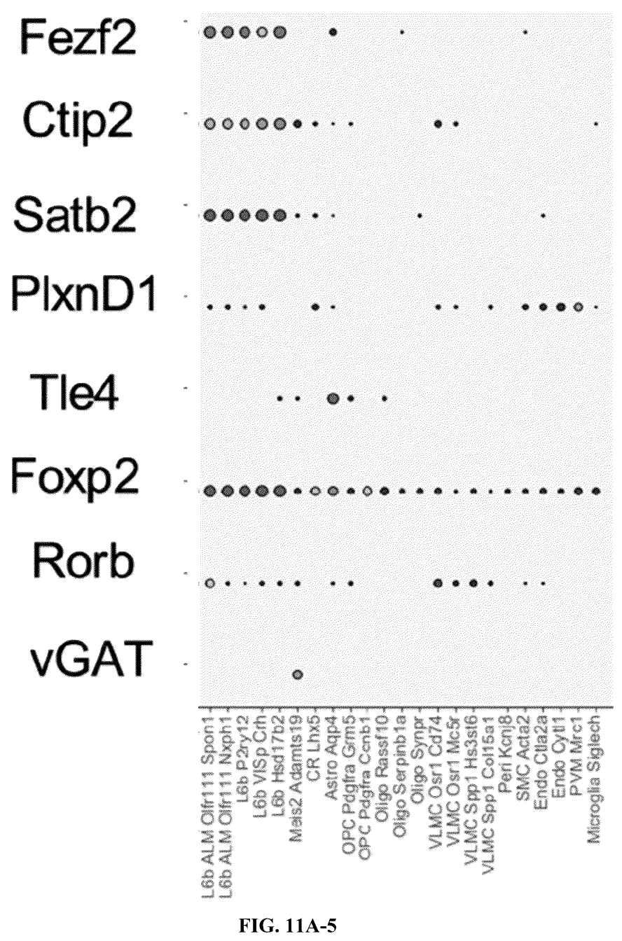

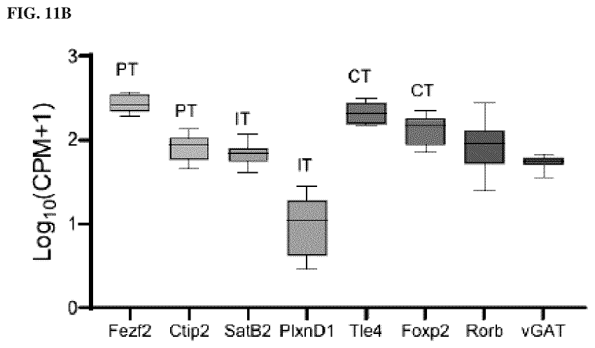

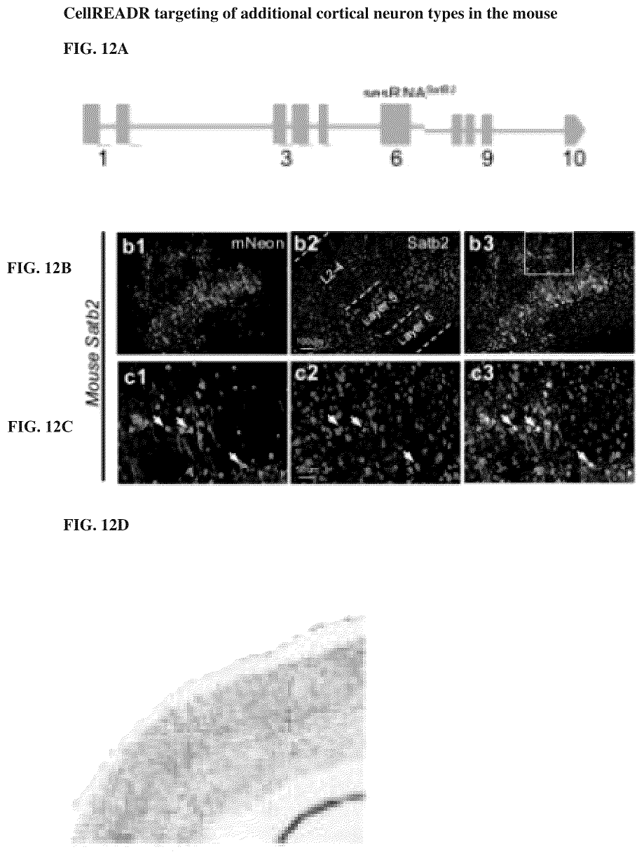

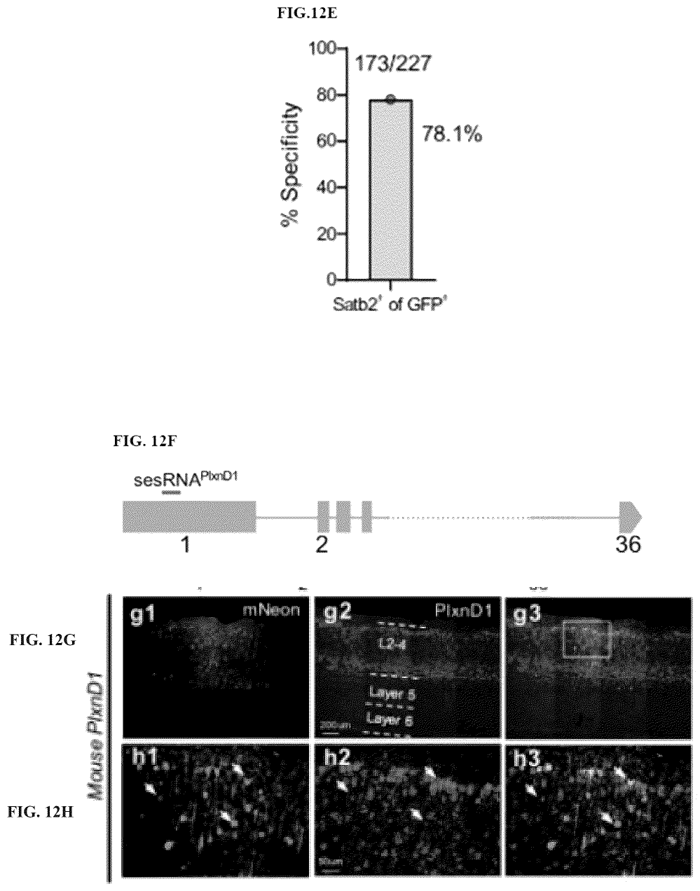

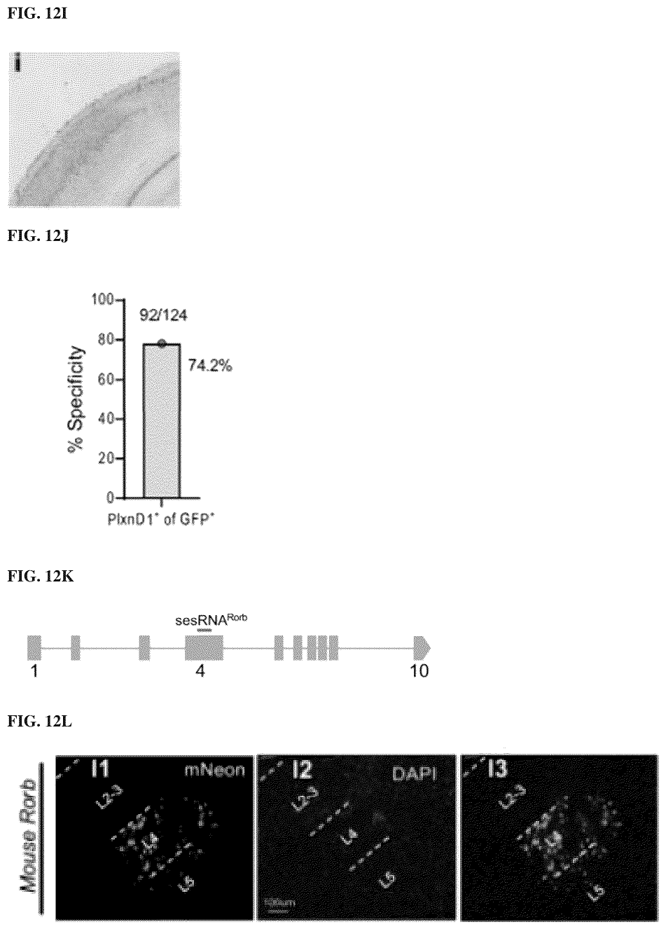

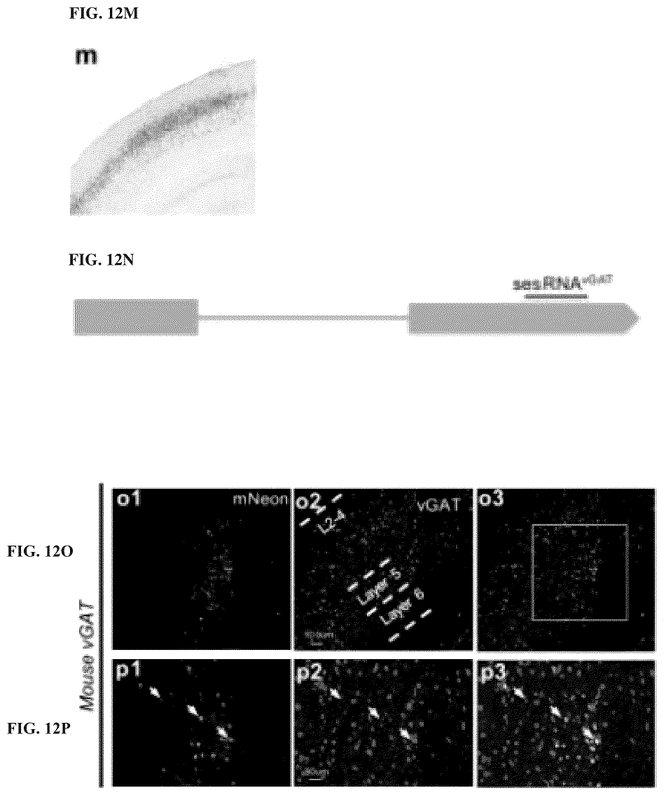

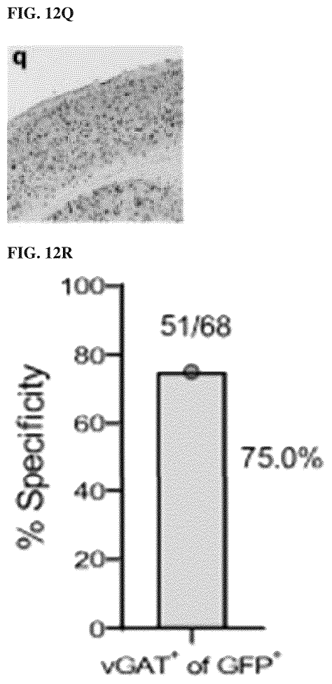

### FIGS. 12A-12R CellREADR Targeting of Additional Cortical Neuron Types in the Mouse

FIG. 12a, Genomic structures of mouse the Satb2 gene with location of a sesRNA as indicated. FIGS. 12b-12c, Cell labeling pattern in S1 by co-injection of binary vectors described in FIG. 4i. (FIG. 12b1) AAVs READR^{Satb2} and TRE-mNeon labeled cells in both upper and deep layers. (b2) Satb2 mRNA in-situ hybridization. (FIG. 12b3) Co-labeling by READR^{Satb2} and Satb2 mRNA. c, Magnified view of boxed region in (FIG. 12b3). Arrows indicate co-labeled cells. FIG. 12d, Satb2 mRNA expression pattern in S1 cortex at P56 from the Allen Mouse Brain Atlas. FIG. 12e, Specificity of READR^{Satb2} measured as the percent of Satb2⁺ cells among mNeon cells. FIG. 12f, Genomic structures of the mouse PlxnD1 gene with location of a sesRNA as indicated. FIGS. 12g-12h, AAVs READR^{PlxnD1} and TRE-mNeon labeled cells in upper layers and L5a in S1 (FIG. 12g1). PlxnD1 mRNA in-situ hybridization (g2). Co-labeling by READR^{PlxnD1} AAVs and PlxnD1 mRNA (FIG. 12g3). FIG. 12h, Magnified view of boxed region in (FIG. 12g3). Arrows show the co-labeled cells. FIG. 12i, PlxnD1 mRNA expression in P56 S1 cortex from the Allen Mouse Brain Atlas. FIG. 12j, Specificity of READR^{PlxnD1} measured as the percent of PlxnD1⁺ cells among mNeon cells. FIG. 12k, Genomic structures of the mouse Rorb gene with location of a sesRNA as indicated. FIG. 12l, AAVs READR^{Rorb} and TRE-mNeon labeled cells in layer 4 (FIG. 1211, FIG. 1213). DAPI staining indicated laminar structure (FIG. 1212). mNeon labeling pattern is consistent with Rorb mRNA expression in P56 S1 cortex from the Allen Mouse Brain Atlas (FIG. 12m); the specificity of Rorb sesRNA is yet to be rigorously quantified due to the lack of a specific antibody to RORB and a in situ probe to Rorb mRNA. FIG. 12n, Genomic structures of mouse the vGAT gene with location of a sesRNA as indicated. FIGS. 12o-12p, binary READR^{vGAT} and TRE-mNeon labeled cells (FIG. 1201). vGAT mRNA in-situ hybridization. (FIG. 1202). Co-labeling by READR^{vGAT} AAVs and vGAT mRNA (FIG. 1203). FIG. 12p, Magnified view of rectangle in (FIG. 1203). Arrows show t, he co-labeled cells. FIG. 12q, vGAT mRNA expression in P56 S1 cortex from the Allen Mouse Brain Atlas. FIG. 12r, Specificity of READR^{GAT} measured as the percent of vGAT⁺ cells among mNeon cells.

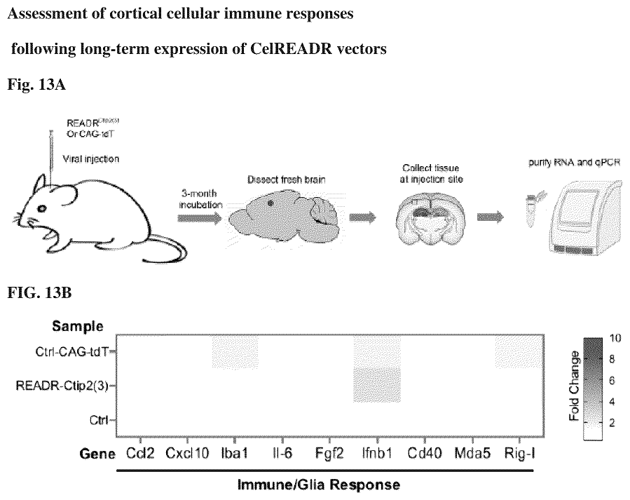

### FIGS. 13A-13B Assessment of Cortical Cellular Immune Responses Following Long-Term Expression of CellREADR Vectors

FIG. 13a, Schematic of evaluation of the long-term effects of CellREADR in vivo. For each mouse, READR^{Ctip2(3)} or CAG-tdT control AAVs were injected into S1 cortex and incubate for three months. Fresh brains were dissected and small pieces of cortical tissue at the injection site were collected. Quantitative PCR was performed immediately. S1 tissues of mice without viral injection were used as control. FIG. 13b, Heatmap of expression level changes of nine genes implicated in glia activation and immunogenicity.

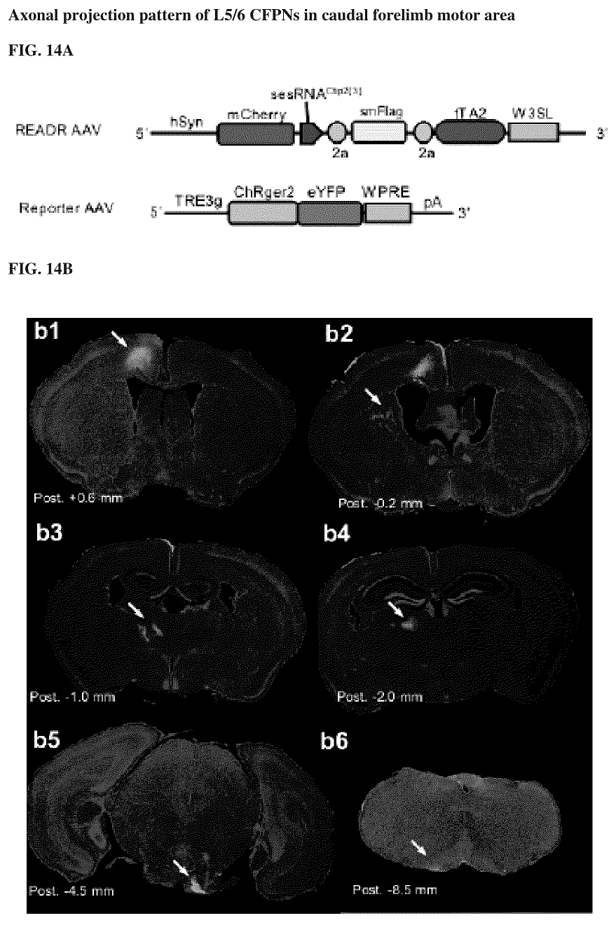

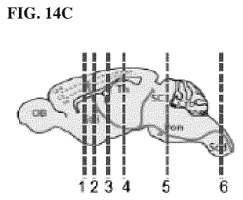

### FIGS. 14A-B Axonal Projection Pattern of L5/6 CFPNs in Caudal Forelimb Motor Area Targeted by AAVs READR^{Ctip2} and TRE-ChRger2-eYFP

FIG. 14a, Schematic of binary AAV vectors of READR^{Ctip2(3)-smFlag/tTA2} and TRE-ChRger2-eYFP for optogenetic activation and axonal projection tracing. FIG. 14b, Axonal projection pattern of CFPNs infected in CFA. Representative images showing projections to striatum (FIG. 14b2), thalamus (FIG. 14b3, FIG. 14b4), pons (FIG. 14b5) and medulla (FIG. 14b6, arrows). FIG. 14c, Schematic locations of coronal sections in FIG. 14b are shown.

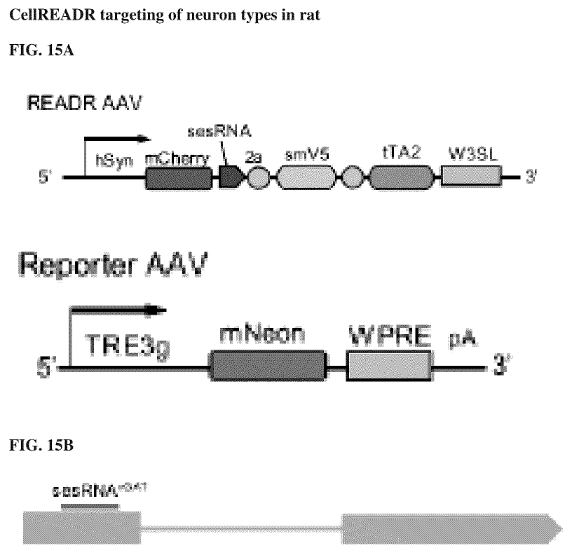

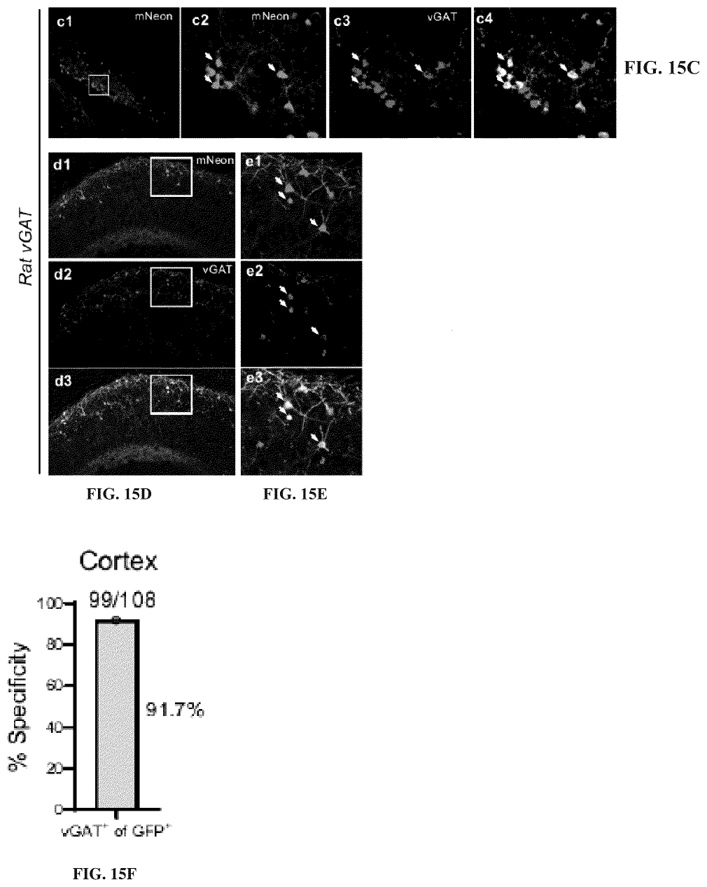

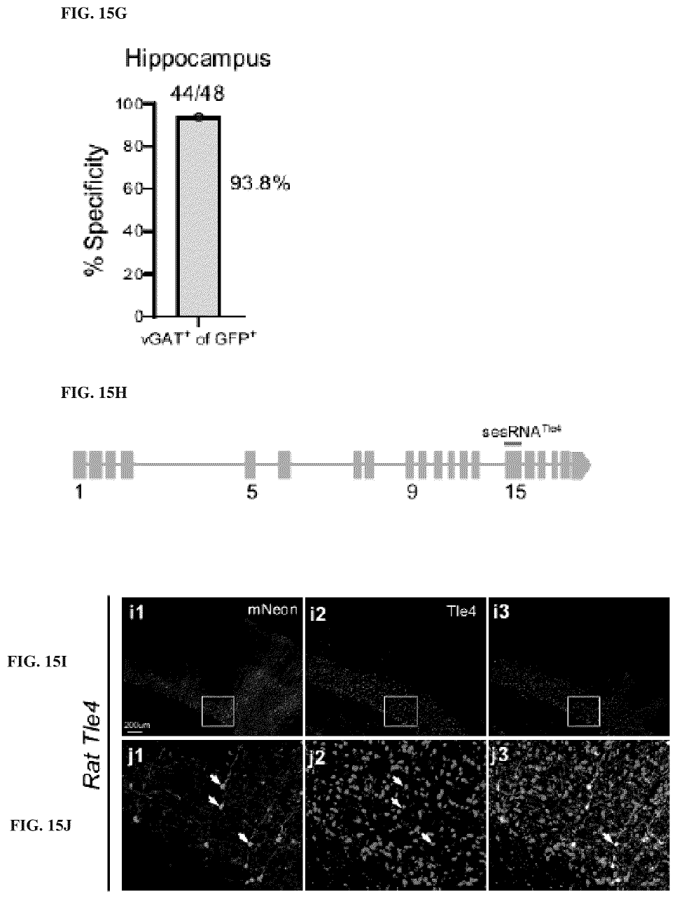

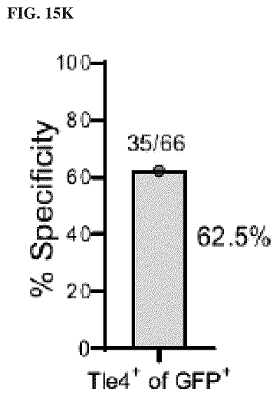

### FIGS. 15A-K. CellREADR Targeting of Neuron Types in Rat

FIG. 15a, Schematic of binary AAV vectors for cell type targeting in rat. In READR vector, a hSyn promoter drives expression of mCherry followed by sequences coding for sesRNA, smFlag and tTA2 effectors. Along with READR, a Reporter vector drives mNeonG expression from a TRE promoter in response to tTA2 from the READR vector. FIG. 15b, Genomic structures of the rat vGAT gene with location of a sesRNA as indicated. FIG. 15c, AAVs READR^{vGAT} and TRE-mNeon were injected into cortical deep layer and hippocampus. Binary vectors labeled cells shown in cortex (FIG. 15c1). FIG. 15c2, Magnified view of boxed region in (FIG. 15c1). vGAT mRNAs were labeled by in-situ hybridization (FIG. 15c3). Co-labeling by mNeon and vGAT mRNA (FIG. 15c4). Arrows showed the co-labeled cells. FIG. 15d, Cell labeling pattern in the hippocampus CA1 region by co-injection of AAVs READR^{vGAT} and TRE-mNeon. FIG. 15e, Magnified view of boxed region in (FIG. 15d). Arrows indicate co-labeled cells. FIGS. 15f-15g, Specificity of rat READR^{vGAT} in rat cortex (FIG. 15f) and hippocampus (FIG. 15g) measured as the percent of vGAT⁺ cells among mNeon cells. FIG. 15h, Genomic structures of the rat Tle4 gene with the location of a sesRNA as indicated. FIGS. 15i-15j, AAVs READR^{Tle4} and TRE-mNeon (FIG. 4i) co-injected into the rat motor cortex labeled cells concentrated in deep layers (FIG. 15i1). Tle4⁺ PNs were labeled by TLE4 antibody staining (FIG. 15i2, FIG. 15i3). FIG. 15j, Magnified view of the boxed region in (FIG. 15i). Arrows indicate co-labeled cells by CellREADR and TLE4 antibody. FIG. 15k, Specificity of rat READR^{Tle4} in rat cortex, measured as the percent of TLE4+ cells among mNeon cells.

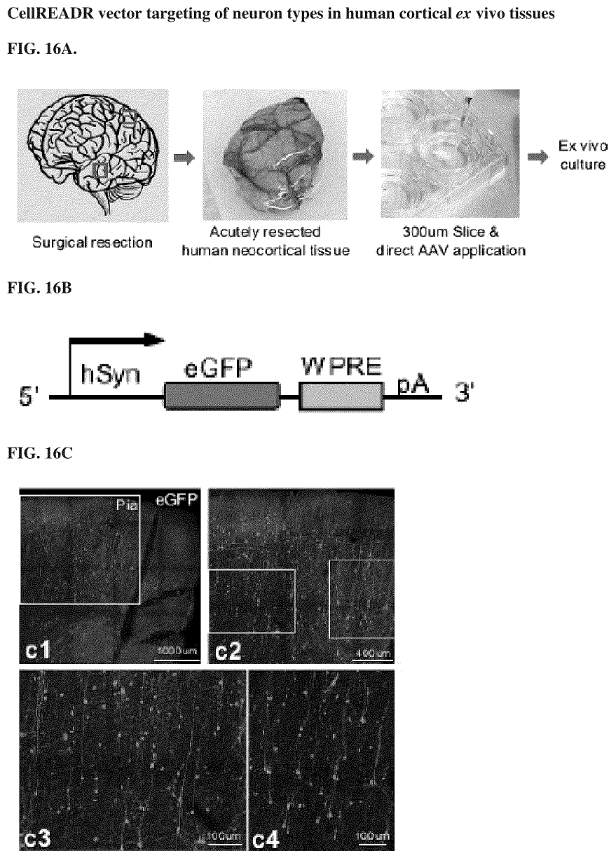

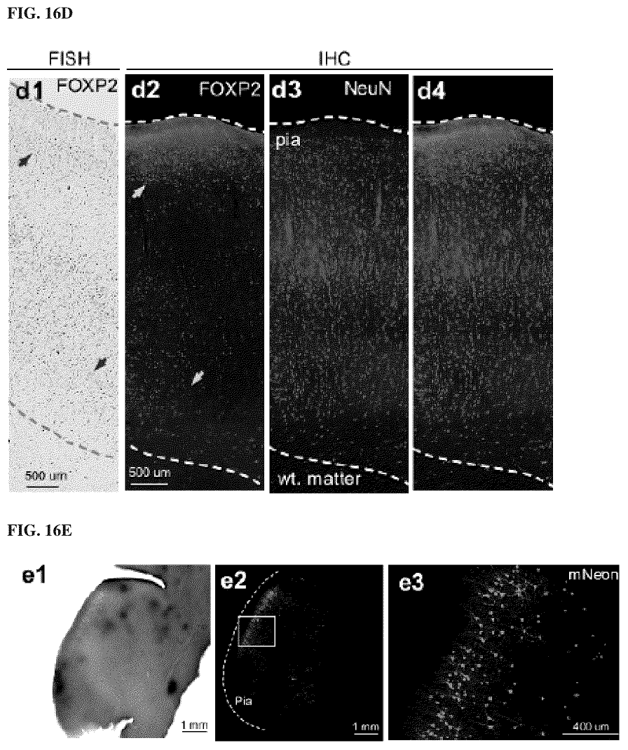

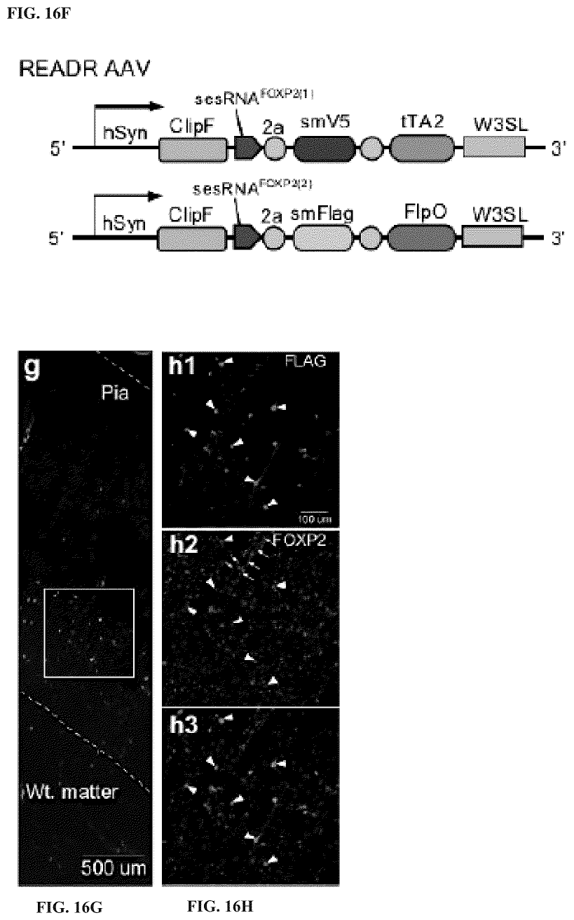

### FIGS. 16A-16I CellREADR Vector Targeting of Neuron Types in Human Cortical Ex Vivo Tissues

FIG. 16a, Schematic of organotypic platform for of human cortical ex vivo tissues. FIG. 16b, Schematic of a hSyn-eGFP viral construct used to drive widespread neuronal cell labeling. FIG. 16c. AAVrg-hSyn-eGFP labeled cells were distributed across all layers, and exhibited diverse morphologies (FIG. 16c1). Insets from FIG. 16c1 (FIG. 16c2) and FIG. 16c2 (FIG. 16c3, FIG. 16c4) depict numerous cells with pyramidal morphologies, including prominent vertically oriented apical dendrites. FIG. 16d, FOXP2 expression in human neocortex. FOXP2 mRNA expression pattern taken from the Allen Institute human brain-map (specimen #4312), showing upper and deep layer expression (arrows) (FIG. 16d1). FOXP2 immunostaining in the current study (magenta) also demonstrated both upper and deep labeling (FIG. 16d2, FIG. 16d4). NeuN immunostaining (red) depicting cortical neurons (FIG. 16d3, FIG. 16d4). Dashed lines delineate pia and white matter. FIG. 16e, READR^{FOXP2(1)} labeling in an organotypic slice derived from the same tissue used in FIG. 16d). Overview of bright field and mNeon native fluorescence in the organotypic slice demonstrating highly restricted labeling, as compared to that observed in FIG. 16d. Inset from FIG. 16e2 (FIG. 16e3) depicting morphologies of upper layer pyramidal neurons. FIG. 16f, Schematics of two singular vectors of READR^{FOXP2}. In READR^{FOXP2 (1)}, the hSyn promoter drives an expression cassette encoding ClipF, sesRNA1, smV5, and tTA2. In READR^{FOXP2(2)}, the hSyn promoter drives an expression cassette encoding ClipF, sesRNA2, smFlag, and FlpO. FIG. 16g, Seven days after application of READR^{FOXP2(2)} AAV on DIV 1, tissue was fixed and FIG. 16h stained with antibodies against FOXP2 and FLAG. FLAG-labeled cells from READR^{FOXP2(2)} exhibited relatively small somata with short apical dendrites (arrowheads). Non-specific background fluorescence signals (e.g. a blood vessel-like profile) are indicated by thin arrows in center panel. FIG. 16i, Quantification of CellREADR specificity measured as the percentage of V5⁺ cells (for READR^{FOXP2(1)}) and FLAG⁺ cells (for READR^{FOXP2(2)}) labeled by FOXP2 immunostaining, respectively.

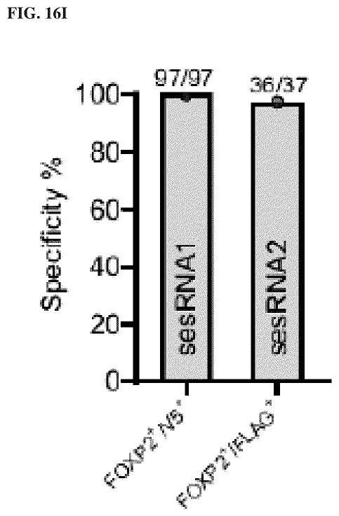

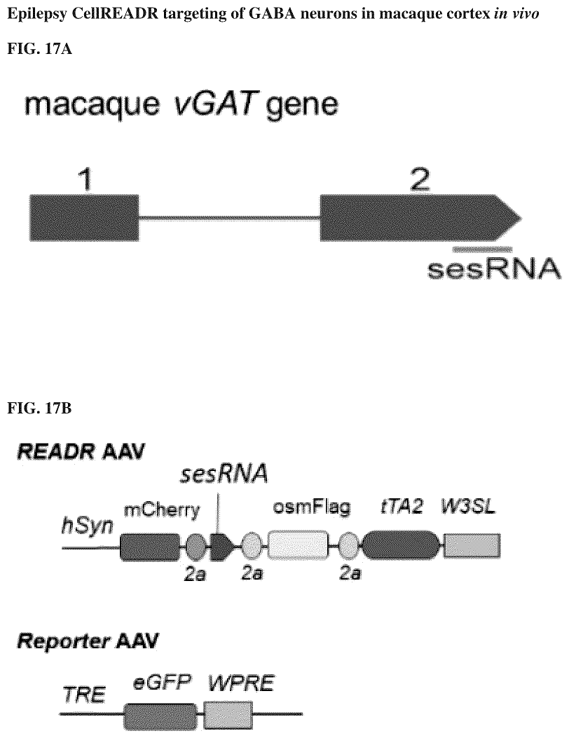

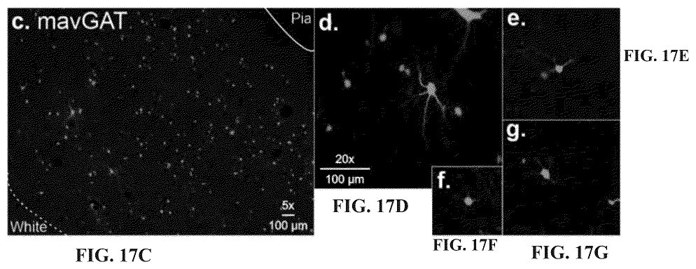

### FIGS. 17A-17G Epilepsy CellREADR Targeting of GABA Neurons in Macaque Cortex In Vivo.

FIG. 17a. schematic of macaque vGAT gene. FIG. 17b. binary CellREADR AAV vectors. FIGS. 17c-17G labeling of neurons with interneuron morphology in macaque primary visual cortex after 2 months of AAV incubation.

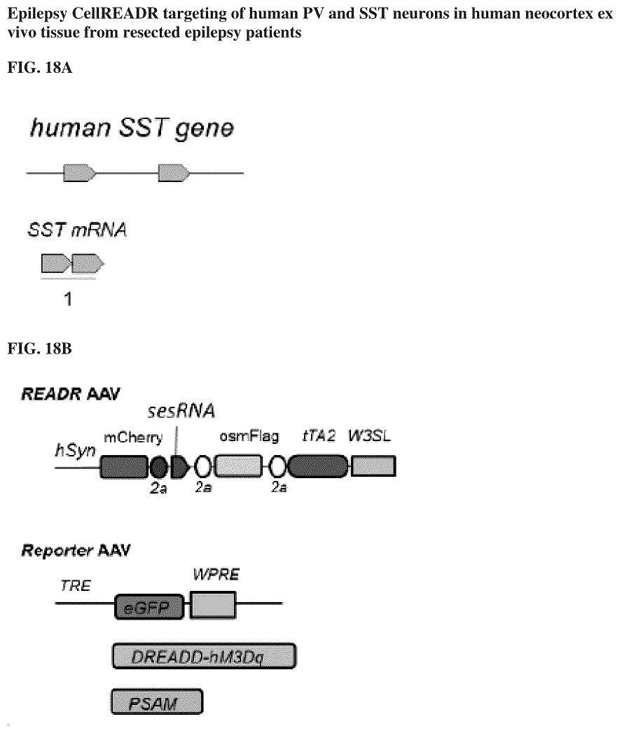

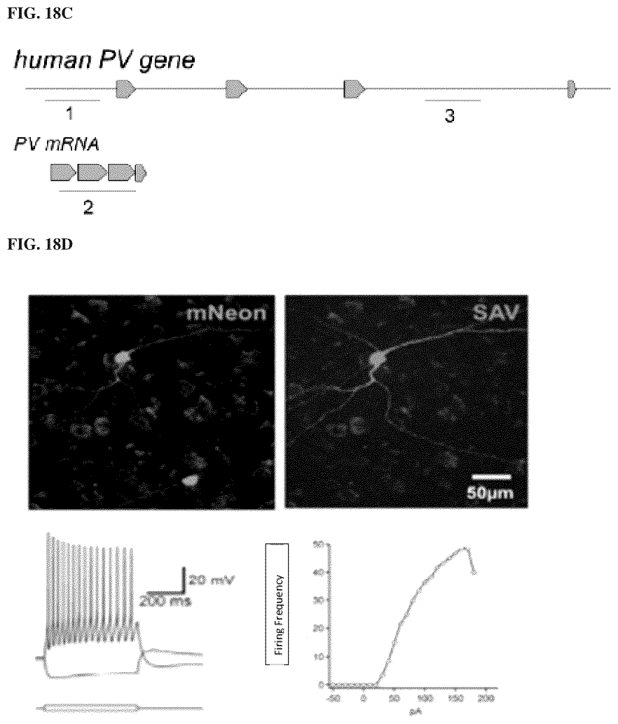

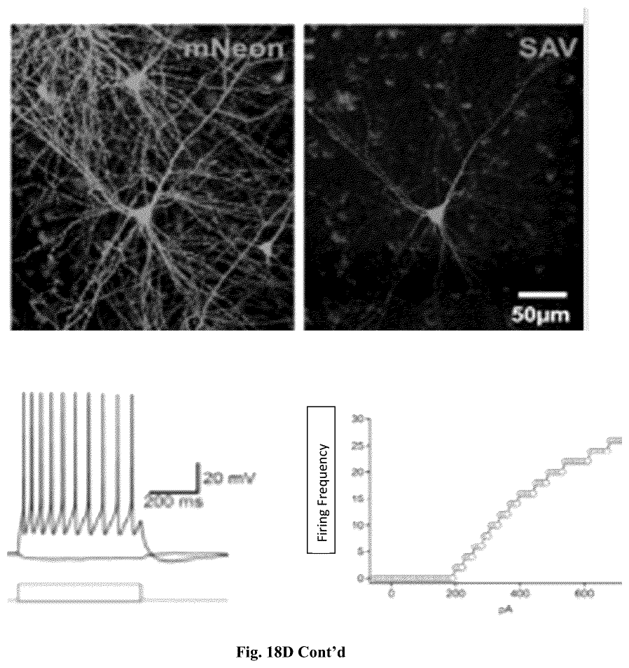

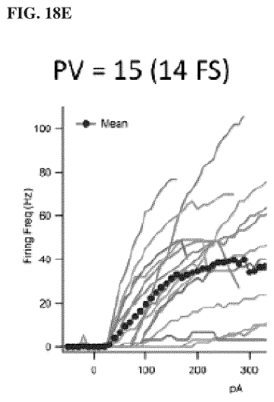

FIGS. 18A-E Epilepsy CellREADR Targeting of Human PV and SST Neurons in Human Neocortex Ex Vivo Tissue from Resected Epilepsy Patients.

FIG. 18a. human SST gene and sesRNA design. FIG. 18b. binary AAV vectors to label and manipulate GABA neuron types. FIG. 18c. human PV gene and sesRNA design. FIG. 18d. examples of morphological labeling and firing pattern of human PV neurons. FIG. 18e. 14 of the 15 neurons targeted by PV sensors show fast spiking, characteristic to PV interneurons.

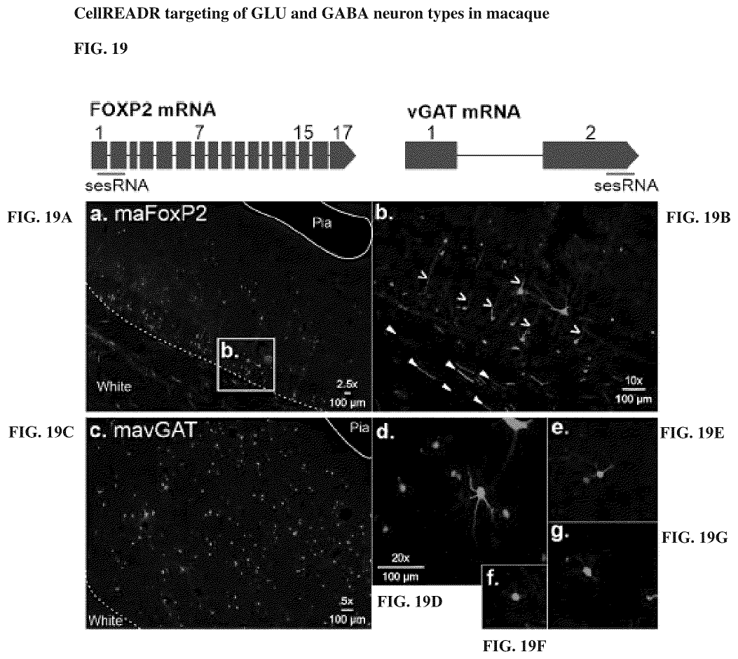

### FIGS. 19A-G CellREADR Targeting of GLU and GABA Neuron Types in Macaque.

FIG. 19a. AAV-READRFOXP2; AAV-TRE3g-mNeon labeled putative FOXP2 cells in V1. Boxed region is magnified in FIG. 19b to show GFP+ pyramids with apical dendrites (open arrowheads) mostly in L6, and axons in white matter (closed arrowheads) suggestive of their efferents. FIG. 19c. AAV-READRVGAT; AAV-TRE3g-mNeon labeled putative GABA cells in V1. FIGS. 19d-19g. Example multipolar cells from c with aspiny dendrites characteristic of diverse GABA INs.

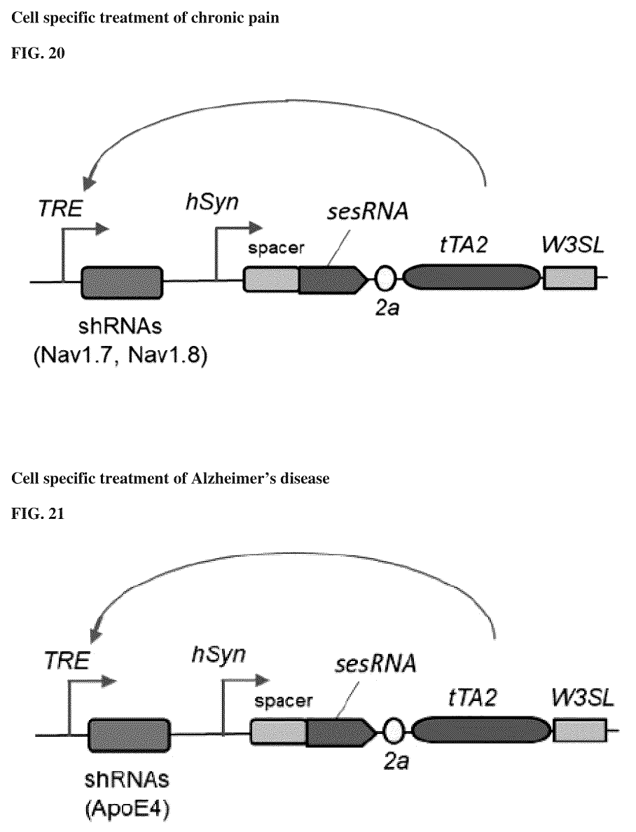

### FIG. 20 Cell Specific Treatment of Chronic Pain

Binary expression vector in which the translation of transcription activator tTA is gated by NaV1.7 or NaV1.8 RNA sensor; tTA then activates the transcription of an shRNA for NaV1.7 or NaV1.8 RNA.

### FIG. 21 Cell Specific Intervention of Alzheimer's Disease

Schematic of a READR RNA consisting of sesRNA targeting AD marker RNA and effector RNA encoding tTA2 which are driven by a hSyn promoter. Upstream of this cassette, shRNAs against ApoE4 RNA are driven by TRE enhancer.

### DESCRIPTION

### Definitions

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Accordingly, one aspect of the present disclosure provides a modular readrRNA molecule comprising: (i) a 5′ region comprising a sensor RNA domain, the sensor domain comprising an ADAR-editable STOP codon; and (ii) a 3′ region comprising an effector RNA (efRNA) domain that is downstream of and in-frame with the sensor domain, wherein the sensor domain of the modular readrRNA molecule specifically binds to a cellular RNA encoded by a gene differentially expressed in a cell, preferably a cell or group of cells of the cortex, through sequence-specific base pairing to form an RNA duplex, wherein the efRNA encodes a protein, and wherein the ADAR-editable STOP codon act as a translation switch thereby allowing for the translation of the efRNA region to produce the effector protein.

In one aspect, the modular readrRNA molecule further encodes a self-cleaving 2A peptide positioned between the sensor domain and effector RNA region. The self-cleaving 2A peptide is selected from the group consisting of one or more of T2A peptide, P2A peptide, E2A peptide, and F2A peptide. Preferably, the 2A peptide is T2A.

In one aspect, the effector (efRNA) domain of the modular readrRNA encodes a detectable protein or detectable protein fragment (a label) and/or a transcriptional activator or repressor.

In one aspect, the gene(s) differentially expressed in cells of the cortex are selected from the group consisting of Fezf2, Ctip2, PlxnD1, Satb2, Rorb and vGAT.

Another embodiment of the invention is a CellREADR “system” comprising at least two components, wherein the first component comprises a modular readrRNA molecule, and wherein the second component comprises a gene that is responsive to the effector domain-encoded protein. In an aspect of the CellREADR system, the efRNA-encoded protein by the modular readrRNA molecule comprises the tetracycline dependent transcription activator tTA2, and wherein the sensor domain of the modular readrRNA molecule specifically binds to a cellular RNA encoded by the Ctip2 gene, and wherein the second component of the CellREADR system comprises a gene operably linked to a TRE-3G eukaryotic inducible promoter that is responsive to the tetracycline dependent transcription activator tTA2. In an aspect of the CellREADR system, the efRNA-encoded protein by the modular readrRNA molecule further comprises smFlag. In an aspect of the CellREADR system, the gene operably linked to a TRE-3G eukaryotic inducible promoter encodes the calcium indicator GCaMP6s. In an aspect of the CellREADR system, the gene operably linked to a TRE-3G eukaryotic inducible promoter encodes the calcium indicator GCaMP6s. In an aspect of the CellREADR system, the gene operably linked to a TRE-3G eukaryotic inducible promoter encodes the light-activated ion channel channelrhodopsin-2.

Another embodiment of the invention is a method of stimulating GCaMP6s signals in forelimb somatosensory cortex as measured by photometry in mice transduced with the CellREADR ^{Ctip2/3} in response to mechanical stimulation of the forepaw, comprising coinfecting the mice with the above referenced CellReadR system.

Another embodiment of the invention is a method of stimulating forelimb movement in response to light stimulation of the CFA of mice transduced with the CellREADR ^{Ctip2/3} comprising coinfecting the mice with the above referenced CellReadR system. In another aspect, a second gene is operably linked the TRE-3G eukaryotic inducible promoter, the second gene (eYFP) encoding enhanced yellow fluorescent protein.

Another embodiment of the invention is a delivery system comprising the modular readrRNA molecule operatively associated with a vehicle for administering the modular readrRNA to an organism, tissue or cell.

Another embodiment of the invention is a delivery system comprising the above referenced CellREADR system operatively associated with a vehicle for administering the above referenced CellREADR system to an organism, tissue or cell.

Another embodiment of the invention is a delivery system, the delivery vehicle is selected from the group consisting of a nanoparticle, a liposome, a vector, an exosome, a micro-vesicle, a gene-gun, and a Selective Endogenous eNcapsidation for cellular Delivery (SEND) system.

Another embodiment of the invention is a delivery system, the delivery vehicle comprising a recombinant viral vector, preferably one of adeno-associated virus (AAV), adenovirus, retrovirus, lentivirus, herpes viral vector, vesicular stomatitis virus, and combinations thereof.

Another embodiment of the invention is a pharmaceutical composition comprising the above referenced modular readrRNA molecule and/or the above referenced CellREADR system and/or the above referenced delivery system, and a pharmaceutically acceptable carrier, excipient and/or diluent.

Another embodiment of the invention is a cell comprising the above referenced modular readrRNA molecule and/or the above referenced CellREADR system, or the above referenced delivery system, where preferentially, the cell is a mammalian cell.

Another embodiment of the invention is a kit comprising the kit comprising the above referenced modular readrRNA molecule, and/or the above referenced CellREADR system and/or the above referenced delivery system.

Another embodiment of the invention is a method of identifying GABAergic neurons in mammalian cortex in vivo, comprising targeting vesicular GABA transporter (vGAT) mRNA in mammalian cortex in vivo, the method comprising administering into the S1 barrel cortex and hippocampus of the mammal (i) a binary AAV vector comprising READR^{vGAT} and (ii) a Reporter^{mNeon}, wherein the READRvGAT comprises [hSyn-mCherry-sesRNA^{vGAT}-smFlag-tTA2] in 5′ to 3′ transcriptional order a human Syn promoter operably linked to RNA encoding a red fluorescent protein mCherry followed by sequences coding for sesRNA targeting vGAT mRNA, a 2a protein, smFlag (smV5), a 2^{nd} 2a protein, a tetracycline dependent transcription activator tTA2 and W3SL wherein the Reporter^{mNeon} comprises [TRE3g-mNeon-WPRE], wherein upon theadministration, vGAT mRNA in the S1 barrel cortex and hippocampus of themammal is co-labeled with magenta and GFP, indicating identification of GABAergic neurons in themammalian cortex. In one aspect, the sesRNA targeting vGAT mRNA is complementary to all or part of exon 1 of vGAT mRNA.

Another embodiment of the invention is a method of identifying GABAergic neurons in mammalian cortex in vivo, comprising targeting Transducin-like enhancer protein 4 (Tle4) mRNA, the method comprising administering into the S1 barrel cortex of the mammal (i) a binary AAV vector comprising READR^{vGAT} and (ii) a Reporter^{mNeon}, wherein the READR^{vGAT}comprises [hSyn-mCherry-sesRNAvGAT-smFlag-tTA2] in 5′ to 3′ transcriptional order a human Syn promoter operably linked to RNA encoding a red fluorescent protein mCherry followed by sequences coding for sesRNA targeting Tle4 mRNA, a 2a protein, smFlag (smV5), a 2^{nd} 2a protein, a tetracycline dependent transcription activator tTA2 and W3SL, wherein the Reporter^{mNeon} comprises [TRE3g-mNeon-WPRE] wherein upon the administration, Tle4 mRNA in the S1 barrel cortex of the mammal was co-labeled with magenta and GFP, indicating identification of GABAergic neurons in the mammalian cortex. In one aspect, the sesRNA targeting Tle4 mRNA is complementary to all or part of exon 15 of Tle4 mRNA. Another embodiment of the invention is a method of determining in an organotypic culture platform cortical layers which express human FOXP2 an evolutionarily conserved gene implicated in human language skill development, comprising administering READR^{FOXP2}/Reporter^{mNeon} AAVs (hSyn-ClipF-sesRNA^{FOXP2}-smV5-tTA2 with TRE3g-mNeon) to human neocortical slices of the organotypic culture platform, wherein 5 days post administration, mNeon-labeled cell bodies observed in upper and deep layers of the organotypic culture platform cortical layers, indicating expression of human FOXP2 in theupper and deep layers.

Another embodiment of the invention is a method to suppress neural activities in some cell types that promote focal epileptic seizure comprising targeting pathophysiological neural ensembles (PNE) comprising cell type targeted modulation of neural activity those that participate in seizure activity, wherein a marker for the neural activity is the rapid upregulation of the immediate early gene (IEG) c-fos, the method comprises administering the READR^{c-fos} operably linked to one of three effector genes selected from the group consisting of: a voltage-gated potassium channel Kv1.1 (KCNA1), the inhibitory DREADD (the hM4D (Gi) inhibitory receptor) and invertebrate glutamate receptor (eGluCL) that is permeable to chloride, wherein during epilepsy, up-regulation of c-fos RNA will trigger effector translation to suppress PNE activities. In one aspect, the effector gene is inhibitory DREADD receptor, and it is activated by an oral drug olanzapine. In another aspect, the effector gene is eGluCL, and it is activated by the drug ivermectin. In another aspect, the effector gene is inhibitory PSEM, and it is activated by the drug varenicline.

Another embodiment of the invention is a method of treating chronic pain, comprising targeting nociceptors comprising RNA sensor RNA sensor targeting RNA encoded by the voltage gated sodium channels TRPV1 or NaV1.8 of the dorsal root ganglia, and mediating effector expression using a CellReadR cell-type specific small hairpin RNAs (shRNA) in a knockdown approach to down-regulate NaV1.7 and NaV1.8 mRNAs in nociceptors.

Another embodiment of the invention is a method of treating Alzheimer's Disease targeting Marker genes/RNAs, including but not limited to in astrocytes: aldehyde dehydrogenase family 1 member L1 (Aldh1L1), glial fibrillary acidic protein (GFAP), excitatory amino acid transporter 1 (EAAT1); and in microglia: C-X3-C Motif Chemokine Receptor 1 (CX3CR1), transmembrane protein 119 (TMEM119), Ionized calcium binding adaptor molecule 1 (IBA-1.) and mediating effector expression using a CellReadR cell-type specific small hairpin RNAs (shRNA) to down-regulate ApoE4.

As used herein, the term “stop codon” refers to a sequence of three nucleotides (a trinucleotide) in DNA or messenger RNA (mRNA) that signals a halt to protein synthesis in the cell.

A “codon” in a messenger RNA corresponds to a nucleotide triplet that encodes an amino acid. Consecutive codons in an RNA are translatable to a protein. In nature, a stop codon is located in the 3′ terminal end of the coding region(s) of a mRNA and signals the termination of translation by binding release factors, which binding causes the ribosomal subunits to disassociate and thereby to release the amino acid chain. There are 64 different trinucleotide codons: 61 specify amino acids and 3 are stop codons (i.e., UAA, UAG and UGA in RNA and TAA, TAG and TGA in DNA).

As used herein, an “editable stop codon” refers to a stop codon that is editable by a cell from a stop codon to a translatable codon. Thus, in RNA, an editable stop codon which is a UAA, a UAG or a UGA is editable by a cell to UII, UIG, or UGI. An editable stop codon functions as a translation switch for any codons downstream of the editable stop codon. Editing of a stop codon occurs in cells in which an endogenous ADAR enzyme is present.

“Editing” of a stop codon occurs when a sensory RNA containing an editable stop codon forms dsRNA with a target RNA, thereby recruiting endogenous ADAR enzyme. ADAR acts at the STOP codon, performs A to I editing and thus converts for example a UAG STOP to a UIG (tryptophan) codon, which permits translation of downstream codons.

The term “ADAR” is a disambiguation that stands for adenosine deaminase acting on RNA. ADAR enzymes bind to double-stranded RNA (dsRNA) and convert adenosine to inosine (hypoxanthine) by deamination. ADAR proteins act post-transcriptionally, changing the nucleotide content of RNA. The conversion from adenosine to inosine (A to I) in the RNA disrupts the normal A:U pairing, destabilizing the RNA. Inosine is structurally similar to guanine (G) which leads to inosine to cytosine (I:C) binding. Inosine typically mimics guanosine during translation but can also bind to uracil, cytosine, and adenosine, though it is not favored.

As used herein, “readrRNA” refers to a molecule having a 5′ region and a 3′ region, where the readrRNA molecule comprises, consists of, or consists essentially of (i) a 5′ region comprising a sensor (ses) domain, the sensor domain comprising at least one ADAR-editable STOP codon; and (ii) an effector RNA (efRNA) region that is downstream and in-frame with the sensor domain.

In a readrRNA, an ADAR-editable stop codon is located within the sensor RNA and upstream of the in-frame effector coding region.

As used herein, an “ADAR-editable STOP codon” refers to a stop codon that is editable in a cell by ADAR. Schneider, M. F., Wettengel, J., Hoffmann, P. C., & Stafforst, T. (2014). Optimal guide RNAs for re-directing deaminase activity of hADAR1 and hADAR2 in trans. Nucleic acids research, 42(10), e87. https://doi.org/10.1093/nar/gku272

As used herein, a “sensor domain” refers to a consecutive set of nucleotides that form a portion of a readrRNA, where the sensor domain also includes at least one editable stop codon and a downstream effector domain. A sensor domain contains consecutive nucleotides that are complementary to an RNA of a specific cell type through sequence-specific base pairing. A sensor domain may comprise any number of nucleotides, comprising at least 10 nucleotides to at least 1000 nucleotides or more. In some embodiments, the sensor domain comprises, consists essentially of or consists of about 100 to about 900 nucleotides. In another embodiment, the sensor domain comprises, consists essentially of or consists of a range of about 200 nucleotides to about 300 nucleotides. A sensor domain may be 10, 25, 50, 75, 100, 150, 200, 250, 300, 350, 400, 450, 500, 550, 600, 650, 700, 750, 800, 850, 900, 950, 1000 or more consecutive nucleotides in length.

The at least one editable stop codon(s) in a sensor domain is/are located anywhere within the sensor domain of the readrRNA. A sensor domain will have a 5′ to 3′ orientation in the readrRNA molecule and includes an upstream (5)′ portion and a downstream (3)′ portion. An editable stop codon may be located in the sensor domain upstream portion or the sensor domain downstream portion. An editable stop codon may be located in the upstream portion of the sensor domain closer to the middle of the sensor domain or an editable stop codon may be located in the downstream portion of the sensor domain closer to the downstream end of sensor domain. For example, if the sensor domain is 600 nucleotides in length and divided spatially into halves, with the first nucleotide representing the 5′ end of the sensor domain, the 300^{th} nucleotide representing middle of the sensor domain, and the last (600^{th}) nucleotide of the sensor domain representing the 3′ end of the sensor domain, an editable stop codon may be located in the upstream portion of the downstream portion or closer to the middle of the sensor domain. If a sensor domain having the same approximate length is divided into quarters, an editable stop codon may be located in the first quarter portion (nucleotides 1-250) of the sensor domain, the second quarter portion of the sensor domain (nucleotides 150-300), the third quarter portion of the sensor domain (nucleotides 300-450), or the fourth quarter portion of the sensor domain (nucleotides 450-600). Thus, for a given length of a sensor domain, an editable stop codon may be located in a selected portion of the sensor domain. Generally, an editable stop codon may be located in the downstream half of a sensor domain, or the downstream quarter of a sensor domain. A selected portion of the sensor domain containing an editable stop codon may be within 10-50 nucleotides of the 3′ end of the sensor domain.

As used herein, an “effector RNA (efRNA)” is RNA that is translatable and encodes an effector protein.

An “effector protein” is a protein encoded by an effector RNA domain and that has an effect on a cell in which it is expressed. An effector protein is translated from an effector RNA in a cell and therefore an effector protein, like the RNA encoding it, is introduced into a cell that may or may not contain the same endogenous protein. An effector protein is a protein having an effect on the cell in which it is translated or, if secreted from the cell, on surrounding cells. No limiting examples of effector proteins include: an enzyme, a detectable protein, a cytokine, a toxin, a polymerase, a transcription or translation factor, a tumor suppressor, a neuronal activator or inhibitor, an apopotic protein or a physiological factor.

As used herein, an “effector RNA (efRNA) region” refers to a portion of a readrRNA comprising an effector RNA that is downstream and in-frame with a sensor domain.

The effector RNA (efRNA) may code for an effector protein of interest. Selection of a desired effector protein is well within the skill of one of ordinary skill in the art and is dependent on the context of the desired use of the readrRNA. For example, if it is desired to treat a given disease, an effector protein may be selected based on its having an inhibitor effect on cells that are critical to establishing and/or prolonging the disease. For example, the effector module of CellREADR (efRNA) can be built to manipulate cells in multiple ways, including enhance activity and function, suppress activity and function, rescue a mutant cell function by re-introducing an intact version of the deleted or mutated protein, alter and edit activity and function, reprogram cell identity, fate, and function, kill and delete a cell type, increase or decrease the production of cell numbers of a type, and cell type-specific genomic editing and gene regulation.

Non-limiting examples of effector are listed in Table 1 below. Table 1 provides a non-limiting list of effector proteins (payloads) useful according to the invention as well as their effect(s) on a cell.

TABLE 1

| Cell manipulations | effector RNA payloads |
|---|---|
| Cell killing | thymidine kinase (TK), cytosine deaminase (CD) |
| Programmed Cell death | Caspase3, Caspase9, Bcl, GSDME, GSDMD, GZMA, GZMA |
| Neural activation | ChR2, DREADD-M3Dq, PSAM |
| Neural inhibition | DREADD-hM4D, NpHR, GtACR1 |
| Cell fate reprogramme | NeuroD, Ptbp1 |
| Cell electrophysiology | NaChBac, Kir2.1; ion channels |
| Protein synthesis inhibitor | Ricin |
| Cell regeneration | EGFs, VEGFs |
| Tumor suppressor | Rb, p53, PTEN |
| immunity enhancer | IFNB, IFNG, TNFA, IL2, IL12, IL15, CD40L |

The term “payload” in the context of an effector RNA “payload” means a protein encoded by an RNA encoding an effector protein.

As used herein, the phrase “cell type” is a somatic cell of an organism, of an organ, of a tissue, a population of cells, or of a cell line, or of a hybridoma, of a homogenous cell population, or a heterogenous cell population, a cell that is resting or quiescent or activated, or transformed or diseased or stressed or inflamed or undergoing heat shock, or is part of the innate immune system, part of the natural immune system, a stem cell, a pluripotent stem cell, an intestinal stem cell, stem cell, a fetal cell, cell that contributes to homeostasis, a cardiac or vascular related cell, a cell of the digestive system, a cell of the nervous system, a cell of the skeletal structure, of a cell of an organ, that can be identified through differential expression of RNA.

Conceivably every cell or group thereof, is different and single cell RNA analysis is bearing evidence of this diversity with respect to a cell's lineage, its activation state, state of development, its state as a result of interaction with other cells, tissues and organs, and soluble molecules, and/or if the cell is diseased, transformed or infected.

As used herein, the term “self-cleaving 2A peptide” or “2A peptides” refers to the class of 18-22 amino acid-long peptides which can induce ribosomal skipping during translation of a protein in a cell. These peptides share a core sequence motif of DxExNPGP and are found in a wide range of viral families and help generating polyproteins by causing the ribosome to fail at making a peptide bond. Suitable examples of 2A peptides include, but are not limited to, T2A, P2A, E2A, F2A, and the like (Liu, Ziqing et al. “Systematic comparison of 2A peptides for cloning multi-genes in a polycistronic vector.” Scientific reports vol. 7,1 2193. 19 May 2017, doi:10.1038/s41598-017-02460-2). One such self-cleaving 2A peptide comprises a T2A peptide.

Several 2A peptides have been identified in picornaviruses, insect viruses and type C rotaviruses. As used herein, T2A is a 2A peptide identified in Thoseaasigna virus 2A; P2A is a 2A peptide identified in porcine teschovirus-1 2A; E2A is a 2A peptide identified in equine rhinitis A virus (ERAV) 2A; and F2A is a 2A peptide identified as a self-cleaving 2A peptides foot-and-mouth disease virus (FMDV). The following table provides DNA and corresponding amino acid sequences of various 2A peptides. Underlined sequences encode amino acids GSG, which are an example of optional additions to the native2A sequence, designed to improve cleavage efficiency; P2A indicates porcine teschovirus-1 2A; T2A, Thoseaasigna virus 2A; E2A, equine rhinitis A virus (ERAV) 2A; F2A, FMDV 2A. This is adapted from Table 1 of Kim J. H. et al. (High Cleavage Efficiency of a 2A Peptide Derived from Porcine Teschovirus-1 in Human Cell Lines, Zebrafish and Mice) PLoS One. 2011; 6(4): e18556. Published online 2011 Apr. 29. doi: 10.1371/journal.pone.0018556.

TABLE 2

| | |
|---|---|
| P2A | GGA AGC GGA GCT ACT AAC TTC AGC CTG CTG AAG CAG GCT GGA |
| | G S G A T N F S L L K Q A G |
| | GAC GTG GAG GAG AAC CCT GGA CCT |
| | D V E E N P G P |
| T2A | GGA AGC GGA GAG GGC AGA GGA AGT CTG CTA ACA TGC GGT GAC |
| | G S G E G R G S L L T C G D |
| | GTC GAG GAG AAT CCT GGA CCT |
| | V E E N P G P |
| E2A | GGA AGC GGA CAG TGT ACT AAT TAT GCT CTC TTG AAA TTG GCT |
| | G S G Q C T N Y A L L K L A |
| | GGA GAT GTT GAG AGC AAC CCT GGA CCT |
| | G D V E S N P G P |
| F2A | GGA AGC GGA GTG AAA CAG ACT TTG AAT TTT GAC CTT CTC AAG |
| | G S G V K Q T L N F D L L K |
| | TTG GCG GGA GAC GTG GAG TCC AAC CCT GGA CCT |
| | L A G D V E S N P G P |

As used herein, a “sequence coding for a self-cleaving 2A peptide” is nucleic acid, preferably RNA, encoding a self-cleaving 2A peptide as described above. According to the invention, the sequence coding for a self-cleaving 2A peptide typically is positioned in between the sensor domain and the effector RNA region.

The term “modular” when used in the context of the phrase “Modular readrRNA Molecule” refers a recombinant readrRNA molecule comprising nucleic acid sequences (preferably RNA sequences) encoding protein domains designed at the nucleic acid level, preferably at the RNA level, where the different protein domains can be assembled in the recombinant readrRNA molecule in the desired order with a specified number of repeats (including 0).

As used herein, “CellREADR” stands for “Cell access through RNA sensing by Endogenous ADAR [adenosine deaminase acting on RNA]”, and it is designed as a single, modular Readr RNA molecule, consisting of a 5′ sensor-edit-switch region (sesRNA) and a 3′ effector protein (or protein fragment) coding region (ef RNA), separated by a link sequence coding for a self-cleaving peptide T2A and an editing mechanism ubiquitous to all animal cells, such as by an ADAR-editable STOP codon. CellREADR provides a mechanism for detecting the presence of cellular RNAs and switching on the translation of effector proteins to monitor and manipulate physiology, functions and/or structure of a cell type. The following table provides cell types of various tissues, with an indication of enriched mRNA.

TABLE 3

| Tissue | Cell type | Enriched RNA Markers |
|---|---|---|
| Adipose subcutaneous | Adipocytes (Subcutaneous) | 605 |
| | Endothelial cells | 167 |
| | Smooth muscle cells | 331 |
| | Adipose progenitor cells | 240 |
| | Macrophages | 340 |
| | Mast cells | 36 |
| | T-cells | 161 |
| | Plasma cells | 100 |
| Adipose visceral | Adipocytes (Visceral) | 557 |
| | Mesothelial cells | 416 |
| | Endothelial cells | 215 |
| | Smooth muscle cells | 117 |
| | Adipose progenitor cells | 351 |
| | Macrophages | 222 |
| | Neutrophils | 64 |
| | Mast cells | 16 |
| | T-cells | 250 |
| | Plasma cells | 148 |
| Breast | Breast glandular cells | 270 |
| | Breast glandular cells | 28 |
| | (progenitors) | |
| | Breast myoepithelial cells | 66 |
| | Adipocytes (Breast) | 612 |
| | Endothelial cells | 288 |
| | Smooth muscle cells | 31 |
| | Fibroblasts | 238 |
| | Macrophages | 126 |
| | T-cells | 208 |
| | Plasma cells | 227 |
| Colon | Colon enterocytes | 369 |
| | Colon enteroendocrine cells | 338 |
| | Enteric glia cells | 240 |
| | Mitotic cells (Colon) | 85 |
| | Endothelial cells | 219 |
| | Smooth muscle cells | 166 |
| | Fibroblasts | 42 |
| | Macrophages | 143 |
| | Neutrophils | 67 |
| | Mast cells | 29 |
| | T-cells | 108 |
| | Plasma cells | 114 |
| Heart muscle | Cardiomyocytes | 916 |
| | Mitotic cells (Heart) | 56 |
| | Endothelial cells | 191 |
| | Smooth muscle cells | 38 |
| | Fibroblasts | 361 |
| | Macrophages | 135 |
| | Neutrophils | 52 |
| | T-cells | 75 |
| | Plasma cells | 78 |
| Kidney | Podocytes | 112 |
| | Proximal tubular cells | 657 |
| | Ascending Loop of Henle cells | 71 |
| | Intercalated cells | 75 |
| | Endothelial cells | 289 |
| | Fibroblasts | 283 |
| | Macrophages | 90 |
| | T-cells | 112 |
| | Plasma cells | 89 |
| Liver | Hepatocytes | 1264 |
| | Cholangiocyte | 57 |
| | NK-cells (Liver) | 33 |
| | Erythroid cells | 28 |
| | Endothelial cells | 330 |
| | Hepatic stellate cells | 316 |
| | Kupffer cells | 97 |
| | Neutrophils | 52 |
| | T-cells | 73 |
| | Plasma cells | 143 |
| Lung | Respiratory ciliated cells | 681 |
| | Alveolar cells type 1 | 132 |
| | Alveolar cells type 2 | 363 |
| | Mitotic cells (Lung) | 153 |
| | NK-cells (Lung) | 55 |
| | B-cells | 11 |
| | Endothelial cells | 97 |
| | Smooth muscle cells | 63 |
| | Fibroblasts | 253 |
| | Macrophages | 160 |
| | Neutrophils | 160 |
| | Mast cells | 17 |
| | T-cells | 89 |
| | Plasma cells | 185 |
| Pancreas | Alpha cells | 221 |
| | Beta cells | 200 |
| | Ductal cells | 115 |
| | Exocrine glandular cells | 39 |
| | Endothelial cells | 47 |
| | Fibroblasts | 72 |
| | Macrophages | 86 |
| | T-cells | 38 |
| | Plasma cells | 141 |
| Prostate | Prostate glandular cells | 438 |
| | Prostate basal glandular cells | 105 |
| | Urothelial cells | 159 |
| | Endothelial cells | 177 |
| | Smooth muscle cells | 351 |
| | Fibroblasts | 285 |
| | Macrophages | 96 |
| | T-cells | 182 |
| | Plasma cells | 143 |
| Skeletal muscle | Skeletal myocytes | 329 |
| | Endothelial cells | 237 |
| | Smooth muscle cells | 41 |
| | Fibroblasts | 338 |
| | Macrophages | 176 |
| | Neutrophils | 35 |
| | Plasma cells | 69 |
| Skin | Keratinocyte (other) | 737 |
| | Keratinocyte (granular) | 208 |
| | Melanocytes | 17 |
| | Hair cortex cells | 48 |
| | Inner root sheath cells | 22 |
| | Outer root sheath cells | 119 |
| | Sebaceous gland cells | 206 |
| | Eccrine sweat gland cells | 246 |
| | Langerhans cells | 20 |
| | Adipocytes (Skin) | 26 |
| | Mitotic cells (Skin) | 190 |
| | Endothelial cells | 213 |
| | Smooth muscle cells | 36 |
| | Fibroblasts | 147 |
| | Macrophages | 56 |
| | Mast cells | 12 |
| | T-cells | 58 |
| | Plasma cells | 99 |
| Stomach | Parietal cells | 74 |
| | Chief cells | 56 |
| | Gastric mucous cells | 379 |
| | Gastric enteroendocrine cells | 47 |
| | Mitotic cells (Stomach) | 170 |
| | Endothelial cells | 84 |
| | Fibroblasts | 166 |
| | Macrophages | 154 |
| | Neutrophils | 22 |
| | T-cells | 23 |
| | Plasma cells | 186 |
| Testis | Spermatogonia | 158 |
| | Spermatocytes | 188 |
| | Early spermatids | 757 |
| | Late spermatids | 584 |
| | Sertoli cells | 614 |
| | Leydig cells | 20 |
| | Peritubular cells | 42 |
| | Endothelial cells | 109 |
| | Macrophages | 60 |
| Thyroid | Parafollicular cells | 47 |
| | Thyroid glandular cells | 1598 |
| | Mitotic cells (Thyroid) | 69 |
| | Endothelial cells | 70 |
| | Smooth muscle cells | 86 |
| | Fibroblasts | 463 |
| | Macrophages | 117 |
| | Neutrophils | 36 |
| | T-cells | 350 |
| | Plasma cells | 182 |

As used herein, a “CellREADR system” includes the following components: (i) a sensor RNA domain which comprises a consecutive set of nucleotides that is complementary to a portion of a selected cellular RNA, (ii) an effector RNA (efRNA) domain encoding an effector protein, the efRNA domain being downstream of and in-frame with the sensor RNA domain, (iii) an ADAR-editable STOP codon that lies within the sensor RNA domain or lies between the sensor and effector RNA domains, and (iv) a second protein coding nucleic acid or a gene optionally including gene control elements, where (iv) that may or may not be physically linked to the sensor RNA and effector RNA domains. A CellREADR System may include an exogenous gene (DNA or RNA) not physically linked to the readrRNA (e.g., on a separate vector). A CellREADR System may include a cell that contains the readrRNA nucleic acid, a nucleic acid encoding a second protein, the cell being used for delivery to a multicellular organism, a plant, an animal, or a human.

As used herein, an “delivery system” refers to a system comprising a vehicle for administering a modular readrRNA molecule and/or CellREADR system, where the vehicle includes but is not limited to a nanoparticle, a liposome, a vector, an exosome, a microvesicle, a gene-gun, a SEND system, and combinations thereof.

A “SEND system” is an mRNA delivery system comprising humanized virus-like particles (VLPs) based on retroelements present in the human genome, (Segel M, et al. Mammalian retrovirus-like protein PEG10 packages its own mRNA and can be pseudotyped for mRNA delivery. Science. 2021; 373:882-889. doi: 10.1126/science.abg6155). The vector of a such a delivery system includes, but is not limited to, a recombinant viral vector such as Adeno-associated virus (AAV), Adenovirus, retrovirus. Lentivirus, Herpesviral vector, vesicular stomatitis virus, and combinations thereof. In one embodiment, the viral vector comprises an AAV vector.

As used herein, «is able to form a duplex with» means that the «corresponding stretch of consecutive nucleotides» can base pair with the stretch of consecutive nucleotides of the sensor domain RNA.

As used herein, “corresponding stretch” means a sequence that is of the same length of nucleotides and matches through base pairing.

“Stretch” indicates a length of consecutive nucleotides that is at least 15 bases or longer; longer includes 20 bases, 25 bases, 30 bases, 40 bases 50 bases, 60 bases, 75 bases, 100 bases, 125 bases, 150 bases, 175 bases, 200 bases, 225, bases, 250 bases, 275 bases, 300 bases, 325 bases, 350 bases, 375 bases, 400 bases, 425 bases, 450 bases, 475 bases, 500 bases, 525 bases, 550 bases, 575 bases, 600 bases, 625 bases, 650 bases, 675 bases, 700 bases, 725 bases, 750 bases, 775 bases, 800 bases, 825 bases, 850 bases, 875 bases, 900 bases, 925 bases, 950 bases, 975 bases, 1000 bases, and longer.

As used herein, a “translation switch” is a component of a readrRNA molecule comprising an ADAR-editable STOP codon component which, upon binding by upstream sensor RNA to complementary target RNA to form a double stranded RNA structure, results in subsequent ADAR mediated editing of the AUG stop codon, resulting in the translation of the downstream RNA that encodes for an effector protein.

As used herein, a “cellular RNA” means an RNA that is present in a given cell, whether the RNA is endogenous to the cell (i.e., transcribed from a gene endogenous to the cell), or is present in the cell because it is transcribed from a gene that has been introduced into the cell, or is transcribed from a pathogen (such as a virus, bacteria, fungus or another micro-organism) that has infected the cell.

As used herein, a “cellular RNA of a cell” means an RNA that is present in a cell that, as a result of possessing specific characteristic, is identifiable because the RNA is known to be present in a cell having those specific characteristics.

As used herein, a “cell state-defining cellular RNA” refers to one or more RNA sequences present in a select cell or group of cells of interest, the presence of which identifies the state of a given cell, including but not limited to, a specified cell physiology, a specified development stage of a cell, a specified transformation of a cell, or activation state of a cell.

That is, the specific physiology of a cell is in large part determined by its expression of a unique repertoire of RNA transcripts. The unique repertoire of RNA transcripts is one means of identifying a specific cell or group of cells of interest, or identifying a specific activation state of a specific cell or group of cells of interest, or of identifying a specific developmental state of a specific cell or group of cells of interest, or identifying any one of numerous physiological states a cell or a specific cell or group of cells of interest.

The terms “polypeptide”, “peptide”, and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component.

In the context of polypeptides and molecules as provided herein, the terms “fusion”, “fused,” “combination,” and “linked,” are used interchangeably herein. These terms refer to the joining together of two more protein components, by whatever means including chemical conjugation or recombinant means. For example, a promoter or enhancer is operably linked to a coding sequence if it affects the transcription of the sequence. Generally, “operably linked” means that the DNA sequences being linked are contiguous, and in reading phase or in-frame. As used herein, the term “in-frame” or “in frame” refers to the joining of two or more open reading frames (ORFs) to form a continuous longer ORF, in a manner that maintains the correct reading frame of the original ORFs. Thus, the resulting recombinant molecule is a single protein containing two or more segments that correspond to polypeptides encoded by the original ORFs (which segments are not normally so joined in nature).

In the context of polypeptides, a “linear sequence” or a “sequence” is an order of amino acids in a polypeptide in an amino to carboxyl terminus direction in which residues that neighbor each other in the sequence are contiguous in the primary structure of the polypeptide. A “partial sequence” is a linear sequence of part of a polypeptide that is known to comprise additional residues in one or both directions.

“Heterologous” means derived from a genotypically distinct entity from the rest of the entity to which it is being compared. For example, a glycine rich sequence removed from its native coding sequence and operatively linked to a coding sequence other than the native sequence is a heterologous glycine rich sequence. The term “heterologous” as applied to a polynucleotide, a polypeptide, means that the polynucleotide or polypeptide is derived from a genotypically distinct entity from that of the rest of the entity to which it is being compared.

The terms “polynucleotides”, “nucleic acids”, “nucleotides” and “oligonucleotides” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The phrase “complementary to” “complement of a polynucleotide” denotes a polynucleotide nucleic acid molecule having a complementary having a base sequence in a 5′ to 3′ or 3′ to 5′ orientation that base pairs with a nucleic acid having a base sequence of the reverse orientation. As described herein, a sensor RNA is complementary to a specified target RNA, with the exception of an obligatory mismatched codon, (preferably AUG in the sensor RNA).

The phrase “portion that is complementary to a cellular RNA” in the context of a sensor domain refers to consecutive nucleotides of a sensor nucleic acid domain that are able to base pair with corresponding consecutive nucleotides of a cellular RNA.

The phrase “portion that is complementary to a messenger RNA (mRNA)” in the context of a sensor domain refers to consecutive nucleotides of a sensor nucleic acid domain that are able to base pair with corresponding consecutive nucleotides of an mRNA.

The term “cellular RNA” refers to a nucleic acid in a cell composed of nucleotides that are substantially ribonucleotides but may include deoxyribonucleotides. Types of cellular RNAs include but are not limited to mRNA, rRNA, tRNA, and microRNA. A cellular RNA will have a length sufficient to form a nucleic acid duplex with a sensor RNA containing a mismatch that attracts ADAR to edit and repair the mismatch. Therefore, a cellular RNA will be at least 10 residues in length, and may be 15, 20, 25, 30, 35, 40, 45, 50, 75, 100, 125, 150, 175, 200, 225, 250, 275, 300, 325, 350, 375, 400, 425, 450, 475, 500, 600, 700, 800, 900, 1000 nucleotides in length or longer.

“Recombinant” as applied to a polynucleotide means that the polynucleotide is the product of various combinations of in vitro cloning, restriction and/or ligation steps, and other procedures that result in a construct that can potentially be expressed in a host cell.

The terms “gene” or “gene fragment” are used interchangeably herein. They refer to a polynucleotide containing at least one open reading frame that is capable of encoding a particular protein after being transcribed and translated. The term “gene” includes not only an open reading frame but also at least a promoter operatively associated with the open reading frame so as to initiate transcription of the open reading frame in the presence of appropriate transcription factors. A gene or gene fragment may be genomic or cDNA, as long as the polynucleotide contains at least one open reading frame, which may cover the entire coding region or a segment thereof. A “fusion gene” is a gene composed of at least two heterologous polynucleotides that are linked together.

“Homology” or “homologous” refers to sequence similarity or interchangeability between two or more polynucleotide sequences or two or more polypeptide sequences. When using a program such as BestFit to determine sequence identity, similarity or homology between two different amino acid sequences, the default settings may be used, or an appropriate scoring matrix, such as blosum45 or blosum80, may be selected to optimize identity, similarity or homology scores. Preferably, polynucleotides that are homologous are those which hybridize under stringent conditions as defined herein and have at least 70%, preferably at least 80%, more preferably at least 90%, more preferably 95%, more preferably 97%, more preferably 98%, and even more preferably 99% sequence identity to those sequences.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. As used herein, the terms “prevent,” “preventing,” “prevention,” “prophylactic treatment” and the like refer to reducing the probability of developing a disease, disorder or condition in a subject, who does not have, but is at risk of or susceptible to developing a disease, disorder or condition. The term “effective amount” or “therapeutically effective amount” refers to an amount sufficient to effect beneficial or desirable biological and/or clinical results.

An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The effects of any particular dosage can be monitored by a suitable bioassay, e.g., assay for tumor growth and/or size among others. The dosage can be determined by a physician and adjusted, as necessary, to suit observed effects of the treatment.

As used herein, the term “administering” an agent, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

The term “biological sample” as used herein includes, but is not limited to, a sample containing tissues, cells, and/or biological fluids isolated from a subject. Examples of biological samples include, but are not limited to, tissues, cells, biopsies, blood, lymph, serum, plasma, urine, saliva, mucus and tears. A biological sample may be obtained directly from a subject (e.g., by blood or tissue sampling) or from a third party (e.g., received from an intermediary, such as a healthcare provider or lab technician).

As used herein the term “condition and/or disease” includes, but is not limited to, any abnormal condition and/or disorder of a structure or a function that affects a part of an organism. It may be caused by an external factor, such as an infectious disease, or by internal dysfunctions, such as cancer, cancer metastasis, genetic disorders/mutations (both congenital and environmental) and the like.

As used herein, a monogenic somatic cell disorder comprising an underlying genetic mutation in a gene, refers to a monogenetic disorder caused by a variant in a single gene. The variant may be present on one or both chromosomes of a pair. Nonlimiting examples of monogenic disorders are cystic fibrosis, Huntington's disease and sickle cell disease.

As is known in the art, a cancer is generally considered as uncontrolled cell growth. The methods of the present invention can be used to treat any cancer, and any metastases thereof, including, but not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, pancreatic cancer, thyroid cancer, hepatic carcinoma, skin cancer, melanoma, brain cancer, neuroblastoma, myeloma, various types of head and neck cancer, acute lymphoblastic leukemia, acute myeloid leukemia, Ewing sarcoma and peripheral neuroepithelioma.

“Contacting” as used herein, e.g., as in “contacting a sample” refers to contacting a sample or cell directly or indirectly in vitro, ex vivo, or in vivo (i.e. within a subject as defined herein). Contacting a sample may include addition of a compound (e.g., a readrRNA molecule as provided herein and/or a delivery system comprising a readrRNA molecule as provided herein) to a sample, or administration to a subject. Contacting encompasses administration to a solution, cell, tissue, mammal, subject, patient, or human. Further, contacting a cell includes adding an agent to a cell culture.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e. living organism, such as a patient).

The terms “decrease”, “reduced”, “reduction”, and “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

The central nervous system (CNS) has three main components: the brain, the spinal cord, and the neurons (or nerve cells).

It is one of 2 parts of the nervous system. The other part is the peripheral nervous system, which consists of nerves that connect the brain and spinal cord to the rest of the body.

The major cell types of the central nervous system comprise neurons, glial cells (astrocytes, oligodendrocytes, ependymal cells, and microglia), choroid plexus cells, cells related to blood vessels and coverings. The peripheral nervous system comprises efferent neurons (motor neurons), afferent neurons (sensory neurons) and Schwann cells.

As used herein, a “subject” means a human or animal. Usually the animal is a vertebrate such as a primate, rodent, domestic animal or game animal. Primates include chimpanzees, cynomologus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of cancer. A subject can be male or female.

A subject can be one who has been previously diagnosed with or identified as suffering from or having a condition in need of treatment (e.g. cancer) or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition, or one or more complications related to the condition or a subject who does not exhibit risk factors.

A “subject in need” of treatment for a particular condition can be a subject having that condition, diagnosed as having that condition, or at risk of developing that condition.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

In some embodiments, a polypeptide, can comprise one or more amino acid substitutions or modifications. In some embodiments, the substitutions and/or modifications can prevent or reduce proteolytic degradation and/or prolong half-life of the polypeptide in a subject. In some embodiments, a polypeptide can be modified by conjugating or fusing it to other polypeptide or polypeptide domains such as, by way of non-limiting example, transferrin (WO06096515A2), albumin (Yeh et al., 1992), growth hormone (US2003104578AA); cellulose (Levy and Shoseyov, 2002); and/or Fc fragments (Ashkenazi and Chamow, 1997). The references in the foregoing paragraph are incorporated by reference herein in their entireties.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

In some embodiments, the expression of a biomarker(s), target(s), or gene/polypeptide described herein is/are tissue-specific. In some embodiments, the expression of a biomarker(s), target(s), or gene/polypeptide described herein is/are global. In some embodiments, the expression of a biomarker(s), target(s), or gene/polypeptide described herein is systemic.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence. Further they need not be physically linked.

“Marker” in the context of the present invention refers to an expression product, e.g., nucleic acid or polypeptide which is differentially present in a sample taken from subjects having diabetes or cancer, as compared to a comparable sample taken from control subjects (e.g., a healthy subject). The term “biomarker” is used interchangeably with the term “marker.”

In some embodiments, the methods described herein relate to measuring, detecting, or determining the level of at least one marker. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell is typically still referred to as “engineered” even though the actual manipulation was performed on a prior entity.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus and a virion.

In some embodiments of any of the aspects, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

In some embodiments of any of the aspects, described herein is a prophylactic method of treatment. As used herein “prophylactic” refers to the timing and intent of a treatment relative to a disease or symptom, that is, the treatment is administered prior to clinical detection or diagnosis of that particular disease or symptom in order to protect the patient from the disease or symptom. Prophylactic treatment can encompass a reduction in the severity or speed of onset of the disease or symptom or contribute to faster recovery from the disease or symptom. Accordingly, the methods described herein can be prophylactic relative to metastasis or tumor formation. In some embodiments of any of the aspects, prophylactic treatment is not prevention of all symptoms or signs of a disease.

As used herein “combination” refers to a group of two or more substances for use together, e.g., for administration to the same subject. The two or more substances can be present in the same formulation in any molecular or physical arrangement, e.g., in an admixture, in a solution, in a mixture, in a suspension, in a colloid, in an emulsion. The formulation can be a homogeneous or heterogenous mixture. In some embodiments of any of the aspects, the two or more substances active compound(s) can be comprised by the same or different superstructures, e.g., nanoparticles, liposomes, vectors, cells, scaffolds, or the like, and the superstructure is in solution, mixture, admixture, suspension with a solvent, carrier, or some of the two or more substances. Alternatively, the two or more substances can be present in two or more separate formulations, e.g., in a kit or package comprising multiple formulations in separate containers, to be administered to the same subject.

A kit is an assemblage of materials or components, including at least one reagent described herein. The exact nature of the components configured in the kit depends on its intended purpose. In some embodiments of any of the aspects, a kit includes instructions for use. “Instructions for use” typically include a tangible expression describing the technique to be employed in using the components of the kit, e.g., to treat a subject or for administration to a subject. Still in accordance with the present invention, “instructions for use” may include a tangible expression describing the preparation of at least one reagent described herein, such as dilution, mixing, or incubation instructions, and the like, typically for an intended purpose. Optionally, the kit also contains other useful components, such as, measuring tools, diluents, buffers, syringes, pharmaceutically acceptable carriers, or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging may also preferably provide an environment that protects from light, humidity, and oxygen. As used herein, the term “package” refers to a suitable solid matrix or material such as glass, plastic, paper, foil, polyester (such as polyethylene terephthalate, or Mylar) and the like, capable of holding the individual kit components. Thus, for example, a package can be a glass vial used to contain suitable quantities of a composition containing a volume of at least one reagent described herein. The packaging material generally has an external label which indicates the contents and/or purpose of the kit and/or its components.

As used herein, the term “nanoparticle” refers to particles that are on the order of about 1 to 1,000 nanometers in diameter or width. The term “nanoparticle” includes nanospheres; nanorods; nanoshells; and nanoprisms; these nanoparticles may be part of a nanonetwork. The term “nanoparticles” also encompasses liposomes and lipid particles having the size of a nanoparticle. Exemplary nanoparticles include lipid nanoparticles or ferritin nanoparticles. Lipid nanoparticles can comprise multiple components, including, e.g., ionizable lipids (such as MC3, DLin-MC3-DMA, ALC-0315, or SM-102), pegylated lipids (such as PEG2000-C-DMG, PEG2000-DMG, ALC-0159), phospholipids (such as DSPC), and cholesterol.

Exemplary liposomes can comprise, e.g., DSPC, DPPC, DSPG, Cholesterol, hydrogenated soy phosphatidylcholine, soy phosphatidyl choline, methoxypolyethylene glycol (mPEG-DSPE) phosphatidyl choline (PC), phosphatidyl glycerol (PG), distearoylphosphatidylcholine, and combinations thereof.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff s Clinical Oncology, 2013 Elsevier; and Fischer D S (ed): The Cancer Chemotherapy Handbook, 4th ed. St. Louis, Mosby-Year Book, 2003).

mCherry is a basic (constitutively fluorescent) red fluorescent protein published in 2004, derived from Discosoma sp.. It is reported to be a very rapidly-maturing monomer with low acid sensitivity; virally expressed mCherry is pseudocolored magenta.

SmV5 refers to spaghetti monster V5;

smFLAG refers to Spaghetti Monster FLAG: 10 copies of an epitope tag FLAG-(DYKDDDDK)

tTA2 is a tetracycline dependent transcription activator

W3SL is truncated woodchuck hepatitis posttranscriptional regulatory element and polyadenylation signal cassette (Choi et al., 2014). Choi J H, Yu N K, Baek G C, Bakes J, Seo D, Nam H J, Baek S H, Lim C S, Lee Y S, Kaang B K (2014).

eYFP is enhanced yellow fluorescent protein.

PT Enhancer is mscRE4 enhancer which labels L5 pyramidal tract (PT) neurons which are a subset of CFPNs.

Calcium-saturated GCaMP6s is 27% brighter than enhanced GFP (EGFP), its parent fluorescent protein.

PlxnD1 (intratelencephalic-IT PNs in L2/3 and L5a), Satb2 (IT PNs across both upper and lower layers), Rorb (L4 pyramidal neurons), and vGAT (pan-GABAergic neurons).

Fezf2 is FEZ Family Zinc Finger 2 gene. Diseases associated with FEZF2 include Partial Fetal Alcohol Syndrome and Thymic Dysplasia.

Ctip2 tip2/Bcl11b is a zinc finger transcription factor with dual action (repression/activation) that couples epigenetic regulation to gene transcription during the development of various tissues. The transcription factor COUP TF1-interacting protein 2 (CTIP2) plays critical roles during axonal extension and pathfinding by subcerebral projection neurons of the cerebral cortex (Arlotta et al., 2005). Here, we report that within the striatum Ctip2 is uniquely expressed by MSN, specifically labeling this critical neuronal population from early postmitotic stages. Loss of Ctip2 function results in a failure of MSN differentiation, disruption of the patch-matrix organization of MSN, and distinct changes in the expression of multiple genes, including novel molecular identifiers of the patch compartment. The defect in patch aggregation also results in abnormal dopaminergic innervation of the striatum. Finally, there is an alteration in the expression of molecules involved in cellular repulsion and the appearance of heterotopias within the mutant striatum, strongly suggesting that the loss of Ctip2 disrupts normal mechanisms of cellular repulsion during development. Journal of Neuroscience 16 Jan. 2008, 28 (3) 622-632; DOI: doi.org/10.1523/JNEUROSCI.2986-07.2008.

Corticofugal projection neurons (CFPNs) that constitute cortical output channels, including PlxnD1 (intratelencephalic-IT PNs in L2/3 and L5a), Satb2 (IT PNs across both upper and lower layers), Rorb (L4 pyramidal neurons), and vGAT (pan-GABAergic neurons),

hSyn refers to a human synapsin 1 gene promoter, which is recognized in the art to confer highly neuron-specific long-term transgene expression from an adenoviral vector in the adult rat brain

TRE-3G refers to a eukaryotic inducible promoter; TRE is made up of Tet operator (tetO) sequence concatemers fused to a minimal promoter, (commonly the minimal promoter sequence derived from the human cytomegalovirus (hCMV) immediate-early promoter); In the absence of Tc or Dox, tTA binds to the TRE and activates transcription of the target gene.

mNeonGreen is a basic (constitutively fluorescent) green/yellow fluorescent protein published in 2013, derived from Branchiostoma lanceolatum.

WPRE is a woodchuck hepatitis virus post-transcriptional regulatory element (WPRE) increases transgene expression from a variety of viral vectors, although the precise mechanism is not known. WPRE is most effective when placed downstream of the transgene, proximal to the polyadenylation signal.

dTomato gene is a gene encoding dTomato, which is a basic (constitutively fluorescent) orange fluorescent protein derived from Discosoma sp.

tdTomato is a genetic fusion of two copies of the dTomato gene; tdTomato is an exceptionally bright red fluorescent protein.

Unless otherwise defined, all technical terms used herein have the same meaning as commonly understood by one of ordinary skill in the art to which this disclosure belongs.

### DETAILED DESCRIPTION

The present disclosure is based, in part, on the discovery of a dual function RNA molecule (readrRNA) which permits (i) targeting of a selected somatic cell based on its transcript profile and (ii) translation in the selected cell of a desired effector protein encoded by the RNA molecule, translation of the effector protein being implemented through RNA editing mediated by adenosine deaminase acting on RNA (ADAR).

### ADAR Mediated RNA Editing

RNA editing is a widespread post-transcriptional process that alters the sequence of RNA encoded by the DNA template, ubiquitous in all metazoan cells. Across the animal kingdom, the most prevalent form of RNA editing is adenosine-to-inosine (A-to-I) conversion, catalyzed by the ADAR (adenosine deaminase acting on RNA) family of enzymes, which has three members in mammals (ADAR1, ADAR2, ADAR3). The edited inosine then base pairs, instead, with cysteine, and is recognized as guanosine (G) by various cellular machinery. ADAR-mediated A-I editing is ubiquitous to all metazoan cells. See FIG. 3.

There are millions of ADAR editing sites in the transcriptomes of humans and animals, only a small fraction of this editing occurs in coding mRNAs, altering protein properties. The vast majority are in non-coding regions, which may influence RNA splicing, microRNA and shRNA functions. Their most essential role though is to protect cells from innate immune response to self-generated dsRNAs while letting the immune system destroy viral dsRNAs during an infection.

readrRNA

The “readrRNA” refers to an RNA based molecule having a 5′ region and a 3′ region, where the readrRNA molecule comprises, consists of, or consists essentially of (i) a 5′ region comprising a sensor (ses) domain, the sensor domain comprising at least one ADAR-editable STOP codon; and (ii) an effector RNA (efRNA) region that is downstream and in-frame with the sensor domain. The dual-function readrRNA of Applicant's invention permits recruitment of the ADAR deaminase to edit a specific site(s) in the readrRNA by formation of a dsRNA having a mismatch with target RNA expressed in a selected somatic cell. Upon ADAR-mediated removal of a stop codon from the readrRNA molecule, translation of a downstream operably linked effector protein encoded by the readrRNA occurs in the selected somatic cell. FIG. 4. In the absence of target RNA in the selected somatic cell, the readrRNA remains inert in the cell.

Because the readrRNA detects or “senses” target RNA in the selected somatic cell, the readrRNA is an integral component of the system comprised by CellREADR (Cell access through RNA sensing by Endogenous ADAR), a programmable RNA sensing technology that leverages RNA editing mediated by ADAR (adenosine deaminase acting on RNA) for coupling the detection of cell-type defining RNAs with the translation of effector protein(s) in a somatic cell.

### Cell-Type Defining RNAs

RNAs are the central and universal mediator of genetic information underlying the diversity of cell types and cell states, which together shape tissue organization and organismal function across species and life spans. Despite advances in RNA sequencing and massive accumulation of transcriptome datasets across life sciences, the dearth of technologies that leverage RNAs to observe and manipulate cell types remains a prohibitive bottleneck in biology and medicine. (Zeng et al. https://doi.org/10.1016/j.cell.2022.06.031)

Cell types are the product of evolution and they are the basic functional units of an organism. The entire repertoire of cell types in the brain and the body is built through a sequential and parallel series of spatially and temporally coordinated developmental events starting from a single fertilized egg, the zygote. This developmental program carries out a remarkable implementation plan that unravels the identities of all cell types which are encoded in the genome through evolution. Transcriptional and epigenetic regulatory programs are unfolded from the genome sequences and drive a cascading series of cell proliferation and differentiation processes, leading to the manifestation of diverse cellular phenotypes. (Zeng https://doi.org/10.1016/j.cell.2022.06.031)

RNA expression profiles underlie arguably all phenotypic features of the cell at the time or state when the cell is characterized and is a one-time snapshot of the cell. A key point of distinction is whether the RNA expressed in a selected cell represent a particular cell state—a transient or dynamically responsive property of a cell to a context—or a cell type, as a cell type can exist in different states. Cell type-specific changes in RNA expression associated with different cell states may be seen during circadian cycles, variable metabolic states, development, aging, or under behavioral, pharmacological, or diseased conditions (Mayr et al., 2019; Morris, 2019). (Zeng et al. https://doi.org/10.1016/j.cell.2022.06.031)

A single-cell transcriptome is only a one-time snapshot of the cell. However, one can compare transcriptomes collected from different time points or different behavioral, physiological, or pathological states. The distinction between cell types and cell states is particularly challenging during development, as cells continually change their states, and at certain key time points, they may switch their cell type identities. However, although not absolute, it is reasonable to assume that transcriptomic changes tend to be more continuous during cell state transitions, while tending to be more abrupt or discrete when cells switch their types. (Zeng https://doi.org/10.1016/j.cell.2022.06.031)

For a definition of Cell-type to be meaningful, it is ideally associated with what the cell type does. Thus, in addition to defining a cell type based on its Cell-type defining RNAs, a cell types is defined by linking its RNA expression to anatomical and functional information. So far, it has been shown that transcriptomic types have excellent correspondence with their spatial distribution patterns. Since the spatial distribution pattern is defined during development, this suggests that transcriptomes may retain the developmental plan. (Zeng et al. https://doi.org/10.1016/j.cell.2022.06.031)

Systematic single-cell transcriptomic, epigenomic and spatially resolved transcriptomic profiling with high temporal resolution, coupled with lineage tracing and other phenotypic characterization, holds tremendous potential to capture key sets of genes and genomic regulatory networks involved in these series of events and begin to resolve the extremely complex spatial and temporal transitions of cell types and states leading to the adult-stage repertoire of cell types (Allaway et al., 2021; Bandler et al., 2022; Bhaduri et al., 2021; Cao et al., 2019b; Chen et al., 2022; Delgado et al., 2022; Di Bella et al., 2021; Klingler et al., 2021; La Manno et al., 2021; Romanov et al., 2020; Schmitz et al., 2022; Sharma et al., 2020; Shekhar et al., 2022; Tiklováet al., 2019; Zhu et al., 2018). (Zeng et al. https://doi.org/10.1016/j.cell.2022.06.031)

### Hierarchical Organization of Transcriptomically Defined Cell Types

As used herein, the term “brain cell” refers to any cell type found in a mammalian brain.

Within each of these major brain structures, there are multiple regions and subregions, each with many cell types. The basic architecture of the mammalian brain (Swanson, 2000, 2012) is composed of: telencephalon, diencephalon, mesencephalon (midbrain), and rhombencephalon (hindbrain). (Zeng https://doi.org/10.1016/j.cell.2022.06.031)

The Telencephalon (consists of five major brain structures (1) cortex, (2) hippocampal formation [HPF], (3) olfactory areas, (4) cortical sub-plate, and (5) cerebral nuclei). The Telencephalon and Diencephalon (including thalamus and hypothalamus are collectively called forebrain. The Mesencephalon (Midbrain) is divided into tectum and tegmentum. The rhombencephalon (hindbrain) is divided into the pons, medulla, and cerebellum. (Zeng https://doi.org/10.1016/j.cell.2022.06.031)

In the cortex, the first (highest) level of branches is the separation of neuronal and various non-neuronal cell classes (FIG. 2A). For neurons, the second level of branches is driven by major brain structures/regions, and the third level comprises various cell subclasses and types within each major brain structure, although there may be cell types crossing or shared between brain structures due to cell migration during development. (Zeng https://doi.org/10.1016/j.cell.2022.06.031)

The cortex is composed of multiple cortical areas (including visual cortex and motor cortex), each mediating sensory, motor, or associational functions. In each of these areas, there are two neuronal classes based on the dominant neurotransmitters they release, glutamatergic and GABAergic, as well as several non-neuronal classes. (Zeng https://doi.org/10.1016/j.cell.2022.06.031)

The glutamatergic excitatory neurons mostly have long-range axon projections to other cortical and/or subcortical regions. They are divided into nine subclasses based on their layer specificity and long-range projection patterns: L2/3 intratelencephalic projecting [IT], L4/5 IT, L5 IT, L6 IT, Car3 IT, L5 extratelencephalic projecting [ET], L5/6 near-projecting [NP], L6 corticothalamic projecting [CT], and L6b. (Zeng https://doi.org/10.1016/j.cell.2022.06.031)

The GABAergic inhibitory neurons mostly have their axon projections confined within the local area. They are divided into six subclasses named after canonical marker genes: Lamp5, Sncg, Vip, Sst, Sst-Chodl, and Pvalb. (Zeng https://doi.org/10.1016/j.cell.2022.06.031)

Within each of the glutamatergic or GABAergic subclasses, as well as each non-neuronal class, there are several transcriptomic clusters or types, resulting in a total of 110 transcriptomic cell types in each cortical area (Brain Initiative Cell Census Network, 2021; Tasic et al., 2018). This organization is highly consistent with the existing knowledge about cortical cell types that have been extensively studied in a variety of phenotypic modalities over the past 50 years (Harris and Shepherd, 2015; Tremblay et al., 2016; Yuste et al., 2020; Zeng and Sanes, 2017), suggesting that single-cell transcriptomics alone can faithfully capture the overall cell type organization at class and subclass levels. (Zeng https://doi.org/10.1016/j.cell.2022.06.031)

However, within cortex cell types that are specific to a cortical area or shared among areas have both been identified. The shared cell types often exhibit gradient distribution or gradient gene expression across areas the coexistence of discrete and continuous variations between types. (Zeng https://doi.org/10.1016/j.cell.2022.06.031)

Discrete variations exist among cell subclasses and major types that are usually at the higher branches of the hierarchy. Continuous variations are usually found among closely related transcriptomic clusters or subtypes at lower branches, such as the many IT neuron types across the cortical depth from L2/3 to L6 (FIG. 2C). Cells at opposite ends of the continuum have clearly distinct transcriptomic profiles, but the transition from one end to the other is gradual among the cells composing the continuum. (Zeng https://doi.org/10.1016/j.cell.2022.06.031)

Integration of transcriptomics and MERFISH, a spatially resolved transcriptomic method, reveals the spatial organization of mouse motor cortex cell types (Zhang et al., 2021a). A major finding of the study is that in addition to the laminar distribution of glutamatergic neuron subclasses as expected, even GABAergic types within each subclass exhibit layer-selective localization, and the continuous variations among individual glutamatergic types or GABAergic types correlate well with their continuous distribution along cortical layers/depth (with a prominent example being that all the excitatory L2/3-L6 IT types line up along the cortical depth from L2/3 to L6). (Zeng https://doi.org/10.1016/j.cell.2022.06.031)

However, it is often unclear if cell types defined by different phenotypic features agree with each other nor which feature is the right one to define cell types.

By linking the targeting specific RNA transcripts with expression of an encoded effector molecule such as a fluorescent protein, the cell readrRNA system provides a system to simultaneously monitor cell type and states thereof, based on transcripts as well as spatially/morphologically.

The invention described herein is based on Watson-Crick base-pairing and RNA editing, CellREADR 1) has inherent and absolute specificity to cellular RNA and cells defined by RNA expression; 2) easy to design, build, use, and disseminate (e.g., DNA vectors); 3) infinitely scalable for targeting all RNA-defined cell types in any tissue; libraries of “cell armamentarium” 4) generalizable to most animal species including human; 5) comprehensive for most cell types and tissues and 6) general across animal species, 7) applicable to human biology and medicine, 8) programmable to achieve intersectional targeting of cells defined by two or more RNAs and multiplexed targeting and manipulation of several cell types in the same tissue.

### CellREADR Technology

The core of the CellREADR technology is the readrRNA which is an RNA sequence specific molecular sensor-switch operably linked to an effector molecule. That is a single modular readrRNA comprises a 5′-prime sense-edit-switch domain (sesRNA) and a 3′-prime effector domain (efRNA). The target specificity of sesRNA is due to its interaction with complementary sequences on target mRNA. The degree of complementarity determines whether there is ADAR-mediated editing of the sesRNA. A sesRNA which is fully complementary to the target RNA induces ADAR-mediated editing of the sesRNA at the ADAR editable stop codon.

readrRNAs can be generated from conventional DNA expression vectors. These vectors consist of a promoter, DNA cassettes coding for sesRNA and efRNA, and 3′ untranslated regions, which can be assembled by routine DNA synthesis and molecular cloning. In one embodiment, the sesRNA coding cassette may be—˜200-300 base pairs, and the effector gene cassette may be—˜1-2 kilo base pairs. These expression vectors can be readily packaged into various viral particles. readrRNAs can also be generated by direct single-strand oligonucleotide synthesis, with incorporation of chemically modified nucleotides if necessary.

Modular readrRNA

Accordingly, one aspect of the present disclosure provides a modular readrRNA molecule comprising, consisting of, or consisting essentially of (i) a 5′ region comprising a sensor domain, the sensor domain comprising at least one ADAR-editable STOP codon; and (ii) an effector RNA (efRNA) region that is downstream and in-frame with said sensor domain. The term “modular” when used in the context of the phrase “Modular readrRNA Molecule” refers a recombinant readrRNA molecule comprising a combination of a much smaller number of linked structural unit, where each structural unit encodes an independently functioning protein molecule.

The modular design of the readrRNA molecule, in which different protein encoding domains are designed at the RNA level and which are assembled in the recombinant readrRNA molecule in a desired order with a specified number of repeats design, enables the production of readrRNA molecules with diverse properties. The translation machinery also has high fidelity so that the desired readrRNA molecule will have the specified amino acid sequence.

In general, a readrRNA molecule is composed of modular domains that confer specific functions, including but not limited to facilitation of interactions between cells, sensing environmental stimuli, effecting a response to environmental stimuli, including effecting spatiotemporal input/output in a biological system.

Sensor Domain of Modular readrRNA

The sensor domain (sesRNA) comprises a set of nucleotides that are complementary to and able to detect a specific cell type through sequence-specific base pairing with an RNA present in the specific cell type. The sensor domain may comprise any number of nucleotides. In some embodiments, the sensor domain comprises at least 10, at least 20, at least 30, at least 40, at least 50, at least 60, at least 70, at least 80, at least 90, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000. In some embodiments, the sensor domain comprises a range of about 100 to about 900 nucleotides. In another embodiment, the sensor domain comprises a range of about 200 nucleotides to about 300 nucleotides. Preferably, the sensor domain (sesRNA) contains ˜200 nucleotides, complementary to and thus can detect a specific cell type RNA through base pairing.

The sensor domain also includes one or more ADAR-editable STOP codons that act as a translation switch (termed herein as the sense-edit-switch RNA (sesRNA)). The sensor domain thus functions as a sense-edit-switch RNA (sesRNA). The sensor RNA comprises a nucleotide sequence that is complementary to a cellular RNA.

The modular readrRNA molecule also may include a sequence coding for a self-cleaving 2A peptide. In such embodiments, the 2A peptide is positioned in between domains, preferably between the sensor domain and the effector RNA region and/or between additional domains/regions that may be present in the readrRNA molecule or CellREADER system, as discussed further herein.

In-Frame Effector RNA (efRNA) Coding Region of Modular readrRNA

Downstream to the sensor domain and, optionally separated by the sequence coding for the self-cleaving 2A peptide, is an in-frame effector RNA (efRNA) coding region. The effector RNA (efRNA) may code for an effector protein of interest, such as a label allowing visualization of the labeled cell. The effector RNA (efRNA) may code for an effector protein that changes the physiology of a cell. For example, in the case of a treating a disease caused by a mutation in single gene, the encoded effector protein can be a corrected copy of the mutated gene. In the case of treating a disease by providing a certain protein, be it endogenous or exogenous to the organism, the protein can be encoded in the effector region.

Selection of a given efRNA is dependent on the desired use of the readrRNA (e.g., treatment of a disease, study of a protein/pathway, etc.) of the readrRNA molecule and can be readily determined by one skilled in the art. For example, the effector module of CellREADR (efRNA) can be built to manipulate cells in multiple ways, including enhance activity and function, suppress activity and function, rescue a mutant cell function by re-introducing an intact version of the deleted or mutated protein, alter and edit activity and function, reprogram cell identity, fate, and function, kill and delete a cell type, increase or decrease the production of cell numbers of a type, and cell type-specific genomic editing and gene regulation

In cells expressing the target RNA, the sesRNA forms dsRNA, which recruits endogenous ADAR enzyme. At the STOP codon, A to I editing converts the STOP to a TI(G)G tryptophan codon, switching on translation of the efRNA, and generation of effector proteins. The resulting fusion protein comprising an N-terminal peptide, T2A and C-terminal effector, which then self-cleaves through T2A, releasing the functional effector protein. In cells that do not express the target RNA, readrRNAs remain inert.

The efRNA-encoded protein may comprise any protein involved in, or that is able to influence cell replication, gene expression, and/or transcription/translation. Suitable examples include, but are not limited to, a transcriptional activator, a transcriptional inhibitor, and a DNA recombinase, and the like.

An effector protein includes but is not limited to A) an enzyme, for example, proteases, phosphatases, glycosylases, acetylases, or lipases, b) a protein that mimics a function of a host cell protein, c) a transcription factor, d) a protein partner that facilitates protein-protein interaction, d) a protein that alters host cell structure and function, for example by facilitating infection (a virulence factors or a toxin) and/or by triggering a defense response, and/or promoting morphogenesis (Cachat, E., Liu, W., Hohenstein, P. et al. A library of mammalian effector modules for synthetic morphology. J Biol Eng 8, 26 (2014). https://doi.org/10.1186/1754-1611-8-26).

For instance, exemplary effector proteins are listed in the Table 4 below.

Thus, effector functions can influence activities of the innate immune cell response, including phagocytosis, secretion of cytokines, trafficking or promoting function, migration, survival, and proliferation of immune cells.

Thus, the encoded effector molecule can be a transactivator or a transrepressor, stimulating or suppressing, respectively, expression of a gene of interest by binding to the promoter/enhance region of the gene of interest, be it an endogenous gene, or an exogenous gene

TABLE 4

| Cell Editing | Examples on cellular property | Payload examples |
|---|---|---|
| Kill and Remove the cell | cell killing | thymidine kinase (TK), cytosine deaminase (CD) |
| | Programmed cell death | CASP3, CASP9, BCL, GSDME, GSDMD, |
| | | GZMA, GZMB |
| | Cell killing by immune | IFNB, IFNG, TNFA, IL2, IL12, IL15, CD40L, |
| | stimulation | GSDME, GSDMD, CXCL9, CXCL10, CXCL11, |
| | | CXCL16 |
| Enhance cell function | Neural activation | ChR2, DREADD-M3Dq |
| | immunity enhancer | IFNB, IFNG, TNFA, IL2, IL12, IL15, CD40L, |
| | | GSDME, GSDMD, CXCL9, CXCL10, CXCL11, |
| | | CXCL16 |
| | physiological editing | NaChBac, Kir2.1 |
| Suppress cell function | Protein synthesis inhibition | Ricin |
| | Neural inhibition | DREADD-hM4D, NpHR, GtACR1 |
| Reprogram the cell | Neuronal cell fate | NEUROD |
| Regenerate the cell | Cell regeneration | EGFs, VEGFs |

administered as part of the cell rear system.

A transcriptional activator is a protein or small molecule that binds to one or more specific regulatory sequences in DNA (or RNA in the case of a retrovirus) and stimulates transcription of one or more nearby genes. Most activators enhance RNA polymerase binding (formation of the closed complex) or the transition to the open complex required for initiation of transcription. Most activators interact directly with a subunit of RNA polymerase.

A transcriptional repressor is sequence-specific DNA binding proteins generally thought to function by recruiting corepressor complexes, which contain multiple proteins including histone modifying enzymes.

As used herein, “modulated” means regulated in the sense of activated or inhibited.

As used herein, a pathogen comprises an organism that causes disease in human beings, A pathogen includes but is not limited to a bacterium, a virus, a parasite, an insect, an algae, a prion and a fungus).

### Reporter Gene of CellREADR System2

Another aspect of the present disclosure provides a CellREADR system, the CellREADR system comprising at least two components, a first component comprising a modular readrRNA molecule as described herein and optionally an additional component(s) comprising a response gene operably linked (though in this embodiment, not physically linked) to the efRNA-encoded protein (e.g., transcriptional regulator, e.g., AP1 and SP1) of the readrRNA molecule. This allows the readrRNA molecule to activate, increase, decrease or repress transcription of another protein encoded on a physically separate, exogenously added nucleic acid molecule, such as caspase molecule which results in cell death.

### ADAR Mediated Programmability and Intersectional Targeting

The sensor and effector modules are combinatorial and easily programmable, which allows to manipulate each cell type in multiple ways and to simultaneously manipulate multiple cell types in a tissue, each in a specific and coordinated way. As provided above, in some embodiments the modular readrRNA molecule comprises, consists of, or consists essentially of a 5′ sensor-edit-switch region (sesRNA) and a 3′ effector coding region (efRNA), separated by an optional link sequence coding for a self-cleaving peptide 2A. In some embodiments, the sesRNA contains about 200 to about 300 nucleotides, complementary to and thus can detect a specific cell type RNA through base pairing while also comprising one or more ADAR-editable STOP codons that acts as a translation switch and wherein downstream is an in-frame effector coding region to generate various effector proteins of interest.

In cells expressing the target RNA, the sesRNA forms dsRNA with the target RNA, which recruits endogenous ADAR enzyme. At the STOP codon, A to I editing converts the STOP to a TI(G)G tryptophan codon, switching on translation of the efRNA, and generation of effector proteins. The resulting fusion protein comprising an N-terminal peptide, 2A and C-terminal effector, which then self-cleaves through 2A, releasing the functional effector protein. Importantly, in cells that do not express the target RNA, the readrRNAs remain inert. As such, the modular readrRNA molecules can thus be deployed as a single RNA molecular and can fit easily into viral vector (e.g., an AAV vector), as ADAR is cell endogenous.

In some situations, the ADAR protein(s) are not highly expressed, or in some cases absent, in the cell. In such cases, the present disclosure provides for the addition of the ADAR protein (e.g., the ADAR2) to be included within the modular readrRNA molecule and/or added to the system via a separate vector. The most fundament feature of CellREADR is that it is entirely RNA sequence based and operates through Watson-crick base pairing which confers numerous highly desirable properties, including, but not limited to, (i) inherent & absolute specificity to cellular RNAs; (ii) easy to design, build, use, and share (DNA vectors); (iii) infinitely scalable libraries of “cell armamentarium”; (iii) comprehensive for most cell types and tissues; (iv) general across animal species; and (v) human biology and medicine. Thus, comprehensive and combinatorial CellREADR sensor-effector libraries can be built for identifying, characterizing and manipulating cell types across organ systems and animal species.

The programmability of the modular readrRNA molecules provided herein confers additional power. Accordingly, another embodiment of the present disclosure provides for the programmability the modular readrRNA molecules and/or intersection targeting using the modular readrRNA molecules as provided herein.

First, two or more RNA sensors can be designed to detect two or more separate cellular RNAs to achieve intersectional targeting of two or more specific cell types. Second, the same RNA sensor can be linked to different effectors to label, record, and manipulate the same cell type. Third, a cohort of multiple RNA sensors can be designed to target several cell types in the same tissue, each expressing a different effector, to coordinately module tissue function. Fourth, RNA sensors can be designed to detect different threshold levels of a target RNA to monitor and manipulate different cell states defined by the RNA levels.

In one embodiment, the present disclosure provides for two sensors that are designed to detect two separate cellular RNAs to achieve intersectional targeting of more specific cell types. In one aspect, each sensor module has a STOP codon, and only when both are removed can the effector molecule be expressed. In one embodiment, each sensor comprises at least one STOP codon. In another embodiment, the same sensor can be used to expression different effectors to label, record, and manipulate the same cell type. In yet another embodiment, a plurality of sensors is designed to target several cell types in the same tissue, each expressing a different effector, to thereby coordinate module tissue function.

Thus, the RNA sensing domain has the capacity to detect any cellular RNA and thus the ability to access any RNA-defined cell types and cell states in any human tissues. The effector domain has the capacity to encode any protein and thus the ability to monitor, manipulate, and edits many cellular properties.

### Generality of Targeting Cell Types and Cell States

The RNA sensor domain can detect RNA markers that define cell types and cell states. Recent advances in single cell RNA sequencing are generating massive datasets in all human and animal tissues^{1,2,3}. Several major efforts are driving the progress, including the Human Cell Atlas project (world wide web at humancellatlas.org); the NIH Human Biomolecular atlas program at (commonfund.nih.gov/hubmap); the BRAIN Initiative Cell Census Network (biccn.org/); and the Allen Brain Cell Atlas (portal.brain-map.org/).

All the single cell transcriptome datasets are publicly accessible. RNA markers will be identified for most if not all major human cell types^{1,2,3}. Furthermore, RNA markers will be identified for many diseased cell states 4. All these RNA markers can be used by CellREADR to target cell types and cell states. Some of these markers are listed in Table 5.

TABLE 5

| Cell types/Tissue | Genes |
|---|---|
| Retina | RHO |
| Heart | NPPA |
| smooth muscle | MYH1 |
| adrenal gland | CYP11B1 |
| parathyroid gland | GCM2 |
| thyroid gland | TG |
| pituitary gland | TSHB |
| lung | SFTPA1 |
| bone marrow | CTSG |
| lymphoid | CD1B |
| liver | ALB |
| gallbladder | FGF19 |
| testis | LELP1 |
| epididymis | DEFB106A |
| prostate | TGM4 |
| seminal vesicle | SEMG2 |
| adipose tissue | GYG2 |
| Inhibitory Neuron | VGAT |
| Excitaroy Neuron | CAMK2 |
| Neuron | NEUN |

### REFERENCES

1. Regev A, Teichmann S A, Lander E S, Amit I, Benoist C, Birney E, Bodenmiller B, Campbell P, Carminci P, Clatworthy M, Clevers H, Deplancke B, Dunham I, Eberwine J, Eils R, Enard W, Farmer A, Fugger L, Gottgens B, Hacohen N, Haniffa M, Hemberg M, Kim S, Klenerman P, Kriegstein A, Lein E, Linnarsson S, Lundberg E, Lundeberg J, Majumder P, Marioni J C, Merad M, Mhlanga M, Nawijn M, Netea M, Nolan G, Pe'er D, Phillipakis A, Ponting C P, Quake S, Reik W, Rozenblatt-Rosen O, Sanes J, Satija R, Schumacher T N, Shalek A, Shapiro E, Sharma P, Shin J W, Stegle O, Stratton M, Stubbington M J T, Theis F J, Uhlen M, van Oudenaarden A, Wagner A, Watt F, Weissman J, Wold B, Xavier R, Yosef N; Human Cell Atlas Meeting Participants. (2017) The Human Cell Atlas. Elife. 2017 Dec. 5; 6:e27041. doi: 10.7554/eLife.27041. PMID: 29206104.
2. Osumi-Sutherland D, Xu C, Keays M, Levine A P, Kharchenko P V, Regev A, Lein E, Teichmann S A. (2021) Cell type ontologies of the Human Cell Atlas. Nat Cell Biol. 2021 November; 23(11):1129-1135. doi: 10.1038/s41556-021-00787-7. Epub 2021 Nov. 8. PMTD: 34750578 Review.
3. Haniffa M, Taylor D, Linnarsson S, Aronow B J, Bader G D, Barker R A, Camara P G, Camp J G, Chedotal A, Copp A, Etchevers H C, Giacobini P, Gottgens B, Guo G, Hupalowska A, James K R, Kirby E, Kriegstein A, Lundeberg J, Marioni J C, Meyer K B, Niakan K K, Nilsson M, Olabi B, Pe'er D, Regev A, Rood J, Rozenblatt-Rosen O, Satija R, Teichmann S A, Treutlein B, Vento-Tormo R, Webb S; Human Cell Atlas Developmental Biological Network. (2021) A roadmap for the Human Developmental Cell Atlas. Nature. 2021 September; 597(7875):196-205. doi: 10.1038/s41586-021-03620-1. Epub 2021 Sep. 8. PMID: 34497388.
4. Jagadeesh K A, Dey K K, Montoro D T, Mohan R, Gazal S, Engreitz J M, Xavier R J, Price A L, Regev A. (2022) Identifying disease-critical cell types and cellular processes by integrating single-cell RNA-sequencing and human genetics. Nat Genet. 2022 October; 54(10):1479-1492. doi: 10.1038/s41588-022-01187-9. Epub 2022 Sep. 29. PMID: 36175791

### Delivery

CellREADR can be deployed as a single RNA molecular, as ADAR is cell endogenous. And can fit easily into AAV viral vector . . . in <4.7 Kbs. In practice, the entire readrRNA is several kilobases, depending on what specific sensors and effectors are incorporated into the molecule, and thus is deliverable to cells through a delivery system. In cells expressing the target RNA, sesRNA forms a dsRNA with the target, which recruits ADARs to assemble an editing complex. At the editable STOP codon, ADARs convert A to I, which pairs with the opposing C in the target RNA. This A->G substitution converts a TAG STOP codon to a TI(G)G tryptophan codon, switching on translation of efRNA. The in-frame translation generates a fusion protein comprising an N-terminal peptide, 2A (if being used), and C-terminal effector, which then self-cleaves through 2A and releases the functional effector protein (see, e.g., FIG. 1a). readrRNA remains inert in cells that do not express the target RNA.

Through this disclosure and the knowledge in the art, the modular readrRNA molecules provided herein, or any components thereof, nucleic acid molecules thereof, and/or nucleic acid molecules encoding or providing components thereof, as well as any CellREADR systems as provided herein, can be delivered by various delivery systems. Examples of such delivery systems include, but are not limited to, DNA or RNA transfection method: chemical reagents (PEI, lipofectamine, calcium phosphate etc.,) or electroporation, DNA expression vectors can be packaged into Liposome nanoparticles. readrRNAs can be transcribed or synthesized in vitro and packaged into Liposome nanoparticles, nanoparticles, liposomes, recombinant viral vectors (Viral vectors: Adeno-associated virus (AAV), lenti-virus, and vesicular stomatitis virus are preferred viral vehicles), electroporation exosomes, microvesicles, gene-guns, the Selective Endogenous eNcapsidation for cellular Delivery (SEND) system, (an mRNA delivery system comprising humanized virus-like particles (VLPs) based on retroelements present in the human genome, (Segel M, et al. Mammalian retrovirus-like protein PEG10 packages its own mRNA and can be pseudotyped for mRNA delivery. Science. 2021; 373:882-889. doi: 10.1126/science.abg6155), combinations thereof, and the like.

The modular readrRNA molecules and/or any of the RNAs (e.g., sesRNA, efRNA, etc.) and/or any accessory proteins and/or CellREADR systems can be delivered using suitable vectors, e.g., plasmids or recombinant viral vectors, such as adeno-associated virus (AAV), adenovirus, retrovirus, lentivirus, herpes viral vector, vesicular stomatitis virus, and other viral vectors or combinations thereof. The proteins, e.g., sesRNA, efRNA, efRNA response genes, protein encoding or non-encoding RNAs (e.g., sgRHA, shRNA, etc.), Cell READR systems, etc., can be packaged into one or more vectors, e.g., plasmids or viral vectors. For example, in some embodiments, a second expression vector that comprises an efRNA response gene operably linked to the efRNA-encoded protein is co-delivered with the readrRNA molecule and/or CellREADR system, wherein upon successful translation of the modular readrRNA molecule and effector RNA results in successful binding and activation of the reporter product. In some embodiments, the efRNA response gene comprises a reporter gene (e.g., reporter genes including, but not limited to, GFP, mRuby, mCherry, ChR2, DTA, Gcamp, TK, interferon, etc.). In other embodiments, the efRNA response gene comprises a secondary effector gene.

For bacterial applications, if applicable, the nucleic acids encoding any of the components of the modular readrRNA molecule systems described herein can be delivered to the bacteria using a phage. Exemplary phages include, but are not limited to, T4 phage, μ, λ phage, T5 phage, T7 phage, T3 phage, Φ29, M13, MS2, Qβ, and ΦX174. In such embodiments, the addition of exogenous ADAR may be required.

In some embodiments, the vectors, e.g., plasmids or recombinant viral vectors, are delivered to the tissue of interest by, e.g., intramuscular injection, intravenous administration, transdermal administration, intranasal administration, oral administration, or mucosal administration. Such delivery may be either via a single dose, or multiple doses. One skilled in the art understands that the actual dosage to be delivered herein may vary greatly depending upon a variety of factors, such as the vector choices, the target cells, organisms, tissues, the general conditions of the subject to be treated, the degrees of transformation/modification sought, the administration routes, the administration modes, the types of transformation/modification sought, etc.

In some embodiments, the recombinant viral vector comprises an adenovirus vector which can be at a single dose containing at least 1×10⁵ particles (also referred to as particle units, pu) of adenoviruses. In some embodiments, the dose preferably is at least about 1×10⁶ particles, at least about 1×10⁷ particles, at least about 1×10⁸ particles, and at least about 1×10⁹ particles of the adenoviruses.

In some embodiments, the delivery is via a recombinant adeno-associated virus (rAAV) vector. For example, in some embodiments, a modified AAV vector may be used for delivery. Modified AAV vectors can be based on one or more of several capsid types, including AAV1, AV2, AAV5, AAV6, AAV8, AAV 8.2. AAV9, AAV rhlO, modified AAV vectors (e.g., modified AAV2, modified AAV3, modified AAV6) and pseudotyped AAV (e.g., AAV2/8, AAV2/5 and AAV2/6), AAV-PHP.eB and any variants thereof, AAV-PHP.S and any variants thereof, AAV-PHP.V1 and any variants thereof, and the like. Exemplary AAV vectors and techniques that may be used to produce rAAV particles are known in the art.

In some embodiments, the delivery is via plasmids. The dosage can be a sufficient number of plasmids to elicit a response. In some cases, suitable quantities of plasmid DNA in plasmid compositions can be from about 0.1 to about 2 mg. Plasmids will generally include (i) a promoter; (ii) a sequence encoding a modular readrRNA molecule and/or CellREADR system as provided herein, each operably linked to a promoter (e.g., the same promoter or a different promoter); (iii) a selectable marker; (iv) an origin of replication; and (v) a transcription terminator downstream of and operably linked to (ii). The plasmids can also encode other RNA components, but one or more of these may instead be encoded on different vectors. The frequency of administration is within the ambit of the medical or veterinary practitioner (e.g., physician, veterinarian), or a person skilled in the art.

In those cells/systems (e.g., bacterial cells and certain plant cells) where the ADAR protein is not expressed, or is expressed at low levels, plasmids (either alone or those expressing a modular readrRNA molecule and/or a CellREADR system as provided herein), may further comprise a sequence encoding an ADAR gene (e.g., Adar1, Adar2, etc.)

Exogenous ADARs or engineered ADARs (i.e. with improved functionality) may increase the efficiency of CellREADR system. Exogenous ADAR can be delivered into animal (or plant) cells in the following ways
1) ADAR1 or ADAR2 is delivered with a separate construct/DNA vector (from the Readr construct) with CMV, CAG promoter, by transfection or virus infection (AAV, lentiviruses, etc).
2) ADAR1 or ADAR2 is placed in front of SesRNA sequence in the same CellReadr construct/DAN vector, and delivered into cells by transfection or virus infection.
3) ADAR1 or ADAR2 mRNAs are delivered into cells by LNPs, with the CellReadr DNA vector or RNA.
4) ADAR1 or ADAR2 proteins are delivered into cells by LNPs or VLP (viral like particles), with the CellReadr DNA vector or RNA.

In another embodiment, the delivery is via liposomes or lipofection formulations and the like, and can be prepared by methods known to those skilled in the art. Such methods are described, for example, in WO 2016205764 and U.S. Pat. Nos. 5,593,972; 5,589,466; and 5,580,859; each of which is incorporated herein by reference in its entirety.

In some embodiments, the delivery is via nanoparticles or exosomes. For example, exosomes have been shown to be particularly useful in delivery RNA.

Further means of introducing one or more components of the modular readrRNA molecule systems as provided herein to the cell is by using cell penetrating peptides (CPP). In some embodiments, a cell penetrating peptide is linked to the modular readrRNA molecule. In some embodiments, the modular readrRNA molecule and/or any components thereof are coupled to one or more CPPs to effectively transport them inside cells (e.g., plant protoplasts). In some embodiments, the modular readrRNA molecule and/or any components thereof are encoded by one or more circular or non-circular DNA molecules that are coupled to one or more CPPs for cell delivery.

CPPs are short peptides of fewer than 35 amino acids derived either from proteins or from chimeric sequences capable of transporting biomolecules across cell membrane in a receptor independent manner. CPPs can be cationic peptides, peptides having hydrophobic sequences, amphipathic peptides, peptides having proline-rich and anti-microbial sequences, and chimeric or bipartite peptides. Examples of CPPs include, e.g., Tat (which is a nuclear transcriptional activator protein required for viral replication by HIV type 1), penetratin, Kaposi fibroblast growth factor (FGF) signal peptide sequence, integrin 3 signal peptide sequence, polyarginine peptide Args sequence, Guanine rich-molecular transporters, and sweet arrow peptide.

Yet another means of introducing one or more components of the modular readrRNA molecule systems as provided herein to the cell is by using SEND (see, e.g., Segel, M. et al., 2021. Science 373:6557; 882-889, the contents of which are hereby incorporated by reference in its entirety). In such embodiments, retroviral-like proteins, such as PEG10, which directly binds to and secretes its own mRNA in extracellular virus-like capsids, are pseudotyped with fusogens to deliver functional mRNA cargos (i.e., a modular readrRNA molecule as provided herein) to mammalian cells.

Other embodiments of the present disclosure provide for modification of a modular readrRNA molecule as provided herein. Such modifications may be for any purpose, such as increased stability, ability of the modular readrRNA molecule to evade the subject's immunity, and the like. For example, in instances where the modular readrRNA molecule comprises a circular RNA molecule, one such modification may include N6-methyladenosine modification. For example, inclusion of an N6-methyadenosine reader YTHDF2 sequence enables the sequestration of N6-methyladenosine-circularRNA thereby allowing for the suppression of innage immunity (see, e.g., Chen, Y. G. et al., (2019) Molecular Cell (76):1; 96-109, the contents of which are hereby incorporated by reference in its entirety). In other embodiments, such modifications may include the replacing of uridine with pseudouridine to help evade the immune system of a subject (see. e.g., Dolgin, E. (2021) Nature 597; 318-324, the contents of which are hereby incorporated by reference in its entirety).

### Pharmaceutical Compositions

In another aspect, the present disclosure provides compositions comprising one or more of the modular readrRNA molecules as described herein, or a delivery system comprising a modular readrRNA molecule as provided herein (herein used singly or together as “molecules”) and an appropriate carrier, excipient or diluent. The exact nature of the carrier, excipient or diluent will depend upon the desired use for the compositions and may range from being suitable or acceptable for veterinary uses to being suitable or acceptable for human use. The compositions may optionally include one or more additional compounds and/or therapeutic agents.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

Alternatively, other pharmaceutical delivery systems may be employed. Liposomes and emulsions are well-known examples of delivery vehicles that may be used to deliver molecule(s). Certain organic solvents such as dimethyl sulfoxide (DMSO) may also be employed, although usually at the cost of greater toxicity.

The pharmaceutical compositions may, if desired, be presented in a pack or dispenser device which may contain one or more unit dosage forms containing the molecule(s). The pack may, for example, comprise metal or plastic foil, such as a blister pack. The pack or dispenser device may be accompanied by instructions for administration.

The molecule(s) described herein, or pharmaceutical compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

Determination of an effective dosage of molecule(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound (e.g., efRNA product) that is at or above an IC50 of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Dosage amounts will depend upon, among other factors, the activity of the active compound, the bioavailability of the compound, its metabolism kinetics and other pharmacokinetic properties, the mode of administration, and various other factors, discussed above. Dosage amount and interval may be adjusted individually to provide plasma levels of the compound(s) and/or active metabolite compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

### Cell Manipulation

In order to monitor and/or change the physiology of a cell, or group of cells of interest, the cell(s) of interest are identified on the basis of differential expression of one or more RNA transcripts the cell(s)'s through the SES component of the readrRNA. Through ADAR mediated editing, the operably linked effector molecule(s) are translated and may label the cell fluorescently, and/or effect some desired change in the physiology of the cell(s), including cell death.

In addition to the physical linkage of the SES component of the readrRNA to the encoded effector of the readrRNA, the cells of interest can further comprise a second nucleic acid entity that is under the control of the encoded effector of the readrRNA, comprising a system called a CellREADR system. For example the effector of the ReadrRNA can encode a transactivator that can activate genes either encoded on a second nucleic entity, where the second nucleic entity is endogenous to the cell encoding a gene under the control of the transactivator that is endogenous to the cell, and/or where the second nucleic entity is exogenously added to the cell and encodes a gene(s) under the control of the transactivator that is exogenous and/or endogenous to the cell.

A transactivator or repressor can also silence or decrease expression of specified endogenous genes in a cell by controlling the expression of exogenous genes encoding tight hairpin loops (shRNA) that silence. It is also noted that an alternative to being located on a second nucleic entity, genes under control of the transactivator optionally can be positioned on the readrRNA molecule itself. In the case of cells comprising a mutated gene that is ultimately causing a disease or disorder in a patient, the effector can encode a functioning gene and/or cause expression of nucleic encoding a functioning gene.

Accordingly, cells or tissues express the modular readrRNA molecules, and systems comprising such modular readrRNA molecules. Hence, another aspect of the present disclosure provides a cell comprising a modular readrRNA molecule as provided, or a delivery system comprising a modular readrRNA molecule as provided herein. The readrRNA molecules provided herein can be expressed in prokaryotic and eukaryotic cells. In some embodiments, the cell comprises a eukaryotic cell. In one embodiment, the eukaryotic cell comprises a mammalian cell or a plant cell.

Another aspect of the present disclosure provides an animal model or a plant model comprising the cell as provided herein.

### Treatment

The present disclosure further encompasses methods comprising a readrRNA molecule as provided herein and as provided in the Examples below.

Another aspect of the present disclosure provides a method of detecting the presence or dynamics of cell state-defining cellular RNA and/or switching on the translation of one or more effector proteins, the method comprising, consisting of, or consisting essentially of detecting/hybridizing the target effector RNA with a modular readrRNA molecule as provided herein, or a delivery system comprising a modular readrRNA molecule as provided herein, or a pharmaceutical composition as provided herein, in which the sensor domain detects and binds a specific cell type RNA through sequence-specific base pairing, the one or more ADAR-editable STOP codons act as a translation switch thereby allowing for the translation of the effector RNA that encodes for the effector protein.

In some embodiments, the effector proteins are within a cell.

Detecting/assessing a dynamic state of a cell is critical to detecting a disease in an individual, for diagnosis. Detecting/assessing a dynamic state of a cell is critical to a targeted treatment of a disease in an individual by providing a therapeutic specifically to specified targeted cells where the therapeutic can be most effective and with reduced pleiotropic side effects. However, the effector molecules can subsequently function outside the specified cell, for example in the case of secreted cytokines and interleukins, and T-CARs.

Another aspect of the present disclosure provides a method of treating a condition and/or disease in a subject in need thereof, the method comprising, consisting of, or consisting essentially of administering to the subject a modular readrRNA molecule as provided herein, or a delivery system comprising a modular readrRNA molecule as provided herein, such that the condition and/or disease is treated in the subject.

In some embodiments, the condition and/or disease is selected from the group consisting of cancer, infectious disease, a genetic disorder, and the like.

As used herein, “treating” of a condition and/or disease is ameliorating any condition or symptom associated with the condition and/or disease. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic. In some embodiments of any of the apsects, the administration is subcutaneous.

Examples of somatic cell type-specific gene therapy include cell type-specific complementation of normal mRNA/protein in monogenic diseases, including but not limited to, muscular dystrophy, cystic fibrosis Cystic fibrosis, congenital deafness, Duchenne muscular dystrophy, familial hypercholesterolemia, Hemochromatosis, Neurofibromatosis type 1 (NF1), Sickle cell disease and Tay-Sachs disease.

Treatment of cancer: (e.g. solid tumors; leukemia) includes using readrRNA and cellREADR for detecting cancer cells by their upregulated and/or aberrant forms of RNAs, and to eliminate cancer cells by expression of one or more cell death proteins including, but not limited to caspase, programmed cell death protein 1, Caspase 8, Bcl-2-associated X protein, PDL-L1, Caspase 3, CFLAR, Bcl-xl, SMAC/Diablo, Diablo homolog, Bcl-2homologous antagonist killer, FADD, BECN1, Death receptor 5, PDCD6, RIP1 kinase, RIP3 kinase, and granzyme; or receptors to recruit immune killer cells including, but not limited to, CD16, chemokine receptors (CCR2, CCR5, CXCR3 and CX3CR1) and β2-integrin LFA-1.

readrRNA and cellREADR can be used for treatment of liver diseases (advantage in delivery by lipid nanoparticles via portal vain etc.) including but not limited to chronic hepatitis B, C, non-alcoholic steatohepatitis (NASH); diseases of the heart, as well as infectious disease including but not limited to latent viral infections (intracellular parasites), and resistant infections, e.g., Epstein Bar virus, Herpes, Tuberculosis, Prion, Zika, HIV

readrRNA and cellREADR can be used for treatment of chronic pain: widespread & wide impact; e.g. diabetic neuropathy; NIDA, NINDS HEAL Initiative)>non-addictive pain treatment. readrRNA and cellREADR can modulate ion channel and receptor expression in specific sensory neurons and glia cell types in peripheral ganglia by targeted local AAV vector injection.

readrRNA and cellREADR can be used for treatment of epilepsy which is currently often treated by surgical resection of brain tissues. Epilepsy often results from aberrant neural activities in certain neuronal cell types. That is, Epilepsy is characterized by hyper-synchronized neural activity of pathological brain networks. Brain circuits comprise multiple excitatory and inhibitory cell types, each making unique contribution in information processing and excitation-inhibition balance. Neural activities in some cell types promote while in other cell types suppress seizure at different phases of epilepsy (initiation, maintenance). Using readrRNA and cellREADR for cell type specific modulation of brain circuit activity through RNA sensor(s)-effector(s) (e.g. druggable GPCRs, ion channels etc) is a promising approach to control and treat epilepsy. Thus, readrRNA and cellREADR can be used for cell type-targeted modulation of neural activity to suppress and treat epilepsy.

### Kits

The present disclosure further provides kits comprising the compositions provided herein and for carrying out the subject methods as provided herein. For example, in one embodiment, a subject kit may comprise, consist of, or consist essentially of one or more of the following: (i) a modular readrRNA molecule as provided herein; (ii) a CellREADR system as provided herein; (iii) delivery systems comprising a modular readrRNA and/or CellREADR system as provided herein; (iv) cells comprising a modular readrRNA and/or CellREADR system and/or delivery system comprising a modular readrRNA and/or CellREADR system as provided herein; and/or (v) pharmaceutical compositions as provided herein.

In other embodiments, a kit may further include other components. Such components may be provided individually or in combinations and may provide in any suitable container such as a vial, a bottle, or a tube. Examples of such components include, but are not limited to, (i) one or more additional reagents, such as one or more dilution buffers; one or more reconstitution solutions; one or more wash buffers; one or more storage buffers, one or more control reagents and the like, (ii) one or more control expression vectors or RNA polynucleotides; (iii) one or more reagents for in vitro production and/or maintenance of the of the molecules, cells, delivery systems etc. provided herein; and the like. Components (e.g., reagents) may also be provided in a form that is usable in a particular assay, or in a form that requires addition of one or more other components before use (e.g. in concentrate or lyophilized form). Suitable buffers include, but are not limited to, phosphate buffered saline, sodium carbonate buffer, sodium bicarbonate buffer, borate buffer, Tris buffer, MOPS buffer, HEPES buffer, and combinations thereof. Kit components may also be provided individually or in combinations, and may be provided in any suitable container, such as a vial, a bottle, or a tube. In some embodiments, the kits disclosed herein comprise one or more reagents for use in the embodiments disclosed herein.

In addition to above-mentioned components, a subject kit can further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

Another aspect of the present disclosure provides all that is described and illustrated herein. The following Examples are provided by way of illustration and not by way of limitation.

### EXAMPLES

General Methods common to the below working examples

Supplementary Movie 1. Optogenetic Activation of Mouse Injected with Binary CellREADR Viral Vectors

Optogenetic activation (CFA, 0.5 s) in a mouse injected with binary READR^{Cti}p²³Reporter^{ChR}g^{er2-eYFP} AAVs induces a stepping forelimb movement. The upward movement involves sequentially, elbow, wrist, and digit flexion followed by extension.

Supplementary Movie 2. Optogenetic Activation of Mouse Injected with Binary PT Enhancer Viral Vectors

Optogenetic activation (CFA, 0.5 s) in a mouse injected with PTenhancer Reporter^{ChRger2-eYFP} AAVs induces elbow extension accompanied by digit extension.

### Methods Used in Working Examples

### Plasmids

All constructs were generated using standard molecular cloning procedures. Vector backbones were linearized using restriction digestion, and DNA fragment inserts were generated using PCR or gBlock synthesis (IDT). Information on all plasmids is included in Table 6. All sesRNA inserts are generated by gBlock synthesis (IDT), and sesRNA sequences used in this study are included in Table 7.

### Cell Culture and Transfection

The HeLa cell line was obtained from A. Krainer laboratory (Cold Spring Harbor Laboratory). Mouse neuroblastoma Neuro-2a (N2a) cells were purchased from Millipore-Sigma (Sigma, Cat. 89121404). The HEK293T and KPC1242 cell lines were obtained from D. Fearon laboratory (Cold Spring Harbor Laboratory). HEK293T, Hela and N2a cell lines were cultured in Dulbecco's Modified Eagle Medium (Corning, 10-013-CV) with 10% fetal bovine serum (FBS) (Gibco, Cat. 16000036) under 5% CO2 at 37° C. 1% penicillin-streptomycin were supplemented in the medium. Cells were transfected with the Lipofectamine2000 (Invitrogen, Cat. 11668019) DNA transfection reagent according to the manufacturer's instructions. For interferon treatment, medium containing 1 nM Recombinant Mouse IFN-β (R&D, Cat. 8234-MB) was used and changed every 24 hours during the transfection process. For tetracycline treatment, medium containing indicated tetracycline concentration was used 24 hours after transfection to replace the tetracycline free medium and for 48 hours before analysis. For apoptosis assay, cells were incubated for 72 hours after transfection, the RealTime-Glo™ Annexin V Apoptosis and Necrosis Assay (Promega JA1011) was used to quantify the apoptosis level.

### SURVEYOR Assay

PCR of Genomic DNA (primers for DYRK1A, Forward: GGAGCTGGTCTGTTGGAGAA; Reverse: TCCCAATCCATAATCCCACGTT) is used to amplify the Cas9 target region from a heterogeneous population of modified and unmodified cells, and the PCR products are reannealed slowly to generate heteroduplexes. The reannealed heteroduplexes are cleaved by SURVEYOR nuclease, whereas homoduplexes are left intact (IDT: Alt-R Genome Editing Detection Kit). Cas9-mediated cleavage efficiency (% indel) is calculated based on the fraction of cleaved DNA³¹.

### Animals

Six to twenty weeks old (both male and female) mice were used in this study. Wide type mice were purchased from Jackson Laboratory (C57BL/6J, 000664). Fezf2-CreER and Rosa26-LoxpSSTOLoxp-H2bGFP mice were described previously^{39,56}. To label Fezf2⁺ cortical pyramidal neurons, 200 mg/kg dose of tamoxifen (T56648, Sigma) was administered by intraperitoneal injection two days before AAV injection. Long Evans rats (Rattus norvegicus, aged 12 weeks, 50-250 g) were obtained from Charles River. All the animals were maintained in a 12 h:12 h light:dark cycle with a maximum of five animals per cage for mice and one animal per cage for rats. All animal maintenance and experimental procedures were performed according to the guidelines established by the Cold Spring Harbor Laboratory and Duke Medical Center.

### ADAR1 Knockout Cell Line Construction

ADAR1 Knockout cell line was generated via CRISPR-Cas9 genome editing. The gRNAs were cloned into pSpCas9 (BB)-2A-puro (pX459) (Addgene, Cat. 62988). The gRNA sequences were 5′ GGATACTATTCAAGTCATCTGGG 3′ (gRNA1) and 5′ GTTATTTGAGGCATTTGATG 3′ (gRNA2). Briefly, ADAR1 knockout cells were generated with two gRNAs targeting exon 2, which is shared by both ADAR1-p110 and ADAR1-p150 isoforms. HEK293T cells were seeded into 6 well plates and transfected with 1 ug plasmid mixture of pX459-gRNA1 and pX459-gRNA2 using Lipofectamine 2000 (Thermo Fisher Scientific, Cat. 11668019). On the next day, all the media containing transfection reagents was removed replaced with fresh medium supplemented with puromycin (final conc. 2 ug/ml). Fresh medium with puromycin replaced with old medium every two days for three times. The remaining cells after puromycin selection was harvested with TrypLE (Gibco, Cat. 12605036) and distributed in 96-well plate with 1˜2 cells each well. Expanded single clones were screened for ADAR1 deficiency by western blot and disruption of the ADAR1 genomic locus was confirmed by Sanger sequencing.

### Flow Cytometry Analysis

Two days after transfection, cells were harvested with TrypLE (Gibco, Cat. 12605036), distributed in 96-well round-bottom plate and centrifuged at 500 rpm for 1 min at 4° C. Supernatant was removed and cells were resuspended with 1% PFA buffer and incubated at 4° C. overnight or longer time. The cells were then resuspended with 200 ul Flow Cytometry Staining Buffer (Invitrogen, Cat. 00-4222-26) before analysis using a LSR Fortessa (BD Biosciences). The Fortessa was operated by FACSDIVA (BD Biosciences) software. Data analysis was performed with FlowJo 10 (FlowJo, LLC). For sorting, cells were submitted to the same procedure as for flow cytometry analysis but without 1% PFA treatment and processed using BD FACSAria (BD Biosciences).

### CellREADR Efficiency for Exogenous or Endogenous Transcripts

To assay CellREADR efficiency with fluorescence reporter genes, HEK293T cells or HEK293T ADAR1 knockout cells were first seeded in 24-well plates. After 24 hours, cells were co-transfected with 1.5 μg plasmids in total. At 48 hours of post transfection, cells were collected, fixed with 1% PFA and prepared for FACS analysis. To calculate the CellREADR efficiency from the FACS data, the cells expressing READR vector were gated by fluorescent protein (for example, BFP in FIG. 1b-d) expressing by spacer, the efficiency was calculated as the ratio of double positive cell expressing both efRNA and target RNA among all the target RNA expressing cells within the gate. If the spacer is not a fluorescent protein (FIG. 1h and FIG. 2), the ratio was directly calculated as the ratio of double positive cell expressing both efRNA and target RNA among target RNA-expressing cells. For some controls, the CellREADR efficiency was calculated as the percent of efRNA expressing cells among all the counted cells.

sesRNA Design

The following procedure is used for sesRNA design: 1) sesRNA is complementary, i.e. anti-sense, to a specific cellular coding or non-coding RNA sequence; 2) The optimal length of a sesRNA is 200-300 nucleotides; 3) readrRNAconsists of a sesRNA and efRNA arranged in a continuous translation reading frame; 4) One or more STOP codons (TAG) are placed near the center of a sesRNA (ranging between ˜80220 nucleotide from 5′ end); 5) find 5′-CCA-3′ sequences in the target RNA, which is complementart to a 5′-TGG-3′ sequence in sesRNA; then replace 5′-TGG-3′ with 5′-TAG-3′, so that a mismatch A-C will be introduced when sesRNA base pairs with the target RNA; 6) to ensure that there are no other STOP codons in the sesRNA, all other TAG, TAA, TGA sequences in sesRNA are converted to GAG, GAA, GGA, respectively; preferably the converted STOP codons are not near (i.e. more than 10 bp from) the TAG defined in step 5; 7) there should be no ATG (initiation codon) after TAG defined in step 5 to exclude the possibility of unintended translation initiation; 8) sesRNAs can be directed to any region of a cellular transcript, including exons, introns, UTR regions, or mature mRNA after splicing. 9) avoid sesRNA with complex secondary structures.

### A-to-I Editing Rate by CellREADR

To evaluate the A-to-I editing rate, HEK293T cells were seeded in 12-well plates. At 48-hour post transfection, cells were collected and RNA was extracted. RNAs were purified with RNeasy Mini Kit (Qiagen Cat. 74106) and converted to cDNA using TaqMan™ Reverse Transcription Reagents (Thermo Fisher, N8080234). PCR products covering the whole sesRNA region was generated with CloneAmp HiFi PCR Premix (Takara, Cat. No. 639298), and purified with NucleoSpin Gel and PCR Clean-up kit (Takara, Cat. No. 74061) for Sanger sequencing. Editing efficacy was calculated as the ratio of Sanger peak heights G/(A+G). Three biological replicates were performed.

### Transcriptome-Wide RNA-Sequencing Analysis

The readrRNA^{Ctrl} or readrRNA^{PCNA}, readrRNA^{EEF1A1-CDS}-expressing plasmids with the red fluorescent protein (tdTomato) expression cassette were transfected into 293T cells. The RFP⁺ cells (about 5×10\hat{ }5) were enriched by FACS sorting 48 h after transfection, and RNAs were purified with RNeasy Mini Kit (Qiagen Cat. 74106). To prepare the library, RNA samples were processed with the TrueSeq Stranded mRNA library Prep kit (Illumina, 20020594) and TrueSeq RNA Single Indexes (Illumina, 20020492). Deep sequencing analysis were performed in Illumina NextSeq500 platform at Cold Spring Harbor Laboratory NGS Bioinformatics Center. FastQC was applied to RNA-seq data to check the sequencing quality. All samples passed quality check were mapping to reference genome (GRCh38-hg38). STAR (version 2.7.2) and RSEM (version 1.3.2were used to annotate the genes. TPM values was calculated from RSEM. For all genes with at least one of the libraries above zero reads, the average expression values across biological replicates were compared between samples for detecting differentially expressed genes, using DESeq2³⁶ Genes with adjusted p-values<0.01 and Fold Change>2 or <0.5 were identified as significantly differentially expressed. For global A-to-G editing rate analysis, A-to-G editing rate calculation was performed using RNA Editing Detection Pipeline (REDP) which is a python wrapper for REDItool³⁷. Parameters were set to default values. Alignment step was done using STAR v2.7.8a with default and coordinate-sorted parameters. Genome reference from Gencode GRCH38.p13 was used and concatenated with known sequences from target genes including EEF1A1 and PCNA.

### Western Blot

Primary antibodies against ADAR1 (Santa Cruz, sc-271854, 1:500), GFP antibody (Cell Signaling Technology, Cat. 2956, 1:10000), β-Actin antibody (Cell Signaling Technology, Cat. 3700, 1:10000) were used in this study. Standard western blot protocols are used. Briefly, ˜2×10⁶ cells were lysed and an equal amount of each lysate was loaded for SDS-PAGE. Then, sample proteins were transferred onto polyvinylidene difluoride membrane (Bio-Rad Laboratories) and immunoblotted with primary antibodies against ADAR1 or GFP, followed by secondary antibody incubation (1:10,000) and exposure.

### Luciferase Complementation Assay

Forty-eight hours after transfection, HEK293T cells expressing firefly luciferase gene were washed in PBS, collected by trituration and transferred to 96-well plates. Promega Luciferase Assay System (Promega, E1500) was used. Firefly luminescence was measured using SpectraMax Multi-Mode Microplate Readers (Molecular Devices).

qRT-PCR

RNA was extracted and purified with RNeasy Mini Kit (Qiagen Cat. 74106). RNA was converted to cDNA using TaqMan™ Reverse Transcription Reagents (Thermo Fisher, N8080234). qRT-PCR was performed with Taqman probes on the QuantStudio 6 Flex real-time PCR system. The housekeeping gene TBP was used for normalization. The gene probes are purchased from Thermo Fisher Scientific. (Thermo Fisher Scientific, EEF1A1: Hs01591985, PCNA: Hs00427214, XIST: Hs00300535, ACTB: Hs03023943, Mda5: Mm00459183_m1, Rig-I: Mm01216853 m1, Ifnb1: Mm00439552_s1, 116: Mm00446190 m1, Ccl2: Mm00441242_m1, Fgf2: Mm01285715_m1, Cd40: Mm00441891_m1, Iba1: Mm00479862_g1, Cxcl10: Mm00445235_m1). The housekeeping gene_TBP (Thermo Fisher Scientific, human TBP: Hs00427620, mouse Tbp: Mm01277042_m1) was used for normalization.

### Virus Production

For producing READR and Reporter viruses, HEK293T cells were transiently transfected with READR or Reporter plasmids, AAV serotype plasmids, and pHelper using PEI MAX (catalog 24765, Polysciences Inc.). Seventy-two hours after transfection, the cells were collected in cell culture media, and centrifuged at 4,000 rpm for 15 minutes. The supernatant was discarded, the pellet was resuspended in cell lysis buffer, frozen and thawed three times using a dry ice/ethanol bath. Cell lysate was centrifuged at 4,000 rpm for 20 minutes. The contaminating DNA in the supernatant was removed by adding Benzonase and was incubated at 37° C. for 30 minutes. The viral crude solution was loaded onto an iodixanol density gradient and spun at 60,000 rpm for 90 minutes using a Beckman Ti70 rotor. After the centrifugation, 3-4 ml crude viral preparation was collected from the 40-60% layer with an 18-gauge needle attached to a 10 ml syringe. The viral crude solution was concentrated to 200-250 μl using the Amicon Ultra-15 centrifugal filter (100 kDa), washed with 8 ml PBS once, and concentrated to an appropriate volume. Aliquots were stored at −80° C. until use. Mouse and rat READR and Reporter viruses were packaged as AAV-DJ or AAV-PHP.eB serotypes. PT enhancer-tTA2 viruses were packaged as PHP.eB with vector from Addgene (#163480). Human READR and Reporter viruses were packaged as AAV2-Retro serotype. pAAV(DJ)-hSyn-Adar2-sesRNA^{Fez}f²-tTA2 and pAAV(PHP.eB)-hSyn-ADAR2-sesRNA^{Ctip2(1)}-tTA2 were packaged from Vigene Inc.)

### Immunohistochemistry

Mice or rat were anesthetized (using Avertin) and intracardially perfused with saline followed by 4% paraformaldehyde (PFA) in 0.1 M PBS buffer. Following overnight fixation at 4° C., brains were rinsed three times and sectioned 50 μm thick with a Leica 1000s vibratome. Sections were placed in blocking solution containing 10% Normal Goat Serum (NGS) and 0.1% Triton-X100 in PBS1× for 1 hour, then incubated with primary antibodies diluted blocking solution overnight at 4° C. Anti-GFP (1:1000, Aves, GFP-1020); anti-CTIP2 (1:250, Abcam 18465); anti-TLE4 (1:500, Scbt: sc-365406); anti-Rorb (1:300, Novus Biologicals NBP1-82532) were used. Sections were rinsed 3 times in PBS and incubated for 1 h at room temperature with corresponding secondary antibodies (1:500, Life Technologies). Sections were dry-mounted on slides using Fluoromount (Sigma, F4680) mounting medium.

For human tissue, after viral expression had reached a peak, or following patch clamp recording, cortical slices were fixed overnight in 4% PFA, then rinsed in PBS and stored from 1-7 days in PBS with azide. For biocytin recovery, tissue was permeabilized in PBS with 10% TritonX100, 5% NGS, 100 uM glycine, and 0.5% BSA for 30 minutes at room temperature on a shaker plate. Both primary and secondary antibodies were incubated 24 hours at 4C. Biocytin was either developed directly with Alexa647 conjugated streptavidin, or indirectly with enzyme metallography method using peroxidase-labeled streptavidin treated with silver ions substrate which deposits metallic silver at the active site. Recovered cells were imaged on a Keyence BZ-X800 with a 10× or 20× objective. Large overview images of native mNeon fluorescence were made on a Leica SP8 upright confocal microscope. For immunohistochemical labeling of FOXP2, and NeuN, tissue was cryoprotected for at least 24 hours in 30% sucrose, then re-sectioned at 40 μm with a sliding microtome. Floating sections were permeabilized in PBS with 1% TritonX100, 5% NGS, and 100 uM glycine for 30 minutes at room temperature and labeled overnight at 4C with primary antibodies (rabbit anti-FOXP2 Abcam #16046 1:500, rat anti-Flag 1:500 Novus NBP1-06712, mouse anti-NeuN Ab 1:500, Cell Signaling #94403S). For secondary antibodies, tissues were either incubated with biotinylated anti-rabbit Ab (1:1000, ThermoFisher 31822) for 2 hours at room temperature followed by streptavidin AlexaFluor647 for 1 hour at room temperature (FOXP2 or with goat anti-mouse Ab (1:1000, ThermoFisher 32723) for 2 hours at room temperature (NeuN).

### In Situ Hybridizaion

All probes were ordered from Molecular Instruments (mouse Satb2 cat #PRM128, mouse PlxnD1 cat #PRK885, mouse Fezf2 cat #PRA339, mouse vGAT cat #PRE853, rat vGAT cat #PRN024 and human VGAT cat #PRM351). Mouse brain was sliced into 50 μm thick slices after PFA perfusion fixation and sucrose protection. Hybridization chain reaction in situ was performed via free floating method in 24 well plate. First, brain slices were exposed to probe hybridization buffer with HCR Probe Set at 37 degree for 24 hours. Brain slices were washed with probe wash buffer, incubated with amplification buffer and amplified at 25 C for 24 hours. On day 3, brain slices were washed, counter stained if needed.

### Stereotaxic Viral Injection

Adult mice of 8 weeks or older were anesthetized by inhalation of 2% isofluorane delivered with a constant air flow (0.4 L/min). Ketoprofen (5 mg/kg) and dexamethasone (0.5 mg/kg) were administered subcutaneously as preemptive analgesia and to prevent brain edema, respectively, prior to surgery, and lidocaine (2-4 mg/kg) was applied. Mice were mounted in a stereotaxic headframe (Kopf Instruments, 940 series or Leica Biosystems, Angle Two). Stereotactic coordinates were identified. An incision was made over the scalp, a small burr hole drilled in the skull and brain surface exposed. A pulled glass pipette tip of 20-30 μm containing the viral suspension was lowered into the brain; a 500 nl volume of single or mixed viruses was delivered at a rate of 30 nl/min using a Picospritzer (General Valve Corp) into 300 μm and 700 um (1:1 volume) of injection sites; the pipette remained in place for 10 min preventing backflow, prior to retraction, after which the incision was closed with nylon suture thread (Ethilon Nylon Suture, Ethicon Inc. Germany) or Tissueglue (3M Vetbond), and animals were kept warm on a heating pad until complete recovery. For rat Tle4 cell type targeting, 3:1 mixture of READR^{Tle4} and Reporter AAVs was stereotactically injected into the cortex (0 mm posterior, 3.5 mm lateral), 1000 ul volume was injected at −2 mm ventral and 1000 ul volume was injected at −1 mm ventral, respectively. For rat vGAT targeting, 3:1 mixture of READR^{vGAT} and Reporter AAVs was stereotactically injected into the cortex (−4 mm posterior, 3.5 mm lateral), 1000 ul volume was injected at −2.5 mm ventral and 1000 ul volume was injected at −1.5 mm ventral, respectively. Further experiments of immunohistochemistry or in situ hybridization were performed after 3 weeks of virus incubation.

### Physiology in Mouse

Stereotaxic surgery and viral injection. All surgeries were performed under aseptic conditions and body temperature was maintained with a heating pad. Standard surgical procedures were used for stereotaxic injection and optical fiber implantation. Mice were anesthetized with isoflurane (2-5% at the beginning and 0.8-1.2% for the rest of the surgical procedure) and were positioned in a stereotaxic frame and on top of a heating pad maintained at 34-37° C. Ketoprofen (5 mg/kg) was administered intraperitonially (IP) as analgesia before and after surgery, and lidocaine (2%) was applied subcutaneously under the scalp prior to surgery. Scalp and connective tissue were removed to expose the dorsal surface of the skull. The skin was pushed aside, and the skull surface was cleared using saline. A digital mouse brain atlas was linked to the stereotaxic frame to guide the identification and targeting of different brain areas (Angle Two Stereotaxic System, Leica Biosystems). The following coordinates were used for injections and implantations in the SSp-ul: −0.09 from bregma, 2.46 mm lateral from the midline; CFA: 0.37 mm from bregma, 1.13 mm lateral from the midline.

For viral injection, a small burr hole was drilled in the skull and brain surface was exposed. A pulled glass pipette tip of 20-30 m containing the viral suspension was lowered into the brain; a 500 nl volume was delivered at a rate of 10-30 nl/min using a Picospritzer (General Valve Corp); the pipette remained in place for 5 min preventing backflow, prior to retraction. Injection was made at a depth of 700 m. An optical fiber (diameter 200 m; NA, 0.39) was then implanted in CFA or SSp-ul for optogenetic activation or fiber photometry respectively. The optical fiber was implanted with its tip touching the brain surface. To fix the optical fiber to the skull, a silicone adhesive (Kwik-Sil, WPI) was applied to cover the hole, followed by a layer of dental cement (C&B Metabond, Parkell), then black instant adhesive (Loctite 426), and dental cement (Ortho-Jet, Lang Dental). A titanium head bar was fixed to the skull around lambda using dental cement. Mice were transferred on a heating pad until complete recovery. Further experiments of optogenetic activation or fiber photometry were performed after 8-weeks of virus incubation.
In vivo optogenetic activation. The mice were first briefly anesthetized with isoflurane (2%) to attach a reflective marker on the back of their left paws. Mice were then transferred into a tube, head fixed on a stage, and allowed to fully recover from the anesthesia before stimulation starts. A fiber coupled laser (5-ms pulses, 10-20 mW; X=473 nm) was used to stimulate at 20 and 50 Hz and constantly for 0.5 s. The inter stimulation interval was 9.5 s. Two cameras (FLIR, FL3-U3-13E4C-C), one frontal and one side camera, were installed on the stage to take high frame rate (100 Hz) videos. The two cameras were synchronized by TTL signals controlled by custom-written MATLAB programs. Videos and TTL-signal states were acquired simultaneously using workflows in Bonsai software. Four LED light lamps were used for illumination (2 for each camera).
Behavioral video analysis. The two cameras were calibrated using Camera Calibrator App in MATLAB. For left-paw tracking using MATLAB, the images from the videos were smoothed with a Gaussian lowpass filter (size 9, sigma 1.8). The centroid of the reflective marker on the paw was first detected by a combination of brightness and hue thresholding, then tracked by feature-based tracking algorithms (PointTracker in Computer Vision Toolbox). The tracking results were validated manually, and errors were corrected accordingly. Trials in which mice made spontaneous movements before stimulation onset (within 0.5 s) were excluded from further analysis.
Paw stimulation. To stimulate the left paw, mice were lightly anesthetized with isoflurane (0.75-1%). Body temperature was maintained using a feedback-controlled heating pad. A piezo bender (BA4510, PiezoDrive), driven by a miniature piezo driver (PDu100B, PiezoDrive), was insulated with Kapton tape on which a blunt needle was glued. The tip of the needle was attached to the back of the paw to stimulate it with vibration (100 Hz, sine wave, 1 s). Inter stimulation interval was 10 s.
In vivo fiber photometry and data analysis. A commercial fiber photometry system (Neurophotometrics) was used to record calcium activities from SSp-ul. A patch cord (fiber core diameter, 200 m; Doric Lenses) was used to connect the photometry system with the implanted optical fiber. The intensity of the blue light (λ=470 nm) for GCaMP excitation was adjusted to ˜60 W at the tip of the patch cord. A violate light (λ=415 nm, ˜60 μW at the tip) was used to acquire the isosbestic control signal to detect calcium-independent artifacts. Emitted signals were band-pass filtered and focused on the sensor of a CMOS camera. Photometry signal and stimulation onset were aligned based on TTL signals generated by a multifunctional I/O device (PCIe-6321, National Instruments). Mean values of signals from the ROI were calculated and saved by using Bonsai software, and were exported to MATLAB for further analysis.

To process recorded photometry signals, first a baseline correction of each signal was performed using the adaptive iteratively reweighted Penalized Least Squares (airPLS) algorithm (https://at github.com/zmzhang/airPLS) to remove the slope and low frequency fluctuations in signals. The baseline corrected signals were standardized (Z-score) on a trial-by-trial basis using the median value and standard deviation of 5-s baseline period. The standardized 415-nm excited isosbestic signal was fitted to the standardized 470-nm excited GCaMP signal using robust linear regression. Lastly, the standardized isosbestic signal was scaled using parameters of the linear regression and regressed out from the standardized GCaMP signal to obtain calcium dependent signal.

### Human Organotypic Sample Preparation

Human neocortical tissues (frontal, parietal, temporal) were obtained from pediatirc and adult patients (N=6; ages 6-60) undergoing brain resections for epilepsy. Informed consent was obtained for the donation of tissues under Duke University IRB Protocol 00103019.

Tissue preparation generally followed the methods described in the literature^{41,57,58}. Dissection solution contained (in mM): 75 sucrose, 87 NaCl, 25 glucose, 26 NaHCO3, 2.5 KCl, 1.2 NaH2PO4, 10 MgCl2, 0.5 CaCl2), bubbled with 95/5% 02/CO2 (pH 7.4). Cortex was carefully dissected under ice cold (˜4C) oxygenated dissection solution to remove the pia. Single gyri were blocked for slicing and cut at 300 μm thickness on a Leica VT1200. Slices were rinsed in Hank's Buffered Saline and plated on PTFE membranes (Millipore, cat. #PICMORG50) in 6 well plates. Tissue was cultured in a conditioned media described in the literature⁴⁰. Conditioned media also contained anti-biotic/anti-mycotic (1×, Gibco by ThermoFisher) for the first 7 days in vitro, and cyclosporine (5 ug/mL) for the entire culture period. HEPES (20 mM) was added to the media for the first hour after slicing. AAV retro-grade (AAV-rg) viruses were suspended in conditioned media and applied directly to the slice surface by pipet on the day of plating. Expression of the mNeon reporter was monitored from DIV 3 onward, and tissue was used for immunohistochemistry and patch clamp experiments between DIV 6 and DIV 14.

### Electrophysiology in Human Organotypic Sample

Cultured human neocortical tissues were transferred to a heated recording bath (32-34C) on the platform of an Olympus BX-50 upright microscope. Recording solution contained (in mM): 118 NaCl, 3 KCl, 25 NaHCO3, 1 NaH2PO4, 1 MgCl2, 1.5 CaCl2, 30 glucose fully saturated with 95/5% 02/CO2. Borosilicate patch pipettes were pulled with a resistance of 4.0-5.5 MΩ, and filled with an internal solution containing (in mM): 134 K-gluconate, 10 HEPES, 4 ATP-triphosphate Mg salt, 3 GTP-triphosphate Na salt, 14 Phosphocreatine, 6 KCl, 4 NaCl, pH adjusted to 7.4 with KOH. Biocytin (0.2%) was added to the internal solution to allow for morphological identification after recording. Patched cells were held in whole-cell mode for a minimum of 12 minutes to ensure compete filling with biocytin. Cells expressing mNeon were patch-clamped under visual guidance using an Axopatch 700B amplifier (Molecular Devices). Data was digitized with (Digidata 1550B) and recorded with pClamp 10 (Molecular Devices). Membrane voltage was recorded at 100k Hz and low-pass filtered at 10k Hz. Liquid junction potential was not corrected. Pipette capacitance and series resistance were compensated at the start of each recording, and checked periodically for stability of the recording configuration. Intrinsic membrane properties were measured with a −10 pA current step. Firing rheobase was measured with a ramp current of is duration and 100-300 pA final amplitude. Input-output curves were generated from a series of current step starting at −50 pA and increasing in 10 pA increments until a maximum firing rate was elicited. Data were analyzed using NeuroMatic and in-house routines in Igor Pro (Wavemetrics).

### Microscopy and Image Analysis

Cell imaging in tissue culture was performed on ZEISS Axio Observer (CSHL St. Giles Advanced Microscopy Center). Imaging from serially mounted brain sections was performed on a Zeiss LSM 780 or 710 confocal microscope (CSHL St. Giles Advanced Microscopy Center) and Nikon Eclipse 90i fluorescence microscope, using objectives X63 and x5 for embryonic tissue, and x20 for adult tissue, as well as x5 on a Zeiss Axioimager M2 System equipped with MBF Neurolucida Software (MBF). Quantification and image analysis was performed using Image J/FIJI software. Statistics and plotting of graphs were done using GraphPad Prism 9.

### Statistics

Unpaired two-tailed Student's t-test was used for group comparison. Statistical analyses were performed Prism 9 (GraphPad Software, Inc.). DESeq2 was used to analyze statistical significance of transcriptome-wide RNA-seq data.

Any references, publications, online resource, patent and/or non patent literature referred to herein is hereby incorporated by reference herein in its entirety.

### Overview of Cell Type Access by Programmable RNA Sensing

Diverse cell types in multi-cellular organisms are obligatory intermediates through which genomes construct and orchestrate organismal phenotypes. Deciphering the organizational logic of this biological information flow and its alterations in diseases requires methods that allow specific and comprehensive analysis of cell types across diverse species. The following examples describe CellREADR (Cell access through RNA sensing by Endogenous ADAR), which couples the detection of a somatic cell type-defining RNA to the translation of desired effector proteins, implemented through RNA editing mediated by adenosine deaminase acting on RNA (ADAR). The following examples demonstrate that CellREADR senses specific RNA sequences to translate reporter proteins in human and mouse cell lines, and in distinct neuron types of mouse cerebral cortex when delivered by viral vectors. This RNA-programmable technology highlights the potential for discovering, monitoring, and editing animal cell types in ways that are specific, simple, versatile, and general across organ systems and species, with broad applications in biology, medicine, and biotechnology.

### Example 1. CellREADR Design and Validation in Mammalian Cells

RNA editing is a widespread and robust post-transcriptional mechanism essential to the metazoan gene regulatory toolkit implicated in recoding, splicing, microRNA targeting, and other RNA processing and cellular processes [20,21]. The most prevalent form of RNA editing is adenosine-to-inosine (A->I) conversion, catalyzed by ADARs; inosine is recognized as guanosine (G) by the cellular machinery [21]. ADARs recognize and are recruited by stretches of base-paired double-stranded (ds) RNAs [20], thus can operate as a sequence-guided base editing machine and have been harnessed for transcriptome editing [22-26]. As ADARs are ubiquitous in animal cells [27], the CellREADR was designed as a single and modular readrRNA molecule (FIG. 1a). The 5′ region of readrRNA contains a sensor domain of ˜300 nucleotides, which is complementary to and thus detects a specific cell type RNA through sequence-specific base pairing. This sensor domain contains one or more ADAR-editable STOP codons that act as a translation switch; which led to the nomenclature of this region to the sense-edit-switch RNA (sesRNA). Downstream to the sesRNA and in-frame to the STOP is sequence coding for the self-cleaving peptide 2A, followed in-frame by an effector RNA (efRNA) region coding for various effector proteins. The entire readrRNA is several kilobase, depending on specific sensors and effectors, and thus is deliverable to cells through viral vectors or liposome nanoparticles. In cells expressing the target RNA, sesRNA forms a dsRNA with the target, which recruits ADARs to assemble an editing complex. At the editable STOP codon, ADARs convert A to I, which pairs with the opposing C in the target RNA. This A->G substitution converts a TAG STOP codon to a TI(G)G tryptophan codon, switching on translation of efRNA. The in-frame translation generates a fusion protein comprising an N-terminal peptide, 2A, and C-terminal effector, which then self-cleaves through 2A and releases the functional effector protein (FIG. 1a). readrRNA is expected to remain inert in cells that do not express the target RNA.

### Example 2. Operativity in Human Cells

A proof-of-principle version of CellREADR was built and tested it in the human 293T cell line. An expression vector (PGK-tdT) was used to express the tdTomato gene as an exogenous target RNA and to label the transfected cells. Next, a READR vector (READRtdT-GFP) expressing a readrRNA consisting of a 5′ sensor region to tdT sequence embedded with a TAG STOP codon (sesRNAtdT), a 2A coding sequence, and a 3′ coding cassette for green fluorescence (GFP) was designed FIG. 6. Co-transfection of READRtdT-GFP with PGK-tdT resulted in GFP expression in a subset of tdT+ cells (FIG. 6). To substantiate this result, the GFP coding region of READR vector was replaced with that of a tetracycline inducible transcription activator (tTA2), which drove the expression of a firefly luciferase gene (Luc) from the tetracycline response element (TRE) promoter (FIG. 6). Co-transfection of READRtdT-tTA2 and TRE Luc with PGK-tdT resulted dramatic induction of luminescence compared to empty vector control (FIG. 6). A sparse and weak GFP or luciferase expression, respectively, was observed from READRtdT-GFP alone or READRtdT-tTA2/TRE Luc when co-transfected with an empty PGK vector (FIG. 6b-f), which may have resulted from occasional read-through of STOP codon in the sesRNA [28].

### Example 3. SPACERS

Spacer sequences reduced the leakage of readrRNA in accordance with one embodiment of the present disclosure. Inclusion of an in-frame spacer region of approximately 600 bp before the sesRNA virtually eliminated residual GFP translation, FIG. 2a. FIGS. 6g and 6i a is a schematic READRtdT-GFP vector in which a spacer sequences is inserted before sesRNAtdT coding region. FIG. 6J is a representative FACS analysis of GFP and RFP expression in 293T cells transfected with READRtdT-G′P vector encoding viable length of spacers with (upper) or without (lower) tdT target RNA expression, respectively. (FIG. 6j); longer spacers decreased the efficacy of readrRNA in the presence of tdT target RNA (FIG. 6h), which shows the quantification of conversion ratio calculated as percentage of GFP+ cells among RFP+ cells shown in FIG. 6J.

### Example 4. Quantification

To more quantitatively characterize CellREADR efficiency, a BFP coding cassette was inserted upstream of the sesRNAtdT region in READRtdT-GFP, which functioned as a spacer as well as to label all READRtdT-GFP transfected cells along with CAG-tdT transfected cells (FIG. 1b, c). CellREADR efficiency was calculated as the ratio of GFP+ cells among RFP+ cells gated on BFP+ cells in FACS analysis. In the absence of tdT target RNA, READRtdT-GFP showed no leaky GFP expression. In the presence of tdT RNA, the efficiency of READRtdT-GFP was 15.2% (FIG. 1c, d), while a control READR vector expressing a sesRNACtrl with scramble coding sequence containing a STOP codon showed no GFP translation.

### Example 5. CellREADR Efficiency Correlates with Target RNA Levels

Next, it was examined whether CellREADR efficiency correlated with target RNA levels. The efficiency of READRtdT-GFP increased with increasing amount of CAG-tdT vector in 293T cell transfection. A tetracycline inducible expression vector was further designed in which a ChETA (a variant of light-gated channelrhodopsin-2) coding sequence was fused to Blue Fluorescent Protein (BFP) and driven by TRE, thus BFP fluorescence level indicated Cheta transcript level, which correlated to tetracycline concentration in culture medium. Increase of tetracycline concentration and BFP expression levels increased READRCheta-GFP efficiency in a Cheta RNA dependent manner. FIG. 1e-f. With a constitutive promoter driven BFP-Cheta expression, READRCheta-GFP efficiency reached as high as 41.5%. These results indicated the target RNA level dependence and robustness of CellREADR. To demonstrate the capacity of effector RNAs for mediating physiological function, we constructed READRtdT-Cas9 to express the Cas9 protein for genomic DNA editing29 and READRtdT-taCas3/TEV to express functional Caspase3 protein for programmed cell death30. Efficient gene editing (FIG. 7a-d) and increased cell apoptosis (FIG. 7e-f) was induced, respectively, only with the presence of tdT target RNA.

### Example 6. Role of ADAR Proteins in CellREADR Function

To examine the role of ADAR proteins in CellREADR function, an ADAR1 gene knockout (KO) 293T cell line was generated using CRISPR. As ADAR1 is highly expressed while ADAR2 is barely expressed in 293T cells, ADAR1KO 293T essentially represented an ADAR-null cell line. Removal of ADAR1 largely eliminated READRtdT-GFP functionality (FIG. 5h, i,), which was rescued by exogenous expression of either the p¹10 or p150 ADAR1 isoform as well as the ADAR2 protein (FIG. 5h, i). Overexpression of ADAR2 in wide type cells increased READR^{tdT-GFP} efficiency by ˜25-30% (FIG. 5h, i). Sanger sequencing confirmed A-to-I/G editing at the intended site that converted the TAG STOP codon to a TGG tryptophan codon (FIG. 5j, k). Since ADAR expression can be induced by interferon stimulation [32, 33], it was further confirmed that ADAR1-p110 and ADAR1-p150 ADAR were induced by interferon stimulation in wild type 293T cells, and demonstrated that READRtdT-GFP efficiency increased accordingly.

### Example 7. CellREADR Function in Wide Range of Cells

In addition to 293T cells, CellREADR functionality was demonstrated in several other cell lines originated from different tissues or species, including human HeLa, mouse N2a, and mouse KPC1242 cell lines. Collectively, these results demonstrate that by leveraging cell-endogenous ADARs, CellREADR can couple the detection of a cellular RNA to the translation of an effector protein in a way that is specific, efficient, and robust.

### Example 8 sesRNAs Properties Confer CellREADR Programmability

As sesRNA is a key component of CellREADR, several properties of sesRNAs were explored using an ADAR2 overexpression READRAdar2-tdT-GFP vector as a robust assay. It was found that sesRNA of less than 100 nucleotides were largely ineffective, while sesRNAs of increasing length positively correlated with the CellREADR efficiency, with an optimal length of 200-250 nt.

### Example 9 Role of Mismatches in Programmability

In terms of sequence mismatch with target RNA, sesRNAs of ˜200 nt tolerated up to 10 mismatches (5% of sequence length) or in-frame indels without major decrease of READRAdar2-tdT-GFP efficienc (FIG. 2 by; this property confers flexibility for sesRNA design (see Methods).

However, mismatches near the editing site significantly reduced CellREADR efficiency. To explore the effect of locations along target transcript on sesRNAs design, an EF1a-Cheta-tdT expression vector was used and tested 5 sesRNAs targeting the promotor, different coding regions, and 3′ UTRs, respectively. All 4 sesRNAs targeting the transcript region exhibited robust efficiency, except the one targeting the promoter. Finally, it was examined whether inclusion of more STOP codons may further increase sesRNA stringency (i.e. reduce basal translation in the absence of target RNA). Using luciferase as a sensitive indicator of leaky translation, sesRNAs with two STOP codons showed reduced leakage with comparable efficiency compared to those with one STOP codon, although 3 STOP codons significantly reduced the efficiency. FIG. 2d-f. Together, these results suggest strategies to enhance stringency, efficiency, and flexibility of CellREADR.

### Example 10. CellREADR is Inherently Programmable

Built on Watson-Crick base-pairing, CellREADR is inherently programmable, conferring potential for intersectional targeting of two or more cellular RNAs for more specific cell type definition. To explore this property, dual- or triple-sesRNA arrays were designed that target different regions of the same transcript. All of the tested sesRNA arrays exhibited robust CellREADR efficacy, comparable to that of a single sesRNA.

To examine the possibility for intersectional targeting of two target RNAs, a sesRNA array consisting of tandemly arranged sesRNAtdT and sesRNACheta (sesRNAtdT/Cheta) was designed. Whereas single target RNA, tdTomato or Cheta, failed to induce GFP or Luc expression with readrRNAtdT/Cheta, significant GFP expression or increased luminescence were observed, respectively, when both tdTomato and Cheta vectors were transfected. This result demonstrated the potential of intersectional cell type targeting based on the programmability of CellREADR. FIG. 2.

### Example 11. Endogenous RNA Sensing by CellREADR

Compared with synthetic genes expressing exogenous RNAs, endogenous genes often have more complex genomic structures, including numerous exons, introns, and regulatory elements; transcribed endogenous RNAs undergo multiple and elaborate post-transcriptional steps such as splicing and chemical modifications before being processed as mature mRNAs. To examine the capacity of CellREADR for targeting cell endogenous RNAs, EEF1A1 a housekeeping gene highly expressed in 293T cells, was first selected and systematically designed a set of sesRNAs targeting its various exons, introns, 5′ and 3′ UTR, and mRNAs. FIG. 3. These sesRNAs were effective in sensing all intended EEF1A1 regions to switch on efRNA translation; exons were better targets than UTRs and introns. Notably, sesRNAs complementary to an mRNA region joined from two exons achieved comparable efficacy as to those contained within exons, indicating that CellREADR acted on mRNAs as well as pre-mRNAs. CellREADR efficacy to EEF1A1 RNAs increased with incubation time after cell transfection. Several other endogenous RNAs were further tested in 293T cells, including a highly expressed ACTB, moderately expressed PCNA, and a long non-coding RNA XIST. Robust CellREADR efficacy for all these RNAs (FIG. 3d) were observed. Exogenous ADAR2 overexpression resulted in rather modest increase of CellREADR efficacy by about 30% (FIG. 3c, d). Together these results suggest that cell endogenous ADARs enable efficient and robust CellREADR functionality on cell endogenous RNAs.

### Example 12. Effect of CellREADR on the Levels of Targeted Transcripts

CellREADR did not alter the levels of targeted transcripts assayed by quantitative PCR (qPCR) (FIG. 3e); this result also ruled out the possible effect of RNA interference induced by readrRNAs. RNA-sequencing analysis was further performed to evaluate the transcriptomic effects of CellREADR by comparing the transcriptome profiles of 293T cells expressing sesRNA^{EEF1A} or sesRNA^{PCNA} with those expressing a scrambled sesRNACtrl. Correlation analysis showed that sesRNAEEF1A or sesRNAPCNA expression had no significant impact on global transcriptome (FIG. 3f), and DESeq2 analysis revealed no significant differential gene expression between the sesRNA^{EEF1A} or sesRNA^{PCNA} and sesRNA^{Ctrl} group, demonstrating that CellREADR had no significant effect on cellular transcriptome.

### Example 13. Further Endogenous RNA Sensing by CellREADR

Compared with synthetic genes expressing exogenous RNAs, endogenous genes often have more complex genomic structures, including numerous exons, introns, and regulatory elements; transcribed endogenous RNAs undergo multiple and elaborate post-transcriptional steps such as splicing and chemical modifications before being processed as mature mRNAs34. To examine the capacity of CellREADR for detecting cell endogenous RNAs, EEF1A1, a housekeeping gene highly expressed in 293T cells was first selected, and systematically designed a set of sesRNAs targeting its various exons, introns, 5′ and 3′ UTR, and mRNAs (FIG. 3a, b). These sesRNAs were effective in sensing all intended EEF1A1 regions to switch on efRNA translation; exons appeared to be better targets than introns (FIG. 3c). Notably, sesRNAs complementary to a mRNA region joined from two spliced exons achieved comparable efficiency as to those contained within an exon, indicating that sesRNA can be designed to target both pre-mRNA and mRNA sequence. CellREADR efficacy to EEF1A1 RNAs increased with incubation time after cell transfection.

### Example 14. Sensitivity

To examine the sensitivity of CellREADR to target RNA levels, several other endogenous RNAs were further tested in 293T cells, including a highly expressed ACTB, moderately expressed PCNA, and a long non-coding RNA XIST. Several endogenous RNAs with expression ranging from high (ACTB, PCNA), modest (TP53, XIST), to low (HER2, ARC) levels were selected. Robust CellREADR efficacy for all these RNAs (FIG. 3d) were observed. Exogenous ADAR2 overexpression resulted in rather modest increase of CellREADR efficacy by about 30% (FIG. 3c, d). CellREADR was able to reliably detect even low levels ARC RNAs, although at much lower efficiency. Together these results suggest that cell endogenous ADARs enable efficient and robust CellREADR functionality on cell endogenous RNAs.

### Summary of Examples 1-14

The CellREADR was tested in human and mouse cell lines by building a reporter RNA editing assay in human 293Tcells, where the target RNA is made from an RFP expression vector, the Sensor RNA is anti-sense to the RFP target, and is then follow by a tTA transcription activator, which activates and amplifies GFP expression in the a third vector. When transfected into the cell, sensor RNA pairs with RFP RNA, and ADAR is recruited to edit the STOP codon, switching on translation of tTA that activates GFP, thereby turning the cell from red to green. The editing can also be quantified by Fluorescence Activated Cell Sorting [FACS] assay, where the upper right quadrant is the RFP/GFP positive cells.

This conversion is ADAR mediated as it did not happen in the ADAR1 KnockOut cell line, which can be then rescued by expression Adar2. Sanger sequencing proved that it is indeed due to an intended A-I (G) conversion that lifted the STOP codon.

This system also works in human Hela cells, and mouse N2a cells and on cell endogenous RNAs. When different regions of the Ef1a gene and pre-mRNA were used as targets, it was found that exons and coding regions are better sensing targets than intron and untranslated regions. In addition, this worked for five other cellular RNAs including a non-coding RNA.

### Example 15 Neuronal Cell Type Targeting with CellREADR

CellREADR as used to access specific cell types in animal tissues, including neuronal tissues and cells comprises both a singular vector system and a binary vector system (FIG. 4b). In the singular vector system, i.e., a singular READR vector comprises for example, a hSyn promoter which drives transcription of gene encoding mCherry red fluorescent protein followed by sesRNA and efRNA coding for smFlag and tTA2. This singular READR vector directly couples RNA detection with effector gene expression; its efficiency and specificity is evaluated by co-localization of mCherry, smFlag, and the target mRNA or protein.

The binary system includes an addition Reporter vector, which contains a TRE promoter driving mNeonGreen (mNeon) or other effector proteins in response to the tetracycline dependent transcription activator (tTA2) translated from the READR vector (FIG. 4b). The binary vectors provide amplification of effector gene expression as well as the combinatorial flexibility of expressing different effector genes in different cell types by pairing READR and Reporter vectors.

### In Vitro Targeting of Fezf2 or Ctip2

A set of READR vectors targeting mRNAs that define major glutamatergic projection neuron (PN) types of the mouse cerebral cortex was first tested and screened in 293T cells by co-expressing individual sesRNAs targeting neuronal RNA sequences in 293T cells, in this case Ctip2 and Fezf2, sequences (FIG. 9a, b, d). The zinc-finger transcription factor Fezf2 labels the vast majority of layer 5b (L5b) and L6 corticofugal projection neurons (CFPNs) that constitute cortical output channels, while Ctip2 predominantly labels a subset of L5b/L6 CFPNs neurons^{38,39}. All sesRNAs exhibited variable but significant CellREADR efficiency in 293T cells in the presence of exogenous Fezf2 or Ctip2 target sequences (FIG. 9e-f).

### In Vivo Targeting of Fezf2 or Ctip2

AAV vectors were used to administer these sesRNAs into the mouse cortex by focal injection into the whisker barrel somatosensory cortex (S1) or motor cortex (M1). When delivered with binary vectors, Fezf2 sesRNA1 showed 86.4% specificity, and Ctip2 sesRNA3 and 8 reached to over 90% specificity assayed by Ctip2 antibody staining. (FIG. 9j-k).

Ctip2 sesRNA3 was the focus of the following experiments. When the primary somatosensory mouse cortex (S1 cortex) was infected with singular READRAAV^{Ctip2/3}, FLAG immunofluorescence was concentrated in deep layers (FIG. 4c); co-localization of CTIP2 and FLAG immune-labeled cells (FIG. 4c, d) showed a specificity of 94.1% (FIG. 4e). In S1 cortex co-infected with binary READR^{Ctip2/3}/Reporter ^{mNeon} AAVs, mCherry expression from the READR vector labeled cells across cortical layers, while mNeon expression from the Reporter vector specifically labeled L5b and some L6 neurons (FIG. 4f); CTIP2 immunofluorescence showed a specificity of 90.4% (FIG. 4g). The efficiency of READR^{Ctip2/3}, calculated as the ratio of GFP cells among mCherry and Ctip2 double positive cells, reached 84.2% (FIG. 4h).

### ADAR2 Overexpression

To test whether ADAR2 overexpression from the READR vector might enhance CellREADR functionality, the mCherry module in the READR vector was replaced with an ADAR2 cDNA. Mouse cortex co-infected with binary READR^{Ctip2/3}/Reporter^{mRuby3} or binary READ^{Fezf2}/Reporter^{mRuby3} was tested for expression of sesRNAs for Fezf2 and Ctip2 FIG. 10a-c), and it was found that ADAR2 overexpression did not notably enhance the specificity or efficiency compared with binary vectors that relied on endogenous ADAR (FIG. 10c-i).

### Example 16—RNA Expression Levels and CellREADR

In addition to Ctip2 and Fezf2 mRNAs that mark CFPNs, (corticofugal projection neurons which send their axons to areas outside the cortex, including the thalamus and the spinal cord), sesRNAs that target several other mRNAs expressed at a range of levels⁴⁰ were constructed in binary vectors to (FIG. 11), including PlxnD1 (intratelencephalic projection neurons (IT PNs) in L2/3 and L5a), Satb2 (IT PNs across both upper and lower layers), Rorb (L4 pyramidal neurons), and vGAT (pan-GABAergic neurons), respectively. Several sesRNAs that achieved at or above 75% specificity were identified (FIG. 12). These results indicate that CellREADR is a robust method, and specific cell type targeting can be achieved by testing multiple sesRNAs for each target.

### Example 17—Long-Term Effects of CellREADR

To evaluate the long-term effects of CellREADR sesRNAs in vivo, READR^{Ctip2/3} and a control CAG-tdT AAVs were monitored in a mouse cortex for 3 months. Quantitative RT-PCR assay on expression of nine genes implicated in glia activation and immunogenicity showed no significant change in READR^{Ctip2/3}-infected versus control tissues (FIG. 13).

### Example 18 Recording and Control of Neuron Type with CellREADR

CellREADR efficacy was used to record and control neuronal function in mice. As a benchmark, CellREADR was compared to transcription enhancer-based cell type targeting approach. The mscRE4 enhancer was identified, to be operative in L5 pyramidal track (PT) neurons, a subset of CFPNs¹⁸. Thus, the efficacy of mscRE4 enhancer (i.e., PT enhancer) with Ctip2 sesRNA3 was compared by using the same tTA-TRE binary vectors for expressing genetic-encoded calcium indicator GCaMP6s and light-activated ion channel channelrhodopsin2 (FIG. 4i-p).

In mice coinfected with PT enhancer-tTA2Reporter^{GCaMP6s} or READR^{Cti}p^{2/3}/Reporter^{GCaMP6s} AAVs in the mouse forelimb somatosensory cortex, mechanical stimulation of the forepaw induced reliable and time-locked increase of GCaMP6s signals in forelimb somatosensory cortex, as measured by fiber photometry, in both PT enhancer and READR^{Ctip2/3} infected mice (FIG. 4i-1). This result indicates that effector gene expression levels achieved by CellREADR were sufficient to monitor cell physiology in live animals.

In mice co-infected with PT enhancer-tTA2 Reporter^{ChRger2-eYFP} or READR^{Cti}p^{2/3}Reporter^{ChR}g^{er2-eYFP} AAVs in the caudal forelimb motor area (CFA), light stimulation of CFA induced reliable and time-locked forelimb movement, which appeared stronger in READR^{Ctip2/3} compared to PT enhancer infected mice (FIG. 4m-p, Data Movie. 1). This result indicates that effector gene expression levels achieved by CellREADR were sufficient to control cell function in behaving animals. Anatomical analysis of eYFP expression confirmed L5 PT neuron labeling by READR^{Ctip2/3}, as revealed by axonal projections in subcortical targets including striatum, thalamus, pons, and medulla (FIG. 14).

### Example 19 CellREADR Targeting of Neuron Types Across Mammalian Species

A series of experiments in rat and human brain was performed to demonstrate the generalizability of CellREADR across mammalian species. GABAergic neurons were targeted in rats by using binary AAV vectors targeting vGAT mRNA, a pan-GABAergic transcript (FIG. 15b). Co-injection of READR^{vGAT} (hSyn-mCherry-sesRNA^{vGAT}-smFlag-tTA2) with Reporter^{mNeon} (TRE3g-mNeon) AAVs (FIG. 15a) into rat S1 barrel cortex and hippocampus specifically labeled GABAergic interneurons in the cortex (FIG. 15c) and hippocampus (FIG. 15d-c). The specificity, assayed by co-labeling of vGAT mRNA (magenta) and READR^{vGAT} (GFP), was 91.7% for cortex and 93.8% for hippocampus, respectively (FIG. 15f-g). Tle4 is a conserved transcription factor that marks L6 corticothalamic (CT) pyramidal cells in rodents. A sesRNA targeting exon15 of Tle4 mRNA (FIG. 15h) delivered by binary READR^{Tle4} AAVs labeled deep layer neurons with a co-labeling by TLE4 antibody at 62.5% (FIG. 15i-k).

### Example 20 Neocortical Oragnotypic Culture Platform

An organotypic culture platform was established for studying CellREADR in human neocortical specimens collected from epilepsy surgeries⁴¹ (FIG. 16a). Infection of these samples with a GFP construct driven by the hSyn promoter (AAVrg-hSyn-eGFP), displayed dense viral labeling across multiple layers, with a multitude of observed morphologies and a predominance of pyramidal neurons characterized by prominent apical dendrites (FIG. 16b, c). Two sesRNAs targeting FOXP2 mRNA (Forkhead box protein P2) (FIG. 5a, b), an evolutionarily conserved gene expressed in cortical and striatal projection neurons and implicated in human language skill development⁴² were developed. In situ hybridization and immunostaining indicate that, unlike in mouse, where FoxP2 expression is restricted to L6 CT neurons³⁹, human FOXP2 is expressed across cortical layers (FIG. 16d). Five days after applying binary READR^{FOXP2}/Reporter^{mNeon} AAVs (hSyn-ClipF-sesRNA^{FOXP2}-smV5-tTA2 with TRE3g-mNeon) to human neocortical slices, mNeon-labeled cell bodies were observed in upper and deep layers (FIG. 5c, Data FIG. 16e). FOXP2 immunostaining showed the specificity of binary READR^{FOXP2} was nearly 80% (FIG. 5d, e). Labeled neurons also exhibited electrophysiological (FIG. 5f) and morphological (FIG. 5g) properties consistent with glutamatergic pyramidal neurons⁴³. Using two singular READR^{FOXP2} vectors, each with a different sesRNA, over 97% specificity of targeting FOXP2 neurons was observed (FIG. 16f-i).

### Example 21 Targeting of Human Neocortical GABAergic Cells

Human neocortical GABAergic cells were targeted using a sesRNA to VGAT (FIG. 5h). Using a binary READR^{VGAT}/Reporter^{mNeon} cellular labeling across neocortical layers was observed (FIG. 5i), with labeled cells exhibiting diverse morphologies characteristic of interneurons, including multipolar and smooth dendrites (FIG. 5j), and strikingly dense, vertically oriented axons (FIG. 5i, k). In situ hybridization for VGAT mRNA showed a specificity of 76.5% (FIG. 5l, m), likely an underestimate due to the potentially reduced sensitivity of in situ hybridization in cultured ex vivo tissues. In support of this possibility, very few labeled cells were observed with pyramidal morphologies (FIG. 5i-l). Targeted patch-clamp recordings of READR^{VGAT}/Reporter^{mNeon} labeled interneurons seven days after virus application revealed various intrinsic properties, including accommodating, fast, and delayed onset firing (FIG. 5n). The physiological properties (FIG. 5n) and partially reconstructed morphologies of the patched cells (FIG. 5o) were consistent with those of mammalian neocortical interneurons⁴⁴. Together, these results demonstrate the cross-species generalizability and utility of CellREADR.

### Example 22 Targeting Neuron Types and Pathological Neural Ensembles for Treating Epilepsy

### Disease and Clinical Background

Epilepsy affects approximately 1% of the population, and in developed countries up to 30% of patients continue to experience seizures despite optimal antiepileptic medication ¹. The most effective treatment of focal epilepsy is surgery, but this is suitable for very few patients because of the unacceptable consequences of removing brain tissue. There is an urgent need to identify novel therapeutic targets and develop new treatment strategies. Gene therapy can regulate neuronal excitability in the refractory epileptic foci while preserving function ¹. However, current lack sufficient cell type and disease state specificity is a major impediment for effective gene therapy².

Focal epilepsy is generated by an imbalance of excitation and inhibition characterized by hyper-synchronized neural activity of pathological brain networks ¹. Brain circuits comprise multiple excitatory and inhibitory cell types, each making unique contribution in information processing and excitation-inhibition balance ^{3,4}. Neural activities in some cell types promote while in other cell types suppress seizure ². Therefore, it is critical to achieve cell type targeted modulation of neural activity at appropriate stages of seizure progress, such as initiation, maintenance etc ^{1,2}. Methods to suppress pathophysiological ensembles—those that actually participate in seizure activity, may be particularly effective. The CellREADR technology can use RNA sensors to achieve cell type and disease state specific modulation of brain circuit activity through expression of druggable modulatory receptors (e.g. GPCRs) and ion channels, thus represent a promising approach to control and treat focal epilepsy.

### Targeting GABAergic Inhibitory Neuron Types

GABAergic interneurons mediate inhibitory control of brain circuits and comprise diverse neuron types⁴. While the vesicular GABA transporter (vGAT) is expressed in all GABA neurons, parvalbumin (PV), somastatin (SST), calretinin (CR), and VIP mark several major subpopulations/types. The role of these interneuron types in the etiology of seizure remain not fully understood. PV neurons have a nuanced role and are shown to either promote or suppress seizure at different phases of epilepsy. CellREADR technology is helpful in targeting GABA interneurons in mouse, rat, human neocortex ⁹ (see Table A), and more recently macaque monkey neocortex (FIG. 17). Sensors to target human PV and SST neurons (FIG. 18) are used in all the human ex vivo cortex tissues which were resected from temporal or frontal lobes of epilepsy patients.

TABLE A

| Species | sesRNA # | Target Region |
|---|---|---|
| Mouse | MS-vGAT-ses1 | Exon1 |
| | MS-vGAT-ses2 | Exon2 |
| | MS-vGAT-ses3 | intron1 |
| | MS-vGAT-ses4 | intron2 |
| | MS-vGAT-ses5 | 5UTR |
| | MS-vGAT-ses6 | 3UTR |
| Rat | RAT-vGAT-ses1 | Exon2 |
| | RAT-vGAT-ses2 | Exon2 |
| | RAT-vGAT-ses3 | Exon2 |
| | RAT-vGAT-ses4 | Exon1 |
| Monkey | MNKY-vGAT-ses1 | Exon2 |
| | MNKY-vGAT-ses2 | Exon2 |
| | MNKY-vGAT-ses3 | Exon2 |
| | MNKY-vGAT-ses4 | Exon1 |
| Human | Hu-vGAT-ses1 | Exon2 |
| | Hu-vGAT-ses2 | Exon2 |
| | Hu-SST-ses1 | CDS |
| | Hu-SST-ses2 | CDS |
| | Hu-PV-ses1 | 5UTR |
| | Hu-PV-ses2 | CDS |
| | Hu-PV-ses3 | intron3 |
| | Hu-CR-ses1 | CDS |
| | Hu-CR-ses2 | CDS |

### Effector for GABA Inhibitory Neurons and Expected Outcome

A chemogenetic approach using two types of ligand-gated mechanisms to activate SST neuron-mediated inhibition to suppress seizure activity ¹. Chemogenetics relies on the modification of an endogenous receptor or the production of a modified chimeric receptor that responds to an exogenous ligand.

First, excitatory DREADD (designer receptors exclusively activated by designer drugs; hM3Dq excitatory receptor)¹⁰. DREADD can be specifically activated by the FDA approved drug clozapine-n-oxide or olanzapine ¹¹. The dose of the drugs can be titrated in order to suppress seizures without adverse effects.

Second, excitatory PSAM (pharmacologically selective actuator module/pharmacologically selective effector molecule) ¹². A new version PSAM has been developed that is sensitive to varenicline ¹³, a drug approved for smoking cessation.

The advantage of chemogenetic approach is that the gene therapy is inert until the drug is given, so it is possible to turn the gene therapy “off and on”. Moreover, the magnitude of the effect depends upon the dose of the ligand. It is therefore possible to titrate the dose of the drug to avoid any potential side-effects but yet to optimize efficacy. The CellREADR-based cell specific chemogenetic strategy can be generalized to target other GABAergic and glutamatergic neuron types for treating seizure and other neurological diseases.

### Targeting Pathophysiological Neural Ensembles (PNEs)

A particularly effective strategy to manage or treat epilepsy is to suppress or control the very neurons and neural ensembles that generate hyper-activities, i.e. pathophysiological neural ensembles (PNEs). CellREADR is useful for this. Hyper- and synchronized neural activities lead to rapid up-regulation of the immediate early gene (IEG) c-fos ^{14, 15}, which marks mostly glutamatergic excitatory neurons that constitute PNEs. c-fos RNA sensors have been constructed to target and modulate PNEs. A panel of 4 sensors has been tested, and many of which already show promising results in neuronal culture. In addition to c-fos, there is an extended list of other IEGs ¹⁵ that can also be used to target PNEs.

### Effector Genes

Three types of effector genes are coupled to c-fos sensors.

First is a voltage-gated potassium channel Kv1.1 (KCNA1) ¹⁶ which reduces spiking in neurons of PNEs. This will suppress the firing of neurons within PNEs.

Second is the inhibitory DREADD (the hM4D (Gi) inhibitory receptor)¹⁰, which can be activated by an oral drug olanzapine.

Third is an invertebrate glutamate receptor (eGluCL) that is permeable to chloride ^{17, 18}. eGluCLs bind glutamate released during seizure activity, and also can be activated by an FDA approved drug ivermectin; the resultant chloride current inhibits neuronal activity. Ivermectin can also be given to reduce neuronal activity either chronically or as a rescue medication.

A unique advantage of this approach of targeting PNEs is that it is a “closed loop” control. Under normal physiological condition, c-fos are not prominently expression, thus there is no effector expression. During epilepsy, up-regulation of c-fos RNA will trigger effector translation to suppress PNE activities. The strategy can be generalized to target other IEGs for treating seizure and other neurological diseases.

### Delivery

All the above CellREADR vectors are delivered to epileptic foci using AAV vectors (serotype 2/9 and php.eb). During a conventional epilepsy surgery, instead of removing the brain tissues in the epileptic foci, therapeutic CellREADR AAVs are injected to the foci, preserving the brain tissue.

### Expected Outcome and Improvement Over Current Treatments

With respect to the SST sensors and CellREADR vectors, oral administration of olanzapine (for excitatory DREADD) or varenicline (for excitatory PSEM) activates SST interneurons to suppress focal epilepsy. With c-fos sensors and CellREADR vectors, oral administration of olanzapine (for inhibitory DREADD) or varenicline (for inhibitory PSEM) reduces the firing of glutamatergic neurons in PNEs to suppress epilepsy. Together, these RNA therapeutics which are specific for a specified cell type and specified disease state will transform the management and treatment of epilepsy.

### REFERENCES

1. Walker M C, Kullmann D M. (2020) Optogenetic and chemogenetic therapies for epilepsy. Neuropharmacology. 2020 May 15; 168:107751. doi: 10.1016/j.neuropharm.2019.107751. Epub 2019 Sep. 5. PMID: 31494141 Review.
2. Magloire V, Mercier M S, Kullmann D M, Pavlov I. (2019) GABAergic Interneurons in Seizures: Investigating Causality With Optogenetics. Neuroscientist. 2019 August; 25(4):344-358. doi: 10.1177/1073858418805002. Epub 2018 Oct. 15. PMID: 30317911 Free PMC article. Review.
3. Huang Z J. (2014) Toward a genetic dissection of cortical circuits in the mouse. Neuron. 2014 Sep. 17; 83(6):1284-302. doi: 10.1016/j.neuron.2014.08.041. PMID: 25233312 Free PMC article. Review.
4. Huang Z J, Paul A. (2019) The diversity of GABAergic neurons and neural communication elements. Nat Rev Neurosci. 2019 September; 20(9):563-572. doi: 10.1038/s41583-019-0195-4. Epub 2019 Jun. 20. PMID: 31222186 Free PMC article. Review.
5. Taniguchi H, He M, Wu P, Kim S, Paik R, Sugino K, Kvitsiani D, Fu Y, Lu J, Lin Y, Miyoshi G, Shima Y, Fishell G, Nelson S B, Huang Z J. (2011) A resource of Cre driver lines for genetic targeting of GABAergic neurons in cerebral cortex. Neuron. 2011 Sep. 22; 71(6):995-1013. doi: 10.1016/j.neuron.2011.07.026. Epub 2011 Sep. 21. PMID: 21943598
6. He M, Tucciarone J, Lee S, Nigro M J, Kim Y, Levine J M, Kelly S M, Krugikov I, Wu P, Chen Y, Gong L, Hou Y, Osten P, Rudy B, Huang Z J. (2016) Strategies and Tools for Combinatorial Targeting of GABAergic Neurons in Mouse Cerebral Cortex. Neuron. 2016 Sep. 21; 91(6):1228-1243. doi: 10.1016/j.neuron.2016.08.021. Epub 2016 Sep. 8. PMID: 27618674
7. Vormstein-Schneider D, Lin J D, Pelkey K A, Chittajallu R, Guo B, Arias-Garcia M A, Allaway K, Sakopoulos S, Schneider G, Stevenson O, Vergara J, Sharma J, Zhang Q, Franken T P, Smith J, Ibrahim L A, Mastro K J, Sabri E, Huang S, Favuzzi E, Burbridge T, Xu Q, Guo L, Vogel I, Sanchez V, Saldi G A, Gorissen B L, Yuan X, Zaghloul K A, Devinsky O, Sabatini B L, Batista-Brito R, Reynolds J, Feng G, Fu Z, McBain C J, Fishell G, Dimidschstein J. (2020) Viral manipulation of functionally distinct interneurons in mice, non-human primates and humans. Nat Neurosci. 2020 December; 23(12):1629-1636. doi: 10.1038/s41593-020-0692-9. Epub 2020 Aug. 17. PMID: 32807948
8. Mich J K, Graybuck L T, Hess E E, Mahoney J T, Kojima Y, Ding Y, Somasundaram S, Miller J A, Kalmbach B E, Radaelli C, Gore B B, Weed N, Omstead V, Bishaw Y, Shapovalova N V, Martinez R A, Fong O, Yao S, Mortrud M, Chong P, Loftus L, Bertagnolli D, Goldy J, Casper T, Dee N, Opitz-Araya X, Cetin A, Smith K A, Gwinn R P, Cobbs C, Ko A L, Ojemann J G, Keene C D, Silbergeld D L, Sunkin S M, Gradinaru V, Horwitz G D, Zeng H, Tasic B, Lein E S, Ting J T, Levi B P. (2021) Functional enhancer elements drive subclass-selective expression from mouse to primate neocortex. Cell Rep. 2021 Mar. 30; 34(13):108754. doi: 10.1016/j.celrep.2021.108754. PMID: 33789096
9. Qian Y, Li J, Zhao S, Matthews E A, Adoff M, Zhong W, An X, Yeo M, Park C, Yang X, Wang B S, Southwell D G, Huang Z J. (2022) Programmable RNA sensing for cell monitoring and manipulation. Nature. 2022 Oct. 5. doi: 10.1038/s41586-022-05280-1. Online ahead of print. PMID: 36198803
10. Armbruster, B. N., Li, X., Pausch, M. H., Herlitze, S., Roth, B. L., 2007. Evolving the lock to fit the key to create a family of G protein-coupled receptors potently activated by an inert ligand. Proc. Natl. Acad. Sci. 104, 5163-5168. https://doi.org/10.1073/pnas. 0700293104.
11. Weston, M., Kaserer, T., Wu, A., Mouravlev, A., Carpenter, J. C., Snowball, A., Knauss, S., von Schimmelmann, M., During, M. J., Lignani, G., Schorge, S., Young, D., Kullmann, D. M., Lieb, A., 2019. Olanzapine: a potent agonist at the hM4D(Gi) DREADD amenable to clinical translation of chemogenetics. Sci Adv 5 eaaw1567. https://doi. org/10.1126/sciadv.aawl567.
12. Magnus, C. J., Lee, P. H., Atasoy, D., Su, H. H., Looger, L. L., Sternson, S. M., 2011. Chemical and genetic engineering of selective ion channel-ligand interactions. Science 333, 1292-1296. https://doi.org/10.1126/science.1206606.
13. Magnus, C. J., Lee, P. H., Bonaventura, J., Zemla, R., Gomez, J. L., Ramirez, M. H., Hu, X., Galvan, A., Basu, J., Michaelides, M., Sternson, S. M., 2019. Ultrapotent chemoge-netics for research and potential clinical applications. Science 364. https://doi.org/10.1126/science.aav5282.
14. Labiner D M, Butler L S, Cao Z, Hosford D A, Shin C, McNamara J O. (1993) Induction of c-fos mRNA by kindled seizures: complex relationship with neuronal burst firing. J Neurosci. 1993 February; 13(2):744-51. doi: 10.1523/JNEUROSCI.13-02-00744.1993.
15. Tyssowski K M, Gray J M. The neuronal stimulation-transcription coupling map. (2019) Curr Opin Neurobiol. 2019 December; 59:87-94. doi: 10.1016/j.conb.2019.05.001. Epub 2019 Jun. 1. PMID: 31163285 Free PMC article. Review.
16. Snowball A, Chabrol E, Wykes R C, Shekh-Ahmad T, Cornford J H, Lieb A, Hughes M P, Massaro G, Rahim A A, Hashemi K S, Kullmann D M, Walker M C, Schorge S. (2019) Epilepsy Gene Therapy Using an Engineered Potassium Channel. J Neurosci. 2019 Apr. 17; 39(16):3159-3169. doi: 10.1523/JNEUROSCI.1143-18.2019. Epub 2019 Feb. 12. PMID: 30755487
17. Frazier, S. J., Cohen, B. N., Lester, H. A., 2013. An engineered glutamate-gated chloride (GluCl) channel for sensitive, consistent neuronal silencing by ivermectin. J. Biol. Chem. 288, 21029-21042. https://doi.org/10.1074/jbc.M112.423921.
18. Lieb, A., Qiu, Y., Dixon, C. L., Heller, J. P., Walker, M. C., Schorge, S., Kullmann, D. M., 2018. Biochemical autoregulatory gene therapy for focal epilepsy. Nat. Med. 24, 1324-1329. https://doi.org/10.1038/s41591-018-0103-x

### Example 23

CellREADR targeting of neuron types in a non-human primate.

We developed RNA sensors for macaque monkey Foxp2 and vGAT, which marks glutamatergic pyramidal neuron (a subset) and GABAergic interneurons, respectively. We then generated binary AAV6 vectors expressing EGFP conditional to RNA sensing. Using these AAV CellREADR vectors targeting macaque FOXP2 and vGAT mRNAs (FIG. 17), we injected binary READR^{FOXP2}/Reporter^{mNeon} and READR^{FOXP2}/Reporter^{mNeon} AAVs into area V1 of macaque cortex. Fluorescent microscopy of READR^{FOXP2}-infected cortex revealed mNeon in infragranular pyramidal neurons, their apical dendrites, and efferent axons in white matter, indicate of glutamatergic pyramidal neurons. mNeon from READR^{vGAT}-infected cortex was restricted to morphologically diverse, putative inhibitory neurons throughout the cortical laminae. Therefore, CellREADR is effective in non-human primate in vivo.

### Example 24 Cell Specific Treatment of Chronic Pain

### Disease and Clinical Background

The incidence of chronic pain is estimated to be 20-25% worldwide. Few patients with chronic pain obtain complete relief from the drugs that are currently available, and more than half report inadequate relief 1. While an intact central nervous system (CNS) is required for the conscious perception of pain, and changes in the CNS are clearly evident in chronic pain states, ongoing peripheral nociceptive inputs play a major role in the maintenance of chronic pain ¹. Nociceptors in the skin, muscle, joints and viscera that selectively respond to noxious or potentially tissue-damaging stimuli and mediate peripheral pain inputs to the CNS. Despite detailed characterization of the mechanisms that underlie nociceptor excitability, in combination with the identification of several ion channels, receptors and second messenger signaling molecules that serve as points of convergence, effective therapeutic interventions for the management of pain that do not have deleterious side effects has remained elusive¹. The anatomical, physiological and functional heterogeneity among nociceptive afferents, rooted in their differential gene expression, has contributed to difficulty in the identification of new therapeutic agents and several notable failures of preclinical data to translate to an effective clinical intervention¹.

Among the signaling transduction mechanisms within nociceptors following sensory stimulation, the firing of action potential is the final output leading to afferent signals to CNS. Nociceptive action potentials are mainly driven by voltage gated sodium channels NaV1.7 and NaV1.8. Human genetic studies have highlighted a key role for NaV1.7 in nociceptive processing: gain-of-function mutations are associated with pain syndromes such as erythromelalgia and paroxysmal extreme pain disorder, whereas loss-of-function mutations are associated with congenital insensitivity to pain². Therefore, nociceptors represent key therapeutic targets for chronic pain. Previous CRISPR-dCas9-based approach for Nav1.7 down regulation achieved long-term pain relief without changing motor function in mice ³. The CellREADR technology enables the modulation of sodium channels and other signal transduction mechanisms within specific types of nociceptors to achieve therapeutic efficacy while reducing side effects.

### Targeting Nociceptors of Dorsal Root Ganglia and Effector Expression

NaV1.8 and TRPV1 are specifically expressed in nociceptors. RNA sensor for TRPV1 and NaV1.8 are used to establish specific access to nociceptors. A CellREADR based cell-type specific small hairpin RNAs (shRNA) is used in a knockdown approach to down-regulate NaV1.7 and NaV1.8 mRNAs in nociceptors. A binary expression vector FIG. 20) in which the translation of transcription activator tTA is gated by NaV1.7 or NaV1.8 RNA sensor is produced; tTA then activates the transcription of an shRNA for NaV1.7 or NaV1.8 RNA. This binary CellREADR vector is packaged into AAV9.

Delivery by Intrathecal Injection into Spinal Fluid Via Lumbar Puncture

These AAV vectors are delivered by intrathecal injection into the spinal fluid, to infect neurons in the dorsal root ganglia. CellREADR is expressed specifically in RNA sensor defined nociceptors.

### Expected Outcome and Improvement Over Current Treatments

CellREADR mediated modulation of NaV1.7 and NaV1.8. and other signal transduction mechanisms within specific types of nociceptors are more efficacious in achieving therapeutic effects with less side effects.

### Targeting Astrocytes and Satellite Glia Cells

Astrocytes are critical for maintaining the homeostasis of the CNS. A number of neurological disorders, including chronic pain, may result from astrocyte ‘gliopathy’⁴. Astrocytes can regulate nociceptive synaptic transmission via neuronal-glial and glial-glial cell interactions, as well as the involvement of spinal and supraspinal astrocytes in the modulation of pain signaling and the maintenance of neuropathic pain⁴. In addition, astrocyte-mediated neuroinflammation is a key mechanism underlying the maintenance of chronic pain⁴. Targeting the specific pathways that are responsible for astrogliopathy represents a novel approach to develop therapies for chronic pain.

Multiple cell types interact with nociceptive neurons and contribute to the pathogenesis of pain through distinct signaling mechanisms, including satellite glial cells, T cells, oligodendrocytes and microglia ⁵. CellREADR is used to modulate neuronal-glial interactions for chronic pain treatment by targeting specific glial cell types.

Satellite glia cells are localized in the dorsal root ganglia in the peripheral nervous system and express markers such as Kir4.1 and FABP7⁶. RNA sensors for FABP7 target satellite glial cells. Potassium channel Kir4.1 is downregulated in pathological pain conditions⁶. The function of Kir4.1 is restored by expressing the potassium channel to FABP7+ satellite glial cells.

vGluT1 is downregulated in the spinal cord in pathological conditions, leading to hyperactivation of glutamate receptors and chronic pain⁴. RNA sensors for Aldhl11 (an astroglial specific marker) to target astrocytes and Fabp7 to target satellite glial cells. The vesicular glutamate transporter vGluT1 is expressed into Aldhl1+ astrocytes. Notably, intrathecal route can target not only sensory neurons in DRG, but also satellite glial cells in DRG and astrocytes in the spinal cord.

CellREADR is a useful approach to specifically label satellite glial cells in DRG and astrocytes in the spinal cord following intrathecal delivery. Furthermore, the CellREADR-based approach can restore the function of satellite glial cells and astrocytes in pathological conditions, leading to a long-term relief and reversal of chronic pain.

### REFERENCES

1. Gold M S, Gebhart G F. (2010) Nociceptor sensitization in pain pathogenesis. Nat Med. 2010 November; 16(11):1248-57. doi: 10.1038/nm.2235. Epub 2010 Oct. 14. PMID: 20948530
2. Waxman, S. G. Channel, neuronal and clinical function in sodium channelopathies: from genotype to phenotype. Nat. Neurosci. 10, 405-409 (2007).
3. Moreno A M, Alemin F, Catroli G F, Hunt M, Hu M, Dailamy A, Pla A, Woller S A, Palmer N, Parekh U, McDonald D, Roberts A J, Goodwill V, Dryden I, Hevner R F, Delay L, Gongalves Dos Santos G, Yaksh T L, Mali P. (2021) Long-lasting analgesia via targeted in situ repression of Nav1.7 in mice. Sci Transl Med. 2021 Mar. 10; 13(584):eaay9056. doi: 10.1126/scitranslmed.aay9056. PMID: 33692134
4. Ji R R, Donnelly C R, Nedergaard M. (2019) Astrocytes in chronic pain and itch. Nat Rev Neurosci. 2019 November; 20(11):667-685. doi: 10.1038/s41583-019-0218-1. Epub 2019 Sep. 19. PMID: 31537912
5. Ji R R, Chamessian A, Zhang Y Q. (2016) Pain regulation by non-neuronal cells and inflammation. Science. 2016 Nov. 4; 354(6312):572-577. doi: 10.1126/science.aaf8924. PMID: 27811267
6. Hanani M, Spray D C. (2020) Emerging importance of satellite glia in nervous system function and dysfunction. Nat Rev Neurosci. 2020 September; 21(9):485-498. doi: 10.1038/s41583-020-0333-z. Epub 2020 Jul. 22. PMID: 32699292.

### Example 24 Cell Specific Intervention of Alzheimer's Disease

### Disease and Clinical Background

Neuroinflammation is a central mechanism involved in neurodegeneration as observed in Alzheimer's disease (AD), the most prevalent form of neurodegenerative disease 1, 2. Apolipoprotein E4 (APOE4), the strongest genetic risk factor for AD, directly influences disease onset and progression by interacting with the major pathological hallmarks of AD including amyloid-P plaques, neurofibrillary tau tangles, as well as neuroinflammation 1, 2. In the CNS under basal conditions, astrocytes produce most apoE; in the setting of brain injury or neurodegeneration, APOE expression is strongly upregulated in microglia.

Microglia and astrocytes, the two major immune cells in the brain, exist in an immune-vigilant state providing immunological defense as well as housekeeping functions that promote neuronal well-being. Under disease conditions, these immune cells become progressively dysfunctional in regulating metabolic and immunoregulatory pathways, thereby promoting chronic inflammation-induced neurodegeneration; an important component of this pathologic process is elevated microglia-mediated synaptic phagocytosis 1, 2.

Astrocyte-derived apoE particles are significantly larger and thus contain more lipids compared with apoE secreted by microglia. Astrocyte-secreted apoE4, either alone or with other molecules such as C1q, could serve as an opsonin for specific microglia receptors to activate microglia response. In a P301S Tau mouse model of AD, removing astrocytic APOE4 markedly reduced tau-mediated neurodegeneration and decreased phosphorylated tau (pTau) pathology as well as disease-associated gene signatures in neurons, oligodendrocytes, astrocytes, and microglia 3. Removal of astrocytic APOE4 further decreased tau-induced synaptic loss and microglial phagocytosis of synaptic elements, suggesting a key role for astrocytic apoE in synaptic degeneration 3.

### Target Cell Types

Astrocytes and microglia in the brain, especially the cerebral cortex. Marker genes/RNAs include
astrocytes: aldehyde dehydrogenase family 1 member L1 (Aldh1L1), glial fibrillary acidic protein (GFAP), excitatory amino acid transporter 1 (EAAT1)
microglia: C-X3-C Motif Chemokine Receptor 1 (CX3CR1), transmembrane protein 119 (TMEM119), Ionized calcium binding adaptor molecule 1 (IBA-1)

### Target Gene

shRNAs against ApoE4 RNA to achieve down-regulation in astrocytes and/or microglia in the brain.

FIG. 21 illustrates the construct where a first module of a readrRNA is driven by the hSyn promoter followed by a spacer, an SES RNA specific for one of the above target RNAs, and an tTA2 transcriptional activator which upon ADAR editing is translated and binds the TRE which is located upstream of the first module of the readrRNA and which drives the expression of small hairpin RNA with complementarity to ApoE4 RNA.

### Delivery

AAV vectors are used to first test in P300 tau mouse model and recapitulate the result of Wang and Holtzman Neuron 2021, i.e. to demonstrate the reduction of tau-mediated neurodegeneration and synaptic phagocytosis.

In AD patients along the progression from preclinical to early and late stages, amyloid deposition begins in temporobasal and frontomedial areas, and successively affects the remaining associative neocortex, primary sensory-motor areas and the medial temporal lobe, and finally the striatum4, 5. AAV vectors are injected to the temporobasal and frontomedial areas in early stage patients to reduce ApoE4 production from astrocytes and microglia in these areas.

### Expected Outcome

Reduction of ApoE4 production from astrocytes and microglia in the temporobasal and frontomedial areas attenuate neuroinflammation responses and protect against tau-mediated neurodegeneration and synaptic phagocytosis by microglia. Together these treatments attenuate or prevent the progression of AD.

### REFERENCES

1. Parhizkar S, Holtzman D M. 2022, APOE mediated neuroinflammation and neurodegeneration in Alzheimer's disease. Semin Immunol. 2022 Feb. 26:101594. doi: 10.1016/j.smim.2022.101594. Online ahead of print. PMID: 35232622
2. Chen Y, Strickland M R, Soranno A, Holtzman D M. Apolipoprotein E: Structural Insights and Links to Alzheimer Disease Pathogenesis. Neuron. 2021 Jan. 20; 109(2):205-221. doi: 10.1016/j.neuron.2020.10.008. Epub 2020 Nov. 10. PMID: 33176118
3. Wang C, Xiong M, Gratuze M, Bao X, Shi Y, Andhey P S, Manis M, Schroeder C, Yin Z, Madore C, Butovsky O, Artyomov M, Ulrich J D, Holtzman D M. Selective removal of astrocytic APOE4 strongly protects against tau-mediated neurodegeneration and decreases synaptic phagocytosis by microglia. Neuron. 2021 May 19; 109(10):1657-1674.e7. doi: 10.1016/j.neuron.2021.03.024. Epub 2021 Apr. 7. PMID: 33831349
4. Grothe M J, Barthel H, Sepulcre J, Dyrba M, Sabri O, Teipel S J; In vivo staging of regional amyloid deposition. Alzheimer's Disease Neuroimaging Initiative. Neurology. 2017 Nov. 14; 89(20):2031-2038. doi: 10.1212/WNL.0000000000004643. Epub 2017 Oct. 18. PMID: 29046362
5. Levin F, Jelistratova I, Betthauser T J, Okonkwo O, Johnson S C, Teipel S J, Grothe M J. In vivo staging of regional amyloid progression in healthy middle-aged to older people at risk of Alzheimer's disease. Alzheimers Res Ther. 2021 Oct. 21; 13(1):178. doi: 10.1186/s13195-021-00918-0. PMID: 34674764

TABLE 6
Plasmids encoding (i) targeted exogenous transcripts for proof of principle
experiments (ii) readrRNA or (iii) genes responsive to (but not physically
linked) to encode effector genes, e.g., transactivators are described herein.

| Constructs (backbone} | Note on construct |
|---|---|
| pCAG [symbol] | Addgene# 8302 [symbol] |
| pCAG-B [symbol] | Backbone from Addgene# 100844, Gcamp6s was replaced |
| | with BFP-2a-eGFP |
| pTRE-BFP- [symbol] | Backbone from Addgene# 71782, BFP-2a-Cheta was |
| | inserted after TRE elements |
| pCAG-BFP- [symbol] | Backbone from Addgene# 100844, Gcamp6s was replaced |
| | with BFP-2a Cheta |
| pCAG- [symbol] | Backbone from Addgene# 100844, Gcamp6s was replaced |
| | with mCherry-sesRAN-2a- [symbol] |
| pCAG- [symbol] | |
| pCAG-spacer [symbol] | from plasmid [symbol] spacer sequence was from CasR [symbol] coding |
| | regions of Addgene# [symbol] from ATG [symbol] |
| pCAG-ADAR [symbol] | from plasmid [symbol] ADAR2 sequence was from Addgene# [symbol] |
| pCAG- [symbol] | |
| pCAG- [symbol] | from plasmid [symbol] sequence was from Addgene# 174554 |
| pCAG-ADAR [symbol] | |
| pCAG-BFP- [symbol] | |
| [symbol] -Cheta- [symbol] | Addgene# 37786 |
| pCAG-BFP-sesRNA- [symbol] | from plasmid [symbol] sequence was from Addgene# 71394 |
| pBF1a-Cheta- [symbol] | |
| pCAG-BFP-sesRNA-2a- [symbol] | |
| pBF1a-Cheta- [symbol] | |
| pCAG-BFP-sesRNA-2a- [symbol] | |
| pBF1a-Cheta- [symbol] | |
| pBF1a-Cheta | from Addene# [symbol] Cheta- [symbol] was replaced with Cheta |
| pCAG- [symbol] | |
| pCAG-BFP-sesRNA-2a- [symbol] | |
| pCAG-BFP-sesRNA-2a- [symbol] | |
| pCAG-BFP-sesRNA-2a- [symbol] | |
| pCAG- [symbol] | Backbone from Addgene# 112668, [symbol] was reaplced with |
| | [symbol] -sesRNA- [symbol] |
| pAAV- [symbol] | [symbol] from Addgene# 59756 |
| pAAV-TRE [symbol] | |
| pAAV-TRE [symbol] | [symbol] from Addgene# 180844 |
| pAAV-TRE [symbol] | [symbol] from Addgene# 127239 |
| pAAV-TRE [symbol] | TRE3g backbone was kindly gifted from Nelson lab at |
| | Brandeis University |
| pAAV- [symbol] | [symbol] sequence was from Addgene# 174554 |
| pAAV-TRE [symbol] | TRE3g backbone was kindly gifted from Nelson lab at |
| | Brandeis University |
| PKG- [symbol] | |
| CAG-sesRNA | |
| PKG-Empty | |
| PKG- [symbol] | |
| CAG- [symbol] | |
| TRE [symbol] | TRE3g backbone was kindly gifted from Nelson lab at |
| | Brandeis University |
| pCAG-spacer-sesRNA-2a- [symbol] | Spacer sequence was from CasBx coding regions of |
| | Addgene# 109049, different length was used from ATG |
| | start as indicated |
| pCAG- [symbol] | |
| pCAG-BFP-sesRNA-2a- [symbol] | |
| TRE [symbol] | |
| pCAG-BFP-sesRNA-2a-Cas9- | Cas9 from addgene# 62988 |
| 2a-eGFP | |
| pCAG-BFP-sesRNA-2a- [symbol] | [symbol] Casp3- [symbol] from addgene# 45580 |
| pTRE-BFP-2a- [symbol] | |
| pCAG-BFP-2a-Cheta | |
| pCAG-mCherry-sesRNA-2a- | |
| eGFP | |
| CMV-ADAR [symbol] | From addgene# 117928, GFP coding region was deleted |
| CMV-ADAR [symbol] | From addgene# 117927, GFP coding region was deleted |
| pCAG-BFP-2a- [symbol] | Target Gene was amplified from mice [symbol] DNA, Cheta was |
| | used as a [symbol] . Results in Extended Data [symbol] |
| pCAG-mCherry- [symbol] | |
| pAAV- [symbol] | |
| pAAV-TRE [symbol] | |
| pAAV- [symbol] | |
| pAAV-TRE [symbol] | |
| pAAV-TRE [symbol] | |
| pAAV- [symbol] | |
| pAAV- [symbol] | [symbol] from previous study (Matho et al Nature 2011) |
| pAAV- [symbol] | [symbol] from addgene# 71837 |

UGT1A1 gene as effector
ATGGCTCGCACAGGGTGGACCAGCCCCATTCCCCTATGTGTTTCTCTGCTGCTGACCTGTGGCTTTGCTGAGGCAG
GGAAGCTGCTGGTAGTGCCCATGGATGGGAGTCACTGGTTCACCATGCAGTCGGTGGTGGAGAAACTTATCCTCAG
GGGGCATGAGGTGGTTGTAGTCATGCCAGAGGTGAGTTGGCAACTGGGAAAATCACTGAATTGCACAGTGAAGACT
TACTCAACCTCATACACTCTGGAGGATCTGGACCGGGAATTCATGGATTTCGCCGATGCTCAATGGAAAGCACAAG
TACGAAGTTTGTTTTCTCTATTTCTGAGTTCATCCAATGGTTTTTTTAACTTATTTTTTTCGCATTGCAGGAGTTT
GTTTAATGACCGAAAATTAGTAGAATACTTAAAGGAGAGTTCTTTTGATGCGGTGTTTCTTGATCCTTTTGATGCC
TGTGGCTTAATTGTTGCCAAATATTTCTCCCTCCCCTCTGTGGTCTTCGCCAGGGGAATAGCTTGCCACTATCTTG
AAGAAGGTGCACAGTGCCCTGCTCCTCTTTCCTATGTCCCCAGAATTCTCTTAGGGTTCTCAGATGCCATGACTTT
CAAGGAGAGAGTACGGAACCACATCATGCACTTGGAGGAACATTTATTTTGCCAGTATTTTTCCAAAAATGCCCTA
GAAATAGCCTCTGAAATTCTCCAAACACCTGTCACAGCATATGATCTCTACAGCCACACATCAATTTGGTTGTTGC
GAACAGACTTTGTTTTGGACTATCCCAAACCCGTGATGCCCAATATGATCTTCATTGGTGGTATCAACTGCCATCA
GGGAAAGCCATTGCCTATGGAATTTGAAGCCTACATTAATGCTTCTGGAGAACATGGAATTGTGGTTTTCTCTTTG
GGATCAATGGTCTCAGAAATTCCAGAGAAGAAAGCTATGGCAATTGCTGATGCTTTGGGCAAAATCCCTCAGACAG
TCCTGTGGCGGTACACTGGAACCCGACCATCGAATCTTGCGAACAACACGATACTTGTTAAGTGGCTACCCCAAAA
CGATCTGCTTGGTCACCCGATGACCCGTGCCTTTATCACCCATGCTGGTTCCCATGGTGTTTATGAAAGCATATGC
AATGGCGTTCCCATGGTGATGATGCCCTTGTTTGGTGATCAGATGGACAATGCAAAGCGCATGGAGACTAAGGGAG
CTGGAGTGACCCTGAATGTTCTGGAAATGACTTCTGAAGATTTAGAAAATGCTCTAAAAGCAGTCATCAATGACAA
AAGTTACAAGGAGAACATCATGCGCCTCTCCAGCCTTCACAAGGACCGCCCGGTGGAGCCGCTGGACCTGGCCGTG
TTCTGGGTGGAGTTTGTGATGAGGCACAAGGGCGCGCCACACCTGCGCCCCGCAGCCCACGACCTCACCTGGTACC
AGTACCATTCCTTGGACGTGATTGGTTTCCTCTTGGCCGTCGTGCTGACAGTGGCCTTCATCACCTTTAAATGTTG
TGCTTATGGCTACCGGAAATGCTTGGGGAAAAAAGGGCGAGTTAAGAAAGCCCACAAATCCAAGACCCATTGA
(SEQ ID NO: 148)
VEGFA Coding Sequence as effector
ATGGCGGCTTCTCAGGCGGTGGAGGAAATGCGGAGCCGCGTGGTTCTGGGGGAGTTTGGGGTTCGCAATGTCCATA
CTACTGACTTTCCCGGTAACTATTCCGGTTATGATGATGCCTGGGACCAGGACCGCTTCGAGAAGAATTTCCGTGT
GGATGTAGTACACATGGATGAAAACTCACTGGAGTTTGACATGGTGGGAATTGACGCAGCCATTGCCAATGCTTTT
CGACGAATTCTGCTAGCTGAGGTGCCAACTATGGCTGTGGAGAAGGTCCTGGTGTACAATAATACATCCATTGTTC
AGGATGAGATTCTTGCTCACCGTCTGGGGCTCATTCCCATTCATGCTGATCCCCGTCTTTTTGAGTATCGGAACCA
AGGAGATGAAGAAGGCACAGAGATAGATACTCTACAGTTTCGTCTCCAGGTCAGATGCACTCGGAACCCCCATGCT
GCTAAAGATTCCTCTGACCCCAACGAACTGTACGTGAACCACAAAGTGTATACCAGGCATATGACATGGATCCCCC
TGGGGAACCAGGCTGATCTCTTTCCAGAGGGCACTATCCGACCAGTGCATGATGATATCCTCATCGCTCAGCTGCG
GCCTGGCCAAGAAATTGACCTGCTCATGCACTGTGTCAAGGGCATTGGCAAAGATCATGCCAAGTTTTCACCAGTG
GCAACAGCCAGTTACAGGCTCCTGCCAGACATCACCCTGCTTGAGCCCGTGGAAGGGGAGGCAGCTGAGGAGTTGA
GCAGGTGCTTCTCACCTGGTGTTATTGAGGTGCAGGAAGTCCAAGGTAAAAAGGTGGCCAGAGTTGCCAACCCCCG
GCTGGATACCTTCAGCAGAGAAATCTTCCGGAATGAGAAGCTAAAGAAGGTTGTGAGGCTTGCCCGGGTTCGAGAT
CATTATATCTGTAAGAAAGATTTGCTGGCTGCGGTGGCTCACACCTGTAATCCCAGCACTTTGGGAGGCTGAGGCG
GGTGGATCACGAGGTCAGGAGATCGAGACCATCCTGGCTAA (SEQ ID NO:149) [symbol]
indicates data missing or illegible when filed

| ses + D42 + | | | |
|---|---|---|---|
| A1:D3 + | Target | | SEQ ID |
| A1:C7 | gene | sesRNA sequence | NO: |
| tdT | tdTomato | ACTCCACCAGGTAGTGGCCGCCGTCCTTCAGCTTCAGGGCCT | SEQ ID |
| | | GGTGGATCTCGCCCTTCAGCACGCCGTCGCGGGGGTACAGGC | NO: 1 |
| | | GCTCGGTGGAGGCCTCCCAGCCCATAGTCTTCTTCTGCATTA | |
| | | CGGGGCCGTCGGGGGGGAAGTTGGTGCCGCGCATCTTCACCT | |
| | | TGTAGATCAGCGTGCCGTCCTGCAGGG | |
| Ctrl | Not | AGGCAAGCCCTACGAGGGCACCCAGACCATGAGAATCAAGGT | SEQ ID |
| | Target | GGTCGAGGGCGGCCCTCTCCCCTTCGCCTTCGACATCCTGGC | NO: 2 |
| | | TACTAGCTTCCTCTACGGCAGCAAGACCTTCATCAACCACAC | |
| | | CCAGGGCATCCCCGACTTCTTCAAGCAGTCCTTCCCTGAGGG | |
| | | CTTCACATGGGAGAGAGTCACCACATA | |
| Cheta | Cheta | AGCCCTCGGGGAAGGACAGCTTCTTGTAATCGGGGATGTCGG | SEQ ID |
| | | CGGGGTGCTTCACGTACGCCTTGGAGCCGTACAGGAACTGGG | NO: 3 |
| | | GGGACAGGATGTCCCAGGCGAAGGGCAGGGGGCCGCCCTTGG | |
| | | TCACCTTCAGCTTAGCGGTCTGGGTGCCCTCGTAGGGGCGGC | |
| | | CCTCGCCCTCGCCCTCGATCTCGAACTCGTGGCCGTTCATGG | |
| | | AGCCCTCCATGCGCACCT | |
| 25nt-tdT | tdTomato | CCTCCCAGCCCATAGTCTTCTTCTA | SEQ ID |
| | | | NO: 4 |
| 50nt-tdT | tdTomato | GCTCGGTGGAGGCCTCCCAGCCCATAGTCTTCTTCTGCATTA | SEQ ID |
| | | CGGGGCCG | NO: 5 |
| 100nt-tdT | tdTomato | TCAGCACGCCGTCGCGGGGGTACAGGCGCTCGGTGGAGGCCT | SEQ ID |
| | | CCCAGCCCATAGTCTTCTTCTGCATTACGGGGCCGTCGGGGG | NO: 6 |
| | | GGAAGTTGGTGCCGCA | |
| 150nt-tdT | tdTomato | GGGCCTGGTGGATCTCGCCCTTCAGCACGCCGTCGCGGGGGT | SEQ ID |
| | | ACAGGCGCTCGGTGGAGGCCTCCCAGCCCATAGTCTTCTTCT | NO: 7 |
| | | GCATTACGGGGCCGTCGGGGGGGAAGTTGGTGCCGCGCATCT | |
| | | TCACCTTGTAGATCAGCGTGCCGT | |
| 250nt-tdT | tdTomato | CCATGTAGATGGTCTGGAACTCCACCAGGTAGTGGCCGCCGT | SEQ ID |
| | | CCTTCAGCTTCAGGGCCTGGTGGATCTCGCCCTTCAGCACGC | NO: 147 |
| | | CGTCGCGGGGGTACAGGCGCTCGGTGGAGGCCTCCCAGCCCA | |
| | | TAGTCTTCTTCTGCATTACGGGGCCGTCGGGGGGGAAGTTGG | |
| | | TGCCGCGCATCTTCACCTTGTAGATCAGCGTGCCGTCCTGCA | |
| | | GGGAGGAGTCCTGGGTCACGGTCACCAGACCGCCGTCCTG | |
| 400nt-tdT | tdTomato | TGTCCAGCTTGGTGTCCACGTAGTAGTAGCCGGGCAGTTGCA | SEQ ID |
| | | CGGGCTTCTTGGCCATGTAGATGGTCTGGAACTCCACCAGGT | NO: 9 |
| | | AGTGGCCGCCGTCCTTCAGCTTCAGGGCCTGGTGGATCTCGC | |
| | | CCTTCAGCACGCCGTCGCGGGGGTACAGGCGCTCGGTGGAGG | |
| | | CCTCCCAGCCCATAGTCTTCTTCTGCATTACGGGGCCGTCGG | |
| | | GGGGGAAGTTGGTGCCGCGCATCTTCACCTTGTAGATCAGCG | |
| | | TGCCGTCCTGCAGGGAGGAGTCCTGGGTCACGGTCACCAGAC | |
| | | CGCCGTCCTCGAAGTTCATCACGCGCTCCCACTGGAAGCCCT | |
| | | CGGGGAAGGACAGCTTCTTGTAATCGGGGATGTCGGCGGGGT | |
| | | GCTTCACGTACGCCTTGGAGCG | |
| 1mm-A-tdT | tdTomato | ACTCCACCAGGTAGTGGCCGCCGTCCGTCAGCTTCAGGGCCT | SEQ ID |
| | | GGTGGATCTCGCCCTTCAGCACGCCGTCGCGGGGGTACAGGC | NO: 10 |
| | | GCTCGGTGGAGGCCTCCCAGCCCATAGTCTTCTTCGGCATTA | |
| | | CGGGGCCGTCGGGGGGGAAGTTGGTGCCGCGCATCTTCACCT | |
| | | TGTAGATCAGCGTGCCGTCCTGCAGGG | |
| 1mm-B-tdT | tdTomato | ACTCCACCAGGTAGTGGCCGCCGTCCTTCTGCTTCAGGGCCT | SEQ ID |
| | | GGTGGATCTCGCCCTTCAGCACGCCGTCGCGGGGGTACAGGC | NO: 11 |
| | | GCTCGGTGGAGGCCTCCCAGCCCATAGTCTTCTTCTGCTTTA | |
| | | CGGGGCCGTCGGGGGGGAAGTTGGTGCCGCGCATCTTCACCT | |
| | | TGTAGATCAGCGTGCCGTCCTGCAGGG | |
| 2mm-A-tdT | tdTomato | ACTCCACCAGGTAGTGGCCGCCGTCCTTCAGCTTCAGGGCCT | SEQ ID |
| | | GGTGGATCTCGCCCTTCAGCACGCCGTCGCGGGGGTACAGGC | NO: 13 |
| | | GCTCGGTGGAGGCCTCCCATCCCATAGTCTTCTTCTGCGTTA | |
| | | CGGGGCCGTCGGGGGGGAAGTTGGTGCCGCGCATCTTCACCT | |
| | | TGTAGATCAGCGTGCCGTCCTGCAGGG | |
| 2mm-B-tdT | tdTomato | ACTCCACCAGGTAGTGGCCGCCGTCCTTCTGCTTCAGGGCCT | SEQ ID |
| | | GGTGGATCTCGCCCTTCAGCACGCCGTCGCGGGGGTACAGGC | NO: 14 |
| | | GCTCGGTGGAGGCCTCCCAGCCCATAGTCTTCTTCTGCTTTA | |
| | | CGGGGCCGTCGGGGGGGAAGTTGGTGCCGCGCATCTTCACCT | |
| | | TGTAGATCAGCGTGCCGTCCTGCAGGG | |
| 4mm-A-tdT | tdTomato | ACTCCACCAGGTAGCGGCCGCCGTCCTTCAGCTTCAGGGCCT | SEQ ID |
| | | GGTGGATCTCGCCCTTCAGCACGCCGTCGTGGGGGTACAGGC | NO: 15 |
| | | GCTCGGTGGTGGCCTCCCAGCCCATAGTCTTCTTCTGCATTA | |
| | | CGGGGCCGTCGGGGGGGAAGTTGGTGCCGCGCATCTTCACCT | |
| | | TCTAGATCAGCGTGCCGTCCTGCAGGG | |
| 4mm-B-tdT | tdTomato | ACTCCACCAGGTAGTGGCCGCCGTCCTTCGGCTTCAGGGCCT | SEQ ID |
| | | GGTGGATCTCGCCCTTCAGAACGCCGTCGCGGGGGTACAGGC | NO: 16 |
| | | GCTCGGTGGAGGCCTCCCAGCCCATAGTCTTCTTCTGCATTA | |
| | | CGGGGCCGTAGGGGGGGAAGTTGGTGCCGCGCATCTTCACCT | |
| | | TGTAGAGCAGCGTGCCGTCCTGCAGGG | |
| 10mm-A-tdT | tdTomato | ACTCCACCAGGTGGTGGCCGCCGTCCTTCATCTTCAGGGACT | SEQ ID |
| | | GGTGGATCTCGCCCTTCAGCACGCCGTCGAGGGGGTACAGGC | NO: 17 |
| | | GCGCGGTGGAGGCCTCCCAGCCCATAGTCTTCTTCGGCATTA | |
| | | CGGGGACGTCGGGGGGGAAGTTGGTGCTGCGCATCTTCACAT | |
| | | TGTAGATCATCGTGCCGTCCTGCAGGG | |
| 10mm-B-tdT | tdTomato | ACTCCACCAGGTAGTGGCCGACGTCCTTCAGCTTCAGGTCCT | SEQ ID |
| | | GGTGGATCTCGCCCTTCCGCACGCCGTCGCGGTGGTACAGGC | NO: 18 |
| | | GCCCGGTGGAGGTCTCCCAGCCCATAGTCTTCTTCTTCATTA | |
| | | CGGGGCCGTCGGGGGGTAAGTTGGGGCCGCGCATCTTCACCT | |
| | | TGTAGATCAGTGTGCCGTCCTGCAGGG | |
| 20mm-tdT | tdTomato | ACTCCACCAGGCAGTGGCCGACGTCCTTCGGCTTCAGGTCCT | SEQ ID |
| | | GGCGGATCTCGCCCTTCCGCACGCCGCCGCGGTGGTACAGGC | NO: 19 |
| | | GCTCAGTGGAGGTCTCCCAGCCCATAGTCTTCATCTTCATTA | |
| | | CGGGGCCGTCGCGGGGTAAGTTGGGGCCGCCCATCTTCACGT | |
| | | TGTAGATCAGTGTGCCGACCTGCAGGG | |
| 1nt-insert | tdTomato | ACTCCACCAGGTAGTGGCCGCCGTCCTTCAGCTTCAGGGCCT | SEQ ID |
| in frame | | GGTGGATCTCGCCCTTCAGCACGCCGTCGCGGGGGTACAGGC | NO: 20 |
| | | GCTCGGTGCGAGGCCTCCCAGCCCATAGTCTTCTTCTGCATT | |
| | | ACGGGGCCGTCGGGGGGGAAGTTGGTGCCGCGCATCTTCACC | |
| | | TTGTAGATCAGCGTGCCGTCCTGCAGGG | |
| 1nt-del in | tdTomato | ACTCCACCAGGTAGTGGCCGCCGTCCTTCAGCTTCAGGGCCT | SEQ ID |
| frame | | GGTGGATCTCGCCCTTCAGCACGCCGTCGCGGGGGTACAGGC | NO: 22 |
| | | GCTCGGTGGAGGCCTCCCAGCCCATAGTCTTCTTCTGCATTA | |
| | | CGGGCCGTCGGGGGGGAAGTTGGTGCCGCGCATCTTCACCTT | |
| | | GTAGATCAGCGTGCCGTCCTGCAGGG | |
| mm (TGC- | tdTomato | ACTCCACCAGGTAGTGGCCGCCGTCCTTCAGCTTCAGGGCCT | SEQ ID |
| TAG) | | GGTGGATCTCGCCCTTCAGCACGCCGTCGCGGGGGTACAGGC | NO: 23 |
| | | GCTCGGTGGAGGCCTCCCAGCCCATAGTCTTCTTCTGCATTA | |
| | | CGGGGCCGTCGGGGGGGAAGTTGGTGCCGCGCATCTTCACCT | |
| | | TGTAGATCAGCGTGCCGTCCTGCAGGGC | |
| mm (TGT- | tdTomato | ACTCCACCAGGTAGTGGCCGCCGTCCTTCAGCTTCAGGGCCT | SEQ ID |
| TAG) | | GGTGGATCTCGCCCTTCAGCACGCCGTCGCGGGGGTACAGGC | NO: 24 |
| | | GCTCGGTGGAGGCCTCCCAGCCCATGGTCTTCTTCTGCATTA | |
| | | CGGGGCCGTCGGGGGGGAAGTTGGTAGCGCGCATCTTCACCT | |
| | | TGTAGATCAGCGTGCCGTCCTGCAGGGAGGAGTCCTGGGTCA | |
| | | CGGTCACCAGACCGCCGTCCTCGAAGTTCATCACGCGCTCCC | |
| | | ACT | |
| Promotor | Promotor | AGTTTTAAACAGAGAGGAATCTTTGCAGCTAATGGACCTTCT | SEQ ID |
| | of | AGGTCTGGAAAGGAGTGGGAATTGGCTCCGGTGCCCGTCAGT | NO: 25 |
| | plasmid | GGGCAGAGCGCACATCGCCCACAGTCCCCGAGAAGTTAGGGG | |
| | | GAGGGGTCGGCAATTGAACCGGTGCCTAGAGAAGGTGGCGCG | |
| | | GGGAAAACTGGGAAAGTGAAGTCGTGTACTGGCTCCGCCTTT | |
| | | TTCCCGAGGGTGGGGGAGAACCGTATAT | |
| tdT2 | tdTomato | AGCCCTCGGGGAAGGACAGCTTCTTGTAATCGGGGATGTCGG | SEQ ID |
| | | CGGGGTGCTTCACGTACGCCTTGGAGCCGTACAGGAACTGGG | NO: 29 |
| | | GGGACAGGATGTCCCAGGCGAAGGGCAGGGGGCCGCCCTTGG | |
| | | TCACCTTCAGCTTAGCGGTCTGGGTGCCCTCGTAGGGGCGGC | |
| | | CCTCGCCCTCGCCCTCGATCTCGAACTCGTGGCCGTTCATGG | |
| | | AGCCCTCCATGCGCACCT | |
| WPRE | WPRE | CGGGGAGGCGGCCCAAAGGGAGATCCGACTCGTCTGAGGGCG | 135 |
| | of | AAGGCGAAGACGCGGAAGAGGCCGCAGAGCCGGCAGCAGGCC | |
| | plasmid | GCGGGAAGGAAGGTCCGCTAGATTGAGGGCCGAAGGGACGTA | |
| | | GCAGAAGGACGTCCCGCGCAGAATCCAGGTGGCAACATAGGC | |
| | | GAGCAGCCAAGGAAAGGACGAGGATTTCCCCGACAACACCAC | |
| | | GGAATTGTCAGTGCCCAACAGCCGAGCCCCTGTCCAGCAGCG | |
| | | GGCAAGGCAGGCGGCGATGAGTTCCGCCGTGGCAATAGGGAG | |
| | | GGG | |
| 2TAG | tdTomato | ACTCCACCAGGTAGTGGCCGCCGTCCTTCAGCTTCAGGGCCT | 30 |
| | | GGTGGATCTCGCCCTTCAGCACGCCGTCGCGGGGGTACAGGC | |
| | | GCTCGGTGGAGGCCTCCCAGCCCATAGTCTTCTTCTGCATTA | |
| | | CGGGGCCGTCGGGGGGGAAGTTGGTAGCGCGCATCTTCACCT | |
| | | TGTAGATCAGCGTGCCGTCCTGCAGGGAGGAGTCCTGGGTCA | |
| | | CGGTCACCAGACCGCCGTCCTCGAAGTTCATCACGCGCTCCC | |
| | | ACT | |
| 3TAG | tdTomato | ACTCCACCAGGTAGTGGCCGCCGTCCTTCAGCTTCAGGGCCT | 31 |
| | | GGTGGATCTCGCCCTTCAGCACGCCGTCGCGGGGGTACAGGC | |
| | | GCTCGGTAGAGGCCTCCCAGCCCATAGTCTTCTTCTGCATTA | |
| | | CGGGGCCGTCGGGGGGGAAGTTGGTAGCGCGCATCTTCACCT | |
| | | TGTAGATCAGCGTGCCGTCCTGCAGGGAGGAGTCCTGGGTCA | |
| | | CGGTCACCAGACCGCCGTCCTCGAAGTTCATCACGCGCTCCC | |
| | | ACT | |
| tdT1-tdT2 | CDS | ACTCCACCAGGTAGTGGCCGCCGTCCTTCAGCTTCAGGGCCT | 136 |
| | | GGTGGATCTCGCCCTTCAGCACGCCGTCGCGGGGGTACAGGC | |
| | | GCTCGGTGGAGGCCTCCCAGCCCATAGTCTTCTTCTGCATTA | |
| | | CGGGGCCGTCGGGGGGGAAGTTGGTGCCGCGCATCTTCACCT | |
| | | TGTAGATCAGCGTGCCGTCCTGCAGGGCTTCCTCTACGGCAG | |
| | | CAAGACCTTCATCAACCACACCCAGGGCATGGATGTCGGGGG | |
| | | GTGCTTCACGTACGCCTTGGAGCCGTACAGGAACTGGGGGGA | |
| | | CAGGATGTCCCAGGCGAAGGGCAGGGGGCCGCCCTTGGTCAC | |
| | | CTTCAGCTTAGCGGTCTGGGTGCCCTCGTAGGGGCGGCCCTC | |
| | | GCCCTCGCCCTCGATCTCGAACTCGTGGCCGTTCATGGAGCC | |
| | | CTCCATGCGCACCT | |
| WPRE-Cheta | WPRE- | ACTCCACCAGGTAGTGGCCGCCGTCCTTCAGCTTCAGGGCCT | 137 |
| | Cheta | GGTGGATCTCGCCCTTCAGCACGCCGTCGCGGGGGTACAGGC | |
| | | GCTCGGTGGAGGCCTCCCAGCCCATAGTCTTCTTCTGCATTA | |
| | | CGGGGCCGTCGGGGGGGAAGTTGGTGCCGCGCATCTTCACCT | |
| | | TGTAGATCAGCGTGCCGTCCTGCAGGGCTTCCTCTACGGCAG | |
| | | CAAGACCTTCATCAACCACACCCAGGGCATGGATGTCGGCGG | |
| | | GGTGCTTCACGTACGCCTTGGAGCCGTACAGGAACTGGGGGG | |
| | | ACAGGATGTCCCAGGCGAAGGGCAGGGGGCCGCCCTTGGTCA | |
| | | CCTTCAGCTTAGCGGTCTGGGTGCCCTCGTAGGGGCGGCCCT | |
| | | CGCCCTCGCCCTCGATCTCGAACTCGTGGCCGTTCATGGAGC | |
| | | CCTCCATGCGCACCT | |
| tdT1-Cheta | CDS | ACTCCACCAGGTAGTGGCCGCCGTCCTTCAGCTTCAGGGCCT | 138 |
| | | GGTGGATCTCGCCCTTCAGCACGCCGTCGCGGGGGTACAGGC | |
| | | GCTCGGTGGAGGCCTCCCAGCCCATAGTCTTCTTCTGCATTA | |
| | | CGGGGCCGTCGGGGGGGAAGTTGGTGCCGCGCATCTTCACCT | |
| | | TGTAGATCAGCGTGCCGTCCTGCAGGGCTTCCTCTACGGCAG | |
| | | CAAGACCTTCATCAACCACACCCAGGGCATGCGCAGGTAGTG | |
| | | TCCCAACAACCCCCAACAATTTTTACTCATCAGATCAATAAT | |
| | | CGTGTGACCTACGGTGGAGCCATAGACGCTCAGGACGCCAAA | |
| | | ACCTTCGGGCCCCAAAATGAAGAGAATTAGGAACATACCCCA | |
| | | GCTCACGAAAAACAGCCATGCCATGCCGGTCACGACCTGGCG | |
| | | GCACCGACCCTTTGGCACAGT | |
| WPRE-tdT- | CDS | CGGGGAGGCGGCCCAAAGGGAGATCCGACTCGTCTGAGGGCG | 139 |
| Cheta | | AAGGCGAAGACGCGGAAGAGGCCGCAGAGCCGGCAGCAGGCC | |
| | | GCGGGAAGGAAGGTCCGCTAGATTGAGGGCCGAAGGGACGTA | |
| | | GCAGAAGGACGTCCCGCGCAGAATCCAGGTGGCAACATAGGC | |
| | | GAGCAGCCAAGGAAAGGAAATCAAGGTGGTCGAGGGCGGCCC | |
| | | TCTCCCCTTCGCCTTCGACATCCTGGCTACACTCCACCAGGT | |
| | | AGTGGCCGCCGTCCTTCAGCTTCAGGGCCTGGTGGATCTCGC | |
| | | CCTTCAGCACGCCGTCGCGGGGGTACAGGCGCTCGGTGGAGG | |
| | | CCTCCCAGCCCATAGTCTTCTTCTGCATTACGGGGCCGTCGG | |
| | | GGGGGAAGTTGGTGCCGCGCATCTTCACCTTGTAGATCAGCG | |
| | | TGCCGTCCTGCAGGGCTACGGCAGCAAGACCTTCATCAACCA | |
| | | CACCCAGGGCATGCGCAGGTAGTGTCCCAACAACCCCCAACA | |
| | | ATTTTTACTCATCAGATCAATAATCGTGTGACCTACGGTGGA | |
| | | GCCATAGACGCTCAGGACGCCAAAACCTTCGGGCCCCAAAAT | |
| | | GAAGAGAATTAGGAACATACCCCAGCTCACGAAAAACAGCCA | |
| | | TGCCATGCCGGTCACGACCTGGCGGCACCGACCCTTTGGCAC | |
| | | AGT | |
| EEF1A1-3′ | EEF1A1 | CTTCAGTCTCCACCCACTCAGTGTGGGGAAACTCCATCGCAT | 140 |
| UTR | | AAAACCCCTCCCCCCAACCTAAAGACGACGTACTCCAAAAGC | |
| | | TCGAGAACTAATCGAGGTGCCTGGACGGCGCCCGGTACTCCG | |
| | | TAGAGTCACATGAAGCGACGGCTGAGGACGGAAAGGCCCTTT | |
| | | TCCTTTGTGTGGGTGACTCACCCGCCCGCTCTCCCGAGCGCC | |
| | | GCGTCCTCCATTTTG | |
| EEF1A1-5′ | EEF1A1 | CTCATAGGGTGTACACTAGCCACTACACTTATTTCTTATGTC | 141 |
| UTR | | ATGGCAAATAGTCAACTTTCACTGCCCAGTCATTTTAACCCA | |
| | | CGTTTCAACATGCACATCCCAGTAATTTAGAAACATTTTGTT | |
| | | TCCAAAGATTCACTTAACATTGGTTTAGCAACAGGAAGCTTT | |
| | | CTATGCAACACAAGGACTCAGTTTTTGGCCTGTTTTAGTGAC | |
| | | AGGCAATCAGCAACATGCTGCATTTCTCTCCAGTGTTGT | |
| EEF1A1- | EEF1A1 | GTAAGGATTAAGAGTCGTTACTTGGTTACTAAAACACAAACT | 142 |
| intron | | CCAGCTTCAATTTCCTTGTCCCCAGCCCTTAATTAGCAGTTT | |
| | | CCACTTTACAACTCCAAGTCCAAAGTGATTTTAGTCACTTTG | |
| | | GGTTACAGAAGCAACCAAAAATCAAACTTTTATAAGTCGGAT | |
| | | CTTAACTATGAACATCCAAATCTACTCACTAGCAATACGATT | |
| | | ACAGAAGTCACCAAAAGC | |
| EEF1A1- | EEF1A1 | GTTAGCACTTGGCTCCAGCATGTTGTCACCATTCCAACCAGA | 40 |
| Exon3 | | AATTGGCACAAATGCTACTGTGTCGGGGTTGTAGCCAATTTT | |
| | | CTTAATGTAAGTGCTGACTTCCTTAACAATTTCCTCATATCT | |
| | | CTTCTGGCTGTAGGGTAGCTCAGTGGAATCCATTTTGTTAAC | |
| | | ACCGACAATGAGTTGTTTCACACCCAGTGTGTAAGCCAGAAG | |
| | | GGCATGCTCTCGGGTCTGCCCATTCTTGGAGATACCAGC | |
| EEF1A1- | EEF1A1 | AGTGAAGCCAGCTGCTTCCATTGGTGGGTCATTTTTGCTGTC | 41 |
| Exon5 | | ACCAGCAACGTTGCCACGACGAACATCCTTGACAGACACATT | |
| | | CTTGACATTGAAGCCCACATTGTCCCCAGGAAGAGCTTCACT | |
| | | CAAAGCTTCATGGTGCATTTCGACAGATTTTACTTCCGTTGT | |
| | | AACGTTGACTAGAGCAAAGGTGACCACCATACCGGGTTTGAG | |
| | | AACACCAGTCTCCACTCGGCCAACAGGAACAGTACCAAT | |
| EEF1A1-eie | EEF1A1 | GTCTGTCTCATATCACGAACAGCAAAGCGACCTATTAAAAAA | 42 |
| | | AAAGTTAATTATTACCCAAAGTACTGTTCAGTTGTATTTTTC | |
| | | ATCTTTAACACAACTTTTTTACATTTAAGTAGTCATCCTTAC | |
| | | CCAAAGGTGGATAGTCTGAGAAGCTCTCAACACACATAGGCT | |
| | | TGCCAGGAACCATATCAACAATGGCAGCATCACCAGACTTCA | |
| | | AGAATTGAGGGCCATCTTCCAGCTTTTTACCAGAACGGC | |
| EEF1A1-CDS | EEF1A1 | GCCATCTTCCAGCTTTTTACCAGAACGGCGATCAATCTTTTC | 43 |
| | | CTTCAGCTCAGCAAACTTGCATGCAATGTGAGCCGTGTGGCA | |
| | | ATCCAATACAGGGGCATAGCCGGCGCTTATTTGGCCTAGATG | |
| | | GTTCAGGATAATCACCTGAGCAGTGAAGCCAGCTGCTTCCAT | |
| | | TGGTGGGTCATTTTTGCTGTCACCAGCAACGTTGCCACGACG | |
| | | AACATCCTTGACAGACACATTCTTGACATTGAAGCCCAC | |
| ACTB | ACTB | GGGTCATCTTCTCGCGGTTGGCCTTGGGGTTCAGGGGGGCCT | 143 |
| | | CGGTCAGCAGCACGGGGTGCTCCTCGGGAGCCACACGCAGCT | |
| | | CATTGTAGAAGGTGTGGTGCCAGATTTTCTCCATGTCGTCCC | |
| | | AGTTGGGGACGATGCCGTGCTCGATAGGGTACTTCAGGGGGA | |
| | | GGATGCCTCTCTTGCTCTGGGCCTCGTCGCCCACATAGGAAT | |
| | | CCTTCTGACCCATGCCCACCATCACGC | |
| ACTB-CDS | ACTB | CTGGGTGCCAGGGCAGTGATCTCCTTCTGCATCCTGTCGGCA | 144 |
| | | ATGCCAGGGTACATGGTGGTGCCGCCAGACAGCACTGTGTTG | |
| | | GCGTACAGGTCTTTGCGGATGTCCACGTCACACTTCATGATG | |
| | | GAGTTGAAGGTAGTTTCGTAGATGCCACAGGACTCCATGCCC | |
| | | AGGAAGGAAGGCTGGAAGAGTGCCTCAGGGCAGCGGAACCGC | |
| | | TCATTGCCAATGGTGATGACCTGGCCGTCAGGCAGCTCG | |
| XIST | XIST | AAAATATGGAGGACGTGTCAAGAAGACACTAGGAGAAAGTAT | 145 |
| | | AGAATTGAAAAAAATATTTTATGGAATTTAGGTGATTTTTTT | |
| | | AAAGAAATACGCCATAAAGGGTGTTAGGGGACTAGAAAATGT | |
| | | TCTAGAAAGAACCCCAAGTGCAGAGAGATCTTCAGTCAGGAA | |
| | | GCTTCCAGCCCCGAGAGAGTAAGAAATATGGCTGCAGCAGCG | |
| | | AATTGCAGCGCTTTAAGAACTGAA | |
| PCNA | PCNA | CTGGTGAGGTTCACGCCCATGGCCAGGTTGCGGTCGCAGCGG | 146 |
| | | GAGGTGTCGAAGCCCTCAGACCGCAGGGTGAGCTGCACCAAA | |
| | | GAGACGTAGGACGAGTCCATGCTCTGCAGGTTTACACCGCTG | |
| | | GAGCTAATATCCCAGCAGGCCTCGTTGATGAGGTCCTTGAGT | |
| | | GCCTCCAACACCTTCTTGAGGATGGAGCCCTGGACCAGGCGC | |
| | | GCCTCGAAC | |
| Ctip2-ses1 | Ctip2 | GAAGCGCAGGCTGCCGTTCTCAGAGGAATGTTCCGACGATGT | 56 |
| | | GGCGAAAGGCGACTGGCGCGCATCCGTGAAGCCCAGGAATGG | |
| | | GTCCTTCATGAAGTGGCGCGATGCTGCGTAGCCCACGAGCCA | |
| | | CTGCGAGTACACGTTCTCAGATGGGATGAGGGCGGCAGGTAG | |
| | | CAGCTCCAGGTCTTTCTCCACCTTGATGCGCTTGGCCGCGTG | |
| | | CAGCGCGGGACCACCGAGCCCAGGGCTGGGCAGCGGTGCTGG | |
| | | CTTGCGTGGGAAGAGAGCTGGAAAGGGCTCTGCGCCTGGCGC | |
| | | GAAGGCCCCGCCGCGCCCGTTCACTGCACCCGGTGCACCCGC | |
| | | GTCCCCACAGCCAACAGCTCCGGCATCACCCGTGTCGCCTGC | |
| | | ACGCTT | |
| Ctip2-ses2 | Ctip2 | CACCGCAGCGGCTCTGTTACCGGACAACTCACACTGGCATCC | 57 |
| | | AAAGGGAGCCTCCATCTGACCCTCACCCTGAGTCCCGTCGCC | |
| | | CGAGATGGGGCGTGCACCACAGCAAGGCAGCGGGAAACAGGG | |
| | | TAGGAGAACGCCCAGGGCACGCAGAGGGGAAGTAATCACGGA | |
| | | GGTGGGAGGGTGGGAAGAGGAGGCAGCTATGGGGGCCATCGA | |
| | | TGGCAGCTGGGCAGGCCTGCACGGCCCTGGAGAAAAAACAAT | |
| Ctip2-ses3 | Ctip2 | ACATTAAGAATCGGTCTGTTCCCAGGAGTTCTGTCTCCAAGG | 59 |
| | | TCACACCATGTCTGGGAAGGAAAACTGTTCCTTCACGCAAAT | |
| | | GCTCGCAGCAACTTCCCAGTACCCTTTATCCTTGTGTAGCCA | |
| | | CCTGCCTCGCTCTCTACACTGCAGAGACTGATGAAGCACTGT | |
| | | GAGCCAGGGCCATGGAGCAGGAGAAGGGCTGCTTTCACGGAA | |
| | | CCACAGTCAGCAAGACATCGGGGATTACGTGATTTCCAGCGA | |
| | | GGGTTCAAGTATCGAGTCCATAAGAAAGGTGAGTCC | |
| Ctip2-ses4 | Ctip2 | TTCCGACGATGTGGCGAAAGGCGACTGGCGCGCATCCGTGAA | 60 |
| | | GCCCAGGAATGGGTCCTTCATGAAGTGGCGCGATGCTGCGTA | |
| | | GCCCACGAGCCACTGCGAGTACACGTTCTCAGATAGGATGAG | |
| | | GGCGGCAGGTAGCAGCTCCAGGTCTTTCTCCACCTTGATGCG | |
| | | CTTGGCCGCGTGCAGCGCGGGACCACCGAGCCCAGGGCT | |
| Ctip2-ses5 | Ctip2 | TGCAATGTTCTCCTGCTTGGGACAGATGCCTTTCGTGGGGGA | 61 |
| | | CAGGAGGTGGTCATCTTCATCAGGGGTGACCTGGATCCCGAT | |
| | | CTCCACTAGCTCAGATACTCTCCTGAGCTCAGAGCGAGAGGA | |
| | | GGGAGGTAGACTGCTCTTGTCCAGGACCTTGTCGTAGCAGGG | |
| | | GCCCAGGCCTCCACACTGTTTCTTCTTGTGCTCTATAAAAAC | |
| | | CAGGATGTCCCCCAGCGGGAAGTTCATCTGACACTGGCCACA | |
| | | GGTGAGGAGATCAGGGTCGGGGCCTCCCACCATCAGCCCCAG | |
| | | GCTGCTAGGCTCCTCTATCTCCAGACCCTCGTCTTCCTCGAG | |
| | | GATGGTAGC | |
| Ctip2-ses6 | Ctip2 | TTCCGACGATGTGGCGAAAGGCGACTGGCGCGCATCCGTGAA | 62 |
| | | GCCCAGGAATGGGTCCTTCATGAAGTGGCGCGATGCTGCGTA | |
| | | GCCCACGAGCCACTGCGAGTACACGTTCTCAGATAGGATGAG | |
| | | GGCGGCAGGTAGCAGCTCCAGGTCTTTCTCCACCTTGATGCG | |
| | | CTTGGCCGCGTGCAGCGCGGGACCACCGAGCCCAGGGCTTTT | |
| | | GTGGTCATCTTCATCAGGGGTGACCTGGATCCCGATCTCCAC | |
| | | TGGCTCAGATACTCTCCTGAGCTCAGAGCGAGAGGAGGGAGG | |
| | | TAGACTGCTCTTGTCCAGGACCTTGTCGTAGCAGGGGCCCAG | |
| | | GCCTCCACACTGTTTCTTCTTGTGCTCTATAAAAACCAGGAT | |
| | | GTCCCCCAGCGGGAAGTTCATCTGACACTGGCC | |
| Ctip2-ses7 | Ctip2 | AGTCCTCCAGAAATATACAGATGCATTGTGCCCGCCCGGCAC | 63 |
| | | ACAGGAAAGGAAGAACACCGACTGAGACATAGGAGAAGGGTG | |
| | | TCAAAATATTGTTTGGTCAGAAAAGAAACAAAAAGGGACAAA | |
| | | GGAAATAAACTGAGAAGCAAAACAAAATAGGGATAGGCGGGG | |
| | | GGCAAACTGAAATTGAAATTGAGAATTCAAACTAAGCAGGGA | |
| | | GGGGTGCTATCCCTGGGAGGGATCCACTGCCCGCCTCGAGCA | |
| | | GGCCTGGCAAGGCCTCTGCTCCTTACAGCCTTTGATAAAAAT | |
| | | AATAAA | |
| Ctip2-ses8 | Ctip2 | GAGCTTGCTCGCCTGCGAGCACGCATGGTCGCACAGCTGGCA | 64 |
| | | CTTGTAGGGCTTCTCGCCCGTGTGGCTGCGCCGGTGCACGAT | |
| | | GAGATTGCTCTGGAACTTGAAGGTCTTGCCGCAGAACTCACA | |
| | | GGACTTGCTCTTGGCAGGCGGCTGCGGTGGCGGTGTGCCCGC | |
| | | AGGCATGGGTGGCAGCGGTGGCGTGCTGAGGAACGGGGACTT | |
| | | GGGACTGGGCTGGAAAGGGTTCAGCAGCCGGTGCATAGGGTT | |
| | | GCCACGGCCTGGGGACACGGGCGGCGGCGTGGAGCTGTTGCC | |
| | | GGCCAGTTCTCGCAGCCGCCGGGAGAAGTCCATGGCAGGAGA | |
| | | GTCTATGGCCATGGGGTTCAGGCGCATGACTCGGTCGAAGGC | |
| | | ACTGGGGTGCTGGGCCACGAGCCCCATCTCCTCTGCACTGAG | |
| | | GCGGTGTGGGTCCAAGTGATGGCGTAGCGGTGGGCTGAAGAG | |
| | | CGGTGGCGTACCTAGCAAGCGGCCCTCACCGAAGCCAGGGTG | |
| | | GTCCCGCAGGATGGGGCCCGTCATGCGCAGCAGGTTGAAAGG | |
| | | ATTGCTGTCCCCCAGGAAATTCATGAGTGGGGACTGCGCCAC | |
| | | GGTCTCCGGCCCGAGCGGTGGGGGATGGTGAGCCTGGGCGTG | |
| | | AGCGAGGTGCTGGCCGGCCCAGGCTCCAGGTAGATTCGGAAG | |
| | | CCATGTGTGTTCTGTGCGTGCTGCAGCAGGAACCAGGCGCTG | |
| | | TTGAAGGGCTGCTTGCATGTTGTGCAAATGTAGCTGGAAGGC | |
| | | TCATCTTT | |
| Fezf2-ses1 | Fezf2 | CGCAGCCAGGCTGGCCAGTTTGGCGTTCTCCAGCAGAAAAAG | 65 |
| | | CTTGGGGTGAGCAGCCAGGGAAGTGGGGGCCTGTGCGTTGAG | |
| | | GAGGCCGGATAGGAAAAGGTGGCCTCCGAGGAGCTCCGAAGG | |
| | | TGGGTAAGCGGTGGAGTCCAGGTAGTTGAAGTAGTAGAGAGA | |
| | | GCCGCTGGCAGGCAGCCCCAC | |
| Fezf2-ses2 | Fezf2 | GAACTTGTCCGCAGCCAGGCTGGCCAGTTTGGCGTTCTCCAG | 66 |
| | | CAGAAAAAGCTTGGGGTGAGCAGCCAGGGAAGTGGGGGCCTG | |
| | | TGCGTTGAGGAGGCCGGATAGGAAAAGGTGGCCTCCGAGGAG | |
| | | CTCCGAAGGTAGGTAAGCGGTGGAGTCCAGGTAGTTGAAGTA | |
| | | GTAGAGAGAGCCGCTGGCAGGCAGCCCCACAGCCTG | |
| Fezf2-ses3 | Fezf2 | GCACCTAGTCGGCCCGGTCACCCATTTGCATTCAAAGGAACA | 67 |
| | | GAAGCAGAACAAAGTGCACCCAAGGGTACCAAATTACCGCCC | |
| | | ATTAACCCGGCGCGCAAAGGAACCCACCAGCTATTGCCCTAG | |
| | | GACTATGAAAGGGGGAAGAAGGGGGAGGCTGTACAAAGGAAA | |
| | | GGAGAAGGGGTAGAAGAAAGGAGTCTCAAATTAACACTCCCA | |
| | | GCCCTTCCCTAGACCCAGGCCTTCGCCACGCGTGCTGGGAAC | |
| | | TGGGCACTGAAAGAGCAAAGTGAAAGAGGGCCGGAAGAACCA | |
| | | CCTCGCTTTTACCTCTTTCCCCCACCGCCAAGGAGATGCGTT | |
| | | CCGAGCCATGCAGCGTGTCTCTTCTGTCACATTTTG | |
| Fezf2-ses4 | Fezf2 | CTTGCCGCACACTTCGCAGGTGAAGTTTTTGGGTTTGCTGTC | 68 |
| | | AGTAGAGCCCCCCGGGAGTTTGCTGTGGCTCTTGACTCCCCC | |
| | | TCGTTCAGCTGTCAAGGCCGAGTTCTCCTTCAGCACCTGCTC | |
| | | CAGTGGCGCATGCAAGCGCTCCTTATGGGGATAGGAAGCTGG | |
| | | GTGGGGGAACTTGTCCGCAGCCAGGCTGGCCAGTTTGGCGTT | |
| | | CTCCAGCAGAAAAAGCTTGGGGTGAGCAGCCAGGGAAGTGGG | |
| | | GGCCTGTGCGTTGAGGAGGCCGGATAGGAAAAGGTGGCCTCC | |
| | | GAGGAGCTCCGAAGGTAGGTAAGCGGTGGAGTCCAGGTAGTT | |
| | | GAAGTAGTAGAGAGAGCCGCTGGCAGGCAGCCCCACAGCCTG | |
| | | GTTGATGACCTGCGGTTTGATGACCCTGCCGGCGGGCAGCGC | |
| | | AGAAGGCGCGAGGCCCAGTTCGGCCTTGCAGCACACGCCACA | |
| | | GTTGGTTTTGCACAAGCCGCTGGCGCCGCACACTGGGGCCCC | |
| | | ACCGCCGCTGCCTCCTCCTCCACCGCCGCCCGCCCGGAGGCT | |
| | | GCTCTTCCAGAACTCCGAGTAACTGAGCAGCGTCTTGGACGG | |
| | | CACCTCGTAGCCGAGAGGCTGGAGAGGGATCATACAGGGCAG | |
| | | CGGCGAGCAGAGGTTGAGCAGTTTCTTGCTCTGGCTGCTGTC | |
| | | TGCCTCGAACGCAGCAGGCCGGGGCTCGAAAGGCGCTCGGGG | |
| | | CTCGGACGTCTTGGCCATGATGCGCTCGATAGAGAAAGC | |

| | | seq id |
|---|---|---|
| SesRNA_ID | sesRNA sequence | no |
| human- | AGGGATGTCTGCTCCACCTGCAGCTCCGACAGTTTGCCCTGCAAATGGTTGCGCTG | 77 |
| NPPB-Ses1 | CTCCTGTAACCCGGACGTTTCCAAGTCCGAGGCTGAACCGGGGCTGCCCAGCGGGT | |
| | AGGAACGACCTCCCAGGAAAGCCAGATGCAAGAAGAGCAGGAGCAGGAGCGCCCGG | |
| | GAAGGTGCTGTCTGGGGATCCATCTCTCTGGAGGGACTGCGGAGGCTGCTGCTGCT | |
| | G | |
| human- | TCCGGTCCATCTTCCTCCCAAAGCAGCCAGACCCTTGCACCATCTTGGGGCTTCGT | 78 |
| NPPB-Ses2 | GGTGCCCGCAGGGTGTAGAGGACCATTITGCGGTGCCCACGGATGCCCTCGGTAGC | |
| | TACCTCCCGGGACTTCCAGACACCTGTGGGACGGGGGCTCTCCTGGAGGGGCTCCA | |
| | GGGATCTCTGCTCCACCTGCAGCTCCGACAGTTTGCCCTGCAAATGGTTGCGCTGC | |
| | T | |
| Human- | GGCAATCACGGTGGCCGCACAGACCTACAGGACAGCCCCGCCATCCCCGCCCCCGA | 79 |
| VIM-ses1 | GGGACCATCCCTTTGTCTCGCTCCCTCCACCGCCTTCCCCTCCTTCCTTCTCCCGC | |
| | CCGGTGATTGGCAGCCTGCCAGAAGGGGCAGGAAACTTTCTGAAAGTTTGGAGGAC | |
| | TAGCTCTCATTGTGCCCAAGGGCCTTCAACTGCACACAAAGTGGTAGTTTTAAGAA | |
| | ATCTGTAACTGGAAACGGAGCGTCCTTGGGCAATGTGTGGGGACAGAGGAGGAAAT | |
| | GCGAACTGCAAGGTCTGGGT | |
| Human- | TTTCGGGGACCAGATGTGATCAGCGACCTCCGTGGGACTGGGAAACTCAGCTTTGG | 80 |
| ses1 | ACTCAGGGCAAAGAGCTATGAGGGGAGAAGCCCATTTCCAGCTGGAGAACTCTCCT | |
| | TCCTACCACACACATGGATGGCCTAGAAACCGATCTTGCCCAGATCCCAGGGGACT | |
| | GAGTGCCACAGTTTCCCACTTGTTAAGTCAAAGCCGTGTTTCCTTCATAGCCCACT | |
| | GAGGACCCCACCCTGCATTTCTGCTGGAAGGAGCCTCGTGGAGGCTGTCCTCGTGC | |
| | CCCAGGCCCAGGCAGCCAAG | |
| Human- | TGGCCACCCCATCCAAACTCACTCCAAGTCATGCCACCTCAGACAGGGAGAAGCCT | 81 |
| HER2-ses1 | GACTGAAGGACCCCTCCGCCCATGTCCCTTGCACACTGCAGGTTTAACAGCAGGAG | |
| | AAAGCTTGCGTGCTTAGGAAGTAGAGGGATTTCAGGGGAAAGGCTTCTTCTGACTA | |
| | GGCCCACCCTGTGCATTCAGAGGCTGCCCCAGCGTGCCAGCCCTGGGGAGCTCGCC | |
| | CGCCAGTACAGGGTGTTGGCTGGCAAAGAAAGGCTTCCTTTGTCCCAAAGGCACAA | |
| | CTCAGGGCAAAGGGGTAAAA | |
| Human- | AGGGAAGGGAGTGAAGAGCACAAAGAAATATCCTGGCAAGAGGGCAGGCCAGCTGT | 82 |
| CD3E-ses1 | GAGGGTGGGACTACTGATGATGCTTCAAAGGAAGAGGAAGCAGCAATATTTTAGGA | |
| | CTGGGTACCAGCAGAGAAGGCAGGGAGGAGGGAGCGGGGGATCAGGCTAGAGGGCG | |
| | CGAGTTCACCATGAGGCTGAGGAACGATTCTCTCTCGTGGGGTCCAAGACTAGCCC | |
| | AGGAAACAGGGAGTCGCAGGGGGACTGGAGAGGAGACCTGGGCCAGCGGGAGGCAG | |
| | TGTTCTCCAGAGGGTCAGAT | |
| mouse- | GAAGTTTCACTCCTTCCGCATTTATTGGAGAAAACAGGCAGAAGGTTGGGAGAGGG | 83 |
| Ses1 | GCTCTACCGGTGGCCAGTGGAGCAGCTGGGGTGGGAAGACTGGGCCTAGCACATCT | |
| | CACTTAGCCGCAGGAGCCGGTTTCTCCTCAAGGTTCATTCAACTGCTGAAGAAGTT | |
| | CACCAGACTAGTTCCTGCTGTCCTGACAAGGGGTGTCAGCTGCTCGTGTGTCTTCT | |
| | CAAAGTATGC | |
| mouse- | CACTGGGTGTGCATCACCCTGAACTGGCCGTCTTGTTTCAGGATGTGGAAGTCACA | 84 |
| Ahsg-Ses3 | GTCTCCCTCCACCGCGTGCTCAGTCAGCTGCCTCACAGAACAGTTTGCCAGCGGGG | |
| | TGGGGTCCAAAGCATAGCAAGTGGTCTCCAGTGTGTCAACTTCCATCTCATACACC | |
| | ACTCCGAAGGGCCGCCGAGACCACACCTTGACTTTGTCGATCTCATTCAAGACCTG | |
| | TTTGAATCCCTCAAGAAGA | |
| mouse- | GAACTGTGGTGGCTGGGCAGCTCCCACCCACCCACCCACCCACCAACCAACCACAA | 85 |
| Ahsg-ses8 | GGCAGCACTCACGTGCTCAGTCAGCTGCCTCACAGAACAGTTTGCCAGCGGGGTGG | |
| | GGTCCAAAGCATAGCAAGTGGTCTCCAGTGTGTCAACTTCCATCTCATACACCACT | |
| | CCGAAGGGCCGCTGTGGAGCCGGACAGAAGGGAGAGGTGAGCAAACGCCCTGCTCC | |
| | GCTGATCCAATGCATCGCACCTAGTGTGT | |
| mouse-Alb- | GATGTCTTCTGGCAACTTCATGCAAATAGTGTCCCATACAGGTGGTTGGGTTTTCC | 86 |
| ses4 | TTACAGGAGGTGCACATGGCCTCAGCCTCTGGCCTTTCAAATGGTGGCAGGCTAGG | |
| | GTTGTCATCTTTGTGTTGCAGGAAACATTCGTTTCTTTCGGGCTCTTGTTTTGTAC | |
| | AGCAGTCAGCCAGTTCACCATAGTTTTCACGGAGGTTTGGAATGGCACACAACTTA | |
| | TCTCCAAAAAGAGTGTGAAGGGATTTGTCACAGTTGGCGGCAGACT | |
| mouse-Alb- | GGTGTCTTCTTGAATATTCATACAAGAACGTGCCCAGGAAGACATCCTTGGCCTCA | 87 |
| ses6 | GCATAGTTCTTGCACACTTCCTGGTCCTCAACAAAATCAGCAGCAATGGCAGGCAG | |
| | ATCAGCAGGCATAGTGTCATGCTCCACCTCACTACGACAGTGGGCTTTCTTCAACA | |
| | GTGGTTTATCGCAGCAAGTCTGCAGTTTGCTGGAGATACTCGCCTGGTTTTCACAC | |
| | ATGTACTTGGCAAGTTCCGCCCTGTCATCTGCGCATTCCAGCAGGT | |
| Human- | TTCTCTGCAATTGAAGCCGAATCTGCCCTAATCCCCACCCCTGGGTTCAGACTTCT | 88 |
| APOA2-ses1 | GTGGGACCTCTCATCTTCCCTTCTTTCTCTCCACAGTTCCACAGCCCCTGAACCCC | |
| | TTGCCCTGAGACTTACTTAGCCTCGGCCTGAAGCTCTGGGCTCTGGACCTTCTCCA | |
| | TCAGGTCCTTGCCATAGTCAGTCACGGTCTGGAAGTACTGAGAAACCAGGCTCTCC | |
| | ACACGTGGCTCCTTTGCCTGTCTCCGAA | |
| Human- | GAGCTGCCAGTAACACATGGGAGTCTGGGGGTGAGCCAGCTGGAGGGAGTCCAGGT | 89 |
| AHSG-ses1 | GCGCCAAGTGGAGGGGACGGAGGTGCATCTGGGTCCACCACGGGTGTGGGGACTGC | |
| | TTCATTAGCACCTTCTGGTTGGGGCTGTGAGCTCACGGGCTGTGTTTGGAACACCA | |
| | TGCAGGTCACTGCAACCTCTGCCCCACCAAGCTTCTCACAGAGTGTTGCCTTACAA | |
| | AAGCCATATTGCTTTTCTGCCAGCAGGTTACACTTGGCTGCCTCTGTGGCCTCTTG | |
| | AGCAACACAGTCAGTGCCAG | |
| Human- | TTTACTAGATTAAAAAACATTGAAATAGGCCGGGCGCGGTGGCTCACTCCTGTAAT | 90 |
| ALB-ses1 | CCCAGCACTTTGGGAGGCCGAGGTGGGCAGATCACGATGTCAGGAGATTGAGACCA | |
| | TCCTGGCACACGGTGAAACCCCATCTCTACTAAAAATACAAAAGATTAGCCGGGCG | |
| | GGATGGCGGGCGCCTGTAGTCCCAGCTACTCGGGAGGCTGAGGCAGGAGAATAGCG | |
| | TGAACCCGGGAGGCGGAGTTTGCAGGGAGCCGAGATTGCGCCACTGCACTCCAGCC | |
| | TGGGCGACAGAGCGAGACCC | |
| HIV-ENV- | TTATATCCTCATGCATCTGTTCTACCATGTCATTTTTCCACATGTGAAAATTTTCT | 91 |
| ses1 | GTCACATTTACCAATACTACTTCTTGTGGGTTGGGGTCTGTAGGTACACAGGCATG | |
| | TGTGGCCCAAACATTATGTACCTCTGTATCATATGCTTTGGCATCTGGTGCACAAA | |
| | ATAGAGTGGTGGTTGCTTCCTTCCACACAGGTACCCCATAATAGACTG | |
| COVID19- | GGAGAACCCTGGACCTGAATTCGTAGTTTACTGCGCAACTACTCATAATGACACTG | 92 |
| Spike-ses1 | TTTTTATCTGTAATGGGTTCAGGGTAGTAATAGGAACTGCCTGTGAACTTCCATTC | |
| | TCCATCATCTTGAACAAAATATCCAGCTTTAGGTGCGAATCCTCTATCACCAGAAA | |
| | TGCAAAGTCCAGGACTCACATTTGCGGTTGTAAAGGATATTAGCACATAGCTGAAG | |
| | TGTATAAAATAGAAGCCATAAGGCGCATTCTGGACAAGAGAGAATATATGATTACC | |
| | ATTGCCACAGAAATTAATACGCGTGGTTTGGCTCTTAACGCACTCATTGACCTTTT | |
| | CTATGGCCTGAGCAGCACTAACTTTAATGGCGCGCCAGAGGGCAGAGGAAG | |
| COVID19- | GGAGAACCCTGGACCTGAATTCCAAAGTCCAGGACTCACATTTGCGGTTGTAAAGG | 93 |
| Spike-ses2 | ATATTGGCACAGAGCTGAAGTGTATAAAATATAAGCCAGAAGGCGCATTCTGGACA | |
| | AGAGATAATATAGGATTACCATTGCCACAGAAATTAATACGCGTGGTTTAGCTCTT | |
| | AACGCACTCATTGACCTTTTCTTATGGCCTGAGCAGCACTAACTTTAATAAGCGTA | |
| | CTATCACTAAGTTGCTTGGATATATACGCATTAAGTGCAGTGAACCTGCCATTAAT | |
| | AAGACGATCTATCTGGGCTTTTGCTTCTACAGCCTCAAGCCGAGTGGCGCGCCAGA | |
| | GGGCAGAGGAAG | |
| COVID19- | GGAGAACCCTGGACCTGAATTCGGAGGCGGTTTGCATATTATCCAAGAGGTTATTA | 94 |
| Spike-ses3 | ACCTCATTAAGAATGGCATTAACATTAACACAGAAAGAGCCATACTCAACCAACTG | |
| | CTGCCTGCATGCAGTGTTATCACCACAGACAAATGCAGCACAATCTATAGTCACCT | |
| | TTAGAGATCTAGTTTGAATGAACTCCTCGTGGTGCCCAATAGTAAAATTGGTTGGT | |
| | ATTTGCATCTCATAGAATCCATCAACGGATTGGACACTATCATTAACTTTCCAGGA | |
| | GGCGCGCCAGAGGGCAGAGGAAG | |
| COVID19- | GGAGAACCCTGGACCTGAATTCCACACAAATTGGGGGTTTTACATCCGTGTGCCAA | 95 |
| Spike-ses4 | AACCCTATAAGCTTATTACCATTAGTGTTAGGCTTACAATCAGTGTAAGGGAACAG | |
| | ACAAATGGTATACTGGCACACGGAGGCCATTATAACACCATTATATAGCTCTATTA | |
| | CAACGGTATAGGAAGTATAGCCAAACAAACTACCTATAACTATAGTAGGAAAATAT | |
| | GCAGTTGCACCGGATGGCGTACTTGTCTTAAGGTTCTGCACCTTCGCAAATATGCC | |
| | ATCATTAAACTGACTGAAATAGGGTGGTTGAAACCACGACAAGCTAACGGCGCGCC | |
| | AGAGGGCAGAGGAAG | |
| COVID19- | GGAGAACCCTGGACCTGAATTCGCCAGCACCCATACGGAGATCACAATTAGGAAGC | 96 |
| Spike-ses5 | GCCTCATCCGTGCGGTTATCAGCATTAACAACACAACCCAAATAACTATCAAAGTA | |
| | ATTAAGTAGGTTCTCCTCACGGGAAATATTATTGCTAAAAACATAGCTACAATTTA | |
| | TATTACGATAGAGCAGAGCCGGTTCGGGTGCATCTTTGTGAAATGCAGCAGAAACA | |
| | CGGCCACTATAACAGCTCCTTATCGTATAAGTCTTGTTAGTGGTAAGATCACGAAA | |
| | ACCGGCGCGCCAGAGGGCAGAGGAAG | |
| COVID19- | GGAGAACCCTGGACCTGAATTCAATGCATCAGCATTAACATTAAGCGTGAAATTTC | 97 |
| Spike-ses6 | GCTTTAACACACAAATTGGGGGTTTTACATCCGTGTGCCAAAACCCTATAAGCTTA | |
| | TTACCATTAGTGTTAGGCTTACAATCAGTGGAAGGTAACAGACAAATGGTATACTA | |
| | GCACACTGAGGCCAATTATAACACCATTATATAGCTCTATTACAACGGTATAGGAA | |
| | GTATAGCCAAACAAACTACCTATAACTATAGTAGGAAAATATGCAGTTGCACCGGA | |
| | TGGCGTACTTGTCTTAAGGTTCTGCACCTTCGCAAATATGCCATCATTAAACTGAC | |
| | TGGCGCGCCAGAGGGCAGAGGAAG | |
| COVID19- | GGAGAACCCTGGACCTGAATTCGCAGTTAACCTGCCATTAATAAGACGATCTATCT | 98 |
| Spike-ses7 | GGGCTTTTGCTTCTACAGCCTCAAGCCGAGTTAGAATTTCTTGTAAAGAAGCACTA | |
| | ATAGCACCAAACCTGTTAGAAAGTGGATTTAGTAAGTTATTGAGTGCTTCAGCATT | |
| | TGCATTAACAACGGACTAGATCTTACCTAAAGCAGAATTGGTTGCATCAAACCCAT | |
| | CCTAGATAGCACCCAGCGCATTGTTAAAAGCACTAGCAATCATCTTTTGGTTCTCA | |
| | CTAAGCACATTCATAGTGACACCTAAACCATTAATTCTATATGGAACACTTAAACT | |
| | AAATGGCACACCGGCAGCTGCTGACCACGGTGGCGCGCCAGAGGGCAGAGGAAG | |
| COVID19- | GGAGAACCCTGGACCTGAATTCGCTTTTGCTTCTACAGCCTCAAGCCGAGTTAGAA | 99 |
| Spike-ses8 | TTTCTTGTAAAGAAGCACTAATAGCACCAAACCTGTTAGAAAGTGGATTTAGTAAG | |
| | TTATTGAGTGCTTCAGCATTTGCATTAACAACGGACTGGATCTTACCTAAAGCAGA | |
| | ATTGGTTGCATCAAACCCATCCTAGATAGCACCCAGCGCATTGTTAAAAGCACTAG | |
| | CAATCATCTTTTAGTTCTCACTAAGCACATTCATAGTGACACCTAAACCATTAATT | |
| | CTATATGGAACACTTAAACTAAATGGCACACCGGCAGCTGCTGACCACGGTGGGAA | |
| | CATAGCTGCCGCAGTAGCACCGGTTGTGGGCGCGCCAGAGGGCAGAGGAAG | |
| COVID19- | GGAGAACCCTGGACCTGAATTCAGTTGCATATTATCCAAGAGGTTATTAACCTCAT | 100 |
| Spike-ses9 | TAAGAATGGCATTAACATTAACACAGAAAGAGCCATACTCAACCAACTGCTGCCTG | |
| | CATGCAGTGTTATCACCACAGACAAATGCAGCACAATCTATAGTCACCTTTGGAGA | |
| | TCTAGTTGGAATGAACTCCTCATAGTGCCCAATAGTAAAATTGGTTGGTATTTGCA | |
| | TCTCATATAATCCATCAACGGATTAGACACTATCATTAACTAACATCGGAGTGTAT | |
| | GGCTCAAATGTAGTTAACCGAGAGCCAGTAGAAACTGATCGGTCAGCCCTGCGTGA | |
| | TTTTGAAGGCGCGCCAGAGGGCAGAGGAAG | |
| COVID19- | GGAGAACCCTGGACCTGAATTCGAGGAAATATTATGTATCACAATACTGTCCTGGT | 101 |
| Spike- | GTCCTCCATACTCATCACAACAATTTCCAAAATCTAACTCCTCCTTAAAGTCGGGT | |
| ses10 | GGATTAGGTATTGAAGTGTTCAAGAAAACTTCAGGTGCCAAAAGTTGCTTGGATAT | |
| | ATACGCATTAAGTGCAGTTAACCTGCCATTAATAAGACGATCTATCTAGGCTTTTG | |
| | CTTCTACAGCCTCAAGCCGAGTTAGAATTTCTTGTAAAGAAGCACTAATAGCACCA | |
| | AACAAACTTAAACTAAATGGCACACCGGCAGCTGCTGACCACGGTGGGAACATAGC | |
| | TGCCGCAGTAGCAAAACCGTCTGGCAGTCTCGAGCTTATAGTAACACCCTGCATTA | |
| | ATGCACTAGCAACTTGTAGTTGCGGCGCGCCAGAGGGCAGAGGAAG | |
| COVID19- | GGAGAACCCTGGACCTGAATTCGAGGAAATATTATGTATCACAATACTGTCCTGGT | 102 |
| Spike- | GTCCTCCATACTCATCACAACAATTTCCAAAATCTAACTCCTCCTTAAAGTCGGGT | |
| ses11 | GGATTAGGTATTGAAGTGTTCAAGAAAACTTCAGGTGCCAAAAGTTGCTTGGATAT | |
| | ATACGCATTAAGTGCAGTTAACCTGCCATTAATAAGACGATCTATCTAGGCTTTTG | |
| | CTTCTACAGCCTCAAGCCGAGTTAGAATTTCTTGTAAAGAAGCACTAATAGCACCA | |
| | AACAAAAAGGTGTCGAGAAGAGGAGAACAATATGCTAAATGTTGTTCTCGTCTCCT | |
| | CGACACCAAACTTAAACTAAATGGCACACCGGCAGCTGCTGACCACGGTGGGAACA | |
| | TAGCTGCCGCAGTAGCAAAACCGTCTGGCAGTCTCGAGCTTATAGTAACACCCTGC | |
| | ATTAATGCACTAGCAACTTGTAGTTGCGGCGCGCCAGAGGGCAGAGGAAG | |
| COVID19- | GGAGAACCCTGGACCTGAATTCGAGGAAATATTATGTATCACAATACTGTCCTGGT | 103 |
| Spike- | GTCCTCCATACTCATCACAACAATTTCCAAAATCTAACTCCTCCTTAAAGTCGGGT | |
| ses12 | GGATTAGGTATTGAAGTGTTCAAGAAAACTTCAGGTGCCAAATCGACAAAGCCAAC | |
| | ATCAGATAATTTGACCTTGTCAAATAACAAATCCTCTATAGCAGAAAAACTCATCC | |
| | GTGCGGTTATCAGCATTAACAACACAACCCAAATAACTATCAAAGTAATTAAGTAG | |
| | GTTCTCCTCACGGGAAATATTATTGCTAAAAACATAGCTACAATTTATATTACGAT | |
| | AGAGCAGAGCAAACCATTTAACAATATATTAGCAAAAATTTGGCAGCGATCATTAA | |
| | CAAGGCAGGTATCATGTAAATGGTTTTTGCCAAAGACGCCAGCATCATTAAAGCCA | |
| | TACCTCCTATTCCAAGACGAGGGGTTATGGGGCGCGCCAGAGGGCAGAGGAAG | |
| COVID19- | GGAGAACCCTGGACCTGAATTCGAGGAAATATTATGTATCACAATACTGTCCTGGT | 104 |
| Spike- | GTCCTCCATACTCATCACAACAATTTCCAAAACGTATAAGTCTTGTTAGTGGTAAG | |
| ses13 | ATCACGAAAACCATTCAAATTACCATTAACATCATACAGAAGGGTTTGCCAGCTAT | |
| | TATAATAGTCAAACCTTATGTGGATCAGCATTGCCACAATTATCTTCTAAAACACC | |
| | TAAGCCTTCACAATGGTCACAAAGCCTACTTCGGGGTACAGCAAACTTATCAACTG | |
| | AAATACTACCAAAGCACCTGCCATACACTTTAGAAGCATCGATATTATTACAAAAC | |
| | AAACTCTCAGCCTGAACATAACGTAACAGGCTGCTTAAATAAAAAGGTGTCGAGAA | |
| | GAGGAGAACAATATGCTAAATGTTGTTCTCGTCTCCTCGACACCAAAGCTCTGGGT | |
| | CTTACATTTTATTTCACTGGTATAACTACTAGCACAATCAACAGCACTAGTAATAA | |
| | AACAAATTGGGGGTTTTACATCCGTGTGCCAAAACCCTATAAGCTTATTACCATTA | |
| | GTGTTAAAAACTTCAACGGTCTCAGTGCTAATGCTTGGAGCACTAACATTAGCACC | |
| | GTTGGCGCGCCAGAGGGCAGAGGAAG | |
| COVID19- | GGAGAACCCTGGACCTGAATTCGTCTTGTTAGTGGTAAGATCACGAAAACCATTCA | 105 |
| Spike- | AATTACCATTAACATCATACAGAAGGGTTTGCAAACCCTTATGTGGATCAGCATTG | |
| ses14 | CCACAATTATCTTCTAAAACACCTAAGCCTTCACAATGGTCACCCAAATTAGCAGT | |
| | CTGCAGAAATCCAGAGTTACCAAGCTGTAAATCAACTTGCCTACTTCGGGGTACAG | |
| | CAAATAAAGTCTTACGCTCCCAGTTGAGAGGGGAGGGGACTGACCTAGCAGTAAGC | |
| | CACTCCTCTATAAAAAGCTTATTACCATTAGTGTTAGGCTTACAATCAGTGGAAGG | |
| | TAACAGACAAATGGTATACTAGCACACTGAGGCCAATTATAACACCATTATATAGC | |
| | TCTATTACAACGGTATAGGAAGTATAGCCAAACAAACTACCTATAACTATAGTAGG | |
| | AAATATAACCACGACAAGCTAACTGAGTTAGTTCCCGTAAGAGCGAGGTTTCTAAA | |
| | CTTAGAACCATCGACCGGGAAAAGCTGGATACATCTAAAATCACCAATATACCCTA | |
| | AACAAGAGGGCAAAAATAGAATAAACACGAACAGCGGCGCGCCAGAGGGCAGAGGA | |
| | AG | |
| COVID19- | GGAGAACCCTGGACCTGAATTCATATTATGTATCACAATACTGTCCTGGTGTCCTC | 106 |
| Spike- | CATACTCATCACAACAATTTCCACACAAATTCTTAAACCATTTATCTAACTCCTCC | |
| ses15 | TTAAAGTCGGGTGGATTAGGTATTGAAGTGTTCAAGAAAACTAAATATCCAGCTTT | |
| | AGGTGCTAATCCTCTATCACCAGAAATGCAAAGTCCAGGACTCACATTTGCGAAAA | |
| | AGGTGTCGAGAAGAGGAGAACAATATGCTAAATGTTGTTCTCGTCTCCTCGACACC | |
| | AAAGCATTCTGGACAAGAGATAATATAGGATTACCATTGCCACAGAAATTAATACG | |
| | CGTGGTTTAGCTCTTAACGCACTCATTGACCTTTTCTTATGGCCTGAGCAGCACTA | |
| | ACTTTAATAAGAAAGTGGAAGAGGAGAACAATAGGCTAACTGTTGTTCTCGTCTCC | |
| | CACTTTAAACTGCTGCCTGCATGCAGTGTTATCACCACAGACAAATGCAGCACAAT | |
| | CTATAGTCACCTTTAGAGATCTAGTTTGAATGAACTCCTCGTGGTGCCCAATAGTA | |
| | AAATTGGTTGGTATTTGCATCTCATAAAATTGTTAGTGGTAAGATCACGAAAACCA | |
| | TTCAAATTACCATTAACATCATACAGAAGGGTTTGCCAGCTAAAACCAGTAACCAC | |
| | TTCAGTATTAGGCAACTGCAAATCTGTGGAACATGTGGTACCACTATTAAAAACGT | |
| | CATGTTGGTTTTTGCCAAAGACGCCAGCATCATTAAAGCCATACCTCCTATTCCAA | |
| | GACGAGAAAGGCGCGCCAGAGGGCAGAGGAAG | |
| Human- | AAAGCATGCGGTAAGAAACTTAGGGAAGAAACTATGAGAGTACTTCTTAGGGTGAG | 107 |
| CFOS-ses1 | ACTCAGAGTCTGAGTTGGGATGGAATGGGCTTGGAGCCAGGGCTCCCAGTTACGGA | |
| | TGTGCAGCCAGCCCCATCAGTAGCTTCATCCTCTGTACTGGGCTCCTGCATCTCCG | |
| | GGGCTGCTTCCCACCCAGCCCCCACATTCCCAGGAAGAGTACGCTAGAGTTCCTCA | |
| | CCTGTTCCACCTTGCCCCTCCTGCCAATGCTCTGCGCTCGGCCTCCTGTCATGGTC | |
| | TTCACAACGCCAGCCCTGGA | |
| Human- | GATCCTCAGCAAGAGAACAAAGAAGAGCCCAGTCTGAAAGCATGCGGTAAGAAACT | 108 |
| CFOS-ses2 | TAGGGAAGAAACTATGAGAGTACTTCTTAGGGTGAGACTCAGAGTCGGAGTTGGGA | |
| | TGGAATGGGCTTAGAGCCAGGGCTCCCAGTTACGGTTGTGCAGCCAGCCCCATCAG | |
| | TGGCTTCATCCTCTGTACTGGGCTCCTGCATCTCCGGGGCTGCTTCCCACCCAGCC | |
| | CCCACATTCCCAGGAAGAGTACGCT | |
| Human- | ACTGGGAACAATACACACTCCATGCGTTTTGCTACATCTCCGGAAGAGGTAAGGAC | 109 |
| CFOS-ses3 | TCGAGTCCACACATGGATGCTTTCAAGTCCTCGAGGCCCACAGCCTGGTGTGTTTC | |
| | ACGCACAGATAAGGTCCTCCCTAGGTCTACAGGAACCCTCTAGGGAAGATGTGTTT | |
| | CTCCTCTCTGTAATGCACCAGCTCGGGCAGTAGCACTTGTGGGTGCCGGCTGCCTC | |
| | CCCTTCCCTGCCCCCTCACAGGGCCAGCAGCGTGGGTGAGCTGAGCGAGTCAGAGG | |
| | AAGGCTCATTGCTGCTGCTG | |
| Human- | TTAACCTCAGTAATACTTTATCTTTACTATTGGAGGCATGTCACATGGAGTCCATG | 110 |
| KCNA1- | TTGTCTACTTACATCTGCTGCATGAACAGTCCCAAAACAGTGCGCTGAAGAGAAGA | |
| ses1 | AAGGTGCTTCTGCAAGGTTTGGAGAGGAGACAACTTTGACCTAGCCGGCTGGCTTG | |
| | GCGCAGGGGCGGTGCGGAGTCAGCGGAGAGCCCGAGTGGGAATGCGGCAGGTCGTC | |
| | TTTGCAGTGCCCTGGCGGGAGCCGCGGC | |
| mouse- | CCTGGTCCTTGGAGCCAGAGGGTGGCAGAGGAGCGCCGCCGCGCTGATAATGAATG | 111 |
| vGAT-ses1 | TCTCCCTCGACGGGAGCTTCTGCGCCCTCGTCTCCGCAGGGCTCGCCTTCCGATTT | |
| | CAGGATGTCCATCTGCAGGCCCTGGCGATGCTCAAAGTCGAGATCGTCGCAGTGCG | |
| | CGAAGCCCACCGCTTCCTCATCGGTAGCCGCCTGAAACCCCATCCTAGCAAACATG | |
| | CCGCTCACCTTGGCCTGGGACTTGTTGGACACGGAGGTGGCCACATTGGTCAGCTT | |
| | GCTGCGGAGCAGGGTGGCCA | |
| mouse- | TCCTCTGCGTTGGTTCGGTGGGCCTCGATGAGACCCTCGAGTGAATGCACGAAGCC | 112 |
| vGAT-ses2 | GGACACGCTGCAGATGCCGCCGATGACGAAGATGGCCACATCGAAGAAGACCTAGT | |
| | GCCACAGCAGCTTGCGCCAGAGAAGGCGCAAGTAGAAGAGGCTGGGCAGCAGGAAG | |
| | CAGAGGCCGGCGCCTGTGAGGCTGCCCGTGAGGCCCATGAGCAGCGCGAAGTGTGG | |
| | CACGGAGATGGCCATGAGCAGCGTG | |
| mouse- | AAACGGAAGGAAGCCACAAAGGAAGAAAATACTCAGACGCTTTAGGTAGTAGACAC | 113 |
| vGAT-ses3 | AGGGTCTTGGGGAACGGAGACGAGGTGTAGATATAGGTTTGGAAATAAGAACTACA | |
| | TAGAAGAAGACTAGAGTTGGGAAACCCTAAGATGGGTGTTAAATGGGATCTGCAGA | |
| | GAGGATCAGAATAGAGACCCCTCCAACCCACAAGCCCTACGCTGCAGGAAGCCTAA | |
| | ACCGCAGGTCTCTAGAAACTGAGGAGAGGGGAGGGCCAAAAGCTGCGCAATATGGT | |
| | TTCTAATTTAC | |
| mouse- | GTTTCTGCGGTGCAGCCGGGATTTCCAGCAAGAGGAGCTAGTCTTCGCAGCTTCGT | 114 |
| vGAT-ses4 | ACTGCGGAGTCCCTCTACCATAGACGTTCCTATGTACCTGTATCTGTATGTGTCCA | |
| | ACAGAGCTGCACCAGCCAAGTAGGTGCTGTCTGCTTTAGTGTGTGTGTGCGTGTGC | |
| | GTGCGTGTGCGTGCATGTGTGTGTGCTCACGAGTTAAGATTAAGCTAATGCAGAGG | |
| | CACTTGCTGTGTAAAGGTATTGCTGCTATTGCAGTGTCTTGTGGTATAGTCAAGTG | |
| | CATTTAGGGTAACCCTGCGAACTATAAATAGATGCACGTCATATGCCTGTTTGTTA | |
| | GTGAGGAAGGGGACCGTGTCTAGCCCTGTCCGCGTGCACGACTTCTGTTTGTGTCA | |
| | CGAG | |
| mouse- | GCGGCGGCGGGGGGGGAGAAAAGGACAGAAGGGTCTGGGGACGCGGCGGGCGCGCG | 115 |
| vGAT-ses5 | TCAAGGCAGCGAGGACTGCTATCTCCGCTGGGTCTCCGCGCGGCGCCCCAGGGCGC | |
| | TCTGGCTCATGACCCTCGCGCCTGACGTTCGAGGCTAGCCCCGGTCACGACAGTCT | |
| | GGCTCTTGGTAGGACCGGCCCGAGAGGCGACGAGTGCACTGCAGAGCTGGTGCGGG | |
| | TGCCGGCTGGGCTGCGGAGGGCGGCGGCGACGGCAGCGTCAGAACAGCTACGCGAG | |
| | GC | |
| mouse- | TATACATTTCATCTTATTCACCACGAGCACACCACACGCACAGTATACAGTTCCAC | 116 |
| vGAT-ses6 | GACCGGATACATTGCACAAGATGAGTTTGGGTTCCTGAAAACCGGCAGGCAAACGG | |
| | GGCCCCTGCCGCGGCTTCTCCAGAGTGAAGTCGCCCGCCCGAAATAGATTTCCCGG | |
| | TCAACCTTTCAGGCCGCAAGGTTGAAATGTCCAGGGCATGGGGAGCGCTCTTGCTA | |
| | GAAATGGCTGGACGCTGTAGATTCCAAGCACTGGCTGGCTGTGAAATGGCCCCACC | |
| | CCACCCTCAAGGTCAAGTTTCCAAGCCTGCGAACTTTCCCTCCCTTCCCACCCCCG | |
| | TCGTTGACAGGAGCCAAAATTTGGTGG | |
| RAT-vGAT- | GGCCACATCGAAGAAGACCTGGTGCCACAGCAGCTTGCGCCAGAGAAGACGCAAGT | 117 |
| ses1 | GGAAGAGGCTGGGCAGCAGGAAGCAGAGGCCGGCTCCCGTGAGGCTGCCCGTGAGG | |
| | CCCATGAGCAGCGCGAAGTGTAGCACGTAGATGGCCATGAGCAGCGTGAAGACCAC | |
| | CAGCGCGCAGCGCAGCGTCAGCCCCCAGGACTTAAGGCGACCGTCGCCACCGTAGC | |
| | AGGCGGGGAAGAAGGCACGACTGCCTTCCTG | |
| RAT-vGAT- | ATGGCCATGAGCAGCGTGAAGACCACCAGCGCGCAGCGCAGCGTCAGCCCCCAGGA | 118 |
| ses2 | CTTAAGGCGACCGTCGCCACCGTACCAGGCGGGGAAGAAGGCACGACTGCCTTCCT | |
| | AGAAGAGAGACTTCTCCAGCACTTCGACGGCCGCGAAGAAGGGCAACGGGTACGAC | |
| | AGCAGCGCCTTGGCCACCAGGAAGATCTTGACCACGGCGCGG | |
| RAT-vGAT- | GACCAGGACTTCTGCGACACGGGCAGCCCCGGGAAACTGTTGTACATGAGGTTGCC | 119 |
| ses3 | GCTCACCACTACGTACAAGATACACGTCATCACCAGCTCGATGATCTAGGCCACAT | |
| | TGACCACGCGGCCGCCCAGCGTGGGGAATCGAGGAGCGCAGCACGCGTTAGCTATC | |
| | GCCACATACGAGTCCCTCACGCGCACCACCTCACCA | |
| RAT-vGAT- | CGTCCCCGCAGGGCTCGCCTTCCGATTTCAGGATGTCCATCTGCAGGCCCTGGCGG | 120 |
| ses4 | TGCTCAAAGTCGAGATCGTCGCAGTGCGCGAAGCCCACCGCCTCCTCATCCGTAGC | |
| | CGCCTGAAACCCCATCCTAGCGAACATGCCGCTCACCTTGGCCTGGGACTTGTTGG | |
| | ACACAGAGGTGGCCACGTTGGTCA | |
| Monkey- | CATGAGCAGCGTGAAGACGACAAGCGCGCAGCGCAGCGTCAGCCCCCAGGACTTCA | 121 |
| vGAT-ses1 | GGCGCCCGTCGCCGCCGTAGCAGGCCGGGAAAAAGGCGCGGCTGCCTTCCTGGAAG | |
| | AGCGACTTCTCCAGAACCTCGACAGCGGCGAAGAATAGCAGAGGATAGGACAACAG | |
| | CGCCTTGGCCACCAGAAAGATGTTGACCACGGCGCGGATGGAGCCAGGCAGGTTAT | |
| | CCGTGATTACCTCCTTGGTCTCGTCGGCCCAGGTGAGGTAGGCGACGAGTGCGAAG | |
| | AGGCCCTTGAGCACGCAGGCGGCGATGTGCGTCCAGTTCATCATGCAGTGGAACTC | |
| | GCTGGG | |
| Monkey- | AACTTGACCTTCTCCCAGGCCCAGTCGCGTGCCCGCGATAGACAGTGGGCTATGAC | 122 |
| vGAT-ses2 | TAGGATATTGATGACGAAGTAGGCCAGAGTGCACAGCAGACTGAACTTGGACACGG | |
| | CCTTGAGGTTCTTAAGGAAGGCGCAAGGCAGGAGCACGGCCGTGGCGATAATGGAC | |
| | CAGGACTTCTGCGACACGGGCAGCCCCGGGAAGCTGTTGTACATGAGGTTGCCACT | |
| | CACCACCACGTAC | |
| Human- | GGCCACATCGAAGAAGACCTGGTGCCACAGCAGCTTGCGCCAGAGAAGGCGCAAGT | 123 |
| vGAT-ses1 | GGAAGAGGCTGGGCAGCAGGAAGCAGAGGCCGGCGCCTGTGAGGCTGCCCGTGAGG | |
| | CCCATGAGCAGCGCGAAGTGTAGCACGTAGATGGCCATGAGCAGCGTGAAGACCAC | |
| | CAGCGCGCAGCGCAGCGTCAGCCCCCAGGACTTAAGGCGACCGTCGCCTCCATAGC | |
| | AGGCGGGGAAGAAGGCGCGACTGCCTTCCTG | |
| Human- | AAAGGACTTCAGGCGCCCGTCGCCGCTGTAGCAGGCCGGGAAAAAGGCGCGGCTGC | 124 |
| vGAT-ses2 | CTTCCTGGAAGAGCGACTTCTCCAGCACCTCGACAGCGGCAAAGAATAGCAGAGGA | |
| | TAGGACAACAGCGCCTTGGCCACCAGAAAGATGTTGACCACGGCGCGGATGGAGC | |
| | CGGGCAGGTTATCCGTGATGACCTCCTTGGTCTCGTC | |
| Human- | CAGGATGTGAAAGTCTTCCAGAAGAAATTCTTGCAGCCAGCTTTGCGTTCTCGGGG | 125 |
| SST-ses1 | TGCCATAGCCGGGTTTGAGTTAGCAGATCTCTGCAGCTCAAGCCTCATTTCATCCT | |
| | GCTCAGCAGCCTAGGACAGATCTTCAGGTTCCAGGGCATCATTCTCCGTCTGGTTG | |
| | GGTTCAGACAGCAGCTCTGCCAAGAAGTACTTGGCCAGTTCCTGCTTCCCCGCGGC | |
| | AGCAGCCAGGGACTTCTGCAGAAACGGACGGAGTCTGGGGTCCGAGGGAGCGCCGG | |
| | TGACACAGCCCAGGGCCAGGACGGTGGACAGCGCAGCCAGCGCGCACTGGAGGCGG | |
| | CAGGACAGC | |
| Human-PV- | CTTTCAGCCACCAGAGTGGAGAGTTCGTCAACCCCAATTTTGCCGTCCCCATCTTT | 126 |
| ses1 | GTCTCCAGCAGCCATCAGCATCTTGGTTTCTTTAGCAGACAGGTCTCTGGCATCTG | |
| | GGGAGAAGCCTTTTAGGATGAATCCCAGCTCATCCTCCTCGATGAAGCCACTTTTG | |
| | TCCTTGTCCAGCATGTGGAACACCTTCTTCACATCATCCGCACTCTTTTTCTTCAG | |
| | GCCGACCATTTAGAAGAACTTTTTGTGGTCGAAGGAGTCGGTAGCGCTAAAGGCTC | |
| | CCACCGCCTTCTTGATGTCCTCAGCGTTCAGCAAGTCTGTCATCGAC | |
| Human-PV- | AAGAAGGCGCGCGGACCTGCTACCACTCCTGCACCGCCAGGCCAGGGGTCCGCGGG | 127 |
| ses2 | ATCCCAGGGGCTGCGGCCAGGGCACGAGGGAAGGGGCCACCTCTAGGATTTAGGGG | |
| | GCACTGGCGTCACCAGCTGGGTCTGGAAAGTCCACCTGCCGTCAAGGACACGCAGG | |
| | AGGTGCGCCGTCTCAGATCTGGGAACCTTGGCGGATGTCCTGCCGCGTGGGGGAAG | |
| | ATCC | |
| Human-PV- | GTGTGCAGAGGTGACGTGTTCAAGGTCACAGCTGGCAAGGGGCAGAGAAGGAGCAG | 128 |
| ses3 | AGCATGGGGGTCTGGGCTTTACACTCAGGCAGCGGATGTTTATGAAGCACCTGCTC | |
| | TGTGTGGGGTATTGGCTAGATACTGGGGCTGGAAGGATGAATGCAACCCAATCCCT | |
| | AGTTTTAAGTTTTACATCCACTTATATGTTGGATGTAAGTAGGTAGAACGTGAAGA | |
| | TTCTAGACAGAAGGTACCCTTGCGAAGGGACCTAGTTTTAAAAAAATTAGCTAACA | |
| | AATACATATATAACATTTACTCTGTGCCAGACACTGTTCTAGGCACTTTACATATT | |
| | AACACATCT | |
| Human-CR- | CCACTCCTGTCTGTGTCGTACTTCCGCCAAGCCTCCATAAACTCGGCGCTGGAGCC | 129 |
| ses1 | CACGTGCTGCCTGAAGCACAGAAGGAAGTTCTCTTCGGTTGGCAGGATCTGCGCCA | |
| | GCTCTGCCATCTCGATTTTCCCATCTGAGTTTTTATCATACTTCTGCATGAACTCC | |
| | TTCATCTTTTCTCCAAAGTTGTCACTCTTTGACATCATGCCAGAGCCTTTCCTTGC | |
| | CTTCTCCAGCTCTTAGAAAAAGTTTTCTAGCTCTTTACCTTCAATATACCCATTTC | |
| | CGTCTGCGTCAAAGTGCTTCCATATTTCCAGGAACTGGGACGCCGTCAGCTCGGCC | |
| | AGGTGCAGGGAAGGGGGCTGCTGCTGCGGGCCAGCC | |
| Human-CR- | CCGCTTCTATCCTTGTCGGAAAATGTGAAGATCGCGTTAAACTCCTCTGAGGTCAG | 130 |
| ses2 | CTTCATGCCCGGAAATTTAAGCAGGAAGTTTTCCTGGACAGGCAGGAGTCGGGACA | |
| | TCTCTGAGAGGCCCAATTTGCCATCCCCGTTCAAGTCAAACATCCGTAGTATGGTT | |
| | TGGGTGTATTCCTAGAGCTTGGGCTCATCGTACGGCCGGTTCGCCTTCTTCAGCAG | |
| | GTCTGACAGGAATCCCTTGAGCTCATTGGCTTCGATTCAGCCACTCCTGTCTGTGT | |
| | CGTACTTCCGCCAAGCCTCCATAAACTCGGCGCTGGAGCCCACGTGCTGCCTGAAG | |
| | CACAGAAGGAAGTTCTCTGGCGCGCCAGCTACTAACTTCAGCCTG | |
| Human- | GTAAAAATGGACGAATACGGCCGGGCGTGGTGGCTCACGCCTGTGTTCCCAGCACT | 131 |
| NAV1.7- | TTGGGAGGCCAAGGTGGGCAGAACACAACGTCAGGAGATCGAGACCATCCTGGCTA | |
| ses1 | ACACGGTGAATCCCCGTCTCTACGAAAAATACAAAAACAAAATTAGCCAGGTGTAG | |
| | TGGCGGGTACCTGTAGTCCCAGCTACCCTGGAGGCTGAGGTGGGAGAATGGCGTGA | |
| | ACCCGGGAGGCGGAGCTTGCAGGGAGCCAAAATCGCGCCACGGCACTCCAGCCTGG | |
| | GCAACAGAGGGAGACTCTGT | |
| Human- | GTTAGAGAAATAAAACTGTATTGGCCAGGCGCTGTGGCTCACGCCTGTAATCGCAG | 132 |
| NAV1.8- | CACTTTGGGAGGCCGAGGCGGGCAGATTACGAGGTCAGGAGATCGAGACCATCCTG | |
| ses1 | GCGAACGCGGTGAAACCCCGTCTCTACTAAAAATACAAAAAATTAGCCGGGCGCCT | |
| | TAGCGGGCACCTGTAGTTCCAGCTACTCCGGAGGCTGAGGCGGGAGAATGGCGTGA | |
| | ACCCGGGAGGCGGAGCTTGCAGGGAGCCGAGATCGCGTCACTGCACTCCAGTCTGG | |
| | GTGACAGAGCAAGACTTCGT | |
| Human- | AAGGCAAACCAAAAAGGGGAAAAAGGATATAAAAGCTGCCCCTCAGTCGAGGTGAC | 133 |
| TRPV1-ses1 | TGTCCAGAAAAGCAATGTAACAGGGACAGTGGAGGGCGTGTGTCCCCATTCTGTCC | |
| | CCACCCTGTCTATAGACACCATCGAGAAAGTCACCCTCCTACTCCCAGACCACTAG | |
| | CTTCTCTGCCTGCTCGGGAGCTCGGGAGGCAGGAGCCTGGTGGGGACGGAGGGAGG | |
| | TGGAGGCAGGGGTGTAGACAGCCCCTCTAAGGGAAAAGGTGGCCTCACACTCCCCA | |
| | GAAGTCAGCCAGCCTGTGGC | |
| Human- | AATGGTTCTCTTTGGGGGCTGGGAGCCTTGGCTCAGGCCTGTAATCCCAGCACTCT | 134 |
| FABP7-ses1 | GGGAGGCCGAGGCAGCAGGCGGGAGCATCACTTTGAGCTCAAGAGCTGTAGGCCAG | |
| | CCTGGGCAACATAGCGAAATCCCATCTCTACAAAAATACAAAAAAATGAGCCCGGC | |
| | GTTGGTGGCTTGCACCTGTGGTCCCAGCTACTTAGGAGGCTGAGGTTGGAGAATCG | |
| | CTTGAGCCCAGGAAGCAGAGGTTGCAGTGAGCCAAGAGTGCACCCCCTGCACTCCA | |
| | GTCTGGGTGACACAGCAAGA | |
| Human- | GAAAUACUGCUUCAGGACAUCGGAGAUCAAGGCCAAGUAGGAGUCCCAGCCAGGCC | 135 |
| ALDH1L1- | UGGUCUGGGUCCAGGGUCAGCAGCUGGGGUCAGUGGUCACUUAGUGGCUGGGUGUA | |
| ses1 | GGACCCAGGCUGUGGUGGGGUCAGGGUAUGUCAUGGGUCACUUGUAGUCAGGUCCA | |
| | AGACUGAAGCCUGGCUGGCAUUGGGUGUUGAGUGUUGGAGUUGAUUCUAGGGCCAG | |
| | GCUGCAGCUUCCCCACGGAGGGAGACUGGCUUUCCUUAACAAACUACACCUCUGUG | |
| | CCUGGCCAGGCCUACAAGCC | |

1. A modular RNA molecule or pharmaceutical composition thereof, comprising:
(i) a 5′ region comprising a sensor domain comprising a stretch of consecutive nucleotides that is complementary to a corresponding stretch of consecutive nucleotides of a cellular RNA of a cell of the mammalian central nervous system or peripheral nervous system, wherein said sensor domain comprises a stop codon editable by ADAR; and
(ii) a 3′ region comprising a domain encoding a protein, wherein said protein coding domain is downstream of and in-frame with said sensor domain,
wherein, upon introduction of said modular RNA into said cell of said comprising an Adar enzyme, said stretch of consecutive nucleotides of said sensor domain and said corresponding nucleotide stretch of said cellular RNA form an RNA duplex comprising said stop codon, wherein said stop codon comprised in said RNA duplex is edited by ADAR in said cell, thereby to permit translation of said protein.

2. The modular RNA molecule of claim 1, wherein said stretch of consecutive nucleotides of said sensor domain is able to form an RNA duplex with at least a portion of an mRNA, said portion comprising a corresponding stretch of consecutive nucleotides.

3. The modular RNA molecule of claim 1, in which said protein coding region encodes an effector protein.

4. The modular RNA molecule of claim 1, in which the molecule further encodes a self-cleaving 2A peptide positioned between the sensor domain and the 3′ protein coding domain.

5. A composition, optionally a pharmaceutical comprising:
i) a first nucleic acid comprising a modular RNA molecule comprising:
(i) a sensor domain comprising a stretch of consecutive nucleotides that is complementary to a corresponding stretch of consecutive nucleotides of cellular RNA of a mammalian cell of the central nervous system or the peripheral nervous system, wherein said sensor domain comprises a stop codon editable by ADAR; and
(ii) a first protein-coding domain encoding an effector protein, wherein said first protein-coding region is downstream of and in-frame with said sensor domain, and
ii) a second nucleic acid comprising a second protein coding domain.

6. The composition of claim 5,
wherein said first and second nucleic acids comprise a single nucleic acid molecule or
wherein said first and second nucleic acids comprise two nucleic acid molecules,
wherein optionally, wherein said first and second nucleic acid are covalently linked.

7. The composition of claim 5,
wherein said second protein coding domain is comprised within a gene comprising a transcription control element, optionally, a promoter, and/or
wherein said effector protein binds to said gene, and/or
wherein expression of said second protein coding domain is modulated by said effector protein, and/or
wherein the first protein-coding domain encodes a transcriptional activator or transcriptional repressor, and/or
wherein the first protein-coding domain encodes a DNA recombinase.

8. The composition of claim 5,
wherein said cell comprises a mammalian cortex cell, and/or
wherein the first protein-coding domain encodes an effector protein comprising a transcriptional activator, and/or
wherein said stretch of consecutive nucleotides of said sensor domain is complementary to a stretch of consecutive nucleotides of a selected cellular RNA of a cortex cell encoded by the Fezf2 gene, and/or wherein said second nucleic acid comprises a second protein coding region under the transcriptional control of an inducible promoter.

9. The composition of claim 5, wherein said cell comprises a mammalian schwann cell or a mammalian satellite cell of the peripheral nervous system, and/or
wherein the first protein-coding domain encodes an effector protein comprising a transcription activator, and/or
wherein said stretch of consecutive nucleotides of said sensor domain is complementary to a stretch of consecutive nucleotides of a selected cellular RNA of a peripheral CNS cell, and/or
wherein said second nucleic acid comprises a second protein coding region under the transcriptional control of an inducible promoter.

10. The composition of claim 5, wherein the first protein-coding region encodes an effector protein selected from the group consisting of:
a cell killing protein selected from the group consisting of: thymidine kinase (TK) and cytosine deaminase (CD),
a programmed cell death protein selected from the group consisting of CASP3, CASP9, BCL, GSDME, GSDMD, GZMA and GZMB,
a neural activating protein selected from the group consisting of: ChR2 and DREADD-M3Dq,
an immunity enhancer protein selected from the group consisting of IFNB, IFNG, TNFA, IL2, IL 12, IL 15 and CD40L,
a physiological editing protein selected from the group consisting of NaChBac and Kir2.1, f) a protein synthesis inhibition protein including ricin,
a neural inhibitor protein selected from the group consisting of DREADD-hM4D, NpHR and GtACR1,
a protein involved in neural cell fate such as NEUROD, and
a protein involved with cell regeneration including Epidermal growth factors.

11. A nucleic acid delivery vehicle or pharmaceutical composition thereof, comprising:
a) the modular RNA molecule of claim 1 or
b) a nucleic acid composition encoding said modular RNA, said composition comprising:
i) a first nucleic acid comprising a modular RNA molecule comprising:
(i) a sensor domain comprising a stretch of consecutive nucleotides that is complementary to a corresponding stretch of consecutive nucleotides of cellular RNA of a mammalian cell of the central nervous system or the peripheral nervous system, wherein said sensor domain comprises a stop codon editable by ADAR; and
(ii) a first protein-coding domain encoding an effector protein, wherein said first protein-coding region is downstream of and in-frame with said sensor domain, and
ii) a second nucleic acid comprising a second protein coding domain.

12. A cell comprising:
(i) the modular RNA molecule of claim 1, or
(ii) a composition comprising:
a) a first nucleic acid comprising a modular RNA molecule comprising:
(i) a sensor domain comprising a stretch of consecutive nucleotides that is complementary to a corresponding stretch of consecutive nucleotides of a cellular RNA, wherein said sensor domain comprises a stop codon editable by ADAR; and
(ii) a first protein-coding domain encoding an effector protein, wherein said first protein-coding region is downstream of and in-frame with said sensor domain, and
b) a second nucleic acid comprising a second protein coding domain, or
(iii) a nucleic acid delivery vehicle comprising the modular RNA molecule of claim 1 or composition comprising:
a) a first nucleic acid comprising a modular RNA molecule comprising:
(i) a sensor domain comprising a stretch of consecutive nucleotides that is complementary to a corresponding stretch of consecutive nucleotides of a cellular RNA, wherein said sensor domain comprises a stop codon editable by ADAR; and
(ii) a first protein-coding domain encoding an effector protein, wherein said first protein-coding region is downstream of and in-frame with said sensor domain, and
b) a second nucleic acid comprising a second protein coding domain,
wherein said cell is optionally a mammalian cell.

13. A kit comprising:
(i) the modular RNA molecule of claim 1, or
(ii) a composition comprising:
a) a first nucleic acid comprising a modular RNA molecule comprising:
(i) a sensor domain comprising a stretch of consecutive nucleotides that is complementary to a corresponding stretch of consecutive nucleotides of a cellular RNA, wherein said sensor domain comprises a stop codon editable by ADAR; and
(ii) a first protein-coding domain encoding an effector protein, wherein said first protein-coding region is downstream of and in-frame with said sensor domain, and
b) a second nucleic acid comprising a second protein coding domain, or
(iii) a nucleic acid delivery vehicle comprising the modular RNA molecule of claim 1 or composition comprising:
a) a first nucleic acid comprising a modular RNA molecule comprising:
(i) a sensor domain comprising a stretch of consecutive nucleotides that is complementary to a corresponding stretch of consecutive nucleotides of a cellular RNA, wherein said sensor domain comprises a stop codon editable by ADAR; and
(ii) a first protein-coding domain encoding an effector protein, wherein said first protein-coding region is downstream of and in-frame with said sensor domain, and
b) a second nucleic acid comprising a second protein coding domain, and packaging therefore.

14. A method of introducing a modular RNA according to claim 1, into a selected cell of a mammal, the method comprising:
contacting a cell of said mammal with said modular RNA molecule, said nucleic acid composition encoding said modular RNA, or said delivery vehicle comprising said modular RNA or said nucleic acid composition encoding said modular RNA, under conditions which permit said cell of said mammal to comprise said modular RNA or said delivery vehicle comprising said modular RNA or said nucleic acid composition encoding said modular RNA,
wherein said modular RNA is according to claim 1,
wherein said nucleic acid composition is a composition comprising:
a) a first nucleic acid comprising a modular RNA molecule comprising:
(i) a sensor domain comprising a stretch of consecutive nucleotides that is complementary to a corresponding stretch of consecutive nucleotides of a cellular RNA, wherein said sensor domain comprises a stop codon editable by ADAR; and
(ii) a first protein-coding domain encoding an effector protein, wherein said first protein-coding region is downstream of and in-frame with said sensor domain, and
b) a second nucleic acid comprising a second protein coding domain, and
wherein said delivery vehicle is a nucleic acid delivery vehicle comprising the modular RNA molecule of claim 1 or a composition comprising:
a) a first nucleic acid comprising a modular RNA molecule comprising:
(i) a sensor domain comprising a stretch of consecutive nucleotides that is complementary to a corresponding stretch of consecutive nucleotides of a cellular RNA, wherein said sensor domain comprises a stop codon editable by ADAR; and
(ii) a first protein-coding domain encoding an effector protein, wherein said first protein-coding region is downstream of and in-frame with said sensor domain, and
b) a second nucleic acid comprising a second protein coding domain.

15. A method of eliciting ADAR editing of a stop codon to allow expression of an in-frame, downstream encoded protein in a cell of a mammal comprising a selected target cellular RNA, the method comprising:
introducing into said cell of said mammal a modular RNA molecule under conditions in which said modular RNA is comprised in said cell, said modular RNA comprising:
(i) a 5′ region comprising a sensor domain comprising a stretch of consecutive nucleotides that is complementary to a corresponding stretch of consecutive nucleotides of said selected cellular RNA, wherein said sensor domain comprises a stop codon editable by ADAR; and
(ii) a 3′ region comprising a domain encoding an effector protein, wherein said protein coding region is downstream of and in-frame with said sensor domain,
wherein upon introduction of said modular RNA molecule to said mammal, an RNA duplex is formed in said cell between said stretch of consecutive nucleotides of said sensor domain and said corresponding stretch of consecutive nucleotides of said cellular RNA,
wherein said RNA duplex comprises said ADAR-editable stop codon, and
wherein ADAR edits said stop codon comprised in said RNA duplex, thereby permitting translation of said protein, and
wherein said protein is produced in said mammal.

16. A method for treating a disease or disorder in a mammal, the method comprising:
providing an agent, said agent comprising
(i) a modular RNA molecule according to claim 1 (i) a modular RNA molecule according to claim 1; or
(ii) a nucleic acid composition comprising:
a) a first nucleic acid comprising a modular RNA molecule comprising:
(i) a sensor domain comprising a stretch of consecutive nucleotides that is complementary to a corresponding stretch of consecutive nucleotides of a cellular RNA, wherein said sensor domain comprises a stop codon editable by ADAR; and
(ii) a first protein-coding domain encoding an effector protein, wherein said first protein-coding region is downstream of and in-frame with said sensor domain, and
b) a second nucleic acid comprising a second protein coding domain; or
(iii) a delivery vehicle comprising the modular RNA molecule of claim 1 or a composition comprising:
a) a first nucleic acid comprising a modular RNA molecule comprising:
(i) a sensor domain comprising a stretch of consecutive nucleotides that is complementary to a corresponding stretch of consecutive nucleotides of a cellular RNA, wherein said sensor domain comprises a stop codon editable by ADAR; and
(ii) a first protein-coding domain encoding an effector protein, wherein said first protein-coding region is downstream of and in-frame with said sensor domain, and
b) a second nucleic acid comprising a second protein coding domain; and
administering said agent to said mammal in a therapeutically effective amount to permit translation of said 3′ encoded protein or said effector protein in selected cells of said mammal, thereby to produce said protein in said cells,
wherein production of said protein in said cells provides for treatment of said disease or disorder in said mammal.

17. A method of stimulating a L5b/L6 corticofugal projection neuron signal in the forelimb somatosensory cortex of a mouse, as measured by photometry, in response to mechanical stimulation of the forepaw, comprising:
administering to said mouse the composition of claim 5,
wherein the sensor domain comprises a stretch of consecutive nucleotides that is complementary to a corresponding stretch of consecutive nucleotides of a cellular RNA encoding Ctip2 or Fezf,
wherein the first protein-coding domain encodes an effector protein comprises a transcription activator, and wherein said second nucleic acid comprises a gene encoding a reporter protein under the transcriptional control of an inducible promoter,
wherein said administration provides for production of said effector protein and said reporter protein,
thereby permitting stimulation of said L5b/L6 CFPN signal in said forelimb somatosensory cortex of said mouse.

18. A method of stimulating forelimb movement in response to light stimulation of a caudal forelimb area (CFA) of a mouse, the method comprising:
administering to said mouse with the composition of claim 5,
wherein the sensor domain comprises a stretch of consecutive nucleotides that is complementary to a corresponding stretch of consecutive nucleotides of cellular RNA encoding Ctip2,
wherein the first protein-coding domain encodes an effector protein comprising a transcription activator, and wherein said second nucleic acid comprises a gene encoding a light-activated reporter protein under the transcriptional control of an inducible promoter,
wherein said administration provides for production of said effector protein and said light-activated reporter protein, thereby permitting light stimulation of said forelimb movement of said mouse.

19. A cell comprising:
(A) the modular RNA molecule of claim 1 and/or
(B) a composition comprising:
i) a first nucleic acid comprising a modular RNA molecule comprising:
(i) a sensor domain comprising a stretch of consecutive nucleotides that is complementary to a corresponding stretch of consecutive nucleotides of cellular RNA of a mammalian cell of the central nervous system or the peripheral nervous system, wherein said sensor domain comprises a stop codon editable by ADAR; and
(ii) a first protein-coding domain encoding an effector protein, wherein said first protein-coding region is downstream of and in-frame with said sensor domain, and
ii) a second nucleic acid comprising a second protein coding domain, and/or
(C) a delivery system of nucleic acid delivery vehicle comprising:
1. the modular RNA molecule of claim 1 or
2. a nucleic acid composition encoding said modular RNA, said composition comprising:
i) a first nucleic acid comprising a modular RNA molecule comprising:
(i) a sensor domain comprising a stretch of consecutive nucleotides that is complementary to a corresponding stretch of consecutive nucleotides of cellular RNA of a mammalian cell of the central nervous system or the peripheral nervous system, wherein said sensor domain comprises a stop codon editable by ADAR; and
(ii) a first protein-coding domain encoding an effector protein, wherein said first protein-coding region is downstream of and in-frame with said sensor domain, and
ii) a second nucleic acid comprising a second protein coding domain.

20. A kit comprising:
(A) the modular RNA molecule of claim 1 and/or
(B) a composition comprising:
i) a first nucleic acid comprising a modular RNA molecule comprising:
(i) a sensor domain comprising a stretch of consecutive nucleotides that is complementary to a corresponding stretch of consecutive nucleotides of cellular RNA of a mammalian cell of the central nervous system or the peripheral nervous system, wherein said sensor domain comprises a stop codon editable by A DAR; and
(ii) a first protein-coding domain encoding an effector protein, wherein said first protein-coding region is downstream of and in-frame with said sensor domain, and
ii) a second nucleic acid comprising a second protein coding domain, and/or
(C) a delivery system of nucleic acid delivery vehicle comprising:
1. the modular RNA molecule of claim 1 or
2. a nucleic acid composition encoding said modular RNA, said composition comprising:
i) a first nucleic acid comprising a modular RNA molecule comprising:
(i) a sensor domain comprising a stretch of consecutive nucleotides that is complementary to a corresponding stretch of consecutive nucleotides of cellular RNA of a mammalian cell of the central nervous system or the peripheral nervous system, wherein said sensor domain comprises a stop codon editable by ADAR; and
(ii) a first protein-coding domain encoding an effector protein, wherein said first protein-coding region is downstream of and in-frame with said sensor domain, and
ii) a second nucleic acid comprising a second protein coding domain.

21. A method of identifying GABAergic neurons in a mammalian cortex in vivo, the method comprising:
administering to said mammal the composition of claim 5 under conditions permitting transfection of said composition to cells of said S1 barrel cortex and hippocampus and expression of said protein coding regions,
wherein said stretch of consecutive nucleotides of said sensor domain is complementary to a stretch of consecutive nucleotides of a selected cellular vGAT RNA of a cortex cell, and
wherein the first protein-coding domain encodes an effector protein comprising a transcription activator and a second effector protein comprising first reporter protein, and said second nucleic acid comprises a coding region for a second reporter protein under the transcriptional control of an inducible promoter,
wherein said first nucleic acid comprising said modular RNA molecule is under the control of a promoter and wherein said modular RNA molecule comprises a gene encoding a third reporter protein upstream of said sensor domain and under the control of a promoter,
wherein said proteins are produced from said coding regions,
wherein identification of GABAergic neurons in said mammalian cortex comprising vGAT mRNA is permitted upon detection of one or more of said first, second and third reporter proteins,
wherein optionally, said sesRNA targeting vGAT mRNA is complementary to all or part of exon 1 of vGAT mRNA.

22. A method of identifying a GABAergic neuron in a mammalian cortex in vivo, the method comprising
administering to a mammal the composition of claim 5 under conditions permitting transfection of cells of said mammalian cortex by said composition and production of said proteins encoded by said composition,
wherein said stretch of consecutive nucleotides of said sensor domain is complementary to a stretch of consecutive nucleotides of a selected cellular Tle4 RNA of a cortex cell, and
wherein the first protein-coding domain encodes an effector protein comprising a transcription activator and a second effector protein comprising a first reporter protein, and
wherein said second nucleic acid comprises a coding region for a second reporter protein under the transcriptional control of an inducible promoter,
wherein said first nucleic acid comprising said modular RNA molecule is under the control of a promoter and
wherein said modular RNA molecule comprises a gene encoding a third reporter protein upstream of said sensor domain and under the control of a promoter, wherein optionally, said sensor domain is complementary to all or part of exon 15 of Tle4 mRNA, and
wherein detection of one or more of said first, second and third reporter proteins in a cell of said mammalian cortex permits identification of a said TLE4-positive projection neuron, thereby identifying a GABAergic neuron in said mammalian cortex.

23. A method of determining the presence of cells comprising forkhead box protein P2 (FOXP2) RNA in a cortical layer of the mammalian cortex in culture, the method comprising:
contacting a cultured cortical layer of a mammalian cortex with the composition of claim 5,
wherein said stretch of consecutive nucleotides of said sensor domain is complementary to a stretch of consecutive nucleotides of a selected FOXP2 cellular RNA of a cortex cell, and
wherein the first protein-coding domain encodes an effector protein comprising a transcription activator and a second effector protein comprising a gene encoding a first reporter protein, and
wherein said second nucleic acid comprises a coding region for a second reporter protein under the control of an inducible promoter,
wherein said modular RNA molecule further comprises a gene encoding a third reporter protein upstream of said sensor domain, and
detecting one or more of said first, second and third reporter proteins,
wherein said detection on cell bodies of a said cortical layer is indicative of the presence of said cells.

24. A method to suppress a neural activity in a mammal that promotes focal epileptic seizure, said method comprising:
administering to said mammal a therapeutically effective amount of an agent comprising a nucleic acid composition according to claim 5,
wherein said stretch of consecutive nucleotides of said sensor domain is complementary to a stretch of consecutive nucleotides of a selected cellular RNA of a cortex cell encoded by an SST gene, and
wherein the first protein-coding domain encodes a) a transcription activator and b) a first effector protein, and
wherein said second nucleic acid comprises a coding region for a second effector protein under the control of an inducible promoter induced by said transcriptional activator,
wherein said modular RNA molecule further comprises a gene encoding a third effector protein upstream of said sensor domain, and
wherein said composition further comprises a gene encoding a DREADD receptor or a gene encoding a PSAM, and
wherein in the presence of a drug effective to activate said receptor encoded by said DREADD or said PSAM gene, said DREADD receptor or said PSAM receptor is activated, thereby permitting production of one or more said effector proteins and treatment of epilepsy in said mammal.

25. A method to suppress in a mammal a neural activity that promotes focal epileptic seizure, said method comprising administering to said mammal in a therapeutically effective amount an agent, said agent comprising a nucleic acid composition according to claim 5,
wherein said first and second nucleic acids comprise two nucleic acid molecules; wherein said stretch of consecutive nucleotides of said sensor domain of said modular RNA is complementary to a stretch of consecutive nucleotides of a selected cfos cellular RNA of a cortex cell,
wherein the first protein-coding domain encodes a first effector protein comprising transcription activator and a second effector protein comprising the gene encoding osmFlag, and
wherein said second nucleic acid comprises a coding region for a first reporter protein under the control of an inducible promoter,
wherein said first nucleic acid comprising said modular RNA molecule is under the control of a promoter and
wherein said modular RNA molecule comprises the gene encoding a second reporter protein upstream of said sensor domain and under the control of said promoter,
wherein said agent further comprises a gene encoding a DREADD receptor or a gene encoding PSAM, and
wherein said administration permits translation of said effector protein in selected cells of said mammal in the presence of a drug that activates said DREADD or said PSAM receptor, thereby to produce said protein in said cells,
wherein production of said protein in said cells is effective to suppress in said mammal a neural activity that promotes focal epileptic seizure.

26. A method of treating chronic pain in a mammal, comprising
administering to said mammal a therapeutically effective amount of an agent a nucleic acid composition according to claim 5;
wherein said first and second nucleic acid are covalently linked,
wherein said sensor domain of said modular RNA comprises a stretch of consecutive nucleotides complementary to a stretch of consecutive nucleotides of selected TRPV 1 or NaV 1.8 cellular RNA, and a said first protein-coding domain encoding an effector protein comprising a transcription activator, said modular RNA further comprises a domain encoding an shRNA that binds to NaV1.7 or NaV1.8 RNA, said domain encoding said shRNA being under the control of an inducible promoter,
wherein production of said transcriptional activator and said shRNA in said cells of said mammal thereby treats chronic pain in said mammal.

27. A method of treating Alzheimer's Disease in a mammal, the method comprising
administering a therapeutically effective amount of an agent to said mammal, said agent comprising a nucleic acid composition according to claim 5;
wherein said first and second nucleic acid are covalently linked,
wherein said sensor domain of said modular RNA comprises said stretch of consecutive nucleotides complementary to a stretch of consecutive nucleotides of selected cellular RNA encoded by a gene selected from the following genes: a) a human astrocyte marker gene of aldehyde dehydrogenase family 1 member L1 (AldhlL1), b) a glial fibrillary acidic protein (GFAP) gene, c) an excitatory amino acid transporter 1 (EAAT1) gene, and d) a microglial gene: C-X3-C Motif Chemokine Receptor 1 (CX3CR1), e) a transmembrane protein 119 (TMEM119), and f) an Ionized calcium binding adaptor molecule 1 (IBA-1), and
wherein said first protein-coding domain of said modular RNA encodes a transcription activator, and
wherein said modular RNA further comprises a second coding domain which encodes a repressor of ApoE4, said second coding domain being under control of an inducible promoter, and
wherein production of said transcription activator and said repressor in said cells thereby treats and/or prevents progression of Alzheimer's Disease in said mammal.
